SEQUENCE LISTING (371 sequences)

SEQ ID NO: 1 - protein, 4 aa, Artificial Sequence
  Description of Artificial Sequence: Synthetic peptide
HHHH

SEQ ID NO: 2 - protein, 4 aa, Artificial Sequence
  Description of Artificial Sequence: Synthetic peptide
KHKK

SEQ ID NO: 3 - protein, 4 aa, Artificial Sequence
  Description of Artificial Sequence: Synthetic peptide
KKHK

SEQ ID NO: 4 - protein, 4 aa, Artificial Sequence
  Description of Artificial Sequence: Synthetic peptide
KKKH

SEQ ID NO: 5 - protein, 4 aa, Artificial Sequence
  Description of Artificial Sequence: Synthetic peptide
KHKH

SEQ ID NO: 6 - protein, 4 aa, Artificial Sequence
  Description of Artificial Sequence: Synthetic peptide
HKHK

SEQ ID NO: 7 - protein, 4 aa, Artificial Sequence
  Description of Artificial Sequence: Synthetic peptide
KKKK

SEQ ID NO: 8 - protein, 4 aa, Artificial Sequence
  Description of Artificial Sequence: Synthetic peptide
KKRK

SEQ ID NO: 9 - protein, 4 aa, Artificial Sequence
  Description of Artificial Sequence: Synthetic peptide
KRKK

SEQ ID NO: 10 - protein, 4 aa, Artificial Sequence
  Description of Artificial Sequence: Synthetic peptide
KRRK

SEQ ID NO: 11 - protein, 4 aa, Artificial Sequence
  Description of Artificial Sequence: Synthetic peptide
RKKR

SEQ ID NO: 12 - protein, 4 aa, Artificial Sequence
  Description of Artificial Sequence: Synthetic peptide
RRRR

SEQ ID NO: 13 - protein, 4 aa, Artificial Sequence
  Description of Artificial Sequence: Synthetic peptide
KGKK

SEQ ID NO: 14 - protein, 4 aa, Artificial Sequence
  Description of Artificial Sequence: Synthetic peptide
KKGK

SEQ ID NO: 15 - protein, 5 aa, Artificial Sequence
  Description of Artificial Sequence: Synthetic peptide
  MOD_RES (2)..(2): Beta-alanine
  MOD_RES (4)..(4): Beta-alanine
HAHAH

SEQ ID NO: 16 - protein, 5 aa, Artificial Sequence
  Description of Artificial Sequence: Synthetic peptide
  MOD_RES (2)..(2): Beta-alanine
  MOD_RES (4)..(4): Beta-alanine
HAKAH

SEQ ID NO: 17 - protein, 5 aa, Artificial Sequence
  Description of Artificial Sequence: Synthetic peptide
RRRRR

SEQ ID NO: 18 - protein, 5 aa, Artificial Sequence
  Description of Artificial Sequence: Synthetic peptide
KKKKK

SEQ ID NO: 19 - protein, 5 aa, Artificial Sequence
  Description of Artificial Sequence: Synthetic peptide
KKKRK

SEQ ID NO: 20 - protein, 5 aa, Artificial Sequence
  Description of Artificial Sequence: Synthetic peptide
RKKKK

SEQ ID NO: 21 - protein, 5 aa, Artificial Sequence
  Description of Artificial Sequence: Synthetic peptide
KRKKK

SEQ ID NO: 22 - protein, 5 aa, Artificial Sequence
  Description of Artificial Sequence: Synthetic peptide
KKRKK

SEQ ID NO: 23 - protein, 5 aa, Artificial Sequence
  Description of Artificial Sequence: Synthetic peptide
KKKKR

SEQ ID NO: 24 - protein, 5 aa, Artificial Sequence
  Description of Artificial Sequence: Synthetic peptide
  MOD_RES (2)..(2): Beta-alanine
  MOD_RES (4)..(4): Beta-alanine
KAKAK

SEQ ID NO: 25 - protein, 6 aa, Artificial Sequence
  Description of Artificial Sequence: Synthetic peptide
RKKKKG

SEQ ID NO: 26 - protein, 6 aa, Artificial Sequence
  Description of Artificial Sequence: Synthetic peptide
KRKKKG

SEQ ID NO: 27 - protein, 6 aa, Artificial Sequence
  Description of Artificial Sequence: Synthetic peptide
KKRKKG

SEQ ID NO: 28 - protein, 6 aa, Artificial Sequence
  Description of Artificial Sequence: Synthetic peptide
KKKKRG

SEQ ID NO: 29 - protein, 6 aa, Artificial Sequence
  Description of Artificial Sequence: Synthetic peptide
  MOD_RES (6)..(6): Beta-alanine
RKKKKA

SEQ ID NO: 30 - protein, 6 aa, Artificial Sequence
  Description of Artificial Sequence: Synthetic peptide
  MOD_RES (6)..(6): Beta-alanine
KRKKKA

SEQ ID NO: 31 - protein, 6 aa, Artificial Sequence
  Description of Artificial Sequence: Synthetic peptide
  MOD_RES (6)..(6): Beta-alanine
KKRKKA

SEQ ID NO: 32 - protein, 6 aa, Artificial Sequence
  Description of Artificial Sequence: Synthetic peptide
  MOD_RES (6)..(6): Beta-alanine
KKKKRA

SEQ ID NO: 33 - protein, 6 aa, Artificial Sequence
  Description of Artificial Sequence: Synthetic peptide
KKKRKV

SEQ ID NO: 34 - protein, 6 aa, Artificial Sequence
  Description of Artificial Sequence: Synthetic peptide
RRRRRR

SEQ ID NO: 35 - protein, 6 aa, Artificial Sequence
  Description of Artificial Sequence: Synthetic peptide
HHHHHH

SEQ ID NO: 36 - protein, 6 aa, Artificial Sequence
  Description of Artificial Sequence: Synthetic peptide
RHRHRH

SEQ ID NO: 37 - protein, 6 aa, Artificial Sequence
  Description of Artificial Sequence: Synthetic peptide
HRHRHR

SEQ ID NO: 38 - protein, 6 aa, Artificial Sequence
  Description of Artificial Sequence: Synthetic peptide
KRKRKR

SEQ ID NO: 39 - protein, 6 aa, Artificial Sequence
  Description of Artificial Sequence: Synthetic peptide
RKRKRK

SEQ ID NO: 40 - protein, 6 aa, Artificial Sequence
  Description of Artificial Sequence: Synthetic peptide
  MOD_RES (2)..(2): Beta-alanine
  MOD_RES (4)..(4): Beta-alanine
  MOD_RES (6)..(6): Beta-alanine
RARARA

SEQ ID NO: 41 - protein, 6 aa, Artificial Sequence
  Description of Artificial Sequence: Synthetic peptide
  MOD_RES (2)..(2): Beta-alanine
  MOD_RES (4)..(4): Beta-alanine
  MOD_RES (6)..(6): Beta-alanine
KAKAKA

SEQ ID NO: 42 - protein, 7 aa, Artificial Sequence
  Description of Artificial Sequence: Synthetic peptide
PKKKRKV

SEQ ID NO: 43 - protein, 7 aa, Artificial Sequence
  Description of Artificial Sequence: Synthetic peptide
PGKKRKV

SEQ ID NO: 44 - protein, 7 aa, Artificial Sequence
  Description of Artificial Sequence: Synthetic peptide
PKGKRKV

SEQ ID NO: 45 - protein, 7 aa, Artificial Sequence
  Description of Artificial Sequence: Synthetic peptide
PKKGRKV

SEQ ID NO: 46 - protein, 7 aa, Artificial Sequence
  Description of Artificial Sequence: Synthetic peptide
PKKKGKV

SEQ ID NO: 47 - protein, 7 aa, Artificial Sequence
  Description of Artificial Sequence: Synthetic peptide
PKKKRGV

SEQ ID NO: 48 - protein, 7 aa, Artificial Sequence
  Description of Artificial Sequence: Synthetic peptide
PKKKRKG

SEQ ID NO: 49 - protein, 5 aa, Artificial Sequence
  Description of Artificial Sequence: Synthetic peptide
  MOD_RES (2)..(2): Beta-alanine
  MOD_RES (4)..(4): Beta-alanine
RARAR

SEQ ID NO: 50 - protein, 5 aa, Artificial Sequence
  Description of Artificial Sequence: Synthetic peptide
  MOD_RES (2)..(2): Beta-alanine
  MOD_RES (4)..(4): Beta-alanine
RAHAR

SEQ ID NO: 51 - protein, 5 aa, Artificial Sequence
  Description of Artificial Sequence: Synthetic peptide
  MOD_RES (2)..(2): Beta-alanine
  MOD_RES (4)..(4): Beta-alanine
HARAH

SEQ ID NO: 52 - protein, 19 aa, Artificial Sequence
  Description of Artificial Sequence: Synthetic peptide
NLSKRPAAIKKAGQAKKKK

SEQ ID NO: 53 - protein, 9 aa, Artificial Sequence
  Description of Artificial Sequence: Synthetic peptide
PAAKRVKLD

SEQ ID NO: 54 - protein, 11 aa, Artificial Sequence
  Description of Artificial Sequence: Synthetic peptide
RQRRNELKRSF

SEQ ID NO: 55 - protein, 27 aa, Artificial Sequence
  Description of Artificial Sequence: Synthetic peptide
RMRKFKNKGKDTAELRRRRVEVSVELR

SEQ ID NO: 56 - protein, 14 aa, Artificial Sequence
  Description of Artificial Sequence: Synthetic peptide
KAKKDEQILKRRNV

SEQ ID NO: 57 - protein, 8 aa, Artificial Sequence
  Description of Artificial Sequence: Synthetic peptide
VSRKRPRP

SEQ ID NO: 58 - protein, 8 aa, Artificial Sequence
  Description of Artificial Sequence: Synthetic peptide
PPKKARED

SEQ ID NO: 59 - protein, 8 aa, Artificial Sequence
  Description of Artificial Sequence: Synthetic peptide
PQPKKKPL

SEQ ID NO: 60 - protein, 12 aa, Artificial Sequence
  Description of Artificial Sequence: Synthetic peptide
SALIKKKKKMAP

SEQ ID NO: 61 - protein, 5 aa, Artificial Sequence
  Description of Artificial Sequence: Synthetic peptide
DRLRR

SEQ ID NO: 62 - protein, 7 aa, Artificial Sequence
  Description of Artificial Sequence: Synthetic peptide
PKQKKRK

SEQ ID NO: 63 - protein, 10 aa, Artificial Sequence
  Description of Artificial Sequence: Synthetic peptide
RKLKKKIKKL

SEQ ID NO: 64 - protein, 10 aa, Artificial Sequence
  Description of Artificial Sequence: Synthetic peptide
REKKKFLKRR

SEQ ID NO: 65 - protein, 20 aa, Artificial Sequence
  Description of Artificial Sequence: Synthetic peptide
KRKGDEVDGVDEVAKKKSKK

SEQ ID NO: 66 - protein, 17 aa, Artificial Sequence
  Description of Artificial Sequence: Synthetic peptide
RKCLQAGMNLEARKTKK

SEQ ID NO: 67 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFARRRRQ

SEQ ID NO: 68 - protein, 7 aa, Artificial Sequence
  Description of Artificial Sequence: Synthetic peptide
FGFGRGR

SEQ ID NO: 69 - protein, 7 aa, Artificial Sequence
  Description of Artificial Sequence: Synthetic peptide
  MOD_RES (2)..(2): D-phenylalanine
  MOD_RES (5)..(5): D-arginine
  MOD_RES (7)..(7): D-arginine
GFFGRGR

SEQ ID NO: 70 - protein, 7 aa, Artificial Sequence
  Description of Artificial Sequence: Synthetic peptide
  MOD_RES (2)..(2): D-phenylalanine
  MOD_RES (3)..(3): L-naphthylalanine
FFAGRGR

SEQ ID NO: 71 - protein, 7 aa, Artificial Sequence
  Description of Artificial Sequence: Synthetic peptide
  MOD_RES (2)..(2): D-phenylalanine
FFFGRGR

SEQ ID NO: 72 - protein, 7 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFAGRGR

SEQ ID NO: 73 - protein, 4 aa, Artificial Sequence
  Description of Artificial Sequence: Synthetic peptide
  MOD_RES (4)..(4): L-naphthylalanine
FGFA

SEQ ID NO: 74 - protein, 8 aa, Artificial Sequence
  Description of Artificial Sequence: Synthetic peptide
  MOD_RES (2)..(2): D-phenylalanine
  MOD_RES (5)..(5): D-arginine
  MOD_RES (7)..(7): D-arginine
GFFGRGRQ

SEQ ID NO: 75 - protein, 8 aa, Artificial Sequence
  Description of Artificial Sequence: Synthetic peptide
  MOD_RES (2)..(2): D-phenylalanine
  MOD_RES (3)..(3): L-naphthylalanine
FFAGRGRQ

SEQ ID NO: 76 - protein, 8 aa, Artificial Sequence
  Description of Artificial Sequence: Synthetic peptide
  MOD_RES (2)..(2): D-phenylalanine
FFFGRGRQ

SEQ ID NO: 77 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFAGRGRQ

SEQ ID NO: 78 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFARRRRQ

SEQ ID NO: 79 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (4)..(4): Citrulline
  MOD_RES (5)..(5): D-arginine
  MOD_RES (6)..(6): Citrulline
  MOD_RES (7)..(7): D-arginine
FFAXRXRQ

SEQ ID NO: 80 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFAGRGRQ

SEQ ID NO: 81 - protein, 8 aa, Artificial Sequence
  Description of Artificial Sequence: Synthetic peptide
  MOD_RES (2)..(2): D-phenylalanine
FFFGRGRQ

SEQ ID NO: 82 - protein, 8 aa, Artificial Sequence
  Description of Artificial Sequence: Synthetic peptide
FGFGRGRQ

SEQ ID NO: 83 - protein, 8 aa, Artificial Sequence
  Description of Artificial Sequence: Synthetic peptide
  MOD_RES (2)..(2): D-phenylalanine
  MOD_RES (5)..(5): D-arginine
  MOD_RES (7)..(7): D-arginine
GFFGRGRQ

SEQ ID NO: 84 - protein, 8 aa, Artificial Sequence
  Description of Artificial Sequence: Synthetic peptide
FGFGRRRQ

SEQ ID NO: 85 - protein, 8 aa, Artificial Sequence
  Description of Artificial Sequence: Synthetic peptide
FGFRRRRQ

SEQ ID NO: 86 - protein, 7 aa, Artificial Sequence
  Description of Artificial Sequence: Synthetic peptide
  MOD_RES (2)..(2): L-naphthylalanine
FARRRRQ

SEQ ID NO: 87 - protein, 7 aa, Artificial Sequence
  Description of Artificial Sequence: Synthetic peptide
  MOD_RES (1)..(1): D-phenylalanine
  MOD_RES (2)..(2): L-naphthylalanine
  MOD_RES (4)..(4): D-arginine
  MOD_RES (6)..(6): D-arginine
FARRRRQ

SEQ ID NO: 88 - protein, 8 aa, Artificial Sequence
  Description of Artificial Sequence: Synthetic peptide
  MOD_RES (2)..(2): D-phenylalanine
  MOD_RES (3)..(3): L-naphthylalanine
FFARGRGQ

SEQ ID NO: 89 - protein, 6 aa, Artificial Sequence
  Description of Artificial Sequence: Synthetic peptide
  MOD_RES (2)..(2): L-naphthylalanine
FARRRQ

SEQ ID NO: 90 - protein, 6 aa, Artificial Sequence
  Description of Artificial Sequence: Synthetic peptide
  MOD_RES (2)..(2): L-naphthylalanine
FARRRC

SEQ ID NO: 91 - protein, 6 aa, Artificial Sequence
  Description of Artificial Sequence: Synthetic peptide
  MOD_RES (2)..(2): L-naphthylalanine
  MOD_RES (6)..(6): Selenocysteine
FARRRX

SEQ ID NO: 92 - protein, 6 aa, Artificial Sequence
  Description of Artificial Sequence: Synthetic peptide
  MOD_RES (4)..(4): L-naphthylalanine
RRRAFQ

SEQ ID NO: 93 - protein, 6 aa, Artificial Sequence
  Description of Artificial Sequence: Synthetic peptide
  MOD_RES (5)..(5): L-naphthylalanine
RRRRAF

SEQ ID NO: 94 - protein, 6 aa, Artificial Sequence
  Description of Artificial Sequence: Synthetic peptide
  MOD_RES (2)..(2): L-naphthylalanine
FARRRR

SEQ ID NO: 95 - protein, 7 aa, Artificial Sequence
  Description of Artificial Sequence: Synthetic peptide
  MOD_RES (2)..(2): D-naphthylalanine
  MOD_RES (3)..(3): D-arginine
  MOD_RES (5)..(5): D-arginine
  MOD_RES (7)..(7): D-glutamine
FARRRRQ

SEQ ID NO: 96 - protein, 7 aa, Artificial Sequence
  Description of Artificial Sequence: Synthetic peptide
  MOD_RES (2)..(2): D-naphthylalanine
  MOD_RES (3)..(3): D-arginine
  MOD_RES (5)..(5): D-arginine
FARRRRQ

SEQ ID NO: 97 - protein, 7 aa, Artificial Sequence
  Description of Artificial Sequence: Synthetic peptide
  MOD_RES (2)..(2): L-naphthylalanine
FARRRRQ

SEQ ID NO: 98 - protein, 7 aa, Artificial Sequence
  Description of Artificial Sequence: Synthetic peptide
  MOD_RES (1)..(1): D-phenylalanine
  MOD_RES (2)..(2): L-naphthylalanine
  MOD_RES (4)..(4): D-arginine
  MOD_RES (6)..(6): D-arginine
FARRRRQ

SEQ ID NO: 99 - protein, 7 aa, Artificial Sequence
  Description of Artificial Sequence: Synthetic peptide
  MOD_RES (5)..(5): L-naphthylalanine
RRFRARQ

SEQ ID NO: 100 - protein, 7 aa, Artificial Sequence
  Description of Artificial Sequence: Synthetic peptide
  MOD_RES (6)..(6): L-naphthylalanine
FRRRRAQ

SEQ ID NO: 101 - protein, 7 aa, Artificial Sequence
  Description of Artificial Sequence: Synthetic peptide
  MOD_RES (1)..(1): D-arginine
  MOD_RES (5)..(5): L-naphthylalanine
RRFRARQ

SEQ ID NO: 102 - protein, 7 aa, Artificial Sequence
  Description of Artificial Sequence: Synthetic peptide
  MOD_RES (3)..(3): L-naphthylalanine
RRAFRRQ

SEQ ID NO: 103 - protein, 8 aa, Artificial Sequence
  Description of Artificial Sequence: Synthetic peptide
CRRRRFWQ

SEQ ID NO: 104 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFARRRRQ

SEQ ID NO: 105 - protein, 8 aa, Artificial Sequence
  Description of Artificial Sequence: Synthetic peptide
  MOD_RES (3)..(3): L-naphthylalanine
FFARRRRQ

SEQ ID NO: 106 - protein, 8 aa, Artificial Sequence
  Description of Artificial Sequence: Synthetic peptide
  MOD_RES (6)..(6): L-naphthylalanine
RFRFRARQ

SEQ ID NO: 107 - protein, 8 aa, Artificial Sequence
  Description of Artificial Sequence: Synthetic peptide
  MOD_RES (1)..(1): Selenocysteine
XRRRRFWQ

SEQ ID NO: 108 - protein, 8 aa, Artificial Sequence
  Description of Artificial Sequence: Synthetic peptide
CRRRRFWQ

SEQ ID NO: 109 - protein, 8 aa, Artificial Sequence
  Description of Artificial Sequence: Synthetic peptide
  MOD_RES (2)..(2): L-naphthylalanine
FARRRRQK

SEQ ID NO: 110 - protein, 8 aa, Artificial Sequence
  Description of Artificial Sequence: Synthetic peptide
  MOD_RES (2)..(2): L-naphthylalanine
FARRRRQC

SEQ ID NO: 111 - protein, 8 aa, Artificial Sequence
  Description of Artificial Sequence: Synthetic peptide
  MOD_RES (1)..(1): D-phenylalanine
  MOD_RES (2)..(2): L-naphthylalanine
  MOD_RES (4)..(4): D-arginine
  MOD_RES (6)..(6): D-arginine
FARRRRRQ

SEQ ID NO: 112 - protein, 8 aa, Artificial Sequence
  Description of Artificial Sequence: Synthetic peptide
  MOD_RES (2)..(2): L-naphthylalanine
FARRRRRQ

SEQ ID NO: 113 - protein, 9 aa, Artificial Sequence
  Description of Artificial Sequence: Synthetic peptide
  MOD_RES (5)..(5): L-naphthylalanine
  MOD_RES (8)..(8): L-norleucine
RRRRAFDXC

SEQ ID NO: 114 - protein, 5 aa, Artificial Sequence
  Description of Artificial Sequence: Synthetic peptide
  MOD_RES (2)..(2): L-naphthylalanine
FARRR

SEQ ID NO: 115 - protein, 5 aa, Artificial Sequence
  Description of Artificial Sequence: Synthetic peptide
FWRRR

SEQ ID NO: 116 - protein, 5 aa, Artificial Sequence
  Description of Artificial Sequence: Synthetic peptide
  MOD_RES (4)..(4): L-naphthylalanine
RRRAF

SEQ ID NO: 117 - protein, 5 aa, Artificial Sequence
  Description of Artificial Sequence: Synthetic peptide
RRRWF

SEQ ID NO: 118-149 - skipped (32 entries)

SEQ ID NO: 150 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFARRRRQ

SEQ ID NO: 151 - DNA, 25 bp, Artificial Sequence
  Description of Artificial Sequence: Synthetic oligonucleotide
ctgtgggcccaagcgtgtgagggca

SEQ ID NO: 152 - DNA, 25 bp, Artificial Sequence
  Description of Artificial Sequence: Synthetic oligonucleotide
acccactgtgggcccaagcgtgtga

SEQ ID NO: 153 - DNA, 25 bp, Artificial Sequence
  Description of Artificial Sequence: Synthetic oligonucleotide
atgccacccactgtgggcccaagcg

SEQ ID NO: 154 - DNA, 25 bp, Artificial Sequence
  Description of Artificial Sequence: Synthetic oligonucleotide
tgtagatgccacccactgtgggccc

SEQ ID NO: 155 - DNA, 25 bp, Artificial Sequence
  Description of Artificial Sequence: Synthetic oligonucleotide
caccgtgtagatgccacccactgtg

SEQ ID NO: 156 - DNA, 25 bp, Artificial Sequence
  Description of Artificial Sequence: Synthetic oligonucleotide
tgcagcaccgtgtagatgccaccca

SEQ ID NO: 157 - DNA, 25 bp, Artificial Sequence
  Description of Artificial Sequence: Synthetic oligonucleotide
cttgcagcccttgctgttcatggaa

SEQ ID NO: 158 - DNA, 25 bp, Artificial Sequence
  Description of Artificial Sequence: Synthetic oligonucleotide
cccaccttgcagcccttgctgttca

SEQ ID NO: 159 - DNA, 25 bp, Artificial Sequence
  Description of Artificial Sequence: Synthetic oligonucleotide
cacgtcccaccttgcagcccttgct

SEQ ID NO: 160 - DNA, 25 bp, Artificial Sequence
  Description of Artificial Sequence: Synthetic oligonucleotide
tgggccacgtcccaccttgcagccc

SEQ ID NO: 161 - DNA, 25 bp, Artificial Sequence
  Description of Artificial Sequence: Synthetic oligonucleotide
tgggctgggccacgtcccaccttgc

SEQ ID NO: 162 - DNA, 25 bp, Artificial Sequence
  Description of Artificial Sequence: Synthetic oligonucleotide
tgccctgggctgggccacgtcccac

SEQ ID NO: 163 - DNA, 25 bp, Artificial Sequence
  Description of Artificial Sequence: Synthetic oligonucleotide
ctgggtgggaggggacagcagtccg

SEQ ID NO: 164 - DNA, 25 bp, Artificial Sequence
  Description of Artificial Sequence: Synthetic oligonucleotide
ttgaactgggtgggaggggacagca

SEQ ID NO: 165 - DNA, 25 bp, Artificial Sequence
  Description of Artificial Sequence: Synthetic oligonucleotide
ccacgttgaactgggtgggagggga

SEQ ID NO: 166 - DNA, 25 bp, Artificial Sequence
  Description of Artificial Sequence: Synthetic oligonucleotide
cttgtccacgttgaactgggtggga

SEQ ID NO: 167 - DNA, 25 bp, Artificial Sequence
  Description of Artificial Sequence: Synthetic oligonucleotide
gcttccttgtccacgttgaactggg

SEQ ID NO: 168 - DNA, 25 bp, Artificial Sequence
  Description of Artificial Sequence: Synthetic oligonucleotide
cccctgcttccttgtccacgttgaa

SEQ ID NO: 169 - DNA, 25 bp, Artificial Sequence
  Description of Artificial Sequence: Synthetic oligonucleotide
ctggtttcctcttgagcaagtgctg

SEQ ID NO: 170 - DNA, 25 bp, Artificial Sequence
  Description of Artificial Sequence: Synthetic oligonucleotide
cctacctggtttcctcttgagcaag

SEQ ID NO: 171 - DNA, 25 bp, Artificial Sequence
  Description of Artificial Sequence: Synthetic oligonucleotide
cagcccctacctggtttcctcttga

SEQ ID NO: 172 - DNA, 25 bp, Artificial Sequence
  Description of Artificial Sequence: Synthetic oligonucleotide
cagcccagcccctacctggtttcct

SEQ ID NO: 173 - DNA, 25 bp, Artificial Sequence
  Description of Artificial Sequence: Synthetic oligonucleotide
gacttcagcccagcccctacctggt

SEQ ID NO: 174 - DNA, 25 bp, Artificial Sequence
  Description of Artificial Sequence: Synthetic oligonucleotide
ctggggacttcagcccagcccctac

SEQ ID NO: 175 - DNA, 25 bp, Artificial Sequence
  Description of Artificial Sequence: Synthetic oligonucleotide
ctgggattgggggtgagggtcccat

SEQ ID NO: 176 - DNA, 25 bp, Artificial Sequence
  Description of Artificial Sequence: Synthetic oligonucleotide
aatatctgggattgggggtgagggt

SEQ ID NO: 177 - DNA, 25 bp, Artificial Sequence
  Description of Artificial Sequence: Synthetic oligonucleotide
gtcacaatatctgggattgggggtg

SEQ ID NO: 178 - DNA, 25 bp, Artificial Sequence
  Description of Artificial Sequence: Synthetic oligonucleotide
tgggggtcacaatatctgggattgg

SEQ ID NO: 179 - DNA, 25 bp, Artificial Sequence
  Description of Artificial Sequence: Synthetic oligonucleotide
cccattgggggtcacaatatctggg

SEQ ID NO: 180 - DNA, 25 bp, Artificial Sequence
  Description of Artificial Sequence: Synthetic oligonucleotide
ttcagcccattgggggtcacaatat

SEQ ID NO: 181 - DNA, 25 bp, Artificial Sequence
  Description of Artificial Sequence: Synthetic oligonucleotide
ccataaaaatggccccgcacaaact

SEQ ID NO: 182 - DNA, 25 bp, Artificial Sequence
  Description of Artificial Sequence: Synthetic oligonucleotide
cgtacccataaaaatggccccgcac

SEQ ID NO: 183 - DNA, 25 bp, Artificial Sequence
  Description of Artificial Sequence: Synthetic oligonucleotide
ccccacgtacccataaaaatggccc

SEQ ID NO: 184 - DNA, 25 bp, Artificial Sequence
  Description of Artificial Sequence: Synthetic oligonucleotide
atatgccccacgtacccataaaaat

SEQ ID NO: 185 - DNA, 25 bp, Artificial Sequence
  Description of Artificial Sequence: Synthetic oligonucleotide
taggtatatgccccacgtacccata

SEQ ID NO: 186 - DNA, 25 bp, Artificial Sequence
  Description of Artificial Sequence: Synthetic oligonucleotide
agacctaggtatatgccccacgtac

SEQ ID NO: 187 - DNA, 25 bp, Artificial Sequence
  Description of Artificial Sequence: Synthetic oligonucleotide
ctaaagaacccacaaggcacggtaa

SEQ ID NO: 188 - DNA, 25 bp, Artificial Sequence
  Description of Artificial Sequence: Synthetic oligonucleotide
gatgcctaaagaacccacaaggcac

SEQ ID NO: 189 - DNA, 25 bp, Artificial Sequence
  Description of Artificial Sequence: Synthetic oligonucleotide
gtccagatgcctaaagaacccacaa

SEQ ID NO: 190 - DNA, 25 bp, Artificial Sequence
  Description of Artificial Sequence: Synthetic oligonucleotide
ttgaagtccagatgcctaaagaacc

SEQ ID NO: 191 - DNA, 25 bp, Artificial Sequence
  Description of Artificial Sequence: Synthetic oligonucleotide
ccaagttgaagtccagatgcctaaa

SEQ ID NO: 192 - DNA, 25 bp, Artificial Sequence
  Description of Artificial Sequence: Synthetic oligonucleotide
cttgtccaagttgaagtccagatgc

SEQ ID NO: 193 - DNA, 25 bp, Artificial Sequence
  Description of Artificial Sequence: Synthetic oligonucleotide
tctgagcagatagttgagccgagcc

SEQ ID NO: 194 - DNA, 25 bp, Artificial Sequence
  Description of Artificial Sequence: Synthetic oligonucleotide
ctcactctgagcagatagttgagcc

SEQ ID NO: 195 - DNA, 25 bp, Artificial Sequence
  Description of Artificial Sequence: Synthetic oligonucleotide
caggcctcactctgagcagatagtt

SEQ ID NO: 196 - DNA, 25 bp, Artificial Sequence
  Description of Artificial Sequence: Synthetic oligonucleotide
tagcccaggcctcactctgagcaga

SEQ ID NO: 197 - DNA, 25 bp, Artificial Sequence
  Description of Artificial Sequence: Synthetic oligonucleotide
cctcatagcccaggcctcactctga

SEQ ID NO: 198 - DNA, 25 bp, Artificial Sequence
  Description of Artificial Sequence: Synthetic oligonucleotide
tgtcccctcatagcccaggcctcac

SEQ ID NO: 199 - DNA, 25 bp, Artificial Sequence
  Description of Artificial Sequence: Synthetic oligonucleotide
ctgcgcagaaagaaaggagggggag

SEQ ID NO: 200 - DNA, 25 bp, Artificial Sequence
  Description of Artificial Sequence: Synthetic oligonucleotide
ttcacctgcgcagaaagaaaggagg

SEQ ID NO: 201 - DNA, 25 bp, Artificial Sequence
  Description of Artificial Sequence: Synthetic oligonucleotide
tgccgttcacctgcgcagaaagaaa

SEQ ID NO: 202 - DNA, 25 bp, Artificial Sequence
  Description of Artificial Sequence: Synthetic oligonucleotide
ctcgctgccgttcacctgcgcagaa

SEQ ID NO: 203 - DNA, 25 bp, Artificial Sequence
  Description of Artificial Sequence: Synthetic oligonucleotide
gtctgctcgctgccgttcacctgcg

SEQ ID NO: 204 - DNA, 25 bp, Artificial Sequence
  Description of Artificial Sequence: Synthetic oligonucleotide
ccactgtctgctcgctgccgttcac

SEQ ID NO: 205 - DNA, 25 bp, Artificial Sequence
  Description of Artificial Sequence: Synthetic oligonucleotide
caaagctgtttgcgcacagcttggc

SEQ ID NO: 206 - DNA, 25 bp, Artificial Sequence
  Description of Artificial Sequence: Synthetic oligonucleotide
ctaaccaaagctgtttgcgcacagc

SEQ ID NO: 207 - DNA, 25 bp, Artificial Sequence
  Description of Artificial Sequence: Synthetic oligonucleotide
ggctgctaaccaaagctgtttgcgc

SEQ ID NO: 208 - DNA, 25 bp, Artificial Sequence
  Description of Artificial Sequence: Synthetic oligonucleotide
gcgagggctgctaaccaaagctgtt

SEQ ID NO: 209 - DNA, 25 bp, Artificial Sequence
  Description of Artificial Sequence: Synthetic oligonucleotide
ccggagcgagggctgctaaccaaag

SEQ ID NO: 210 - DNA, 25 bp, Artificial Sequence
  Description of Artificial Sequence: Synthetic oligonucleotide
aggggccggagcgagggctgctaac

SEQ ID NO: 211 - DNA, 25 bp, Artificial Sequence
  Description of Artificial Sequence: Synthetic oligonucleotide
ctgcaaggcaagcaggggcatgcat

SEQ ID NO: 212 - DNA, 25 bp, Artificial Sequence
  Description of Artificial Sequence: Synthetic oligonucleotide
tcccactgcaaggcaagcaggggca

SEQ ID NO: 213 - DNA, 25 bp, Artificial Sequence
  Description of Artificial Sequence: Synthetic oligonucleotide
aaggctcccactgcaaggcaagcag

SEQ ID NO: 214 - DNA, 25 bp, Artificial Sequence
  Description of Artificial Sequence: Synthetic oligonucleotide
tcgggaaggctcccactgcaaggca

SEQ ID NO: 215 - DNA, 25 bp, Artificial Sequence
  Description of Artificial Sequence: Synthetic oligonucleotide
tcatgtcgggaaggctcccactgca

SEQ ID NO: 216 - DNA, 25 bp, Artificial Sequence
  Description of Artificial Sequence: Synthetic oligonucleotide
cttgttcatgtcgggaaggctccca

SEQ ID NO: 217 - DNA, 25 bp, Artificial Sequence
  Description of Artificial Sequence: Synthetic oligonucleotide
ctgcgttgcaaagatggctctcttc

SEQ ID NO: 218 - DNA, 25 bp, Artificial Sequence
  Description of Artificial Sequence: Synthetic oligonucleotide
catacctgcgttgcaaagatggctc

SEQ ID NO: 219 - DNA, 25 bp, Artificial Sequence
  Description of Artificial Sequence: Synthetic oligonucleotide
caatccatacctgcgttgcaaagat

SEQ ID NO: 220 - DNA, 25 bp, Artificial Sequence
  Description of Artificial Sequence: Synthetic oligonucleotide
aggtccaatccatacctgcgttgca

SEQ ID NO: 221 - DNA, 25 bp, Artificial Sequence
  Description of Artificial Sequence: Synthetic oligonucleotide
cacagaggtccaatccatacctgcg

SEQ ID NO: 222 - DNA, 25 bp, Artificial Sequence
  Description of Artificial Sequence: Synthetic oligonucleotide
ctctgcacagaggtccaatccatac

SEQ ID NO: 223 - DNA, 25 bp, Artificial Sequence
  Description of Artificial Sequence: Synthetic oligonucleotide
ctggtagtgaaaaagaaggactcag

SEQ ID NO: 224 - DNA, 25 bp, Artificial Sequence
  Description of Artificial Sequence: Synthetic oligonucleotide
atcacctggtagtgaaaaagaagga

SEQ ID NO: 225 - DNA, 25 bp, Artificial Sequence
  Description of Artificial Sequence: Synthetic oligonucleotide
ggaaaatcacctggtagtgaaaaag

SEQ ID NO: 226 - DNA, 25 bp, Artificial Sequence
  Description of Artificial Sequence: Synthetic oligonucleotide
cgggtggaaaatcacctggtagtga

SEQ ID NO: 227 - DNA, 25 bp, Artificial Sequence
  Description of Artificial Sequence: Synthetic oligonucleotide
aactccgggtggaaaatcacctggt

SEQ ID NO: 228 - DNA, 25 bp, Artificial Sequence
  Description of Artificial Sequence: Synthetic oligonucleotide
agaggaactccgggtggaaaatcac

SEQ ID NO: 229 - DNA, 25 bp, Artificial Sequence
  Description of Artificial Sequence: Synthetic oligonucleotide
ccggtgtgtagccccaaggctcata

SEQ ID NO: 230 - DNA, 25 bp, Artificial Sequence
  Description of Artificial Sequence: Synthetic oligonucleotide
ctcacccggtgtgtagccccaaggc

SEQ ID NO: 231 - DNA, 25 bp, Artificial Sequence
  Description of Artificial Sequence: Synthetic oligonucleotide
ctacactcacccggtgtgtagcccc

SEQ ID NO: 232 - DNA, 25 bp, Artificial Sequence
  Description of Artificial Sequence: Synthetic oligonucleotide
gcccactacactcacccggtgtgta

SEQ ID NO: 233 - DNA, 25 bp, Artificial Sequence
  Description of Artificial Sequence: Synthetic oligonucleotide
cccctgcccactacactcacccggt

SEQ ID NO: 234 - DNA, 25 bp, Artificial Sequence
  Description of Artificial Sequence: Synthetic oligonucleotide
gctgtcccctgcccactacactcac

SEQ ID NO: 235 - DNA, 25 bp, Artificial Sequence
  Description of Artificial Sequence: Synthetic oligonucleotide
ctgtggaggccaggacccaggttca

SEQ ID NO: 236 - DNA, 25 bp, Artificial Sequence
  Description of Artificial Sequence: Synthetic oligonucleotide
gatacctgtggaggccaggacccag

SEQ ID NO: 237 - DNA, 25 bp, Artificial Sequence
  Description of Artificial Sequence: Synthetic oligonucleotide
atgtagatacctgtggaggccagga

SEQ ID NO: 238 - DNA, 25 bp, Artificial Sequence
  Description of Artificial Sequence: Synthetic oligonucleotide
caagaatgtagatacctgtggaggc

SEQ ID NO: 239 - DNA, 25 bp, Artificial Sequence
  Description of Artificial Sequence: Synthetic oligonucleotide
ccggtcaagaatgtagatacctgtg

SEQ ID NO: 240 - DNA, 25 bp, Artificial Sequence
  Description of Artificial Sequence: Synthetic oligonucleotide
aaccgccggtcaagaatgtagatac

SEQ ID NO: 241 - DNA, 25 bp, Artificial Sequence
  Description of Artificial Sequence: Synthetic oligonucleotide
ccggcctaggtatttccagtccaga

SEQ ID NO: 242 - DNA, 25 bp, Artificial Sequence
  Description of Artificial Sequence: Synthetic oligonucleotide
cctacccggcctaggtatttccagt

SEQ ID NO: 243 - DNA, 25 bp, Artificial Sequence
  Description of Artificial Sequence: Synthetic oligonucleotide
ggggtcctacccggcctaggtattt

SEQ ID NO: 244 - DNA, 25 bp, Artificial Sequence
  Description of Artificial Sequence: Synthetic oligonucleotide
aagtgggggtcctacccggcctagg

SEQ ID NO: 245 - DNA, 25 bp, Artificial Sequence
  Description of Artificial Sequence: Synthetic oligonucleotide
taggaaagtgggggtcctacccggc

SEQ ID NO: 246 - DNA, 25 bp, Artificial Sequence
  Description of Artificial Sequence: Synthetic oligonucleotide
gaggataggaaagtgggggtcctac

SEQ ID NO: 247 - DNA, 25 bp, Artificial Sequence
  Description of Artificial Sequence: Synthetic oligonucleotide
ctgggatggggttggggaggcacca

SEQ ID NO: 248 - DNA, 25 bp, Artificial Sequence
  Description of Artificial Sequence: Synthetic oligonucleotide
tacacctgggatggggttggggagg

SEQ ID NO: 249 - DNA, 25 bp, Artificial Sequence
  Description of Artificial Sequence: Synthetic oligonucleotide
cgaaatacacctgggatggggttgg

SEQ ID NO: 250 - DNA, 25 bp, Artificial Sequence
  Description of Artificial Sequence: Synthetic oligonucleotide
gcgcccgaaatacacctgggatggg

SEQ ID NO: 251 - DNA, 25 bp, Artificial Sequence
  Description of Artificial Sequence: Synthetic oligonucleotide
agccagcgcccgaaatacacctggg

SEQ ID NO: 252 - DNA, 25 bp, Artificial Sequence
  Description of Artificial Sequence: Synthetic oligonucleotide
cgatcagccagcgcccgaaatacac

SEQ ID NO: 253 - DNA, 25 bp, Artificial Sequence
  Description of Artificial Sequence: Synthetic oligonucleotide
ctcacccaggaaccaggtggtcaga

SEQ ID NO: 254 - DNA, 25 bp, Artificial Sequence
  Description of Artificial Sequence: Synthetic oligonucleotide
cctacctcacccaggaaccaggtgg

SEQ ID NO: 255 - DNA, 25 bp, Artificial Sequence
  Description of Artificial Sequence: Synthetic oligonucleotide
cggggcctacctcacccaggaacca

SEQ ID NO: 256 - DNA, 25 bp, Artificial Sequence
  Description of Artificial Sequence: Synthetic oligonucleotide
tcagacggggcctacctcacccagg

SEQ ID NO: 257 - DNA, 25 bp, Artificial Sequence
  Description of Artificial Sequence: Synthetic oligonucleotide
atgtgtcagacggggcctacctcac

SEQ ID NO: 258 - DNA, 25 bp, Artificial Sequence
  Description of Artificial Sequence: Synthetic oligonucleotide
gtggaatgtgtcagacggggcctac

SEQ ID NO: 259 - DNA, 25 bp, Artificial Sequence
  Description of Artificial Sequence: Synthetic oligonucleotide
ctgtgggcaacagggacagggccac

SEQ ID NO: 260 - DNA, 25 bp, Artificial Sequence
  Description of Artificial Sequence: Synthetic oligonucleotide
aggaactgtgggcaacagggacagg

SEQ ID NO: 261 - DNA, 25 bp, Artificial Sequence
  Description of Artificial Sequence: Synthetic oligonucleotide
gtgccaggaactgtgggcaacaggg

SEQ ID NO: 262 - DNA, 25 bp, Artificial Sequence
  Description of Artificial Sequence: Synthetic oligonucleotide
actctgtgccaggaactgtgggcaa

SEQ ID NO: 263 - DNA, 25 bp, Artificial Sequence
  Description of Artificial Sequence: Synthetic oligonucleotide
tcctcactctgtgccaggaactgtg

SEQ ID NO: 264 - DNA, 25 bp, Artificial Sequence
  Description of Artificial Sequence: Synthetic oligonucleotide
gcttctcctcactctgtgccaggaa

SEQ ID NO: 265 - DNA, 25 bp, Artificial Sequence
  Description of Artificial Sequence: Synthetic oligonucleotide
gttctccaggttgttgtagaagtcc

SEQ ID NO: 266 - DNA, 25 bp, Artificial Sequence
  Description of Artificial Sequence: Synthetic oligonucleotide
ctcacgttctccaggttgttgtaga

SEQ ID NO: 267 - DNA, 25 bp, Artificial Sequence
  Description of Artificial Sequence: Synthetic oligonucleotide
cccagctcacgttctccaggttgtt

SEQ ID NO: 268 - DNA, 25 bp, Artificial Sequence
  Description of Artificial Sequence: Synthetic oligonucleotide
acggtcccagctcacgttctccagg

SEQ ID NO: 269 - DNA, 25 bp, Artificial Sequence
  Description of Artificial Sequence: Synthetic oligonucleotide
ctgccacggtcccagctcacgttct

SEQ ID NO: 270 - DNA, 25 bp, Artificial Sequence
  Description of Artificial Sequence: Synthetic oligonucleotide
cccatctgccacggtcccagctcac

SEQ ID NO: 271 - DNA, 25 bp, Artificial Sequence
  Description of Artificial Sequence: Synthetic oligonucleotide
ctggaggaagcagagcaacagggtc

SEQ ID NO: 272 - DNA, 25 bp, Artificial Sequence
  Description of Artificial Sequence: Synthetic oligonucleotide
tgtccctggaggaagcagagcaaca

SEQ ID NO: 273 - DNA, 25 bp, Artificial Sequence
  Description of Artificial Sequence: Synthetic oligonucleotide
ggccgtgtccctggaggaagcagag

SEQ ID NO: 274 - DNA, 25 bp, Artificial Sequence
  Description of Artificial Sequence: Synthetic oligonucleotide
gtgttggccgtgtccctggaggaag

SEQ ID NO: 275 - DNA, 25 bp, Artificial Sequence
  Description of Artificial Sequence: Synthetic oligonucleotide
tcaccgtgttggccgtgtccctgga

SEQ ID NO: 276 - DNA, 25 bp, Artificial Sequence
  Description of Artificial Sequence: Synthetic oligonucleotide
ttccttcaccgtgttggccgtgtcc

SEQ ID NO: 277 - DNA, 25 bp, Artificial Sequence
  Description of Artificial Sequence: Synthetic oligonucleotide
accagtaaggattcataaagcttcc

SEQ ID NO: 278 - DNA, 25 bp, Artificial Sequence
  Description of Artificial Sequence: Synthetic oligonucleotide
ctcacaccagtaaggattcataaag

SEQ ID NO: 279 - DNA, 25 bp, Artificial Sequence
  Description of Artificial Sequence: Synthetic oligonucleotide
gggcactcacaccagtaaggattca

SEQ ID NO: 280 - DNA, 25 bp, Artificial Sequence
  Description of Artificial Sequence: Synthetic oligonucleotide
gggtggggcactcacaccagtaagg

SEQ ID NO: 281 - DNA, 25 bp, Artificial Sequence
  Description of Artificial Sequence: Synthetic oligonucleotide
gctgagggtggggcactcacaccag

SEQ ID NO: 282 - DNA, 25 bp, Artificial Sequence
  Description of Artificial Sequence: Synthetic oligonucleotide
acagggctgagggtggggcactcac

SEQ ID NO: 283 - DNA, 25 bp, Artificial Sequence
  Description of Artificial Sequence: Synthetic oligonucleotide
ctgcaggagccacaagaagggtaag

SEQ ID NO: 284 - DNA, 25 bp, Artificial Sequence
  Description of Artificial Sequence: Synthetic oligonucleotide
tgccgctgcaggagccacaagaagg

SEQ ID NO: 285 - DNA, 25 bp, Artificial Sequence
  Description of Artificial Sequence: Synthetic oligonucleotide
aagactgccgctgcaggagccacaa

SEQ ID NO: 286 - DNA, 25 bp, Artificial Sequence
  Description of Artificial Sequence: Synthetic oligonucleotide
ggggaaagactgccgctgcaggagc

SEQ ID NO: 287 - DNA, 25 bp, Artificial Sequence
  Description of Artificial Sequence: Synthetic oligonucleotide
acaggggggaaagactgccgctgca

SEQ ID NO: 288 - DNA, 25 bp, Artificial Sequence
  Description of Artificial Sequence: Synthetic oligonucleotide
tgcacacaggggggaaagactgccg

SEQ ID NO: 289 - DNA, 25 bp, Artificial Sequence
  Description of Artificial Sequence: Synthetic oligonucleotide
cttcaccctgtcggcactgctattg

SEQ ID NO: 290 - DNA, 25 bp, Artificial Sequence
  Description of Artificial Sequence: Synthetic oligonucleotide
ctcaccttcaccctgtcggcactgc

SEQ ID NO: 291 - DNA, 25 bp, Artificial Sequence
  Description of Artificial Sequence: Synthetic oligonucleotide
atgccctcaccttcaccctgtcggc

SEQ ID NO: 292 - DNA, 25 bp, Artificial Sequence
  Description of Artificial Sequence: Synthetic oligonucleotide
accaaatgccctcaccttcaccctg

SEQ ID NO: 293 - DNA, 25 bp, Artificial Sequence
  Description of Artificial Sequence: Synthetic oligonucleotide
ccctgaccaaatgccctcaccttca

SEQ ID NO: 294 - DNA, 25 bp, Artificial Sequence
  Description of Artificial Sequence: Synthetic oligonucleotide
cccagccctgaccaaatgccctcac

SEQ ID NO: 295 - DNA, 25 bp, Artificial Sequence
  Description of Artificial Sequence: Synthetic oligonucleotide
ctgcgggaaggcaggagagagaccc

SEQ ID NO: 296 - DNA, 25 bp, Artificial Sequence
  Description of Artificial Sequence: Synthetic oligonucleotide
ctcagctgcgggaaggcaggagaga

SEQ ID NO: 297 - DNA, 25 bp, Artificial Sequence
  Description of Artificial Sequence: Synthetic oligonucleotide
gtgcactcagctgcgggaaggcagg

SEQ ID NO: 298 - DNA, 25 bp, Artificial Sequence
  Description of Artificial Sequence: Synthetic oligonucleotide
taaccgtgcactcagctgcgggaag

SEQ ID NO: 299 - DNA, 25 bp, Artificial Sequence
  Description of Artificial Sequence: Synthetic oligonucleotide
tcccataaccgtgcactcagctgcg

SEQ ID NO: 300 - DNA, 25 bp, Artificial Sequence
  Description of Artificial Sequence: Synthetic oligonucleotide
gggattcccataaccgtgcactcag

SEQ ID NO: 301 - DNA, 25 bp, Artificial Sequence
  Description of Artificial Sequence: Synthetic oligonucleotide
cgtaagctgaggggtctgcgatgtg

SEQ ID NO: 302 - DNA, 25 bp, Artificial Sequence
  Description of Artificial Sequence: Synthetic oligonucleotide
ctgaccgtaagctgaggggtctgcg

SEQ ID NO: 303 - DNA, 25 bp, Artificial Sequence
  Description of Artificial Sequence: Synthetic oligonucleotide
gggcgctgaccgtaagctgaggggt

SEQ ID NO: 304 - DNA, 25 bp, Artificial Sequence
  Description of Artificial Sequence: Synthetic oligonucleotide
ggcctgggcgctgaccgtaagctga

SEQ ID NO: 305 - DNA, 25 bp, Artificial Sequence
  Description of Artificial Sequence: Synthetic oligonucleotide
tcaggggcctgggcgctgaccgtaa

SEQ ID NO: 306 - DNA, 25 bp, Artificial Sequence
  Description of Artificial Sequence: Synthetic oligonucleotide
aatcctcaggggcctgggcgctgac

SEQ ID NO: 307 - DNA, 25 bp, Artificial Sequence
  Description of Artificial Sequence: Synthetic oligonucleotide
ctaggggaaaggaggagagggggca

SEQ ID NO: 308 - DNA, 25 bp, Artificial Sequence
  Description of Artificial Sequence: Synthetic oligonucleotide
tagtactaggggaaaggaggagagg

SEQ ID NO: 309 - DNA, 25 bp, Artificial Sequence
  Description of Artificial Sequence: Synthetic oligonucleotide
acatatagtactaggggaaaggagg

SEQ ID NO: 310 - DNA, 25 bp, Artificial Sequence
  Description of Artificial Sequence: Synthetic oligonucleotide
cgcagacatatagtactaggggaaa

SEQ ID NO: 311 - DNA, 25 bp, Artificial Sequence
  Description of Artificial Sequence: Synthetic oligonucleotide
tggcgcgcagacatatagtactagg

SEQ ID NO: 312 - DNA, 25 bp, Artificial Sequence
  Description of Artificial Sequence: Synthetic oligonucleotide
ccatgtggcgcgcagacatatagta

SEQ ID NO: 313 - DNA, 25 bp, Artificial Sequence
  Description of Artificial Sequence: Synthetic oligonucleotide
cgcatccgcctcgttgggctcgtag

SEQ ID NO: 314 - DNA, 25 bp, Artificial Sequence
  Description of Artificial Sequence: Synthetic oligonucleotide
ctcaccgcatccgcctcgttgggct

SEQ ID NO: 315 - DNA, 25 bp, Artificial Sequence
  Description of Artificial Sequence: Synthetic oligonucleotide
gtccactcaccgcatccgcctcgtt

SEQ ID NO: 316 - DNA, 25 bp, Artificial Sequence
  Description of Artificial Sequence: Synthetic oligonucleotide
ccagggtccactcaccgcatccgcc

SEQ ID NO: 317 - DNA, 25 bp, Artificial Sequence
  Description of Artificial Sequence: Synthetic oligonucleotide
gaaatccagggtccactcaccgcat

SEQ ID NO: 318 - DNA, 25 bp, Artificial Sequence
  Description of Artificial Sequence: Synthetic oligonucleotide
aagcagaaatccagggtccactcac

SEQ ID NO: 319 - DNA, 25 bp, Artificial Sequence
  Description of Artificial Sequence: Synthetic oligonucleotide
tcagggttgtggactcaatcatgcc

SEQ ID NO: 320 - DNA, 25 bp, Artificial Sequence
  Description of Artificial Sequence: Synthetic oligonucleotide
aaggaccagggtaagactagggact

SEQ ID NO: 321 - DNA, 25 bp, Artificial Sequence
  Description of Artificial Sequence: Synthetic oligonucleotide
gtcctggacaaggattgctgaccat

SEQ ID NO: 322 - DNA, 25 bp, Artificial Sequence
  Description of Artificial Sequence: Synthetic oligonucleotide
ctgcttccttgtctacattgaactg

SEQ ID NO: 323 - DNA, 25 bp, Artificial Sequence
  Description of Artificial Sequence: Synthetic oligonucleotide
atacccggcccaggtacttccaatc

SEQ ID NO: 324 - DNA, 25 bp, Artificial Sequence
  Description of Artificial Sequence: Synthetic oligonucleotide
ctggacaaggattgctgaccatagt

SEQ ID NO: 325 - DNA, 25 bp, Artificial Sequence
  Description of Artificial Sequence: Synthetic oligonucleotide
tcccaccgagcaggccttactctga

SEQ ID NO: 326 - DNA, 25 bp, Artificial Sequence
  Description of Artificial Sequence: Synthetic oligonucleotide
gaccacagctcagaccctacctggt

SEQ ID NO: 327 - DNA, 25 bp, Artificial Sequence
  Description of Artificial Sequence: Synthetic oligonucleotide
tcactgtctggctcacatacccata

SEQ ID NO: 328 - DNA, 25 bp, Artificial Sequence
  Description of Artificial Sequence: Synthetic oligonucleotide
gcggtttaaaggcatggctggcgca

SEQ ID NO: 329 - DNA, 25 bp, Artificial Sequence
  Description of Artificial Sequence: Synthetic oligonucleotide
tggacaaagtgcggtttaaaggcat

SEQ ID NO: 330 - DNA, 25 bp, Artificial Sequence
  Description of Artificial Sequence: Synthetic oligonucleotide
gtgaggacatggacaaagtgcggtt

SEQ ID NO: 331 - DNA, 25 bp, Artificial Sequence
  Description of Artificial Sequence: Synthetic oligonucleotide
cagtcctggcagtgaggacatggac

SEQ ID NO: 332 - DNA, 25 bp, Artificial Sequence
  Description of Artificial Sequence: Synthetic oligonucleotide
ccagtcctccagtcctggcagtgag

SEQ ID NO: 333 - DNA, 25 bp, Artificial Sequence
  Description of Artificial Sequence: Synthetic oligonucleotide
cacctgggattcttaaatatagatg

SEQ ID NO: 334 - protein, 497 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGM

SEQ ID NO: 335 - protein, 514 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 336 - protein, 504 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 337 - protein, 488 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 338 - protein, 412 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTVTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 339 - protein, 147 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYETPSPLRITLLVQERRRKKRKSCRGCCQA

SEQ ID NO: 340 - DNA, 25 bp, Artificial Sequence
  Description of Artificial Sequence: Synthetic oligonucleotide
agaacgtaatcatcagtgggttggc

SEQ ID NO: 341 - DNA, 194 bp, Homo sapiens
cgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 342 - DNA, 199 bp, Homo sapiens
acctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 343 - DNA, 199 bp, Homo sapiens; identical to SEQ ID NO: 342

SEQ ID NO: 344 - DNA, 32 bp, Artificial Sequence
  Description of Artificial Sequence: Synthetic oligonucleotide
agctcctccagcagagcccggtattcttcctc

SEQ ID NO: 345 - DNA, 35 bp, Artificial Sequence
  Description of Artificial Sequence: Synthetic oligonucleotide
cgaaccacccgaccccgtcccaaccccgcgtccta

SEQ ID NO: 346 - DNA, 25 bp, Artificial Sequence
  Description of Artificial Sequence: Synthetic oligonucleotide
agctcctccagcagagcccggtatt

SEQ ID NO: 347 - DNA, 25 bp, Artificial Sequence
  Description of Artificial Sequence: Synthetic oligonucleotide
gctcctccagcagagcccggtattc

SEQ ID NO: 348 - DNA, 25 bp, Artificial Sequence
  Description of Artificial Sequence: Synthetic oligonucleotide
ctcctccagcagagcccggtattct

SEQ ID NO: 349 - DNA, 25 bp, Artificial Sequence
  Description of Artificial Sequence: Synthetic oligonucleotide
tcctccagcagagcccggtattctt

SEQ ID NO: 350 - DNA, 25 bp, Artificial Sequence
  Description of Artificial Sequence: Synthetic oligonucleotide
cctccagcagagcccggtattcttc

SEQ ID NO: 351 - DNA, 25 bp, Artificial Sequence
  Description of Artificial Sequence: Synthetic oligonucleotide
ctccagcagagcccggtattcttcc

SEQ ID NO: 352 - DNA, 25 bp, Artificial Sequence
  Description of Artificial Sequence: Synthetic oligonucleotide
tccagcagagcccggtattcttcct

SEQ ID NO: 353 - DNA, 25 bp, Artificial Sequence
  Description of Artificial Sequence: Synthetic oligonucleotide
ccagcagagcccggtattcttcctc

SEQ ID NO: 354 - DNA, 25 bp, Artificial Sequence
  Description of Artificial Sequence: Synthetic oligonucleotide
cgaaccacccgaccccgtcccaacc

SEQ ID NO: 355 - DNA, 25 bp, Artificial Sequence
  Description of Artificial Sequence: Synthetic oligonucleotide
gaaccacccgaccccgtcccaaccc

SEQ ID NO: 356 - DNA, 25 bp, Artificial Sequence
  Description of Artificial Sequence: Synthetic oligonucleotide
aaccacccgaccccgtcccaacccc

SEQ ID NO: 357 - DNA, 25 bp, Artificial Sequence
  Description of Artificial Sequence: Synthetic oligonucleotide
accacccgaccccgtcccaaccccg

SEQ ID NO: 358 - DNA, 25 bp, Artificial Sequence
  Description of Artificial Sequence: Synthetic oligonucleotide
ccacccgaccccgtcccaaccccgc

SEQ ID NO: 359 - DNA, 25 bp, Artificial Sequence
  Description of Artificial Sequence: Synthetic oligonucleotide
cacccgaccccgtcccaaccccgcg

SEQ ID NO: 360 - DNA, 25 bp, Artificial Sequence
  Description of Artificial Sequence: Synthetic oligonucleotide
acccgaccccgtcccaaccccgcgt

SEQ ID NO: 361 - DNA, 25 bp, Artificial Sequence
  Description of Artificial Sequence: Synthetic oligonucleotide
cccgaccccgtcccaaccccgcgtc

SEQ ID NO: 362 - DNA, 25 bp, Artificial Sequence
  Description of Artificial Sequence: Synthetic oligonucleotide
ccgaccccgtcccaaccccgcctcc

SEQ ID NO: 363 - DNA, 25 bp, Artificial Sequence
  Description of Artificial Sequence: Synthetic oligonucleotide
cgaccccgtcccaaccccgcgtcct

SEQ ID NO: 364 - DNA, 25 bp, Artificial Sequence
  Description of Artificial Sequence: Synthetic oligonucleotide
gaccccgtcccaaccccgcgtccta

SEQ ID NO: 365 - DNA, 25 bp, Artificial Sequence
  Description of Artificial Sequence: Synthetic oligonucleotide
acgtaatcatcagtgggttggctct

SEQ ID NO: 366 - DNA, 25 bp, Artificial Sequence
  Description of Artificial Sequence: Synthetic oligonucleotide
ttggcaacatcctctgcagctgaag

SEQ ID NO: 367 - DNA, 19 bp, Artificial Sequence
  Description of Artificial Sequence: Synthetic oligonucleotide
gcaacatcctctgcagctg

SEQ ID NO: 368 - DNA, 25 bp, Artificial Sequence
  Description of Artificial Sequence: Synthetic oligonucleotide
tcaggcttggcaacatcctctgcag

SEQ ID NO: 369 - DNA, 25 bp, Artificial Sequence
  Description of Artificial Sequence: Synthetic oligonucleotide
taatcatcagtgggttggctctctg

SEQ ID NO: 370 - protein, 41 aa, Artificial Sequence
  Description of Artificial Sequence: Synthetic polypeptide
RMRKFKNKGKDTAELRRRRVEVSVELRKAKKDEQILKRRNV

SEQ ID NO: 371 - protein, 7 aa, Artificial Sequence
  Description of Artificial Sequence: Synthetic peptide
PKKKRKV

(19) United States
(12) Patent Application Publication
(10) Pub. No.: US 2025/0051780 A1
(43) Pub. Date: Feb. 13, 2025

(54) COMPOSITIONS AND METHODS FOR MODULATING mRNA SPLICING

(71) Applicant: ENTRADA THERAPEUTICS, INC., Boston, MA (US)

(72) Inventors: Ziqing QIAN, Wellesley, MA (US); Natarajan SETHURAMAN, Hanover, NH (US); Xiulong SHEN, Waltham, MA (US); Haoming LIU, Canton, MA (US); Xiang LI, Belmont, MA (US)

(21) Appl. No.: 18/289,946

(22) PCT Filed: May 9, 2022
(86) PCT No.: PCT/US2022/028357

§ 371 Date: Nov. 8, 2023

Related U.S. Application Data
(60) Provisional application No. 63/186,664, filed on May 10, 2021.
(60) Provisional application No. 63/210,866, filed on Jun. 15, 2021.
(60) Provisional application No. 63/210,882, filed on Jun. 15, 2021.
(60) Provisional application No. 63/239,671, filed on Sep. 1, 2021.
(60) Provisional application No. 63/298,587, filed on Jan. 11, 2022.
(60) Provisional application No. 63/318,201, filed on Mar. 9, 2022.
(60) Provisional application No. 63/321,921, filed on Mar. 21, 2022.
(60) Provisional application No. 63/362,295, filed on Mar. 31, 2022.

(51) Int. Cl. C12N 15/113; A61P 3/00

(52) U.S. Cl. CPC C12N 15/1137; A61P 3/00; C12N 2310/11; C12N 2310/3233; C12N 2310/3513; C12N 2320/33; C12Y 204/01011

(57) ABSTRACT

Compounds include at least one cyclic cell penetrating peptide (cCPP) conjugated to an antisense compound (AC). The AC modulates splicing of an RNA transcript. For example, the AC induces exon skipping. Exon skipping can result in down-regulation of expression or activity of a protein. Exon skipping may cause a frameshift in a resulting mRNA. The frameshift may result in a premature termination codon. The frameshift may result in nonsense mediated decay.

### CROSS REFERENCE TO RELATED APPLICATIONS

This application claims the benefit of U.S. Provisional Application Serial Nos. 63/186,664, filed on May 10, 2021; 63/210,882, filed Jun. 15, 2021; 63/321,921, filed on Mar. 21, 2022; 63/362,295, filed on Mar. 31, 2022; 63/239,671, filed on Sep. 1, 2021; 63/210,866, filed on Jun. 15, 2021; 63/298,587, filed on Jan. 11, 2022; and 63/318,201, filed on Mar. 9, 2022, which provisional applications are incorporated by reference herein in their respective entireties to the extent that they do not conflict with the disclosure presented herein.

### FIELD OF THE INVENTION

Provided herein are compositions and methods for modulating mRNA splicing. In particular, compositions and methods are provided for modulating expression or activity of a protein of interest by inducing exon skipping, for example, to introduce a frameshift in the RNA transcript which may result in nonsense mediated decay of the RNA transcript.

### BACKGROUND

A gene is a deoxyribonucleic acid (DNA) sequence that encodes a functional gene product, such as a protein. The process of converting the code of the gene into the functional gene product includes the steps of transcribing RNA (transcript) from genetic DNA and translating the RNA into a protein RNA is first transcribed from DNA as immature “pre-mRNA” that undergoes processing to become a mature messenger RNA (mRNA) that can be translated into a protein. In eukaryotes, the processing steps include addition of a single-nucleotide modified guanine (G) nucleotide cap to the 5′ end of the RNA; addition of a poly-adenosine sequence to the 3′ end of the RNA (poly-A tail); and RNA splicing.

Splicing refers to a process in which introns (intervening sequences) are removed from the pre-mRNA and exons (coding sequences) are ligated together to form a mature mRNA.

Many mammalian genes are alternatively spliced, wherein different exons in the pre-mRNA sequence are included or excluded in the mature mRNA transcript such that one gene can generate different mRNA messages that are translated into proteins with different sizes and/or functions (isoforms).

Alternative splicing may involve cryptic splice sites within the exonic and/or intronic regions of a transcript. A cryptic splice site is a splice site that is not typically used but may be used when the usual splice site is blocked or unavailable or when a mutation causes a normally dormant site to become an active splice site. In cryptic splicing, the splicing machinery recognizes the cryptic splice site rather than a canonical splice site. Often, cryptic splicing results in the inclusion or exclusion of a portion of or a whole intron or exon sequence in the mRNA.

Antisense modulation of pre-mRNA splicing has been used to restore cryptic splicing, to change levels of alternatively spliced genes (isoform switching), and for exon skipping, for example, to restore a disrupted reading frame or to knockdown the function of an undesired gene (Aartsma-Rus and Ommen, RNA (2007), 13:1609-1624).

Major problems for the use of antisense compounds in therapeutics includes their limited ability to gain access to the intracellular compartment when administered systemically, their limited ability to achieve wide or specifically-targeted tissue distribution, and the challenge of obtaining sufficient specificity for the targeted RNA to minimize off-target effects. Intracellular delivery of antisense compounds can be facilitated by use of carrier systems such as polymers, cationic liposomes or by chemical modification of the construct, for example by the covalent attachment of cholesterol molecules. However, intracellular delivery efficiency is low and tissue distribution can be narrow. In addition, existing technologies remain hampered by off-target interactions. As a consequence, improved delivery systems are still required to increase the effectiveness of these antisense approaches, and there remains an unmet need for effective compositions to deliver antisense compounds to intracellular compartments broadly to all effected tissue types to specifically target a given gene product, so as to treat diseases caused by, for example, aberrant gene transcription, splicing and/or translation.

### SUMMARY

This disclosure generally relates to compounds, compositions, and methods for modulating splicing of target transcripts (for example, pre-mRNA) of genes, such as genes associated with diseases. In embodiments, this disclosure relates to compounds and compositions that include a therapeutic moiety (TM) and a cell penetrating peptide (CPP). The TM can be an antisense compound (AC) that binds the target transcript to modulate splicing of the target transcript. In embodiments, the AC binds to at least a portion of a splice element (SE) or cis-acting splice regulatory element (SRE) of the target transcript, or in proximity to a splice element or a cis-acting splice regulatory element of the target transcript, to modulate splicing of the target transcript. In embodiments, binding of the AC to the target transcript results in downregulation of expression or activity of a protein expressed from the target transcript.

In embodiments, binding of the AC to the target transcript results in skipping of an exon. In embodiments, skipping of an exon results in a frameshift. In embodiments, the frameshift results in a premature stop codon. In embodiments, the frameshift results in nonsense mediated decay. In embodiments, the frameshift results in a premature stop codon and in nonsense mediated decay.

Described herein are methods in which the compounds or compositions described herein are used to treat a disease. In embodiments, the disease is a genetic disease. In embodiments, the compounds or compositions are used to treat the genetic disease by modulating splicing of a gene associated with the disease. In embodiments, the compounds or compositions treat the genetic disease by modulating splicing of a gene transcript associated with the disease. In embodiments, the methods comprise administering the compound or compositions described herein to a subject in need thereof. In embodiments, the subject in need thereof is a patient having, or at risk of having, the genetic disease. In embodiments, the method comprises administering a therapeutically effective amount of the compound or compositions described herein to the subject in need thereof. In embodiments, the genetic disease is a disease associated with aberrant expression of IRF-5, DUX4, or GYS1 or a genetic variant thereof.

The CPP may enhance intracellular deliver of the AC to enhance the effectiveness of the AC to modulate splicing of the target transcript. The CPP can be a cyclic CPP (cCPP).

The compounds described herein may comprise an endosomal escape vehicle (EEV) configured to allow compounds, or moieties thereof, that are internalized into the cell in endosomes to escape the endosomes and enter the cytosol or cellular compartment to allow the AC act on the target transcript and modulate splicing. In embodiments, the EEV comprises the CPP, such as the cCPP.

In embodiments, the cCPP is of Formula (A):

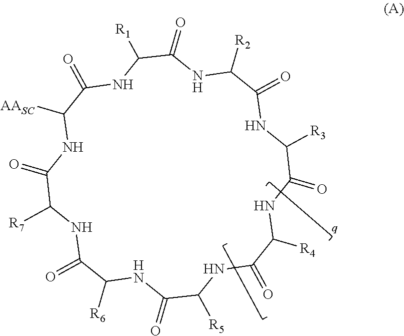

or a protonated form thereof, wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, r-homoarginine, 3-(1-piperidinyl)alanine;
AA_{SC} is an amino acid side chain; and
q is 1, 2, 3 or 4.

In embodiments, the cCPP is of Formula (A) is of Formula (I):

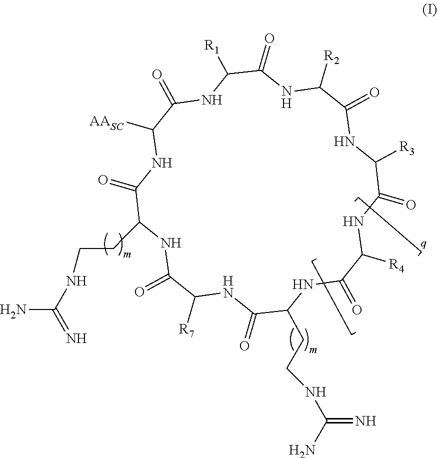

or a protonated form or salt thereof,
wherein each m is independently an integer from 0-3.

In embodiments, the cCPP is of Formula (A) is of Formula (I-1):

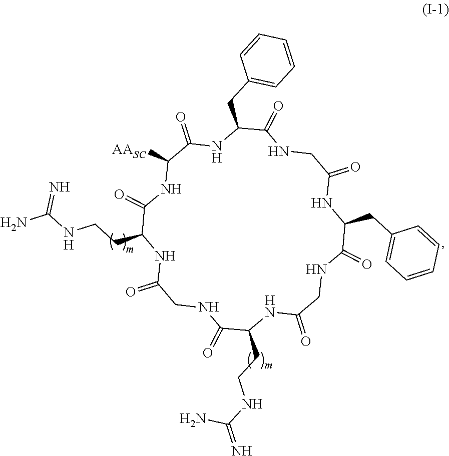

or a protonated form or salt thereof.

In embodiments, the cCPP is of Formula (A) is of Formula (1-2):

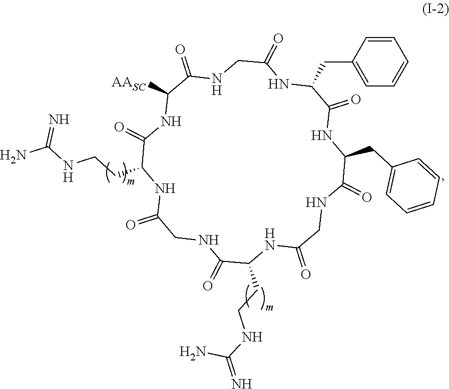

or a protonated form or salt thereof.

In embodiments, the cCPP is of Formula (A) is of Formula (1-3):

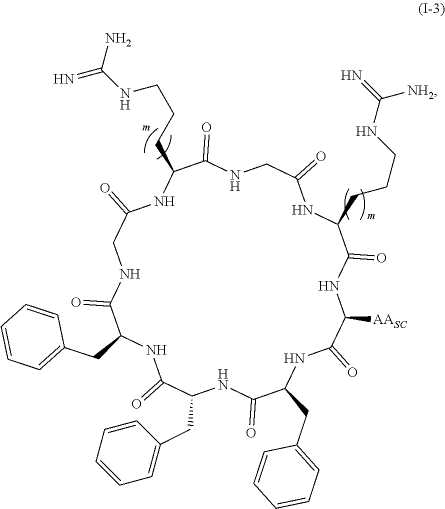

or a protonated form or salt thereof.

In embodiments, the cCPP is of Formula (A) is of Formula (1-4):

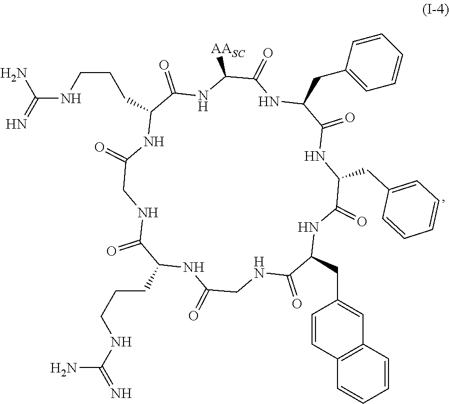

or a protonated form or salt thereof.

In embodiments, the cCPP is of Formula (A) is of Formula (1-5):

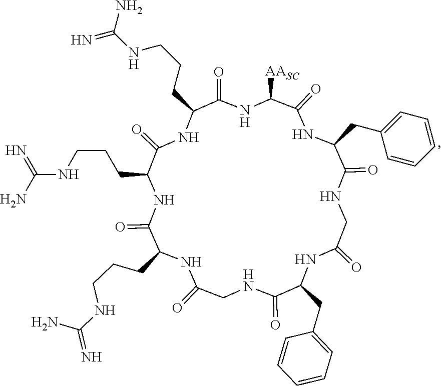

or a protonated form or salt thereof.

In embodiments, the cCPP is of Formula (A) is of Formula (I-6):

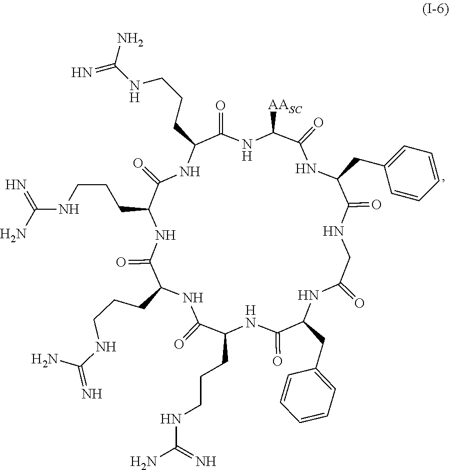

or a protonated form or salt thereof.

In embodiments, the cCPP is of Formula (II):

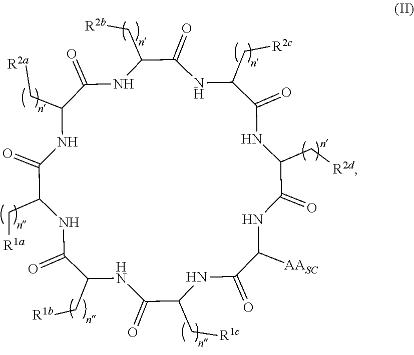

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is

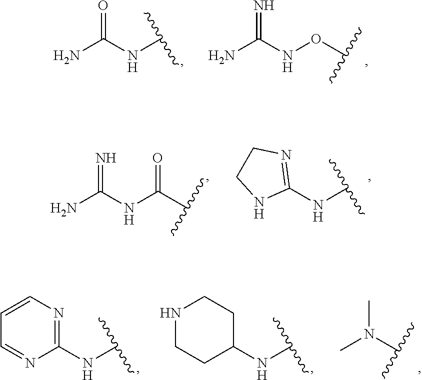

or a protonated form or salt thereof;
at least one of R^{2a}, R^{2b}, R^{2c} and R^{2d} is guanidine or a protonated form or salt thereof;
each n″ is independently an integer from 0 to 5;
each n′ is independently an integer from 0 to 3; and
if n′ is 0 then R^{2a}, R^{2b}, R^{2c} or R^{2d} is absent.

In embodiments, the cCPP of Formula (II) is of Formula (II-1):

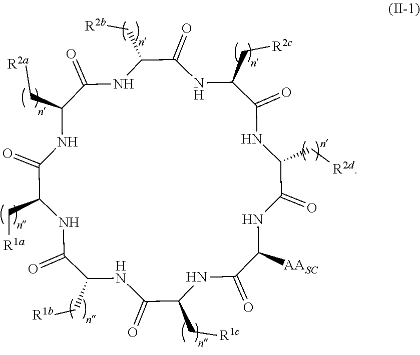

In embodiments, the cCPP of Formula (II) is of Formula (IIa):

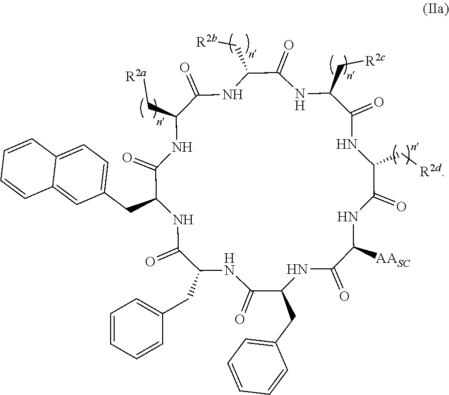

In embodiments, the cCPP of Formula (II) is of Formula (IIb):

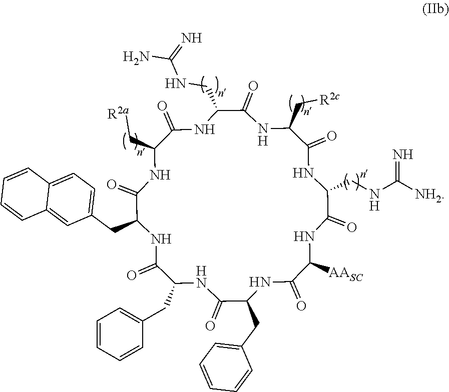

In embodiments, the cCPP of Formula (II) is of Formula (IIc):

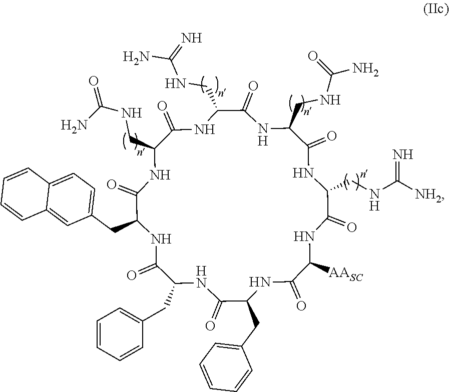

or a protonated form or salt thereof.

In embodiments, the cCPP has the structure:

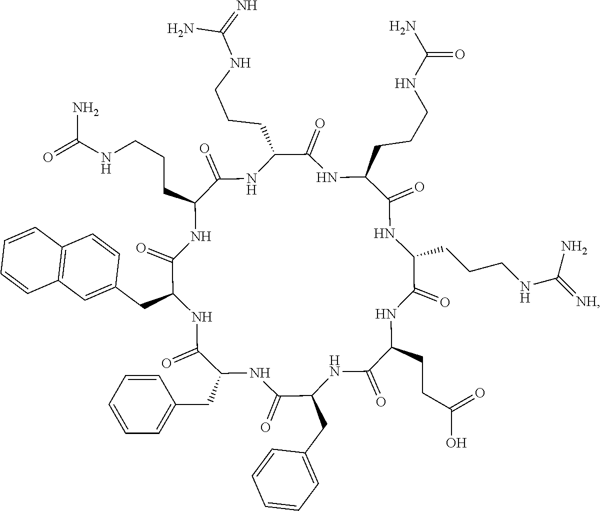 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

or a protonated form or salt thereof, wherein at least one atom of an amino acid side chain is replaced by the therapeutic moiety or a linker or at least one lone pair forms a bond to the therapeutic moiety or the linker.

In embodiments, the cCPP has the structure:

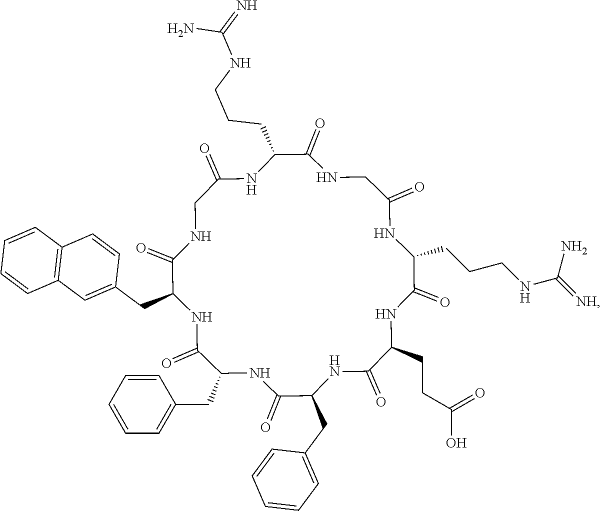 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]

or a protonated form or salt thereof, wherein at least one atom of an amino acid side chain is replaced by the therapeutic moiety or a linker or at least one lone pair forms a bond to the therapeutic moiety or the linker.

In embodiments, the compound comprises an exocyclic peptide (EP). In embodiments, the EP comprises one of the following sequences: KK, KR, RR, HH, HK, HR, RH, KKK, KGK, KBK, KBR, KRK, KRR, RKK, RRR, KKH, KHK, HKK, HRR, HRH, HHR, HBH, HHH, HHHH, KHKK, KKHK, KKKH, KHKH, HKHK, KKKK, KKRK, KRKK, KRRK, RKKR, RRRR, KGKK, KKGK, HBHIBII, JBKBH, RRRRR, KKKKK, KKKRK, RKKKK, KRKKK, KKRKK, KKKKR, KBKBK, RKKKKG, KRKKKG, KKRKKG, KKKKRG, RKKKKB, KRKKKB, KKRKKB, KKKKRB, KKKRKV, RRRRRR, HHHHHH RHRHRH, HRHRHR, KRKRKR, RKRKRK, RBRBRB, KBKBKB, PKKKRKV, PGKKRKV, PKGKRKV, PKKGRKV, PKKKGKV, PKKKRCGV or PKKKRKG, wherein B is beta-alanine.

In embodiments, the compound is of Formula (C):

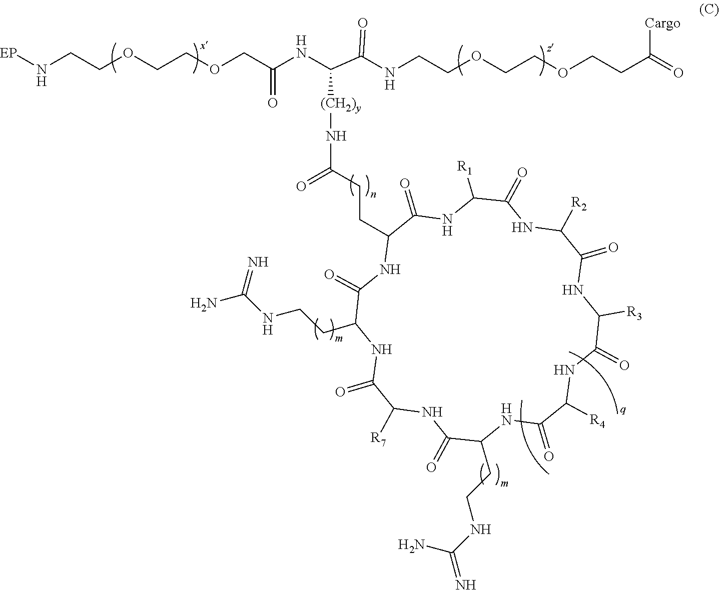

or a protonated form or salt thereof, wherein:
R₁, R₂, and R₃ are each independently H or a side chain comprising an aryl or heteroaryl group, wherein at least one of R₁, R₂, and R₃ is a side chain comprising an aryl or heteroaryl group;
R₄ and R₇ are independently H or an amino acid side chain;
EP is an exocyclic peptide;
each m is independently an integer from 0-3;
n is an integer from 0-2;
x′ is an integer from 1-23;
y is an integer from 1-5;
q is an integer from 1-4;
z′ is an integer from 1-23, and
Cargo is the AC.

In embodiments, the compound comprises the structure of Formula (C-1), (C-2), (C-3), or (C-4):

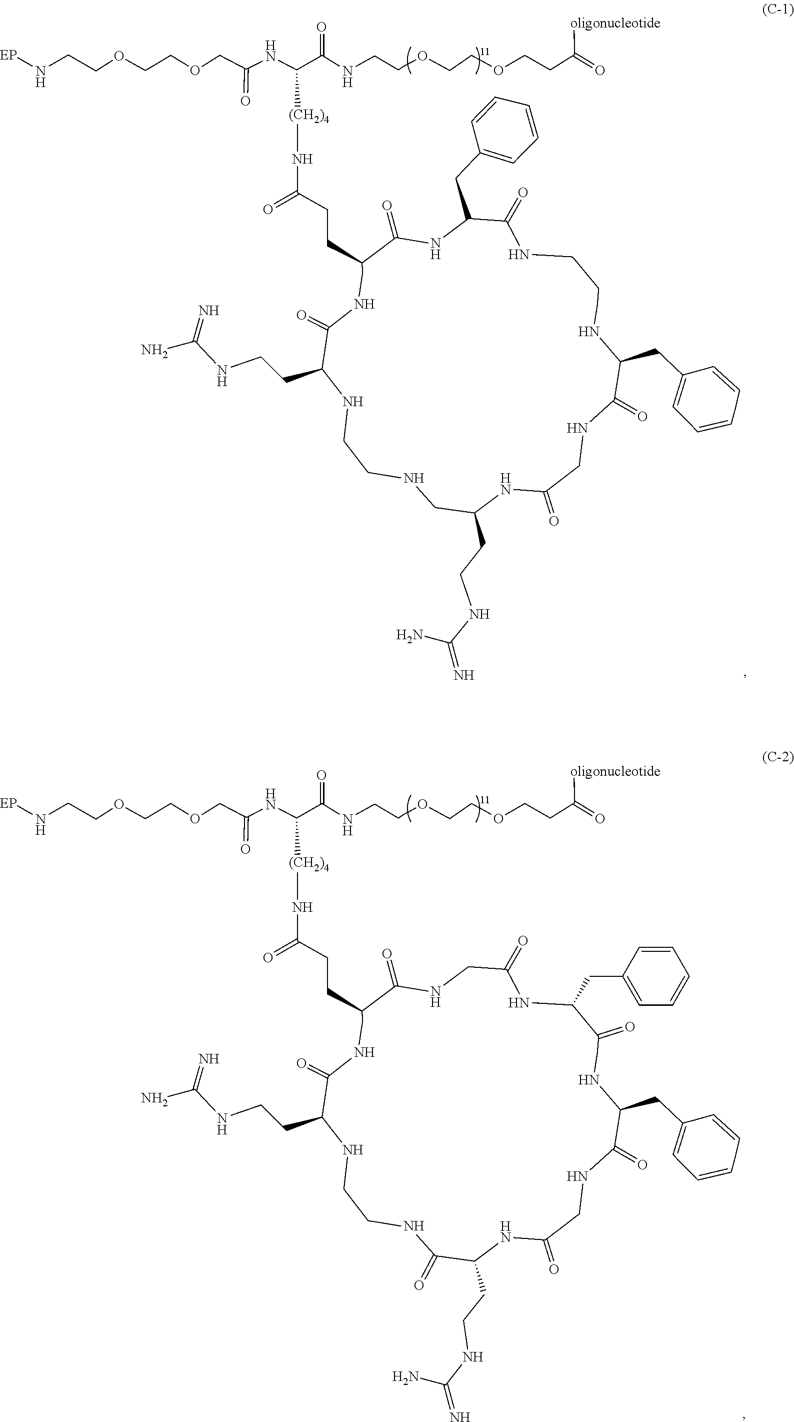

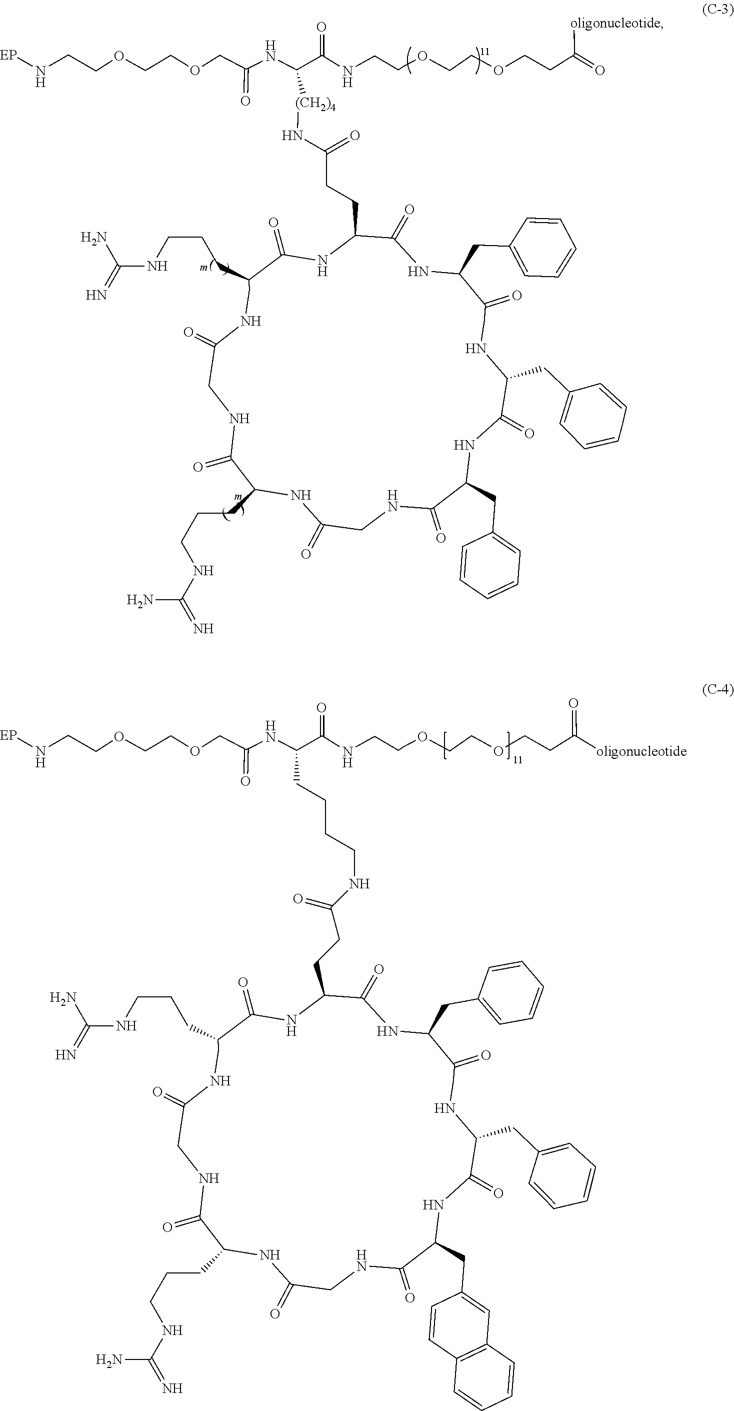

or a protonated form or salt thereof, wherein EP is an exocyclic peptide, and oligonucleotide is the AC.

### BRIEF DESCRIPTION OF THE DRAWINGS

FIGS. 1A-B are schematic drawings showing splicing regulatory elements, including splice sites (A), and general splicing reactions (two transesterification reactions) (B).

FIG. 2 is a schematic drawing showing antisense compound mediated exon skipping to create a premature termination codon which ultimately leads to nonsense mediated decay of the target transcript.

FIG. 3 shows modified nucleotides used in antisense oligonucleotides described herein. Structures 1-3 (1=Phosphorothioate; 2=(S_{C5}—Rₚ)-α,β-CAN; 3=PMO) are phosphate backbone modifications; 4 (2-thio-dT) is a base modification; 5-8 (5=2′-OMe-RNA; 6=2′O-MOE-RNA; 7=2′F-RNA; 8=2′F-ANA) are 2′ sugar modifications; 9-11 are constrained nucleotides; 12-14 (9=LNA; 10=(S)-cET; 11=tcDNA; 12=FHNA; 13=(S)5′-C-methyl; 14=UNA) are additional sugar modification; and 15-18 (15=F-VP; 16=Methyl phosphonate; 17=5′ phosphorothioate; 18=(S)-5′-C-methyl with phosphate) are 5′ phosphate stabilization modifications; 19 is a morpholino sugar. Reformatted from Khvorova, A., et al., Nat. Biotechnol. (2017) March; 35(3): 238-248.

FIGS. 4A-4D provide structures of the adenine (A), cytosine (B), guanine (C), and thymine (D) morpholino subunit monomers used in synthesizing phosphorodiamidate-linked morpholino oligomers (PMOs).

FIGS. SA-D illustrate conjugation chemistries for connecting an antisense compound (AC) to a peptide, such as a cyclic cell penetrating peptide (cCPP). FIG. 5A shows reagents for an amide bond formation reaction between a peptide having an N-hydroxysuccinimide activated ester(top) or a peptide with a free carboxylic acid (bottom) and primary amine at the 5′ end of AC. FIG. 5B shows reagents for amide bond formation reactions of a primary or secondary amine at the 3′ end of the AC with a peptide having a tetrafluophenyl (TFP) activated ester. FIG. 5C shows reagents for the conjugation of peptide-azide to the 5′ cyclooctyne modified AC via copper-free azide-alkyne cycloaddition. FIG. 5D demonstrates other exemplary reagents for conjugation between 3′ modified cyclooctyne ACs or 3′ modified azide ACs and peptides, such as a cCPP, containing linker-azide or linker-alkyne/cyclooctyne moiety, via a copper-free azide-alkyne cycloaddition or cupper catalyzed azide-alkyne cycloaddition, respectively (click reaction).

FIG. 6 shows conjugation chemistry for connecting AC and CPP with an additional linker modality containing a polyethylene glycol (PEG) moiety using conjugation chemistry shown in FIG. 5. Methods of purification are indicated.

FIGS. 7A-D show the level of GYS1 protein (A and C) and GYS1 mRNA (B and D) in the diaphragm (A and B) and heart (C and D) of untreated mice, mice treated with a PMO, and mice treated with various concentrations of an EV-PMO in GAA knockout mouse model (P>0.05=NS; P≤0.05=*; P≤0.01=**P≤0.001=***)

FIGS. 8A-D show plots of the level of GYS1 mRNA levels in the heart (A), diaphragm (13), quadriceps (C), and triceps (D) of untreated mice, mice treated with a PMO, and mice treated with an EEV-PMO at various time points after treatment. (P>0.05=NS; P≤0.05=*; P≤0.01=**; P≤0.001=***)

FIGS. 9A-D show plots of the level of GYS1 protein in the heart (A), diaphragm (B), quadriceps (C), and triceps (D) of untreated mice, mice treated with a PMO, and mice treated with an EEV-PMO at various time points after treatment. (P>0.05=NS; P≤0.05=*; P≤0.01=**; P≤0.001=***)

FIGS. 10A-C are plots showing the level of IRF5 mRNA expression the liver (A), small intestine (B), and tibialis anterior (C) of mice treated with various concentrations of an EEV-PMO. (P>0.05=NS; P≤0.05=*; P≤0.01=**; P≤0.001=***). MPK (mpk)=mg per kg.

FIGS. 11A-B are plots showing the level of IRF5 protein expression in an in vitro experiment where mouse macrophage cells were treated with various concentrations of EEV #1-PMO, EEV #2-PMO, EEV #3-PMO, and EEV #4-PMO. (P>0.05=NS; P≤0.05=*; P≤0.01=**; P≤0.001=***)

FIG. 12 is a plot showing knockdown of GYS1 mRNA levels in the wildtype mouse myoblast cell line C2C12 after treatment with various concentrations of PMO 220 or EEV-PMO 220-814. N:=3, *p<0.05, **p<0.01 relative to 0 (no treatment) by student t-test.

FIGS. 13A-B are plots showing knockdown of GYS1 mRNA levels in mouse myoblasts (A) and mouse fibroblasts (B) after treatment with various concentrations of PMO 220. N=2, *p<0.05 relative to NT (no treatment) by student t-test.

FIGS. 14A-D are plots showing the GYS1 mRNA level in the heart (A), diaphragm (B), triceps (C), and quadriceps (D) after GAA knockout mice were treated with PMO 220 or various concentrations of PMO-EEV 220-814. MPK (mpk)=mg per kg.

FIG. 15 is a plot showing the GYS2 mRNA level in the liver after GAA knockout mice were treated with PMO 220 or various concentrations of PMO-EEV 220-814. MPK (mpk)=mg per kg.

FIGS. 16A-D are plots showing the GYS1 mRNA level in the heart (A), diaphragm (B), triceps (C), and quadriceps (D) after GAA knockout mice were treated with PMO 220 or various concentrations of PMO-EEV 220-1055.

FIGS. 17A-D are plots showing the GYS1 protein level in the heart (A), diaphragm (B), triceps (C), and quadriceps (D) at various time points after GAA knockout mice were treated with 20 mpk of PMO-EEV 220-1055. MPK (mpk)=mg per kg.

FIGS. 18A-D are plots showing the drug exposure level in the heart (A), diaphragm (B), triceps (C), and quadriceps (D) at various time points after GA A knockout mice were treated with 20 mpk of PMO 220 or 20 mpk of PMO-EEV 220-1055. MPK (mpk)=mg per kg.

FIGS. 19A-D are plots showing the GYS1 mRNA level in the heart (A), diaphragm (B), triceps (C), and quadriceps (D) for wild type mice, GAA knockout mice, and GAA knockout mice treated with various concentrations of EEV-PMO 220-1120. MPK (mpk)=mg per kg.

FIGS. 20A-D are plots showing the GYS1 protein level in the heart (A), diaphragm (B), triceps (C), and quadriceps (D) for wild type mice, GAA knockout mice, and GAA knockout mice treated with various concentrations of EEV-PMO 220-1120. MPK (mpk)=mg per kg.

FIGS. 21A-D are plots showing the GYS1 protein level in the heart (A), diaphragm (B), and quadriceps (C) for wild type mice, GAA knockout mice, and GAA knockout mice treated with multiple doses of EEV-PMO 220-1055.

FIGS. 22A-B are plots showing the GYS1 (A) and the GYS2 (B) level in the liver for wild type mice, GAA knockout mice, and GAA knockout mice treated with multiple doses of EEV-PMO 220-1055.

FIGS. 23A-C show the expression levels of IRE-5 in mouse TiA tissue (A), liver tissue (B), and small intestine tissue (C), after mice were treated with two doses of a PMO or EEV-PMO 278-1120. MPK (mpk)=mg per kg.

FIG. 24A-C show the IRF-5 expression levels in mouse liver (A), kidney (B), and tibialis anterior (C) tissue after mice were treated with one dose of PMO 278 or PMO-EEV 278-1120. P>0.05=NS; P≤0.05=*; P≤0.01=**; P≤0.001=***.

FIG. 25A-B show GYS1 protein levels in the quadriceps (A) and triceps (B) using a GYS antibody not specific to GYS1 after mice were treated with various concentration of EEV-PMO construct 220-814.

FIG. 26A-C show GYS1 protein levels in the diaphragm (A), heart (B), and triceps (C) using a GYS1 specific antibody after mice were treated with various concentration of EEV-PMO construct 220-814.

FIG. 27A show the IRF-5 expression levels RAW 264.7 Monocyte/Macrophage cells after treatment with various concentrations of PMO-EEVs 277-1120 and 278-1120. P>0.05=NS; P<0.05=*; P<0.01=**; P≤0.001=**.

FIG. 27B is a bar graph of exon skipping percentage at various time points after RAW 264.7 Monocyte/Macrophage cells were treated with EEV-PMO 278-1120. NT=No treatment.

FIGS. 28A-B are a bar graphs showing the levels of IRF-5 expression (A) and exon 4 skipping percentage (B) in RAW 264.7 Monocyte/Macrophage cells after treatment with various EEV-PMOs at various concentrations followed by R848 stimulation.

FIGS. 29A-B are plots show the IRF-5 exon 4 and exon 5 skipping levels in human THP1 cells after treatment with the various EEV-PMOs at various concentrations.

### DETAILED DESCRIPTION

### Splicing

Pre-mRNA molecules are made in the nucleus and are processed before or during transport to the cytoplasm for translation. Processing of the pre-mRNAs includes addition of a 5′ methylated guanine cap and an approximately 200-250 base poly(A) tail to the 3′ end of the transcript. Pre-mRNA processing also includes splicing, which occurs in the maturation of about 90% to about 95% of mammalian mRNAs. Introns (or intervening sequences) are regions of a primary transcript (or the DNA encoding it) that are not included in the coding sequence of the mature mRNA. Exons are regions of a primary transcript that remain in the mature mRNA when it reaches the cytoplasm. A transcript may have multiple introns and exons. The exons are spliced together to form the mature mRNA sequence. Splice junctions are also referred to as splice sites with the 5′ side of the junction often called the “5′ splice site” or “splice donor site” and the 3′ side called the “3′ splice site” or “splice acceptor site.” In splicing, the 3′ end of an upstream exon is joined to the 5′ end of the downstream exon. Thus, the transcript (e.g., pre-mRNA) has an exon/intron junction at the 5′ end of an intron and an intron/exon junction at the 3′ end of an intron. After the intron is removed, the exons are contiguous at what is sometimes referred to as the exon/exon junction or boundary in the mature mRNA. Cryptic splice sites are those which are less often used but may be used when the usual splice site is blocked or unavailable. Alternative splicing, defined as the splicing together of different combinations of exons, often results in multiple mRNA transcripts from a single gene.

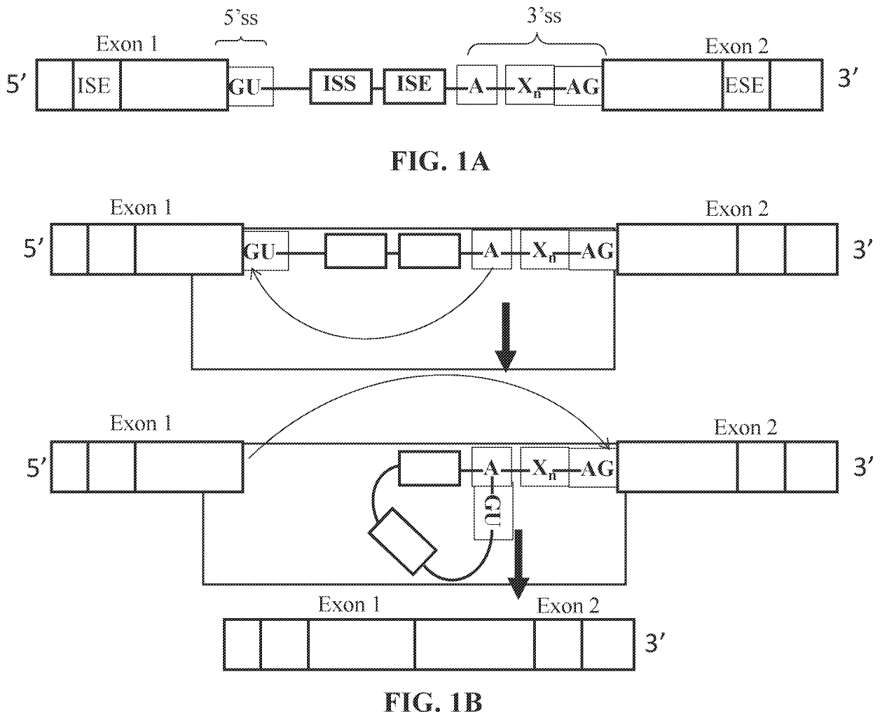

Removal of the introns from pre-mRNA is catalyzed by a spliceosome, a ribonucleoprotein (RNP) complex that includes five small nuclear ribonucleoproteins (snRNPs), and numerous other proteins (Will and Lührmann, Cold Spring Harb. Perspect. Biol. (2011), 3(7):a003707; Havens, et al., Wiley Interdiscip. RNA (2014), 4(3), 247-266. doi:10.1002/wrna.1158). Splicing is governed in part by splice elements (SE). As used herein, “splice elements” are sequence elements found in pre-mRNA that are necessary for splicing, such as canonical splicing, to occur (FIG. 1A). SEs include a 5′ splice site (5′ss) and a 3′ splice site (3′ss). The 5′ ss, also referred to as a donor splice site, includes a nearly invariant “GU” dinucleotide sequence along with less conserved downstream residues. The 5′ splice site also includes an exon/intron junction. As used herein, the exon/intron junction is the nucleotide sequence 10 nucleotides upstream and 10 nucleotides (+10 and −10) from the G of the GU sequence of the 5′ss. The 3′ ss, or acceptor splice site, includes three conserved elements: a branch splice point (DSP) sometimes called the branch point, a polypyrimidine or Py tract, and a terminal “AG.” The BSP is typically an adenosine that is located about 18 to about 40 nucleotides from the 3′ ss. The Py tract typically includes about 15 to about 20 pyrimidine residues, particularly uracil (U) (shown as Xᵣ in FIG. 1A). Atypical branch points exist, however; they are more distant from the 3′ splice site and/or utilize a non-adenosine base (Montes et al. Trends Genet. (2019), 35(i):68-87). The 3′ss also includes an intron/exon junction. As used herein, the intron/exon junction is the nucleotide sequence 10 nucleotides upstream and 10 nucleotides (+10 and −10) from the G of the AG sequence of the 3′ss.

Exons are recognized in most splicing reactions by specific base-pairing interactions with small nuclear RNA (snRNA) components of five small ribonucleoproteins (snRNPs); U1, U2, U4, U5, and U6 (Havens, et al., (2014) Wiley Interdiscip. RNA. 2013, 4(3), 247-266. doi:10.1002/wrna.1158; Wahl M. C. et al., Cell (2009), 136: 701-718). Each snRNP includes a small nuclear RNA that is configured to recognize specific nucleotide sequences and one or more proteins. Exon splicing includes two sequential spliceosome catalyzed transesterification reactions (FIG. 1B). In general, the splicing reaction is initiated by U1 binding to the 5′ss, followed by U2 binding the branch splice point (BPS), and finally U4, U5, and U6 bind near the 5′ and 3′ splice sites. U91 and U4 are then displaced followed by the first transesterification reaction where 2′-OH of a branch-point nucleotide (A as shown in FIG. 1B) within an intron performs a nucleophilic attack on the first nucleotide of the intron at the 5′ splice site (G as shown in FIG. 1B) forming a lariat intermediate. In a second reaction, the 3′-OH of the released 5′ exon performs a nucleophilic attack at the last nucleotide of the intron at the 3′ splice site (G as shown in FIG. 1B) thus joining the exons and releasing the intron lariat. U4, U5, and U6 are released as well.

In addition to SEs, splicing is regulated in part by splicing regulatory elements (SREs). SREs include cis-regulatory elements and trans-acting splicing factors. The cis-regulatory elements and trans-acting splicing factors may promote canonical splicing, alternative splicing, or cryptic splicing.

Cis-regulatory elements are nucleotide sequences within the transcript that suppress or enhance splicing. Trans-acting splicing factors are proteins and/or oligonucleotides that are not located within the transcript and work to enhance or suppress splicing. Cis-regulatory elements generally function to recruit trans-acting splicing factors which activate or suppress splicing. Trans-acting splice factors regulate splicing by associating with cis-regulatory elements. Trans-acting splice factors include serine/arginine rich (SR-rich) proteins and heterogenous nuclear ribonucleoproteins (hnRNPs).

Splicing cis-regulatory elements include exonic splicing enhancer (ESE) sequences, exonic splicing silencers (ESS) sequences, intronic splicing enhancer (ISE) sequences, and intronic splicing silencer (ISS) sequences (FIG. A). ESE sequences promote the inclusion of the exon they reside in into the mRNA. ESS sequences inhibit the inclusion of the exon they residue in into the mRNA. ISE sequences enhance the use of alternate splice sites from their location within an intron. ISS sequences inhibit the use of alternate splice sites from their location within an intron. Typically, ISSs are between 8 and 16 nucleotides in length and are less conserved than the splice sites at exon-intron junctions.

Pre-mRNA splicing may also be regulated by the formation of secondary structures such as terminal stem loops (TSL) within the transcript that may affect the binding of spliceosome or other regulatory proteins. Terminal stem loop sequences may be an SRE and are typically from about 12 to about 24 nucleotides and form a secondary loop structure due to the complementarity, and hence binding, within the 12 to 24 nucleotide sequence.

Each SE and/or cis-acting SRE is separated from an adjacent cis-acting SRE and/or SE by an intervening sequence (IS).

### Exon Skipping

Most eukaryotic pre-mRNA can be spliced differently, often by skipping an exon, to produce distinct mature mRNA isoforms in a process called alternative splicing. The term “alternative splicing” refers to the joining of exons in different combinations (e.g., different 5′ and 3′ splice sites are joined). Alternative splicing can insert or remove amino acids, shift the reading frame, and/or introduce a termination codon, which contributes to the complexity, flexibility, and abundance of genes and proteins expressed from a gene. Alternative splicing can also affect gene expression by removing or inserting regulatory elements, controlling translation, mRNA stability, and/or localization, Mutations that disrupt splicing are estimated to account for up to a third of all disease-causing mutations (Havens, et alt (2014) Wiley Interdiscip. RNA. 2013, 4(3), 247-266. doi:10.1002/wrna.1158; Lim K. H., et al., Proc. Natl. Acad. Sci. USA (2011), 108: 11093-11098; Faustino and Cooper, Genes & Dev. (2003), 17:419-437; and Sterne-Weiler T., et al., Genome Res. (2011), 21: 1563-1571).

Mutations that impact the splicing process can occur in many different ways (Havens, et al., (2014) Wiley Interdiscip. RNA. 2013, 4(3), 247-266. doi:10.1002/wrna.1158). For example, intronic mutations may disrupt the core splice sites (sequences within the 5′ ss or 3′ ss, the Py tract or BPS), resulting in the skipping of an exon(s) upstream or downstream from the mutated splice site (5′ss and/or 3 ss) or the retention of an intron. Often, when a splice site is mutated, a pseudo splice site is activated within a flanking exon or intron, which after splicing results in an alternative transcript. Mutations within an intron can also disrupt or create de novo splicing silencers and/or enhancers and/or create de novo cryptic splice sites. Intronic splice site mutations may account for approximately 10-15% of disease mutations (Havens, et al. (2014) Wiley Interdiscip. RNA. 2013, 4(3), 247-266. doi:10.1002/wrna 1158; Stenson P. D., et al., The Human Gene Mutation Database: 2008 update. Genome Med 2009, 1:13). Mutations that occur within coding exons (exonic mutations), can result in the creation of a de novo cryptic splice site, disruption of an RNA secondary structure that has a regulatory function, and/or disruption of a splicing silencer or enhancer rendering a splice site unrecognizable by a sequence-specific RNA-binding protein that is required for splicing. Analysis of exonic mutations predict that as many as 25% of mutations within exons can alter splicing (Ibid; Proc. Natl. Acad. Sci. USA (2011), 108: 11093-11098). Cryptic splicing is caused by sequences in the pre-mRNA that are not normally used as splice sites, but which are activated by mutations that either inactivate the canonical splice site or create splice sites where one did not exist before (Arechavala-Gomeza, et al., The Application of Clinical Genetics (2014), 4(7), 245-252; Roca X., et al. Genes Dev. (2013); 27(2):129-144). Additionally, alternative splicing, which contributes to the different proteins generated from pre-mRNA, can cause disease by shifting expression from one isoform to a different isoform associated with a disease (Ibid).

Targeting the splicing reaction or splice elements involved in splicing (e.g., SEs and/or SREs) to induce aberrant splicing can be used to disrupt gene expression of proteins involved in disease pathogenesis. For example, splicing can be targeted to cause the skipping of exons, thereby introducing a frameshift or a stop codon that results in a non-functional or truncated protein or degradation of the RNA transcript (Stenson P. D., et al., Genome Med. 2008; 1(13)). Splicing-induced reading frame correction, refraining, and/or nonsense mediated decay of target transcripts provides an opportunity for treating many diseases and disorders.

### Compounds

Disclosed herein, are compounds that modulate the expression and/or activity of a gene of interest. In embodiments, the compounds modulate the splicing of target transcript of a target gene. In embodiments, the compound includes at least one cell penetrating peptide (CPP) and at least one therapeutic moiety (TM) that binds to a target nucleotide sequence. In embodiments, the TM is an antisense compound (AC). In embodiments, the target nucleotide sequence includes a nucleotide sequence that is proximate to or includes at least a portion of a cis-acting splicing regulatory element (SRE) and/or that is proximate to or includes at least a portion of a splicing element (SE).

As used herein, “modulation of splicing” and “modulating splicing” refer to altering the processing of a pre-mRNA transcript such that the spliced mRNA molecule contains either a different combination of exons as a result of exon skipping or exon inclusion, a deletion in one or more exons, or the deletion or addition of a sequence not normally found in the spliced mRNA (e.g., an intron sequence). Modulating splicing may include disrupting or promoting one or more steps of the splicing process. As used herein, the term “splicing process” encompasses all steps of the splicing reactions for example, including, binding of various snRNPs (e.g., U1, U2, U3, U4, and U5) to splicing elements and/or cis-acting splicing regulatory elements, binding of various proteins and/or oligonucleotides to cis-regulatory elements, and the two sequential trans-esterification reactions, as shown in, for example, FIG. 1B.

### Therapeutic Moiety

In embodiments, the present disclosure describes compounds that include one or more therapeutic moieties (TM) that are capable of modulating splicing of a transcript of interest from a gene of interest. In embodiments, a gene of interest may be a disease-causing gene.

The TM binds to (e.g., hybridizes with) a target nucleotide sequence. The target nucleotide sequence is generally contained within a target transcript of a gene of interest. For example, a TM targeting a gene of interest may bind to a target nucleotide sequence (e.g., a splicing element) that is within the target transcript.

The TM may be an antisense compound (AC), one or more of the elements associated with clustered regularly interspaced short palindromic repeats (CRISPR) gene editing machinery, a polypeptide, or combinations thereof.

### Antisense Compounds (ACs)

In embodiments, the therapeutic moiety includes an antisense compound (AC) that can modulate splicing of a target transcript of a target gene. An AC is an oligonucleotide that includes DNA bases, modified DNA bases, RNA bases, modified RNA bases, modified internucleoside linkages, traditional internucleoside linkages, traditional DNA sugars, modified DNA sugars, traditional RNA sugars, modified RNA sugars, or combinations thereof. In embodiments, the AC includes a nucleotide sequence that is complementary to target nucleotide sequence found within a target transcript. In embodiments, the AC includes a nucleotide sequence that is complementary to a target nucleotide sequence that is proximate to or includes at least a portion of a splicing element and/or a splicing regulatory element within a target transcript.

The ACs described herein may contain one or more asymmetric centers and thus give rise to enantiomers, diastereomers, and other stereoisomeric configurations that may be defined, in terms of absolute stereochemistry, as (R) or (S); α or β; or as (D) or (L). Included in the antisense compounds provided herein are all such possible isomers, as well as their racemic and optically pure forms.

In embodiments, the AC induces alternative splicing that leads to the addition or deletion of nucleotides in a target transcript. In some embodiments, the AC induces alternative splicing that leads to the addition or deletion of nucleotides within a single exon of a target transcript. In embodiments, the AC induces alternative splicing that leads to the deletion of nucleotides within a single exon of a target transcript. In embodiments, the deletion of nucleotides within a single exon result in the translation of truncated protein. In embodiments, the truncated protein is less toxic to the cells than the untruncated protein.

In embodiments, the AC is designed to cause exons to be skipped (sometimes called exon skipping) resulting in increased or decreased expression or activity of a target protein and/or a downstream protein that is regulated by the target gene. In embodiments, an AC is provided that generates an mRNA that encodes a truncated protein and/or a nonfunctional protein. In embodiments, an AC is provided that generates an mRNA that encodes a truncated protein and/or a nonfunctional protein through alternative splicing. In embodiments, an AC is provided that triggers degradation of the target transcript, for example, through nonsense mediated decay. In embodiments, an antisense compound (AC) is provided that generates an alternate mRNA isoform that has beneficial properties.

An antisense compound (AC) can be used to modulate splicing in any suitable manner. In embodiments, the AC can be designed to sterically block access to a splice site, or at least a portion of a splicing element (SE) and/or a cis-acting splicing regulatory element (SRE), thereby redirecting splicing to a cryptic or de novo splice site. In embodiments, the AC can be targeted to a splicing enhancer sequence (e.g., ESE an/or ISE) or splicing silencer sequence (e.g., ESS and/or ISS) to prevent binding of trans-acting regulatory splicing factors at the target site and effectively block or promote splicing. In embodiments, the AC can be designed to base-pair across the base of a splicing regulatory stem loop to strengthen the stem-loop structure.

In embodiments, the AC induces the addition or deletion of one or more nucleotides in a resulting processed transcript, such as a mRNA. If the number of nucleotides added or removed from the open reading is divisible by three to produce a whole number, the resultant transcript may be translated into a functioning or non-functioning protein having more or less amino acids than a counterpart protein expressed from a transcript but otherwise has the same amino acid sequence, other than the added or deleted amino acids, as a protein expressed from a transcript that did not have the nucleotides added or removed. If the number of nucleotides added or removed from the open reading frame is not divisible by three to produce a whole number, the open reading frame of the resulting processed transcript, such as an mRNA, is shifted. For example, the number of nucleotides added or deleted to induce a such a “frameshift” alteration may be 1, 2, 4, 5, 7, 8, 10, 11, 13, 14, 16, 17, 19, 20, 22, 23, etc. Due to the triplet nature of the genetic code, the addition or deletion of a number of nucleotides that is not divisible by three, shifts the reading frame of the resulting processed transcript, such as an mRNA, downstream of frameshift. The shifted reading frame may result in nonsense mediated decay, may result in a premature stop co don within the nonsense downstream of the frameshift, and/or may result in expression of a protein having a completely different sequence of amino acids downstream of the frameshift.

In embodiments, the AC induces introduction of a premature termination codon (PTC) into the open reading frame. As used herein, “premature termination codon” is a stop codon in phase with the translational start codon and located upstream of the physiological stop codon that is in phase with the translation start codon. A target transcript having a PTC nay be destabilized and degraded through various mechanisms including nonsense mediated decay.

Nonsense mediated decay is a surveillance mechanism that recognizes initiates exo- and endonucleolytic degradation pathways to remove mRNA transcripts having a PTC in order to prevent the expression of a truncated protein that may have deleterious effects on the cell. Several nonsense mediated decay pathways have been contemplated and reviewed (Lejeune et al., Biomedicines (2020), 10(1):141; Brogna et al., Nature Structural and Molecular Biology (2009), 16, 1084113; Karousis et al., Wiley Interdiscip. Rev. RNA (2016), 7(5): 661-682). In embodiments where the target gene is overexpressed in disease, inducing nonsense mediated decay may be used to reduce the concentration of a target protein, and therefore, treat the disease.

In embodiments, the AC induces exon skipping to result in nonsense mediated decay of the target transcript. This is in contrast to conventional exon skipping which aims to skip an exon to induce expression of a particular protein isoform to correct for missplicing, alternative splicing, and/or to avoid deleterious mutations in specific exons.

In embodiments, the AC induces exon skipping of an exon within a target transcript where the exon has a has a number of nucleotides not divisible by three. In embodiments, the AC induces exon skipping of an exon that has a number of nucleotides not divisible by three resulting in a. PTC within the target transcript. In embodiments, the AC induces exon skipping of an exon that has a number of nucleotides not divisible by three resulting in a PCT within the target transcript which leads to nonsense mediated decay of the target transcript. In embodiments, inducing nonsense mediated decay of a target transcript results in a decreased concentration of the target transcript. In embodiments, inducing nonsense mediated decay of a target transcript results in a decreased concentration of the target protein encoded by the target transcript. In embodiments, inducing nonsense mediated decay of a target transcript results in increased and/or decreased levels of proteins of downstream genes regulated by the target gene.

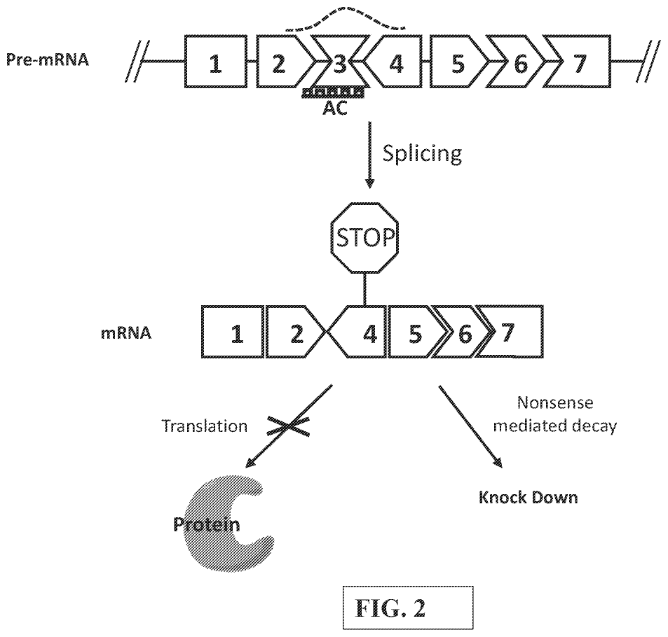

FIG. 2 shows an example of AC induced exon skipping resulting in nonsense mediated decay of a target transcript or premature termination of translation of a protein. The AC binds to pre-mRNA. In the illustrative embodiment, the AC binds the at the intron/exon junction of exon three. In other embodiments, the AC can bind to the target transcript in various other places to induce exon skipping resulting in nonsense mediated decay of the target transcript (discussed elsewhere). The number of nucleotides in exon three is not divisible by three, for example, 52, 106, 232, 365, and the like. Binding of the AC to the intron/exon junction induces exon skipping of exon three through a variety of possible mechanism. For example, the binding of the AC to the intron/exon junction prevents the splicing machinery from accessing the splicing elements. Additionally, or alternatively, the binding of the AC to the intron/exon junction prevents the completion of one or both of the transesterification reactions needed to complete the splicing process. As a result of the AC binding to the target transcript, exon three is skipped and the resultant transcript includes exon two connected with exon four. As a result of the AC binding to the target transcript and skipping exon three, the reading frame in exon four of the resultant transcript is shifted. The shift in reading frame in the illustrated embodiment introduces a PTC in the resulting transcript. As a result of the AC binding to the target transcript, skipping exon three, and exon four having a PTC, the resultant transcript is targeted for and undergoes nonsense mediated decay.

Determining a target sequence and designing an antisense compound (AC) to induce exon skipping can be accomplished using various different methods, including for example those disclosed by Aartsma-Rus, A. et al., Molecular Therapy (2008), 17(3) 548-553; and Aartsma-Rus, A. et al., RNA (2007), 13(10) 1609-1624. In embodiments, the AC hybridizes with target nucleotide sequence that includes at least a portion of a splice element (SE) of target transcript. In embodiments, the AC hybridizes with a target nucleotide sequence that includes an entire SE of a target transcript. In embodiments, the AC hybridizes with a target nucleotide sequence that includes multiple SEs of a target transcript. In embodiments, the AC hybridizes with a target nucleotide sequence that includes multiple SEs of a target transcript and the intervening sequences between the SEs.

In embodiments, the AC hybridizes with target nucleotide sequence that includes at least a portion of a SRE of target transcript. In embodiments, the AC hybridizes with a target nucleotide sequence that includes an entire SRE of a target transcript. In embodiments, the AC hybridizes with a target nucleotide sequence that includes multiple SEs of a target transcript. In embodiments, the AC hybridizes with a target nucleotide sequence that includes multiple SREs of a target transcript and the intervening sequences between the SREs.

In embodiments, the target nucleotide sequence includes the entire SE and/or SRE and one or more flanking sequences that are upstream and/or downstream of the SE and/or SRE of a target transcript. In embodiments, the target nucleotide sequence includes a portion, but not the entirety, of a SE and/or s SRE and one or more flanking sequences that are upstream and/or downstream of the SE and/or the SRE of a target transcript.

In embodiments, the flanking sequence includes 1 or more, 2 or more, 3 or more, 4 or more, 5 or more, 10 or more, 15 or more, or 20 or more bases on one or both sides of an SE and/or SRE. In embodiments, the flanking sequence includes 25 or less, 20 or less, 15 or less, 10 or less, 5 or less, 4 or less, 3 or less, or 2 or less bases on one or both sides of an SE and/or SRE. In embodiments, the flanking sequence includes 1 to 25, 1 to 20, 1 to 15, 1 to 10, 1 to 5, 1 to 4, 1 to 3 or 1 to 2 bases on one or both sides of an SE and/or SRE. In embodiments, the flanking sequence includes 2 to 25, 2 to 20, 2 to 15, 2 to 10, 2 to 5, 2 to 4, or 2 to 3 bases on one or both sides of an SE and/or SRE. In embodiments, the flanking sequence includes 3 to 25, 3 to 20, 3 to 15, 3 to 10, 3 to 5, or 3 to 4 bases on one or both sides of an SE and/or SRE. In embodiments, the flanking sequence includes 4 to 25, 4 to 20, 4 to 15, 4 to 10, or 4 to 5 bases on one or both sides of an SE and/or SRE. In embodiments, the flanking sequence includes 5 to 25, 5 to 20, 5 to 15, or 5 to 10 bases on one or both sides of an SE and/or SRE. In embodiments, the flanking sequence includes 10 to 25, 10 to 20, or 10 to 15 bases on one or both sides of an SE and/or SRE. In embodiments, the flanking sequence includes 15 to 25 or 15 to 20 bases on one or both sides of an SE and/or SRE In embodiments, the flanking sequence includes 20 to 25 bases on one or both sides of an SE and/or SRE. In embodiments, the flanking sequence includes an intervening sequence or a portion thereof.

In embodiments, the AC hybridizes with a target nucleotide sequence that includes at least a portion of a 5′ ss of a target transcript. In embodiments, the AC hybridizes with a target nucleotide sequence that includes at least a portion of the exon/intron junction of a target transcript. In embodiments, the AC hybridizes with a target nucleotide sequence that includes at least a portion of a 3′ ss of a target transcript. In embodiments, the AC hybridizes with a target nucleotide sequence that includes at least a portion of a Py tract, BPS, terminal “AG,” and/or the intron/exon junction of the target transcript.

In embodiments, the AC hybridizes with target nucleotide sequence that includes at least a portion of a splice regulatory element (SRE) of target transcript. In embodiments, the AC hybridizes with a target nucleotide sequence that includes an entire SRE of a target transcript. In embodiments, the AC hybridizes with a target nucleotide sequence that includes multiple SREs of a target transcript. In embodiments, the AC hybridizes with a target nucleotide sequence that includes multiple SREs of a target transcript and the intervening sequences between the SREs of a target transcript. In embodiments, the AC hybridizes with a target nucleotide sequence that includes at least a portion of an ESE of a target transcript. In embodiments, the AC hybridizes with a target nucleotide sequence that includes at least a portion of an ISE. In embodiments, the AC hybridizes with a target nucleotide sequence that includes at least a portion of an ESS of a target transcript. In embodiments, the AC hybridizes with a target nucleotide sequence that includes at least a portion of an ISS of a target transcript.

In embodiments, the AC hybridizes with a target nucleotide sequence that includes at least a portion of a terminal stem loop (TLS) of a target transcript

In embodiments, the AC hybridizes with at least a portion of an aberrant SE and/or SRE of a target transcript where the aberrant SE and/or SRE resulted from a mutation in the target gene.

In embodiments, the AC hybridizes with a target nucleotide sequence that includes at least a portion of a SE and/or SRE, an exon/intron junction, or an intron/exon junction of a target transcript. In embodiments, the AC hybridizes with a target nucleotide sequence that includes an aberrant fusion junction due to a rearrangement or a deletion of a target transcript. In embodiments, the AC hybridizes with particular exons in alternatively spliced mRNAs of a target transcript.

In embodiments, the AC hybridizes with target nucleotide sequence that includes at least a portion of a splice element (SE) of an IRF-5, GYS1, and/or a DUX4 target transcript. In embodiments, the AC hybridizes with a target nucleotide sequence that includes an entire SE of an RF-5, GYS1, and/or a DUX4 target transcript. In embodiments, the AC hybridizes with a target nucleotide sequence that includes multiple SEs of an IRF-5, GYS1, and/or a DUX4 target transcript. In embodiments, the AC hybridizes with a target nucleotide sequence that includes multiple SEs of a target transcript and the intervening sequences between the SEs of an IRF-5, GYS1, and/or a DUX4 target transcript. In embodiments, the AC hybridizes with at least a portion of an SE and a one or more flanking sequences of the SE of an R-5, GYS1, and/or a DUX4 target transcript.

In embodiments, the AC hybridizes with a target nucleotide sequence that includes at least a portion of a 5′ ss of an IRF-5 target transcript. In embodiments, the AC hybridizes with a target nucleotide sequence that includes at least a portion of the exon/intron junction of an IRF-5 target transcript. In embodiments, the AC hybridizes with a target nucleotide sequence that includes at least a portion of a 3′ ss of an IRF-5 target transcript. In embodiments, the AC hybridizes with a target nucleotide sequence that includes at least a portion of a Py tract, BPS, terminal “AG,” and/or the intron/exon junction of an IRF-5 target transcript.

In embodiments, the AC binds to a target nucleotide sequence that does not include at least a portion of an SE or at least a portion of an SRE of a target transcript. In embodiments, the AC binds to a target nucleotide sequence that is in sufficiently close proximity to an SE and/or an SRE to modulate splicing of the target transcript. In embodiments, the AC that binds to a target nucleotide sequence that does not include at least a portion of an SE or at least a portion of an SRE of a target transcript and modulates splicing of the target transcript may bind the target transcript and sterically block bind of a translation factor or trans-acting regulatory factor to the SE or SRE.

In embodiments, the AC binds to a target nucleotide sequence with a 3′ end and/or 5′ end that is 1 or more, 2 or more, 3 or more, 4 or more, 5 or more, 10 or more, 15 or more, or 20 or more nucleotides from the 5′ end and/or 3′ end of a SE and/or and SRE of a target transcript. In embodiments, the AC binds to a target nucleotide sequence with a 3′ end and/or 5′ end that is 25 or less, 20 or less, 15 or less, 10 or less, 5 or less, 4 or less, 3 or less, or 2 or less nucleotides from the 5′ end and/or 3′ end of a SE and/or and SRE of a target transcript. In embodiments, the AC binds to a target nucleotide sequence with a 3′ end and/or 5′ end that is 1 to 25, 1 to 20, 1 to 15, 1 to 10, 1 to 5, 1 to 4, 1 to 3, or 1 to 2 nucleotides form the 5′ end and/or 3′ end of a SE and/or and SRE of a target transcript. In embodiments, the AC binds to a target nucleotide sequence with a 3′ end and/or 5′ end that is 2 to 25, 2 to 20, 2 to 15, 2 to 10, 2 to 5, 2 to 4, or 2 to 3 nucleotides form the 5′ end and/or 3′ end of a SE and/or and SRE of a target transcript. In embodiments, the AC binds to a target nucleotide sequence with a 3′ end and/or 5′ end that is 3 to 25, 3 to 20, 3 to 15, 3 to 10, 3 to 5, or 3 to 4 nucleotides form the 5′ end and/or 3′ end of a SE and/or and SRE of a target transcript. In embodiments, the AC binds to a target nucleotide sequence with a 3′ end and/or 5′ end that is 4 to 25, 4 to 20, 4 to 15, 4 to 10, or 4 to 5 nucleotides form the 5′ end and/or 3′ end of a SE and/or and SRE of a target transcript. In embodiments, the AC binds to a target nucleotide sequence with a 3′ end and/or 5′ end that is 5 to 25, 5 to 20, 5 to 15, or 5 to 10 nucleotides form the 5′ end and/or 3′ end of a SE and/or and SRE of a target transcript. In embodiments, the AC binds to a target nucleotide sequence with a 3′ end and/or 5′ end that is 10 to 25 or 10 to 20 nucleotides form the 5′ end and/or 3′ end of a SE and/or and SRE of a target transcript. In embodiments, the AC binds to a target nucleotide sequence with a 3′ end and/or 5′ end that is 20 to 25 nucleotides form the 5′ end and/or 3′ end of a SE and/or and SRE of a target transcript.

In embodiments, the AC hybridizes with a target nucleotide sequence that is from about 5 to about 50 nucleic acids in length. In embodiments, the AC is the same length as the target nucleotide sequence. In embodiments, the AC is a different length than the target nucleotide sequence. In embodiments, the AC is longer than the target nucleic acid sequence.

In embodiments, the AC is 5 or more, 10 or more, 15 or more, 20 or more, 25 or more, 30 or more, 35 or more, 40 or more, or 45 or more nucleic acids in length. In embodiments, the AC is 50 or less, 45 or less, 40 or less, 35 or less, 30 or less, 25 or less, 20 or less, 15 or less, or 10 or less nucleic acids in length. In embodiments, the AC is 5 to 50, 5 to 45, 5 to 40, 5 to 35, 5 to 30, 5 to 25, 5 to 20, 5 to 15, or 5 to 10 nucleic acids in length. In embodiments, the AC is 10 to 50, 10 to 45, 10 to 40, 10 to 35, 10 to 30, 10 to 25, 10 to 20, or 10 to 15 nucleic acids in length. In embodiments, the AC is 15 to 50, 15 to 45, 15 to 40, 15 to 35, 15 to 30, 15 to 25, or 15 to 20 nucleic acids in length. In embodiments, the AC is 20 to 50, 20 to 45, 20 to 40, 20 to 35, 20 to 30, or 20 to 25 nucleic acids in length. In embodiments, the AC is 25 to 50, 25 to 45, 25 to 40, 25 to 35, or 25 to 30 nucleic acids in length. In embodiments, the AC is 30 to 50, 30 to 45, 30 to 40, or 30 to 35 nucleic acids in length. In embodiments, the AC is 35 to 50, 35 to 45, or 35 to 40 nucleic acids in length. In embodiments, the AC is 40 to 50 or 40 to 45 nucleic acids in length. In embodiments, the AC is 45 to 50 nucleic acids in length. In embodiments, the AC is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleic acids in length.

In embodiments, the AC has 100% complementarity to a target nucleotide sequence. In embodiments, the AC does not have 100% complementarity to a target nucleotide sequence. As used herein, the term “percent complementarity” refers to the number of nucleobases of an AC that have nucleobase complementarity with a corresponding nucleobase of an oligomeric compound or nucleic acid (e.g., a target nucleotide sequence) divided by the total length (number of nucleobases) of the AC. One skilled in the art recognizes that the inclusion of mismatches is possible without eliminating the activity of the antisense compound.

In embodiments, the AC includes 20% or less, 15% or less, 10% or less, 5% or less, or zero mismatches to the target nucleotide sequence. In some embodiments, the AC includes 5% or more, 10% or more, or 15% or more mismatches to the target nucleotide sequence. In embodiments, the AC includes zero to 5%, zero to 10%, zero to 15%, or zero to 20% mismatches to the target nucleotide sequence. In embodiments, the AC includes 5% to 10%, 5% to 15%, or 5% to 20% mismatches to the target nucleotide sequence. In embodiments, the AC includes 10% to 15% or 10% to 20% mismatches to the target nucleotide sequence. In embodiments, the AC includes 10% to 20% mismatches to the target nucleotide sequence.

In embodiments, the AC has 80% or greater, 85% or greater, 90% or greater, 95% or greater, 96% or greater, 97% or greater, 98% or greater, or 99% or greater complementarity to a target nucleotide sequence. In embodiments, the AC has 100% or less, 99% or less, 98% or less, 97% or less 96% or less 95% or less, 90% or less, 85% or less complementarity to a target nucleotide sequence. In embodiments, the AC has 80% to 100%, 80% to 99%, 80% to 98%, 80% to 97% 80% to 96%, 80% to 95%, 80% to 90% or 80% to 85% complementarity to a target nucleotide sequence. In embodiments, the AC has 85% to 100%, 85% to 99%, 85% to 98%, 85% to 97% 85% to 96%, 85% to 95%, or 85% to 90% complementarity to a target nucleotide sequence. In embodiments, the AC has 90% to 100%, 90% to 99%, 90% to 98%, 90% to 97%, 90% to 96%, or 90% to 95% complementarity to a target nucleotide sequence. In embodiments, the AC has 95% to 100%, 95% to 99%, 95% to 98%, 95% to 97%, or 95% to 96% complementarity to a target nucleotide sequence. In embodiments, the AC has 96% to 100%, 96% to 99%, 96% to 98%, or 96% to 97% complementarity to a target nucleotide sequence. In embodiments, the AC has 97% to 100%, 97% to 99%, or 97% to 98% complementarity to a target nucleotide sequence. In embodiments, the AC has 98% to 100% or 98% to 99% complementarity to a target nucleotide sequence. In embodiments, the AC has 99% to 100% complementarity to a target nucleotide sequence. Percent complementarity of an oligonucleotide is calculated by dividing the number of complementarity nucleobases by the total number of nucleobases of the oligonucleotide.

In embodiments, the AC includes 1, 2, 3, 4, or 5 mismatches relative to the target nucleic acid sequence to which the AC hybridizes. In embodiments, the AC includes 1 or 2 mismatches relative to the target nucleic acid sequence to which the AC hybridizes. In embodiments, the AC includes no mismatches relative to the target nucleic acid sequence to which the AC hybridizes.

In embodiments, incorporation of nucleotide affinity modifications allows for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between an AC and a target nucleotide sequence, such as by determining the thermal melting temperature (Tm). Tm or ΔTm can be calculated by techniques that are familiar to one of ordinary skill in the art. For example, techniques described in Freier et al. (Nucleic Acids Research (1997), 25, 22: 4429-4443) allow one of ordinary skill in the art to evaluate nucleotide modifications for their ability to increase the melting temperature of an RNA:DNA duplex.

In embodiments, the AC includes a sequence that hybridizes to the target transcript under stringent conditions and includes a sequence that that does not hybridize to the target transcript under stringent conditions. In embodiments, the AC includes a first sequence that does not hybridize to the target sequence under stringent conditions, a second sequence that does not hybridize to the target sequence under stringent conditions, and a third sequence that does hybridize to the target sequence under stringent conditions, where the third sequence is positioned between the first and the second sequence.

In embodiments, the AC hybridizes with target nucleotide sequence that includes at least a portion of a splice regulatory element (SRE) of an IRF-5, GYS1, and/or a DUX4 target transcript. In embodiments, the AC hybridizes with a target nucleotide sequence that includes an entire SRI of an IRF-5, GYS1, and/or a DUX4 target transcript. In embodiments, the AC hybridizes with a target nucleotide sequence that includes multiple SREs of an IRF-5, GYS1, and/or a DUX4 target transcript. In embodiments, the AC hybridizes with a target nucleotide sequence that includes multiple SREs of an IRF-5, GYS1, and/or a DU X4 target transcript and the intervening sequences between the SREs. In embodiments, the AC hybridizes with at least a portion of an SE and a one or more flanking sequences of the SE of an IRF-5, GYS1, and/or a DUX4 target transcript.

In embodiments, the AC hybridizes with a target nucleotide sequence that includes at least a portion of an ESE of an IRF-5, GYS1, and/or a DUX4 target transcript. In embodiments, the AC hybridizes with a target nucleotide sequence that includes at least a portion of an ISE of an IRF-5, GYS1, and/or a DUX4 target transcript. In embodiments, the AC hybridizes with a target nucleotide sequence that includes at least a portion of an ESS of an IRF-5, GYS1, and/or a DUX4 target transcript. In embodiments, the AC hybridizes with a target nucleotide sequence that includes at least a portion of an ISS of an IRF-5, GYS1, and/or a DUX4 target transcript.

In embodiments, the AC hybridizes with a target nucleotide sequence that includes at least a portion of a terminal stem loop (TS) of an IRF-5, GYS1, and/or a DUX4 target transcript.

In embodiments, the AC hybridizes with at least a portion of an aberrant SE and/or SRE of an IRF-5, GYS1, and/or a DUX4 target transcript where the aberrant SE and/or SRE resulted from a mutation in the IRF-5, GYS1, and/or a DUX4 target transcript.

In embodiments, the AC hybridizes with a target nucleotide sequence that includes at least a portion of an exon-exon junction, intron-exon junction, and/or exon-intron junction an IRF-5. GYS1, and/or a DUX4 target transcript. In embodiments, the AC hybridizes with a target nucleotide sequence that includes an aberrant fusion junction due to a rearrangement or a deletion of a portion of an IRF-5, GYS1, and/or a DUX4 target transcript target transcript. In embodiments, the AC hybridizes with particular exons in alternatively spliced mRNAs in an IRF-5, GYS1, and/or a DUX4 target transcript.

In embodiments, the AC binds to a target nucleotide sequence that does not include at least a portion of an SE or at least a portion of an SRE of an ISS of an IRF-5, GYS1, and/or a DUX4 target transcript. In embodiments, the AC binds to a target nucleotide sequence that is in sufficiently close proximity to an SE and/or an SRE to modulate splicing of an ISS of an IRF-5, GYS1, and/or a DUX4 target transcript.

In embodiments, the AC binds to a target nucleotide sequence with a 3′ end and/or 5′ end that is 1 or more, 2 or more, 3 or more, 4 or more, 5 or more, 10 or more, 15 or more, or 20 or more nucleotides from the 5′ end and/or 3′ end of a SE and/or and SRE of an IRF-5, GYS1, and/or a DUX4 target transcript. In embodiments, the AC binds to a target nucleotide sequence with a 3′ end and/or 5′ end that is 25 or less, 20 or less, 15 or less, 10 or less, 5 or less, 4 or less, 3 or less, or 2 or less nucleotides from the 5′ end and/or 3′ end of a SE and/or and SRE of an IRF-5, GYS1, and/or a DUX4 target transcript. In embodiments, the AC binds to a target nucleotide sequence with a 3′ end and/or 5′ end that is 1 to 25, 1 to 20, 1 to 15, 1 to 10, 1 to 5, 1 to 4, 1 to 3, or 1 to 2 nucleotides form the 5′ end and/or 3′ end of a SE and/or and SRE of an IRF-5, GYS1, and/or a DUX4 target transcript. In embodiments, the AC binds to a target nucleotide sequence with a 3′ end and/or 5′ end that is 2 to 25, 2 to 20, 2 to 15, 2 to 10, 2 to 5, 2 to 4, or 2 to 3 nucleotides form the 5′ end and/or 3′ end of a SE and/or and SRE of an IRF-5, GYS1, and/or a DUX4 target transcript. In embodiments, the AC binds to a target nucleotide sequence with a 3′ end and/or 5′ end that is 3 to 25, 3 to 20, 3 to 15, 3 to 10, 3 to 5, or 3 to 4 nucleotides form the 5′ end and/or 3′ end of a SE and/or and SRE of an IRF-5, GYS1, and/or a DUX4 target transcript. In embodiments, the AC binds to a target nucleotide sequence with a 3′ end and/or 5′ end that is 4 to 25, 4 to 20, 4 to 15, 4 to 10, or 4 to 5 nucleotides form the 5′ end and/or 3′ end of a SE and/or and SRE of an IRF-5, GYS1, and/or a DUX4 target transcript. In embodiments, the AC binds to a target nucleotide sequence with a 3′ end and/or 5′ end that is 5 to 25, 5 to 20, 5 to 15, or 5 to 10 nucleotides form the 5′ end and/or 3′ end of a SE and/or and SRE of an IRF-5, GYS1, and/or a DUX4 target transcript. In embodiments, the AC binds to a target nucleotide sequence with a 3′ end and/or 5′ end that is 10 to 25 or 10 to 20 nucleotides form the 5′ end and/or 3′ end of a SE and/or and SRE of an IRF-5, GYS1, and/or a DUX4 target transcript. In embodiments, the AC binds to a target nucleotide sequence with a 3′ end and/or 5′ end that is 20 to 25 nucleotides form the 5′ end and/or 3′ end of a SE and/or and SRE of an IRF-5, GYS1, and/or a DUX4 target transcript.

In embodiments, the AC hybridizes with a target nucleotide sequence of an IRF-5, GYS1, and/or a DUX4 target transcript that is from about 5 to about 50 nucleic acids in length. In embodiments, the AC is the same length as the target nucleotide sequence. In embodiments, the AC is a different length than the target nucleotide sequence. In embodiments, the AC is longer than the target nucleic acid sequence.

In embodiments, the AC has 100% complementarity to a target nucleotide sequence of an IRF-5, GYS1, and/or a DUX4 target transcript. In embodiments, the AC does not have 100% complementarity to a target nucleotide sequence. As used herein, the term “percent complementarity” refers to the number of nucleobases of an AC that have nucleobase complementarity with a corresponding nucleobase of an oligomeric compound or nucleic acid (e.g., a target nucleotide sequence) divided by the total length (number of nucleobases) of the AC. One skilled in the art recognizes that the inclusion of mismatches is possible without eliminating the activity of the antisense compound.

In embodiments, the AC includes 20% or less, 15% or less, 10% or less, 5% or less, or zero mismatches to the target nucleotide sequence of an IRF-5, GYS1, and/or a DUX4 target transcript. In some embodiments, the AC includes 5% or more, 10% or more, or 15% or more mismatches to the target nucleotide sequence of an IRF-5, GYS1, and/or a DUX4 target transcript. In embodiments, the AC includes zero to 5%, zero to 10%, zero to 15%, or zero to 20% mismatches to the target nucleotide sequence of an IRE-5, GYS1, and/or a DUX4 target transcript. In embodiments, the AC includes 5% to 10%, 5% to 15%, or 5% to 20% mismatches to the target nucleotide sequence of an IRF-5, GYS1, and/or a DUX4 target transcript. In embodiments, the AC includes 10% to 15% or 10% to 20% mismatches to the target nucleotide sequence of an IRK-5, GYS1, and/or a DUX4 target transcript. In embodiments, the AC includes 10% to 20% mismatches to the target nucleotide sequence of an IRF-5, GYS1, and/or a DUX4 target transcript.

In embodiments, the AC has 80% or greater, 85% or greater, 90% or greater, 95% or greater, 96% or greater, 97% or greater, 98% or greater, or 99% or greater complementarity to a target nucleotide sequence of an IRF-5, GYS1, and/or a DUX4 target transcript. In embodiments, the AC has 100% or less, 99% or less, 98% or less, 97% or less 96% or less 95% or less, 90% or less, 85% or less complementarity to a target nucleotide sequence of an IRF-5, GYS1, and/or a DUX4 target transcript. In embodiments, the AC has 80% to 100%, 80% to 99%, 80% to 98%, 80% to 97% 80% to 96%, 80% to 95%, 80% to 90% or 80% to 85% complementarity to a target nucleotide sequence of an IRF-5, GYS1, and/or a DUX4 target transcript. In embodiments, the AC has 85% to 100%, 85% to 99%, 85% to 98%, 85% to 97% 85% to 96%, 85% to 95%, or 85% to 90% complementarity to a target nucleotide sequence of an IRF-5, GYS1, and/or a DUX4 target transcript. In embodiments, the AC has 90% to 100%, 90% to 99%, 90% to 98%, 90% to 97%, 90% to 96%, or 90% to 95% complementarity to a target nucleotide sequence of an IRF-5, CYS1, and/or a DUX4 target transcript. In embodiments, the AC has 95% to 100%, 95% to 99%, 95% to 98%, 95% to 97%, or 95% to 96% complementarity to a target nucleotide sequence of an IRF-5, GYS1, and/or a DUX4 target transcript. In embodiments, the AC has 96% to 100%, 96% to 99%, 96% to 98%, or 96% to 97% complementarity to a target nucleotide sequence of an IRF-5, CYS1, and/or a DUX4 target transcript. In embodiments, the AC has 97% to 100%, 97% to 99%, or 97% to 98% complementarity to a target nucleotide sequence of an IRF-5, GYS1, and/or a DUX4 target transcript. In embodiments, the AC has 98% to 100% or 98% to 99% complementarity to a target nucleotide sequence. In embodiments, the AC has 99% to 100% complementarity to a target nucleotide sequence of an IRF-5, GYS1, and/or a DUX4 target transcript. Percent complementarity of an oligonucleotide is calculated by dividing the number of complementary nucleobases by the total number of nucleobases of the oligonucleotide.

### Antisense Mechanisms

In embodiments, the ACs modulate one or more aspects of protein transcription, translation, and expression. In embodiments, hybridization of the AC to target nucleotide sequence of a target transcript modulates one or more aspects of pre-mRNA splicing. In embodiments, AC hybridization to a target nucleotide sequence of a target transcript restores native splicing to a mutated transcript sequence. In embodiments, AC hybridization to a target nucleotide sequence of a target transcript results in alternative splicing of the target transcript.

In embodiments, AC hybridization results in exon inclusion or exon skipping of one or more exons. In embodiments, exon skipping increases the activity of a protein expressed from the resulting mRNA. In embodiments, exon skipping decreases the activity a protein expressed from the resulting mRNA. In embodiments, skipping one or more exons induces a frameshift in the mRNA transcript. In embodiments, the frameshift results in mRNA that encodes a protein with decreased activity. In embodiments, the frameshift results in a truncated or non-functional protein. In embodiments, skipping one or more exons results in the introduction of a premature termination codon in the mRNA. In embodiments, skipping one or more exons results in degradation of the mRNA transcript by nonsense-mediated decay. In embodiments, the skipped exon sequence includes a nucleic acid deletion, substitution, or insertion. In embodiments, the skipped exon does not include a sequence mutation. In embodiments, antisense oligonucleotide hybridization to a target nucleotide sequence within a target pre-mRNA transcript results in expression of a different protein isoform.

In embodiments, AC hybridization to a target nucleotide sequence of a target transcript prevents inclusion of an intron sequence in the mature mRNA molecule. In embodiments, AC hybridization to a target nucleotide sequence of a target transcript results in increased expression of a protein isoform. In embodiments, AC hybridization to a target nucleotide sequence of a target transcript results in decreased expression of a protein isoform. In embodiments, AC hybridization to a target nucleotide sequence of a target transcript results in expression of a re-spliced protein that includes an inactive fragment of a protein.

In embodiments, the AC includes DNA and hybridization of the AC to the target transcript results in transcript degradation via RNAse H. In embodiments, AC includes a nucleotide modification designed to not support RNase H activity. Nucleotide modifications of antisense compounds that do not support RNase H activity are known and include, but are not limited to, 2′-O-methoxy ethyl/phosphorothioate (MOE) modifications. Advantageously, AC with MOE modifications have increased affinity for target RNA and increase nuclease stability.

In embodiments, the AC regulates transcription, translation, or protein expression through steric blocking. The following review article describes the mechanisms of steric blocking and applications thereof and is incorporated by reference herein in its entirety: Roberts et al., Nature Reviews Drug Discovery (2020) 19: 673-694.

The efficacy of the ACs may be assessed by evaluating the antisense activity effected by their administration. As used herein, the term “antisense activity” refers to any detectable and/or measurable activity attributable to the hybridization of an antisense compound to its target nucleotide sequence. Such detection and/or measuring may be direct or indirect. In embodiments, antisense activity is assessed by detecting and or measuring the amount of the protein expressed from the transcript of interest. In embodiments, antisense activity is assessed by detecting and/or measuring the amount of the transcript of interest. In embodiments, antisense activity is assessed by detecting and/or measuring the amount of alternatively spliced RNA and/or the amount of protein isoforms translated from the target transcript. In embodiments, antisense activity is assessed by detecting and/or measuring the amount of a downstream transcript and/or protein that is regulated by the gene of interest.

### Antisense Compound Design

Design of ACs will depend upon the target gene. Targeting an AC to a particular target nucleotide sequence can be a multistep process. The process usually begins with the identification of gene of interest. The transcript of the gene of interest is analyzed and a target nucleotide sequence is identified. In embodiments, the target nucleotide sequence includes at least a portion of a splice element and/or splice regulatory element. In embodiments, the target gene is IRF-5. In embodiments, the target gene is GYS1. In embodiments, the target gene in DUX4.

One of skill in the art will be able to design, synthesize, and screen ACs of different nucleobase sequences to identify a sequence that results in antisense activity. For example, an antisense compound can be designed that inhibits expression of a target gene. Methods for designing, synthesizing, and screening ACs for antisense activity against a preselected target nucleic acid and/or target gene can be found, for example in “Antisense Drug Technology, Principles, Strategies, and Applications” Edited by Stanley T. Crooke, CRC Press, Boca Raton, Florida, which is incorporated by reference in its entirety for any purpose.

### AC Structure

The AC includes an oligonucleotide and/or an oligonucleoside. Oligonucleotides and/or oligonucleosides are nucleosides linked through internucleoside linkages. Nucleosides include a pentose sugar (e.g., ribose or deoxyribose) and a nitrogenous base covalently attached to sugar. The naturally occurring (traditional) bases found in DNA and/or RNA are adenine (A), guanine (G), thymine (T), cytosine (C), and uracil (U). The naturally occurring (traditional) sugars found in DNA and/or RNA deoxyribose (DNA) and ribose (RNA). The naturally occurring (traditional) nucleoside linkage is a phosphodiester bond. In embodiments, the ACs of the present disclosure may have all natural sugars, bases, and internucleoside linkages.

Chemically modified nucleosides are routinely used for incorporation into antisense compounds to enhance one or more properties, such as nuclease resistance, pharmacokinetics or affinity for a target RNA. In embodiments, the ACs of the present disclosure may have one or more modified nucleosides. In embodiments, the ACs of the present disclosure may have one or more modified sugars. In embodiments, the ACs of the present disclosure may have one or more modified bases. In embodiments, the ACs of the present disclosure may have one or more modified internucleoside linkages.

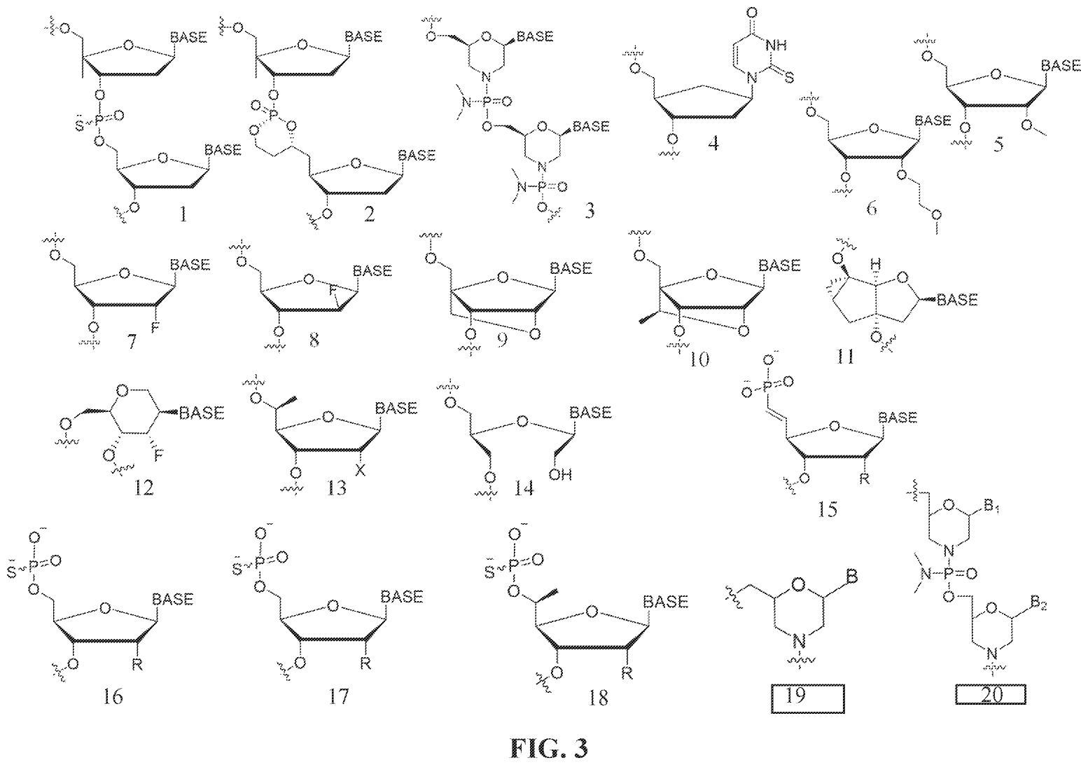

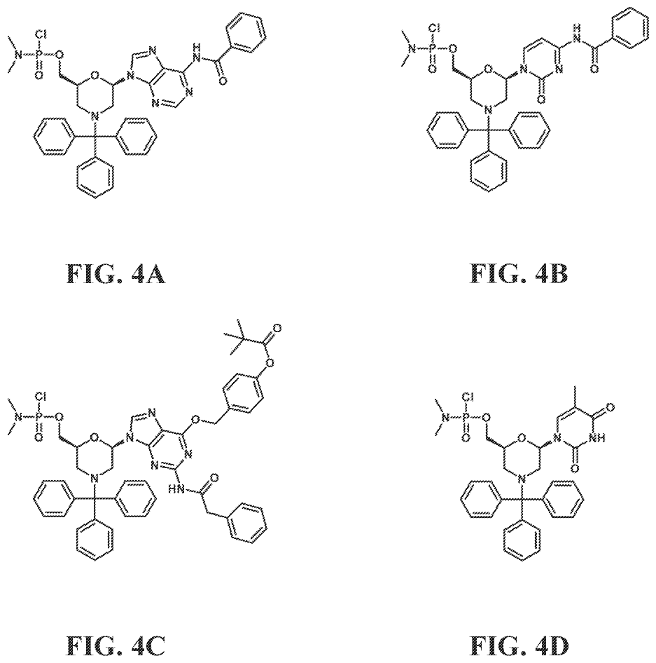

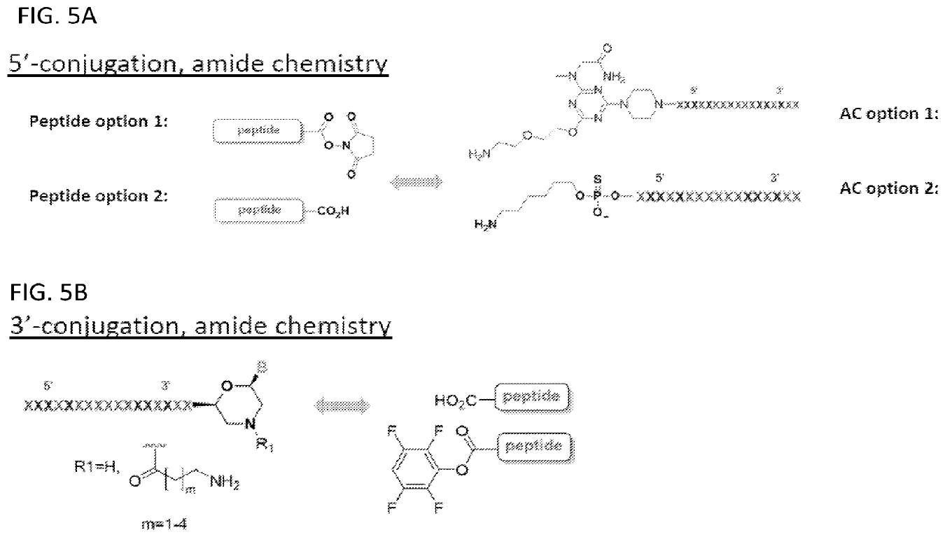

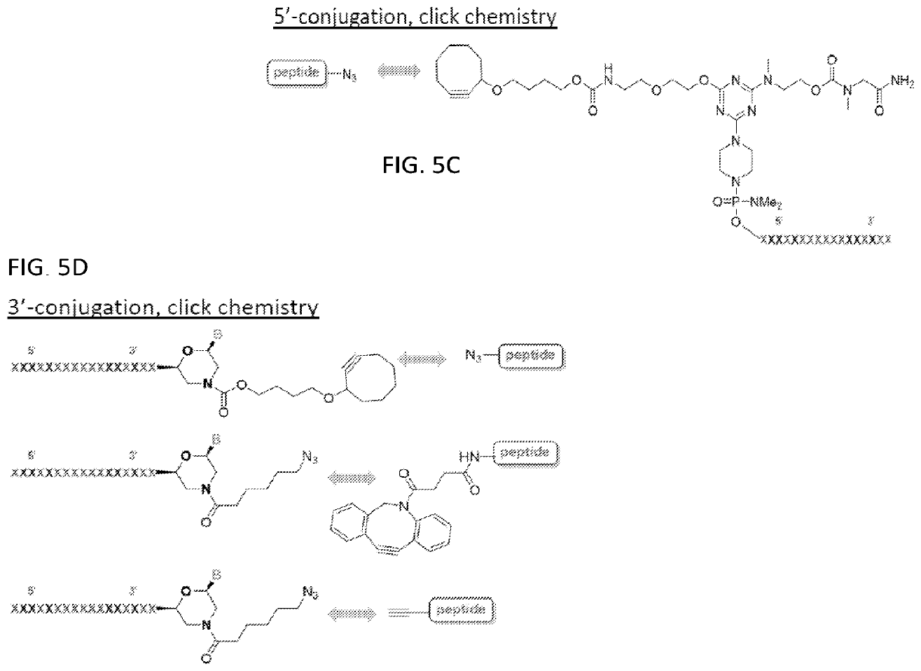

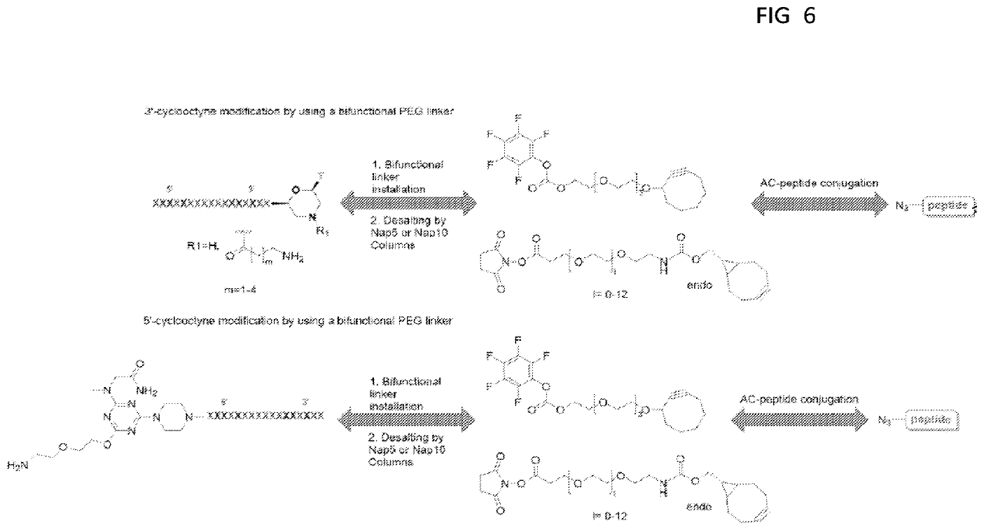

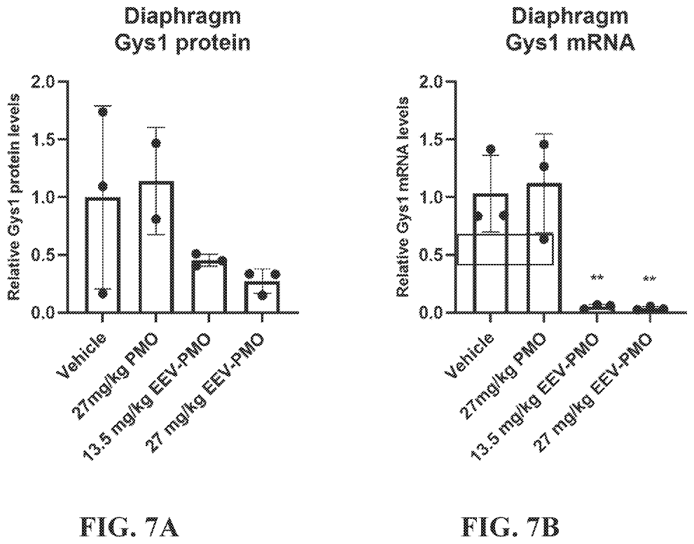

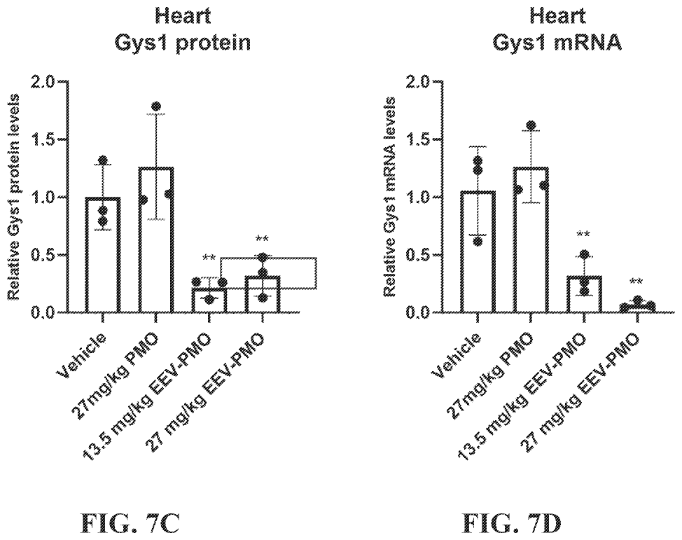

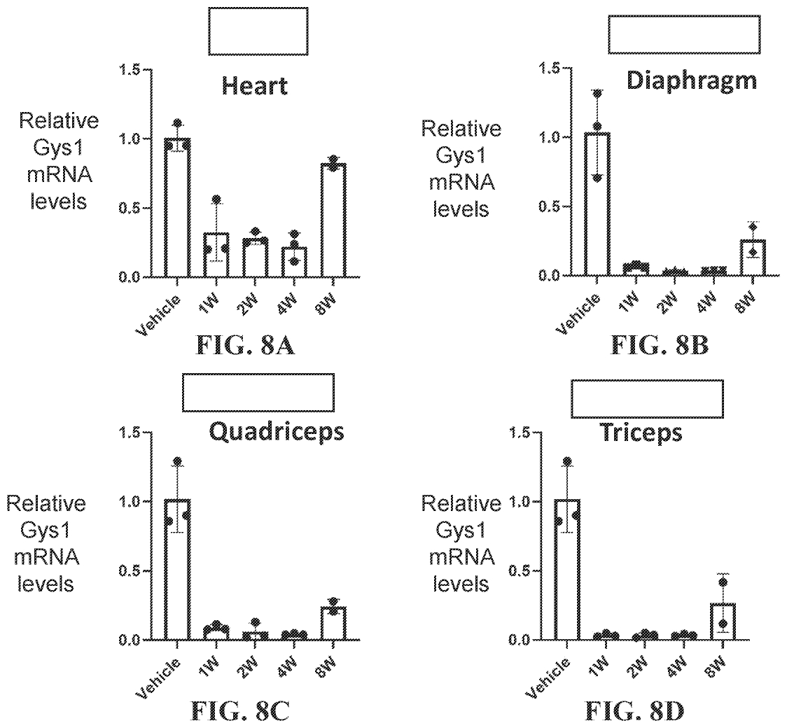

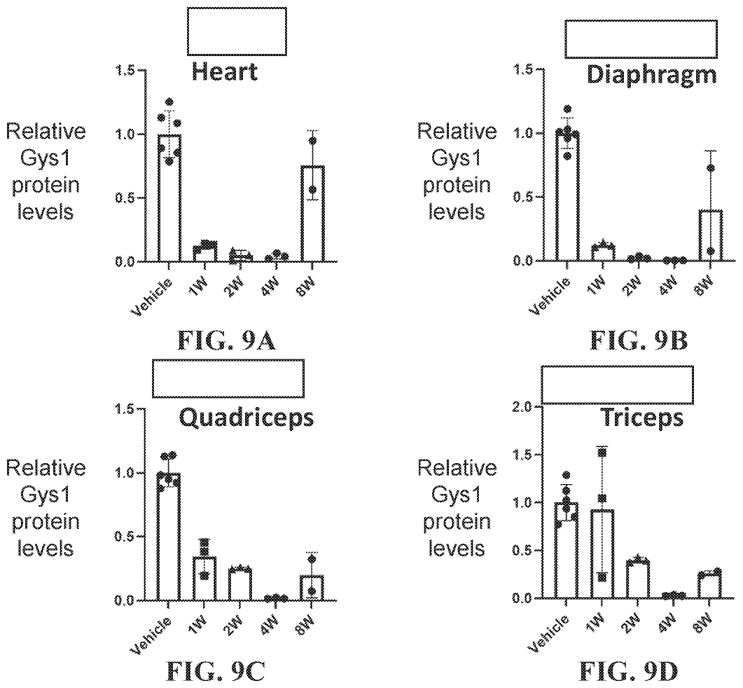

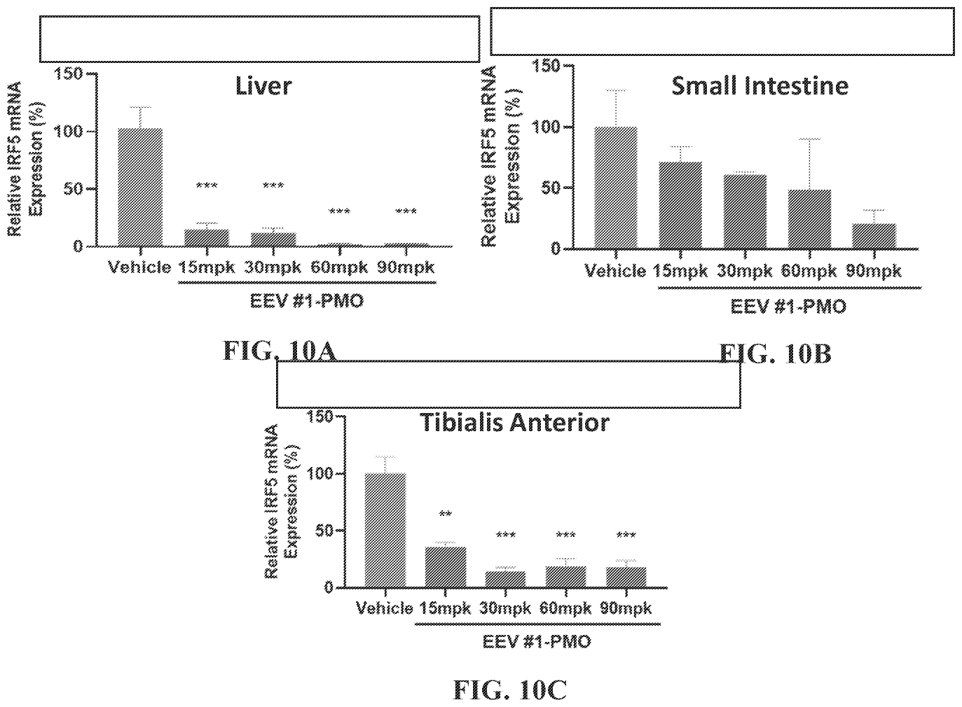

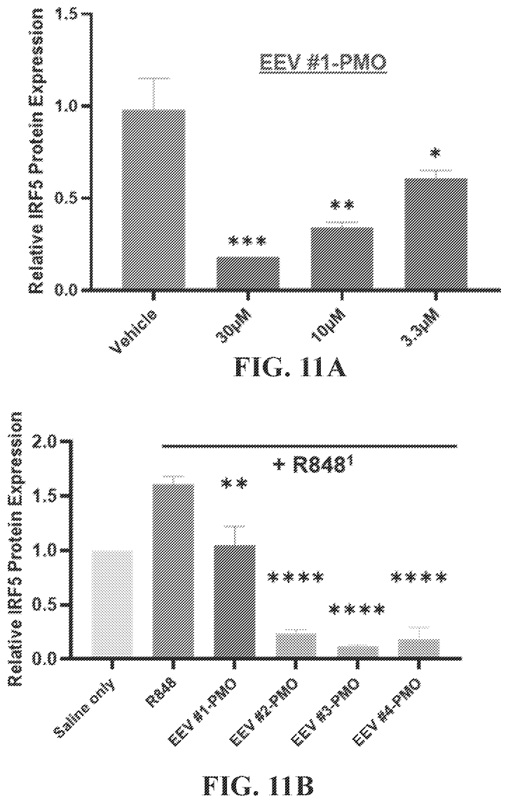

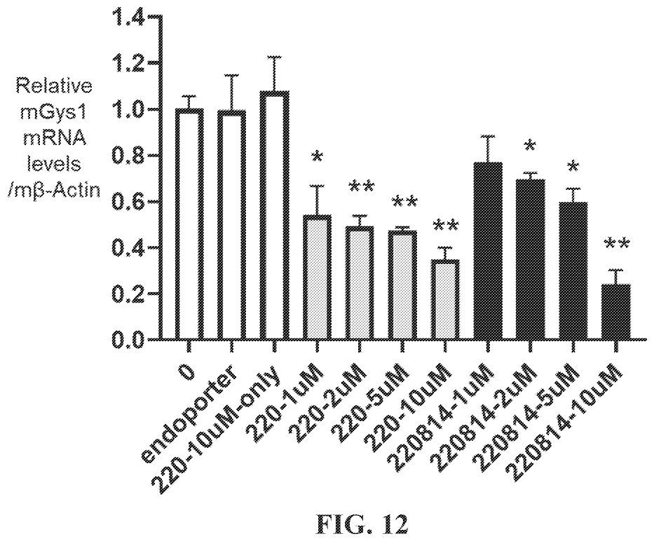

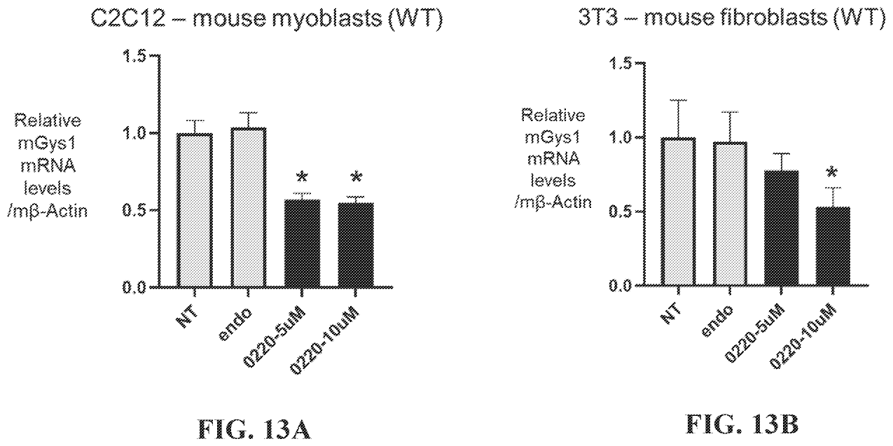

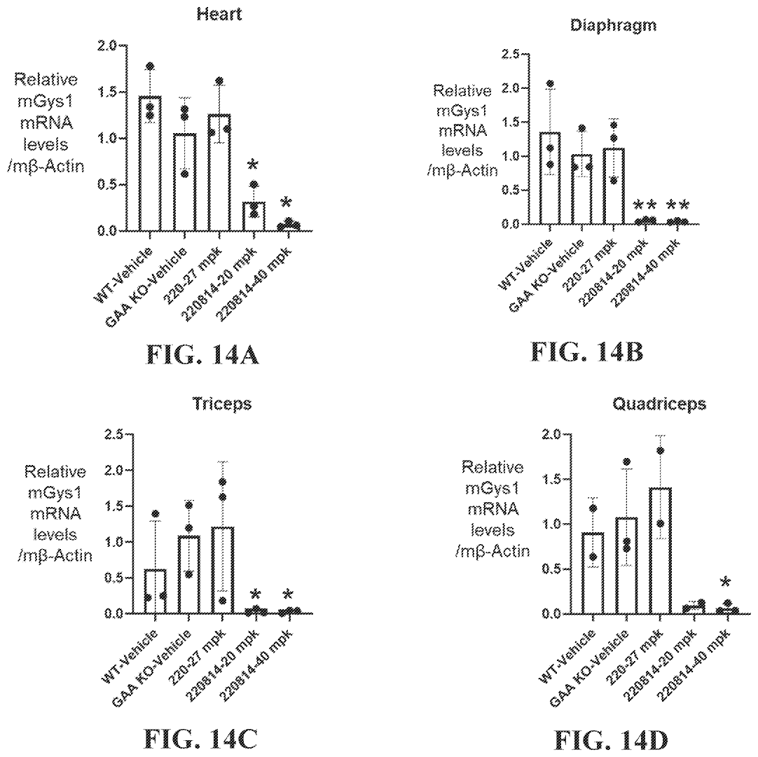

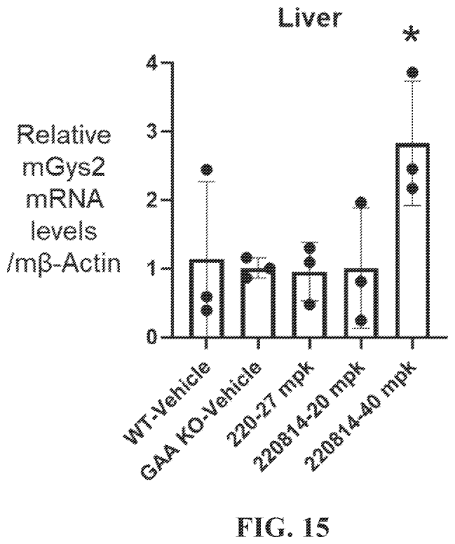

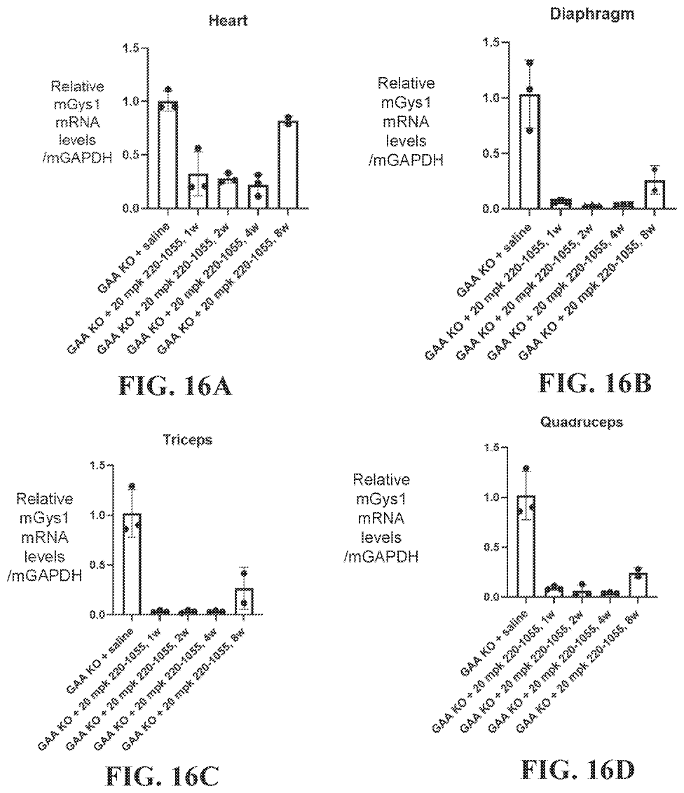

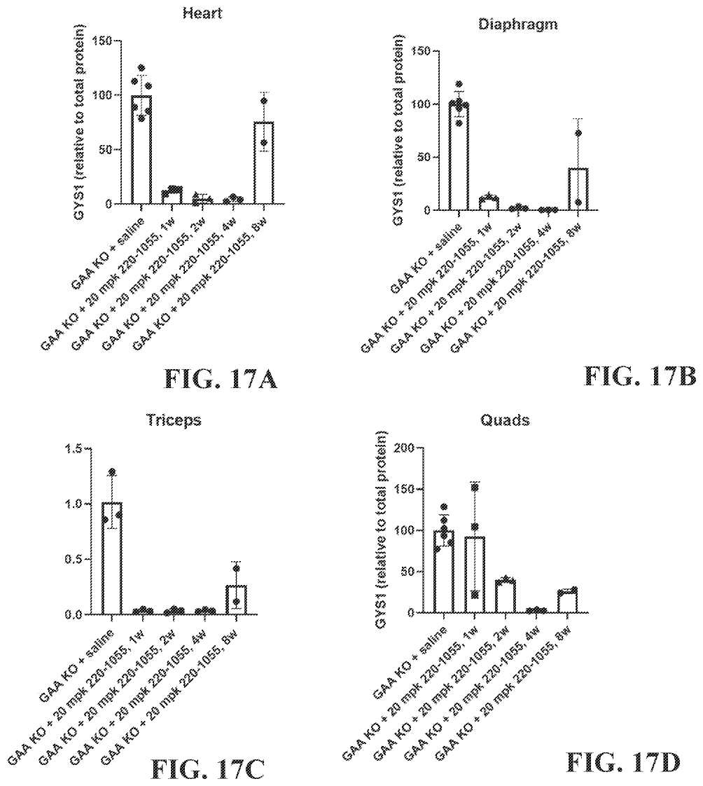

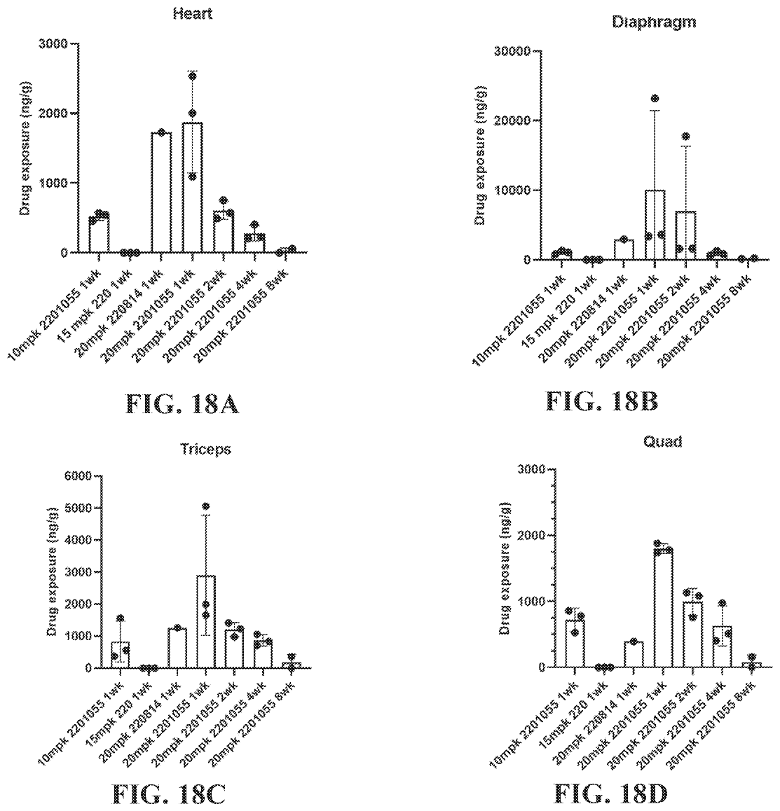

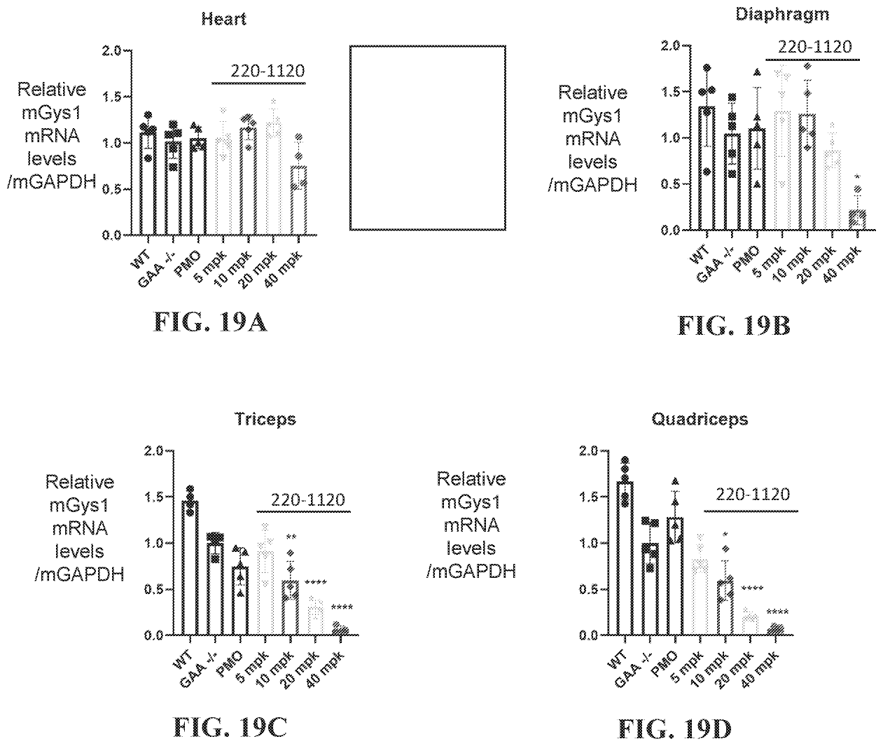

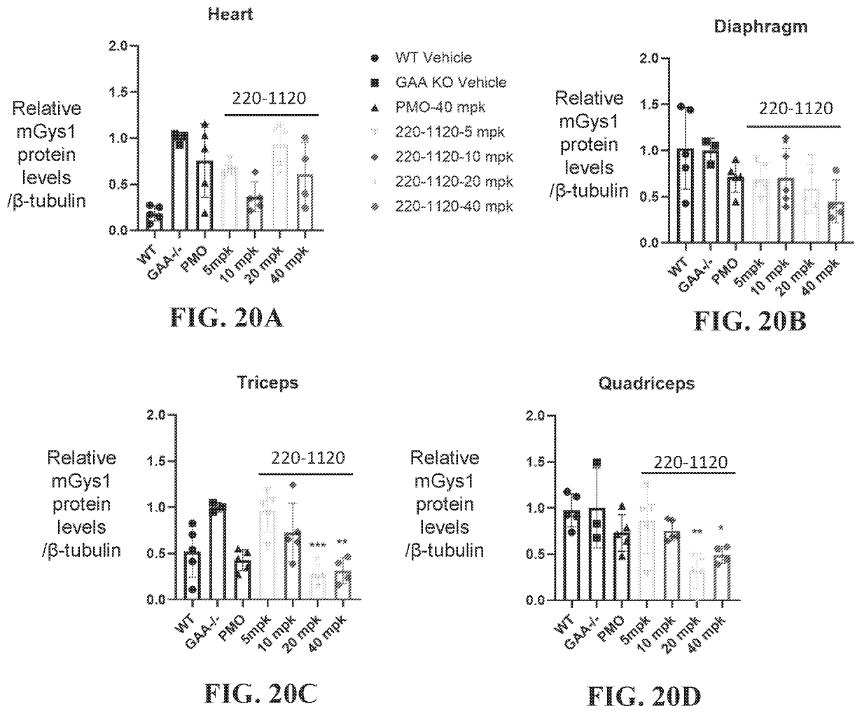

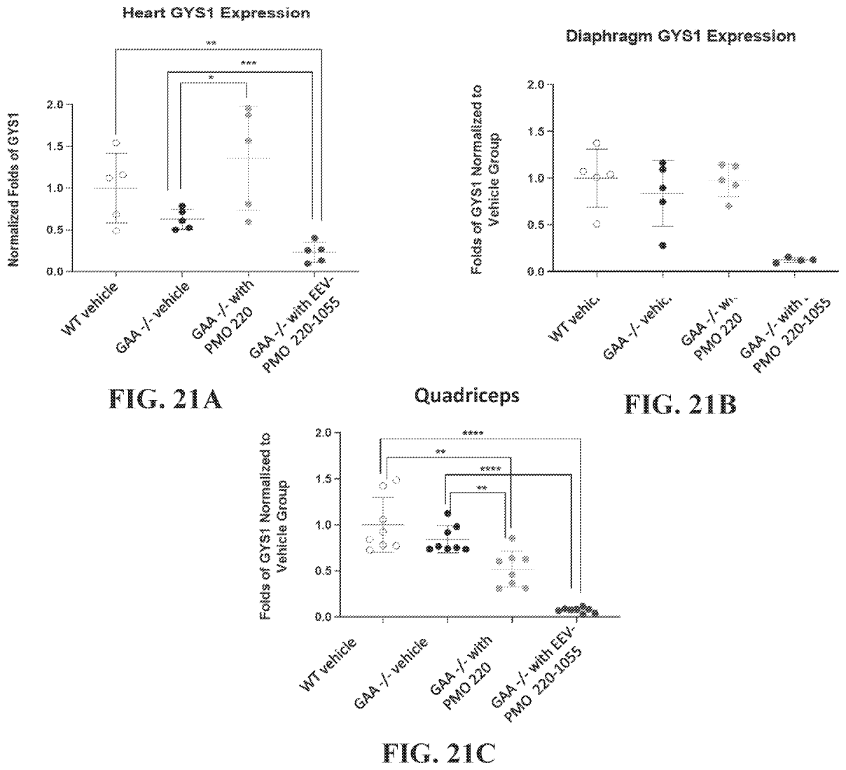

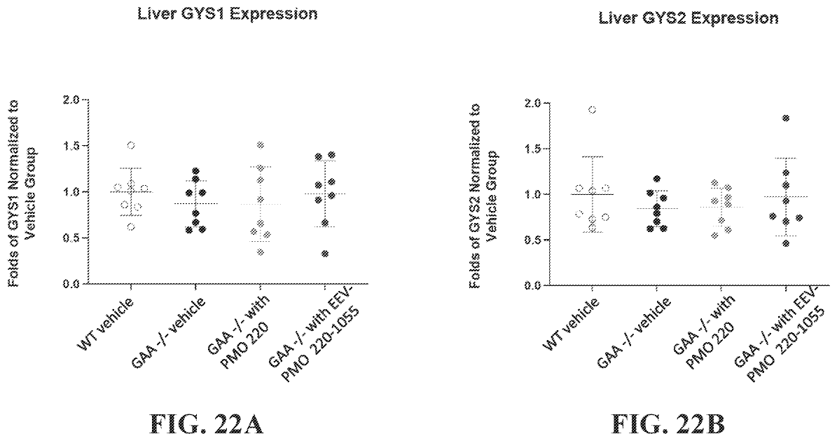

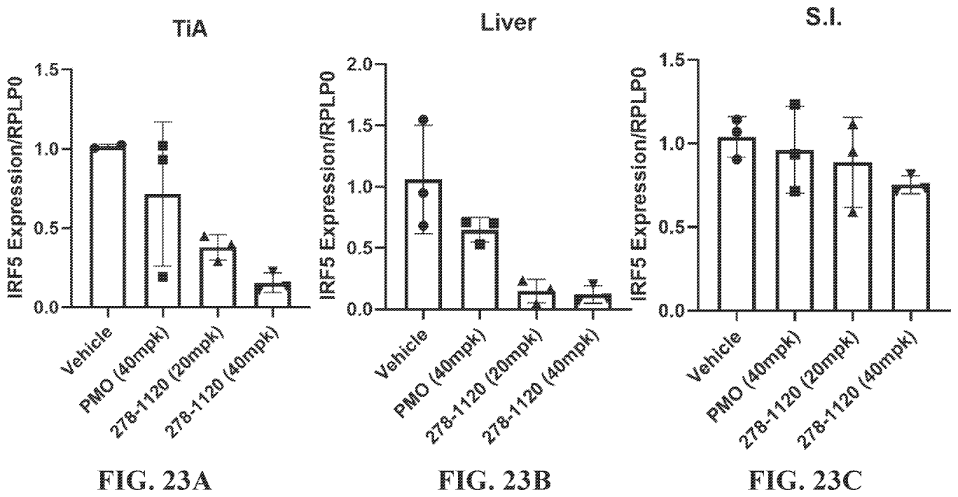

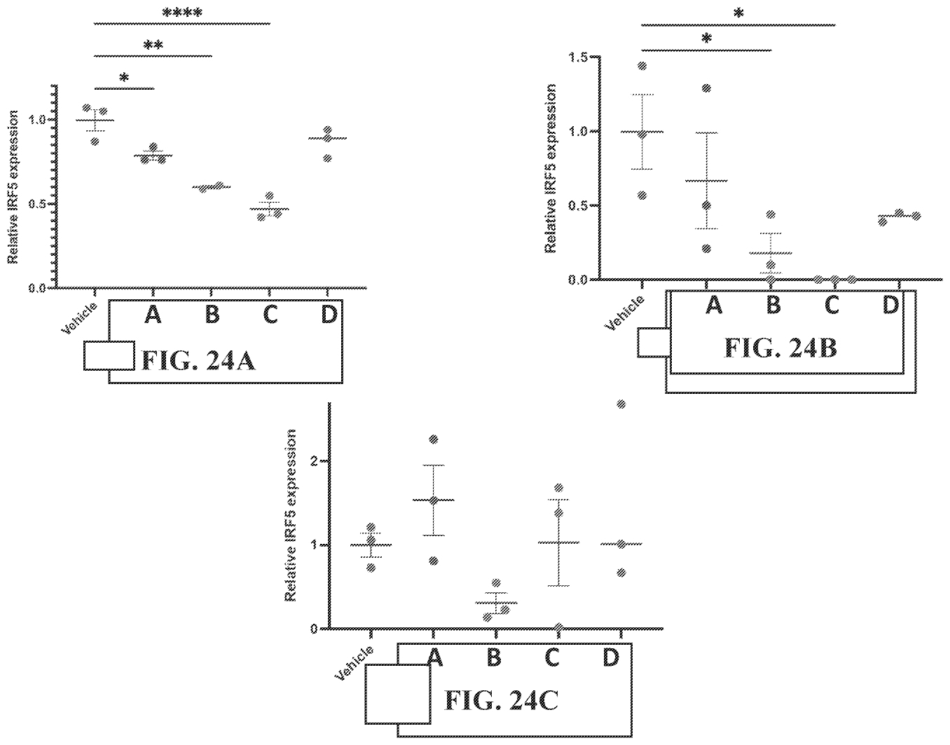

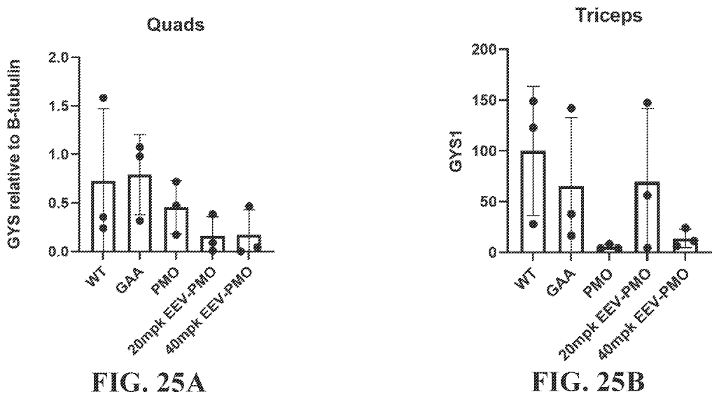

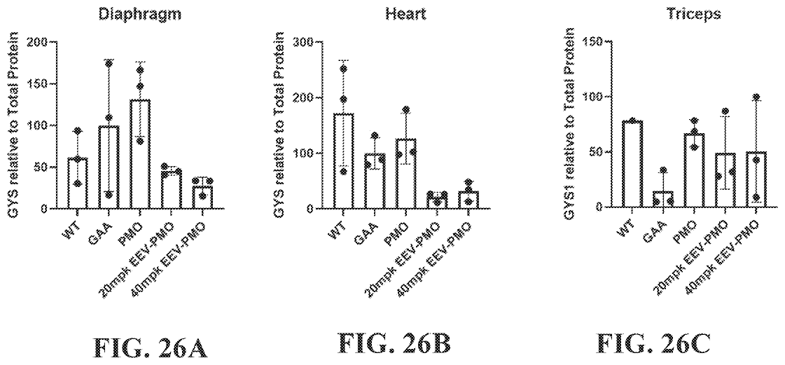

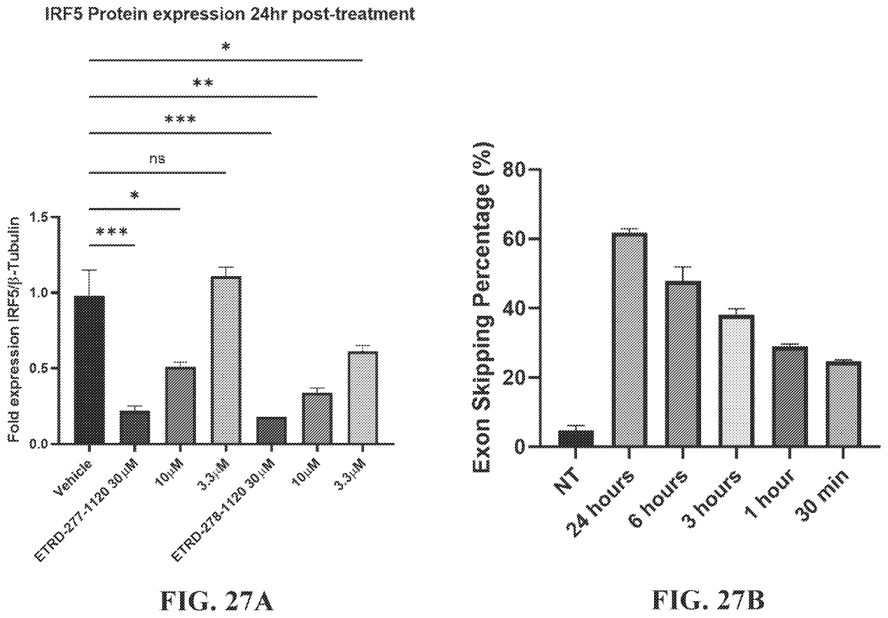

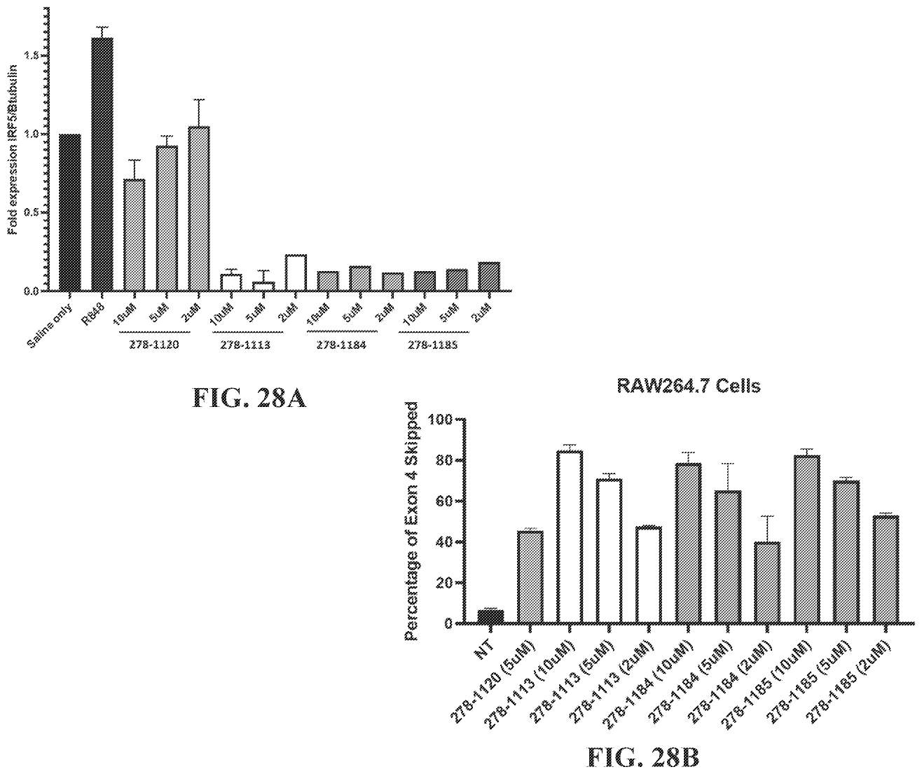

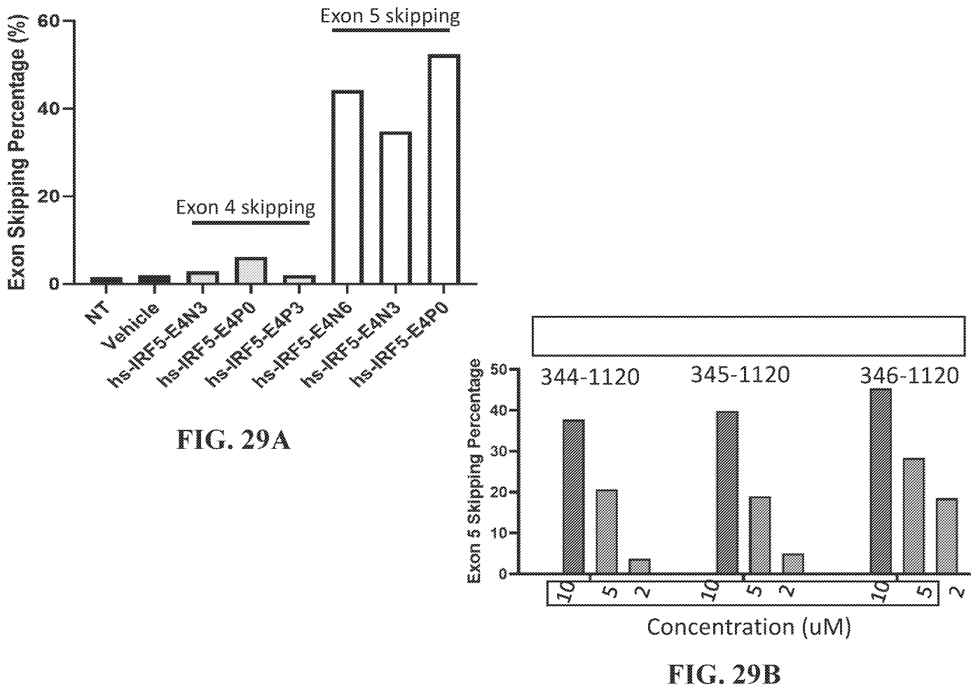

In general, a nucleobase is any group that contains one or more atom or groups of atoms capable of hydrogen bonding to a base of another nucleic acid. In addition to “unmodified” or “natural” nucleobases (A, G, T, C, and U) many modified nucleobases or nucleobase mimetics are known to those skilled in the art are amenable with the compounds described herein Generally a modified nucleobase refers to a nucleobase that is fairly similar in structure to the parent nucleobase, such as for example a 7-deaza purine, a 5-methyl cytosine, 2-thio-dT (FIG. 3) or a G-clamp. Generally, a nucleobase mimetic is a nucleobase that includes a structure that is more complicated than a modified nucleobase, such as for example a tricyclic phenoxazine nucleobase mimetic. Methods for preparation of the above noted modified nucleobases are well known to those skilled in the art.

In embodiments, the AC may include one or more nucleosides having a modified sugar moiety. In embodiments, the furanosyl sugar of a natural nucleoside may have a 2′ modification, modifications to make a constrained nucleoside, and others (see FIG. 3). For example, in embodiments, the furanosyl sugar ring of a natural nucleoside can be modified in a number of ways including, but not limited to, addition of a substituent group, bridging of two non-geminal ring atoms to form a bicyclic nucleic acid (BNA) or a locked nucleic acid; exchanging the oxygen of the furanosyl ring with C or N; and/or substitution of an atom or group such (see FIG. 3). Modified sugars are well known and can be used to increase or decrease the affinity of the AC for its target nucleotide sequence. Modified sugars may also be used increase AC resistance to nucleases. Sugars can also be replaced with sugar mimetic groups among others. In embodiments, one or more sugars of the nucleosides of the AC is replaced with a methylenemorpholine ring as shown as 19 in FIG. 3.

In embodiments, the AC includes one or more nucleosides that include a bicyclic modified sugar (BNA; sometimes called bridged nucleic acids). Examples of BNAs include, but are not limited to LNA (4′-(CH₂)—O-2′ bridge), 2-thio-LNA (4′-(CH₂)—S-2′ bridge), 2′-amino-LNA (4′-(C₂)—NR-2′ bridge), ENA (4′-(CH₂)₂—O-2′ bridge), 4′-(CH₂)₃-2′ bridged BNA, 4′-(CH₂CH(CH₃))-2′ bridged BNA” cEt (4′-(CH(CH₃)—O-2 bridge), and cMOE BNAs (4′-(C(CH₂OCH₃)—O-2′ bridge). Some examples are show in in FIG. 3. BNA's have been prepared and disclosed in the patent literature as well as in scientific literature (Srivastava, et al., J, Am. Chem. Soc. (2007), ACS Advanced online publication, 10.1021/ja071106y; Albaek et al., J. Org. Chem. (2006), 71, 7731-7740; Fluiter, et al. Chembiochem (2005), 6, 1104-1109; Singh et al., Chem. Commun. (1998), 4, 455-456; Koshkin et al., Tetrahedron (1998), 54, 3607-3630; Wahlestedt et al., Proc. Natl. Acad. Sci. U.S.A. (2000), 97, 5633-5638; Kumar et. al., Bioorg. Med. Chem. Lett. (1998), 8, 2219-2222; WO 94/14226; WO 2005/021570; Singh et al., Org. Chem. (1998), 63, 10035-10039; WO 2007/090071; U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807).

In embodiments, the AC includes one or more nucleosides that include a locked nucleic acid (LNA). In LNAs the 2-hydroxyl group of the ribosyl sugar ring is linked to the 4′ carbon atom of the sugar ring thereby forming a 2′-C,4′-C-oxymethylene linkage to form the bicyclic sugar moiety (reviewed in Elayadi et al., Curr. Opinion Invens. Drugs (2001), 2, 558-561; Braasch et al., Chem. Biol. (2001), 8, 1-7; and Orum et al., Curr. Opinion Mol. Ther. (2001), 3, 239-243; see also U.S. Pat. Nos. 6,268,490 and 6,670,461). The linkage can be a methylene (—CH₂—) group bridging the 2′ oxygen atom and the 4′ carbon atom, for which the term LNA is used for the bicyclic moiety; in the case of an ethylene group in this position, the term ENA™ is used (Singh et al., Chem. Commun. (1998), 4, 455-456; ENA™; Morita et al., Bioorganic Medicinal Chemistry (2003), 11, 2211-2226). LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A. (2000), 97, 5633-5638).

An isomer of LNA that has also been studied is alpha-L-LNA which has been shown to have superior stability against a 3′-exonuclease. The alpha-L-LNA's were incorporated into antisense gapmers and chimeras that showed potent antisense activity (Frieden et al., Nucleic Acids Research (2003), 21, 6365-6372).

The synthesis and preparation of the LNA monomers adenine, cytosine, guanine, 5-methyl-cytosine, thymine and uracil, along with their oligomerization, and nucleic acid recognition properties have been described (Koslhkin et al., Tetrahedron (1998), 54, 3607-3630). LNAs and preparation thereof are also described in WO 98/39352 and WO 99/14226.

Analogs of LNAs such as phosphorothioate-LNAs and 2′-thio-LNAs, have also been prepared (Kumar et al., Bioorg. Med. Chem. Lett., 1998, 8, 2219-2222). Preparation of LNA analogs containing oligodeoxyribonucleotide duplexes as substrates for nucleic acid polymerases has also been described (WO 99/14226). Furthermore, synthesis of 2-amino-LNA, a conformationally restricted high-affinity oligonucleotide analog has been described (Singh et al., J. Org. Chem. (1998), 63, 10035-10039). In addition, 2-amino- and 2′-methylamino-LNA's have been prepared and the thermal stability of their duplexes with complementary RNA and DNA strands has been previously reported.

In embodiments, the antisense compound is a “tricyclo-DNA (tc-DNA)”, which refers to a class of constrained DNA analogs in which each nucleotide is modified by the introduction of a cyclopropane ring to restrict conformational flexibility of the backbone and to enhance the backbone geometry of the torsion angle γ. Homobasic adenine- and thymine-containing tc-DNAs form extraordinarily stable A-T base pairs with complementary RNAs.

Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; and 6,600,032; and WO 2005/121371.

### Internucleoside Linkages

Described herein are internucleoside linking groups that link the nucleosides or otherwise modified nucleoside monomer units together thereby forming an oligonucleotide and/or an oligonucleotide containing AC. The ACs may include naturally occurring internucleoside linkages, unnatural internucleoside linkages, or both.

In naturally occurring DNA and RNA, the internucleoside linking group is a phosphodiester that covalently links adjacent nucleosides to one another to form a linear polymeric compound. In naturally occurring DNA and RNA, phosphodiester is linked to the 2′, 3′ or 5′ hydroxyl moiety of the sugar. Within oligonucleotides, the phosphate groups are commonly referred to as forming the internucleoside backbone of the oligonucleotide. In naturally occurring DNA and RNA, the linkage or backbone of RNA and DNA, is a 3 to 5′ phosphodiester linkage. In embodiments, the internucleoside linking groups of the ACs are phosphodiesters. In embodiments, the internucleoside linking groups of the ACs are 3′ to 5′ phosphodiester linkages.

The two main classes of unnatural internucleoside linking groups are defined by the presence or absence of a phosphorus atom. Representative phosphorus containing internucleoside linkages include, but are not limited to, phosphotriesters, methylphosphonates, phosphoramidate, and phosphorothioates. Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H₂—O—); and N,N′-dimethylhydrazine (—CH₂—N(CH₃)—N(CH₃)—). ACs having phosphorus internucleoside linking groups are referred to as oligonucleotides. Antisense compounds having non-phosphorus internucleoside linking groups are referred to as oligonucleosides. Modified internucleoside linkages, compared to natural phosphodiester linkages, can be used to alter, typically increase, nuclease resistance of the antisense compound. Internucleoside linkages having a chiral atom can be prepared as racemic, chiral, or as a mixture. Representative chiral internucleoside linkages include, but are not limited to, alkylphosphonates and phosphorothioates. Methods of preparation of phosphorous-containing and non-phosphorous-containing linkages are well known to those skilled in the art.

In embodiments, two or more nucleosides having modified sugars and/or modified nucleobases may be joined using a phosphoramidate. In embodiments, two or more nucleosides having a methylenemorpholine ring may be connected through a phosphoramidate internucleoside linkage as shown as 20 in FIG. 3 where B₁ and B₂ are modified or natural nucleobases. Antisense compounds that include nucleobases with a methylenemorpholine ring that are linked through phosphoramidate internucleoside linkage may be referred to as phosphoramidate morpholino oligomers (PMOs).

### Conjugate Groups

In embodiments, ACs are modified by covalent attachment of one or more conjugate groups. In general, conjugate groups modify one or more properties of the attached AC including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an AC. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In embodiments, the conjugate group is a polyethylene glycol (PEG), and the PEG is conjugated to either the AC or the CPP (CPP discussed elsewhere herein).

In embodiments, conjugate groups include lipid moieties such as a cholesterol moiety (Letsinger et al., Proc. Natl. Acad. Sci. USA (1989), 86, 6553); cholic acid (Manoharan et al., Bioorg. Med. Chem. Lett. (1994), 4, 1053); a thioether, e.g., hexyl-S-tritylthiol (Manoharan et al., Ann. N.Y. Acad. Sci. (1992), 660, 306; Manoharan et al., Bioorg. Med. Chem. Let. (1993), 3, 2765); a thiocholesterol (Oberhauser et al., Nucl. Acids Res. (1992), 20, 533); an aliphatic chain, e.g., dodecandiol or undecyl residues (Saison-Behmoaras et al., EMBO J. (1991), 10, 111; Kabanov et al., FEBS Lett. (1990), 259, 327; Svinarchuk et al., Biochimie (1993), 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett. (1995), 36, 3651; Shea et al., Nucl. Acids Res. (1990), 18, 3777); a polyamine or a polyethylene glycol chain (Manoharan et al., Nucleosides & Nucleotides (1995), 14, 969); adamantane acetic acid (Manoharan et al., Tetrahedron Lett. (1995), 36, 3651); a palmityl moiety Mishra et al., Biochim. Biophys. Acta. (1995), 1264, 229); or an octadecylamine or hexylamino-carbonyl-oxycholesterol moiety (Crooke et al., J. Pharmacol. Exp Ther. (1996), 277,923).

### Types of Antisense Compounds

Various types of ACs may be used for example, including an antisense oligonucleotide, siRNA, microRNA, antagomir, aptamer, ribozyme, supermir, miRNA mimic, miRNA inhibitor, or combinations thereof.

### Antisense Oligonucleotides

In various embodiments, the antisense compound (AC) is an antisense oligonucleotide (ASO) that is complementary to a target nucleotide sequence. The term “antisense oligonucleotide (ASO)” or simply “antisense” is meant to include oligonucleotides that are complementary to a target nucleotide sequence. The term also encompasses ASOs that may not be fully complementary to the desired target nucleotide sequence. ASOs include single strands of DNA and/or RNA that are complementary to a chosen target nucleotide sequence or a target gene. ASOs may include one or more modified DNA and/or RNA bases, modified sugars, and/or unnatural internucleoside linkages. In embodiments, the ASOs may include one or more phosphoramidate internucleoside linkages. In embodiments, the ASO is phosphoramidate morpholino oligomers (PMOs). ASOs may have any characteristic, be any length, bind to any splice element and effect any mechanism as described relative to an AC. In embodiments, an ASO induces exon skipping to introduce a premature termination codon and ultimately result in nonsense mediated degradation of the target transcript. In embodiments, an ASO is a PMO and induces exon skipping to introduce a premature termination codon and ultimately result in nonsense mediated degradation of the target transcript.

Antisense oligonucleotides have been demonstrated to be effective as targeted inhibitors of protein synthesis, and, consequently, can be used to specifically inhibit protein synthesis by a targeted gene. The efficacy of ASO for inhibiting protein synthesis is well established. To date, these compounds have shown promise in several in vitro and in vivo models, including models of inflammatory disease, cancer, and HIV (Agrawal, Trends in Biotech. (1996), 14:376-387). Antisense can also affect cellular activity by hybridizing specifically with chromosomal DNA.

Methods of producing ASOs are known in the art and can be readily adapted to produce an ASO that binds to a target nucleotide sequence of the present disclosure. Selection of ASOs sequences specific for a given target nucleotide sequence is based upon analysis of the chosen target nucleotide sequence and determination of secondary structure, Tm, binding energy, and relative stability. Antisense oligonucleotides may be selected based upon their relative inability to form dimers, hairpins, or other secondary structures that would reduce or prohibit specific binding to the target nucleotide sequence in a host cell. These secondary structure analyses and target site selection considerations can be performed, for example, using v.4 of the OLIGO primer analysis software (Molecular Biology Insights) and/or the BLASTN 2.0.5 algorithm software (Altschul et ai, Nucleic Acids Res. 1997, 25(17):3389-402).

### RNA Interference

In embodiments, the AC includes a molecule that mediates RNA interference (RNAi). As used herein, the phrase “mediates RNAi” refers to the ability to silence, in a sequence specific manner, a target transcript. While not wishing to be bound by theory, it is believed that silencing uses the RNAi machinery or process and a guide RNA, e.g., an siRNA and/or miRNA compound of from about 21 to about 23 nucleotides. In embodiments, the AC targets the target transcript for degradation. As such, in embodiments, RNAi molecule may be used to disrupt the expression of a gene or polynucleotide of interest. In embodiments, RNAi molecule is used to induce degradation of the target transcript, such as a pre-mRNA or a mature mRNA.

In embodiments, the AC includes a small interfering RNA (siRNA) that elicits an RNAi response. In embodiments, the AC includes a microRNA (miRNA) that elicits an RNAi response.

Small interfering RNAs (siRNAs) are nucleic acid duplexes normally from about 16 to about 30 nucleotides long that can associate with a cytoplasmic multi-protein complex known as RNAi-induced silencing complex (RISC). RISC loaded with siRNA mediates the degradation of homologous transcripts, therefore siRNA can be designed to knock down protein expression with high specificity. Unlike other antisense technologies, siRNA function through a natural mechanism evolved to control gene expression through non-coding RNA. A variety of RNAi reagents, including siRNAs targeting clinically relevant targets, are currently under pharmaceutical development, as described, e.g., in de Fougerolles, A. et al., Nature Reviews (2007) 6:443-453.

The therapeutic applications of RNAi are extremely broad, since siRNA and miRNA constructs can be synthesized with any nucleotide sequence directed against a target protein. To date, siRNA constructs have shown the ability to specifically down-regulate target proteins in both in vitro and in vivo models, as well as in clinical studies.

While the first described RNAi molecules were RNA:RNA hybrids that include both an RNA sense and an RNA antisense strand, it has now been demonstrated that DNA sense:RNA antisense hybrids, RNA sense:DNA antisense hybrids, and DNA:DNA hybrids are capable of mediating RNAi (Lamberton, J. S. and Christian, A. T., Molecular Biotechnology (2003), 24:111-119). In embodiments, RNAi molecules are used that include any of these different types of double-stranded molecules. In addition, it is understood that RNAi molecules may be used and introduced to cells in a variety of forms. Accordingly, as used herein, RNAi molecules encompasses any and all molecules capable of mediating an RNAi in cells, including, but not limited to, double-stranded oligonucleotides that include two separate strands, i.e. a sense strand and an antisense strand, e.g., small interfering RNA (siRNA); double-stranded oligonucleotide that includes two separate strands that are linked together by non-nucleotidyl linker; oligonucleotides that include a hairpin loop of complementary sequences, which forms a double-stranded region, e.g., shRNAi molecules, and expression vectors that express one or more polynucleotides capable of forming a double-stranded polynucleotide alone or in combination with another polynucleotide.

A “single strand siRNA compound” as used herein, is an siRNA compound which is made up of a single molecule. It may include a duplexed region, formed by intra-strand pairing, e.g., it may be, or include, a hairpin or pan-handle structure. Single strand siRNA compounds may be antisense with regard to the target molecule.

A single strand siRNA compound may be sufficiently long that it can enter the RISC and participate in RISC mediated cleavage of a target mRNA. A single strand siRNA compound is at least about 14, at least about 15, at least about 20, at least about 25, at least about 30, at least about 35, at least about 40, or up to about 50 nucleotides in length. In certain embodiments, the single strand siRNA is less than about 200, about 100, or about 60 nucleotides in length.

Hairpin siRNA compounds may have a duplex region equal to or at least about 17, about 18, about 19, about 20, about 21, about 22, about 23, about 24, or about 25 nucleotide pairs. The duplex region may be equal to or less than about 200, about 100, or about 50 nucleotide pairs in length. In certain embodiments, ranges for the duplex region are from about 15 to about 30, from about 17 to about 23, from about 19 to about 23, and from about 19 to about 21 nucleotides pairs in length. The hairpin may have a single strand overhang or terminal unpaired region. In certain embodiments, the overhangs are from about 2 to about 3 nucleotides in length. In embodiments, the overhang is at the same side of the hairpin and in embodiments on the antisense side of the hairpin.

A “double stranded siRNA compound” as used herein, is an siRNA compound which includes more than one, and in some cases two, strands in which interchain hybridization can form a region of duplex structure.

The antisense strand of a double stranded siRNA compound may be equal to or at least about 14, about 15, about 16 about 17, about 18, about 19, about 20, about 25, about 30, about 40, or about 60 nucleotides in length. It may be equal to or less than about 200, about 100, or about 50 nucleotides in length. Ranges may be from about 17 to about 25, from about 19 to about 23, and from about 19 to about 21 nucleotides in length. As used herein, term “antisense strand” means the strand of an siRNA compound that is sufficiently complementary to a target molecule, e.g. the target nucleotide sequence of a target transcript.

The sense strand of a double stranded siRNA compound may be equal to or at least about 14, about 15, about 16, about 17, about 18, about 19, about 20, about 25, about 30, about 40, or about 60 nucleotides in length. It may be equal to or less than about 200, about 100, or about 50, nucleotides in length. Ranges may be from about 17 to about 25, from about 19 to about 23, and from about 19 to about 21 nucleotides in length.

The double strand portion of a double stranded siRNA compound may be equal to or at least about 14, about 15, about 16, about 17, about 18, about 19, about 20, about 21, about 22, about 23, about 24, about 25, about 30, about 40, or about 60 nucleotide pairs in length. It may be equal to or less than about 200, about 100, or about 50, nucleotides pairs in length. Ranges may be from about 15 to about 30, from about 17 to about 23, from about 19 to about 23, and from about 19 to about 21 nucleotides pairs in length.

In embodiments, the siRNA compound is sufficiently large that it can be cleaved by an endogenous molecule, e.g., by Dicer, to produce smaller siRNA compounds, e.g., siRNAs agents.

The sense and antisense strands may be chosen such that the double-stranded siRNA compound includes a single strand or unpaired region at one or both ends of the molecule. Thus, a double-stranded siRNA compound may contain sense and antisense strands, paired to contain an overhang, e.g., one or two 5′ or 3′ overhangs, or a 3′ overhang of 1 to 3 nucleotides. The overhangs can be the result of one strand being longer than the other, or the result of two strands of the same length being staggered. Some embodiments will have at least one 3′ overhang. In embodiments, both ends of an siRNA molecule will have a 3′ overhang. In embodiments, the overhang is 2 nucleotides.

In embodiments, the length for the duplexed region is from about 15 to about 30, or about 18, about 19, about 20, about 21, about 22, or about 23 nucleotides in length, e.g., in the ssiRNA (siRNA with sticky overhangs) compound range discussed above. ssiRNA compounds can resemble in length and structure the natural Dicer processed products from long dsiRNAs. Embodiments in which the two strands of the ssiRNA compound are linked, e.g., covalently linked are also included. In embodiments, hairpin, or other single strand structures which provide a double stranded region, and a 3′ over hangs are included.

The siRNA compounds described herein, including double-stranded siRNA compounds and single-stranded siRNA compounds can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene.

In embodiments, an siRNA compound is “sufficiently complementary” to a target transcript, such that the siRNA compound silences production of protein encoded by the target mRNA. In embodiments, the siRNA compound is “sufficiently complementary” to at least a portion of a target transcript, such that the siRNA compound silences production of the gene product encoded by the target transcript. In another embodiment, the siRNA compound is “exactly complementary” to a target nucleotide sequence (e.g., a portion of a target transcript) such that the target nucleotide sequence and the siRNA compound anneal, for example to form a hybrid made exclusively of Watson-Crick base pairs in the region of exact complementarity. A “sufficiently complementary” to a target nucleotide sequence can include an internal region (e.g., of at least about 10 nucleotides) that is exactly complementary to a target nucleotide sequence. Moreover, in certain embodiments, the siRNA compound specifically discriminates a single-nucleotide difference. In this case, the siRNA compound only mediates RNAi if exact complementary is found in the region (e.g., within 7 nucleotides of) the single-nucleotide difference.

The therapeutic applications of RNAi are extremely broad, since siRNA and miRNA constructs can be synthesized with any nucleotide sequence directed against a target protein. To date, siRNA constructs have shown the ability to specifically down-regulate target proteins in both in vitro and in vivo models, as well as in clinical studies

### MicroRNAs

In embodiments, the AC includes a microRNA molecule. MicroRNAs (miRNAs) are a highly conserved class of small RNA molecules that are transcribed from DNA in the genomes of plants and animals but are not translated into protein. Processed miRNAs are single stranded 17-25 nucleotide RNA molecules that become incorporated into the RNA-induced silencing complex (RISC) and have been identified as key regulators of development, cell proliferation, apoptosis and differentiation They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

### Antagomirs

In embodiments, the AC is an antagomir. Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate backbone and, for example, a cholesterol-moiety at 3′-end. Antagomirs may be used to efficiently silence endogenous miRNAs by forming duplexes that include the antagomir and endogenous miRNA, thereby preventing miRNA-induced gene silencing. An example of antagomir-mediated miRNA silencing is the silencing of miR-122, described in Krutzfeldt et al., Nature (2005), 438: 685-689, which is expressly incorporated by reference herein in its entirety. Antagomir RNAs may be synthesized using standard solid phase oligonucleotide synthesis protocols (U.S. patent application Ser. Nos. 11/502,158 and 11/657,341; the disclosure of each of which are incorporated herein by reference).

An antagomnir can include ligand-conjugated monomer subunits and monomers for oligonucleotide synthesis. Monomers are described in U.S. application Ser. No. 10/916,185. An antagomir can have a ZXY structure, such as is described in PCT Application No. PCT/US2004/07070. An antagomir can be complexed with an amphipathic moiety. Amphipathic moieties for use with oligonucleotide agents are described in PCT Application No. PCT/US2004/07070.

### Aptamers

In embodiments, the AC includes an aptamer. Aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules (Eaton, Curr. Opin. Chem. Biol. (1997), 1: 10-16; Famulok, Curr. Opin. Struct. Biol. (1999), 9:324-9; and Hermann and Patel, Science (2000), 287:820-5). Aptamers may be RNA or DNA based and may include a riboswitch. A riboswitch is a part of an mRNA molecule that can directly bind a small target molecule, and whose binding of the target affects the gene's activity. Thus, an mRNA that contains a riboswitch is directly involved in regulating its own activity, depending on the presence or absence of its target molecule. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer may be prepared by any known method, including synthetic, recombinant, and purification methods, and may be used alone or in combination with other aptamers specific for the same target. Further, the term “aptamer” also includes “secondary aptamers” containing a consensus sequence derived from comparing two or more known aptamers to a given target. In embodiments, the aptamer is an “intracellular aptamer”, or “intramer”, which specifically recognize intracellular targets (Famulok et al., Chem Biol. (2001),8(10):931-939; Yoon and Rossi, Adv. Drug Deliv. Rev. (2018), 134:22-35; each incorporated by reference herein).

### Ribozymes

In embodiments, the AC is a ribozyme. Ribozymes are RNA molecules complexes having specific catalytic domains that possess endonuclease activity (Kim and Cech, Proc. Natl. Acad. Sci. USA (1987), 84(24):8788-92; Forster and Symons, Cell (1987) 24, 49(2):211-20). For example, a large number of ribozymes accelerate phosphoester transfer reactions with a high degree of specificity, often cleaving only one of several phosphoesters in an oligonucleotide substrate (Cech et al., Cell (1981), 27(3 Pt 2):487-96; Michel and Westhof, J. Mol. Biol. (1990), 5, 216(3):585-610; Reinhold-Hurek and Shub, Nature (1992), 14, 357(6374): 173-6). This specificity has been attributed to the requirement that the substrate bind via specific base-pairing interactions to the internal guide sequence (IGS) of the ribozyme prior to chemical reaction.

At least six basic varieties of naturally occurring enzymatic RNAs are known presently. Each can catalyze the hydrolysis of RNA phosphodiester bonds in trans (and thus can cleave other RNA molecules) under physiological conditions, In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis δ virus, group I intron or RNaseP RNA (in association with an RNA guide sequence) or Neurospora VS RNA motif, for example. Specific examples of hammerhead motifs are described by Rossi et al. Nucleic Acids Res. (1992), 20(17):4559-65. Examples of hairpin motifs are described by Hampel et al. (Eur. Pat. Appl. Publ. No. EP 0360257), Hamnpel and Tritz, Biochemistry (1989), 28(12):4929-33; Hampel et al, Nucleic Acids Res. (1990),18(2):299-304 and U.S. Pat. No. 5,631,359. An example of the hepatitis virus motif is described by Perrotta and Been, Biochemistry (1992), 31(47): 11843-52; an example of the RNaseP motif is described by Guerrier-Takada et al., Cell (1983), 35(3 Pt 2):849-57; Neurospora VS RNA ribozyme motif is described by Collins (Saville and Collins, Cell (1990), 61(4):685-96; Saville and Collins, Proc. Natl. Acad. Sci. USA (1991),88(19):8826-30; Collins and Olive, Biochemistry (1993),32(l 1):2795-9); and an example of the Group I intron is described in U.S. Pat. No. 4,987,071 In embodiments, enzymatic nucleic acid molecules have a specific substrate binding site which is complementary to one or more of the target gene DNA or RNA regions, and that they have nucleotide sequences within or surrounding that substrate binding site which impart an RNA cleaving activity to the molecule. Thus, the ribozyme constructs need not be limited to specific motifs mentioned herein.

Ribozymes may be designed as described in Int. Pat. Appl. Publ. No. WO 93/23569 and Int. Pat. Appl. Publ No. WO 94/02595, each specifically incorporated herein by reference, and synthesized to be tested in vitro and in vivo, as described therein. In embodiments, the ribozyme is targeted to a target nucleotide sequence of a target transcript.

Ribozyme activity can be increased by altering the length of the ribozyme binding arms or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem H bases to shorten RNA synthesis times and reduce chemical requirements.

### Supermir

In embodiments, the AC is a supermir. A supermir refers to a single stranded, double stranded, or partially double stranded oligomer or polymer of RNA, polymer of DNA, or both, or modifications thereof, which has a nucleotide sequence that is substantially identical to an miRNA and that is antisense with respect to its target, This term includes oligonucleotides composed of naturally-occurring nucleobases, sugars and covalent internucleoside (backbone) linkages and which contain at least one non-naturally-occurring portion which functions similarly. Such modified or substituted oligonucleotides have desirable properties such as, for example, enhanced cellular uptake, enhanced affinity for nucleic acid target and increased stability in the presence of nucleases. In embodiments, the supermir does not include a sense strand, and in another embodiment, the supermir does not self-hybridize to a significant extent. A supermir can have secondary structure, but it is substantially single-stranded under physiological conditions. A supermir that is substantially single-stranded is single-stranded to the extent that less than about 50% (e.g., less than about 40%, about 30%, about 20%, about 10%, or about 5%) of the supermir is duplexed with itself. The supermir can include a hairpin segment, e.g., sequence, for example, at the 3′ end can self-hybridize and form a duplex region, e.g., a duplex region of at least about 1, about 2, about 3, or about 4 or less than about 8, about 7, about 6, or about 5 nucleotides, or about 5 nucleotides. The duplexed region can be connected by a linker, e.g., a nucleotide linker, e.g., about 3, about 4, about 5, or about 6 dTs, e.g., modified dTs. In another embodiment the supermir is duplexed with a shorter oligo, e.g., of about 5, about 6, about 7, about 8, about 9, or about 10 nucleotides in length, e.g., at one or both of the 3′ and 5′ end or at one end and in the non-terminal or middle of the supermir.

miRNA Mimics

In embodiments, the AC is a miRNA mimic. miRNA mimics represent a class of molecules that can be used to imitate the gene silencing ability of one or more miRNAs. Thus, the term “microRNA mimic” refers to synthetic non-coding RNAs (e.g., the miRNA is not obtained by purification from a source of the endogenous miRNA) that are capable of entering the RNAi pathway and regulating gene expression. miRNA mimics can be designed as mature molecules (e.g. single stranded) or mimic precursors (e.g., pri- or pre-miRNAs). miRNA mimics can include nucleic acid (modified or modified nucleic acids) including oligonucleotides that include, without limitation, RNA, modified RNA, DNA, modified DNA, locked nucleic acids, or 2′-0,4-C-ethylene-bridged nucleic acids (ENA), or any combination of the above (including DNA-RNA hybrids), In addition, miRNA mimics can include conjugates that can affect delivery, intracellular compartmentalization, stability, specificity, functionality, strand usage, and/or potency. In one design, miRNA mimics are double stranded molecules (e.g., with a duplex region of between about 16 and about 31 nucleotides in length) and contain one or more sequences that have identity with the mature strand of a given miRNA. Modifications can include 2′ modifications (including 2-0 methyl modifications and 2′ F modifications) on one or both strands of the molecule and internucleoside modifications (e.g., phosphorothioate modifications) that enhance nucleic acid stability and/or specificity. In addition, miRNA mimics can include overhangs. The overhangs can include from about 1 to about 6 nucleotides on either the 3′ or 5′ end of either strand and can be modified to enhance stability or functionality. In embodiments, a miRNA mimic includes a duplex region of from about 16 to about 31 nucleotides and one or more of the following chemical modification patterns: the sense strand contains 2-O-methyl modifications of nucleotides 1 and 2 (counting from the 5′ end of the sense oligonucleotide), and all of the Cs and Us; the antisense strand modifications can include 2′ F modification of all of the Cs and Us, phosphorylation of the 5′ end of the oligonucleotide, and stabilized internucleoside linkages associated with a 2 nucleotide 3′ overhang.

miRNA Inhibitor

In embodiments, the AC is a miRNA inhibitor. The terms “antimir” “microRNA inhibitor”, “miR inhibitor”, or “miRNA inhibitor” are synonymous and refer to oligonucleotides or modified oligonucleotides that interfere with the ability of specific miRNAs. In general, the inhibitors are nucleic acid or modified nucleic acids in nature including oligonucleotides that include RNA, modified RNA, DNA, modified DNA, locked nucleic acids (LNAs), or any combination of the above.

Modifications include 2′ modifications (including 2′-0 alkyl modifications and 2′ F modifications) and internucleoside modifications (e.g., phosphorothioate modifications) that can affect delivery, stability, specificity, intracellular compartmentalization, or potency. In addition, miRNA inhibitors can include conjugates that can affect delivery, intracellular compartmentalization, stability, and/or potency. Inhibitors can adopt a variety of configurations including single stranded, double stranded (RNA/RNA or RNA/DNA duplexes), and hairpin designs, in general, microRNA inhibitors include contain one or more sequences or portions of sequences that are complementary or partially complementary with the mature strand (or strands) of the miRNA to be targeted, in addition, the miRNA inhibitor may also include additional sequences located 5′ and 3′ to the sequence that is the reverse complement of the mature miRNA. The additional sequences may be the reverse complements of the sequences that are adjacent to the mature miRNA in the pri-miRNA from which the mature miRNA is derived, or the additional sequences may be arbitrary sequences (having a mixture of A, C, C, or U). In embodiments, one or both of the additional sequences are arbitrary sequences capable of forming hairpins. Thus, in embodiments, the sequence that is the reverse complement of the miRNA is flanked on the 5′ side and on the 3 side by hairpin structures. Micro-RNA inhibitors, when double stranded, may include mismatches between nucleotides on opposite strands. Furthermore, micro-RNA inhibitors may be linked to conjugate moieties in order to facilitate uptake of the inhibitor into a cell. For example, a micro-RNA inhibitor may be linked to cholesteryl 5-(bis(4-methoxyphenyl)(phenyl)methoxy)-3 hydroxypentylcarbamate) which allows passive uptake of a micro-RNA inhibitor into a cell. Micro-RNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

### CRISPR Gene-Editing Machine

In embodiments, the therapeutic moiety includes one or more elements of CRISPR gene-editing machinery. As used herein, “CRISPR gene-editing machinery” refers to protein, nucleic acids, or combinations thereof, which may be used to edit a genome. Non-limiting examples of gene-editing machinery include guide RNAs (gRNAs), nucleases, nuclease inhibitors, and combinations and complexes thereof. The following patent documents describe CRISPR gene-editing machinery: U.S. Pat. Nos. 8,697,359, 8,771,945, 8,795,965, 8,865,406, 8,871,445, 8,889,356, 8,895,308, 8,906,616, 8,932,814, 8,945,839, 8,993,233, 8,999,641, U.S. patent application Ser. No. 14/704,551, and U.S. patent application Ser. No. 13/842,859. Each of the aforementioned patent documents is incorporated by reference herein in its entirety.

gRNA

In embodiments, the TM includes a gRNA. A gRNA targets a genomic loci in a prokaryotic or eukaryotic cell.

In embodiments, the gRNA is a single-molecule guide RNA (sgRNA). A sgRNA includes a spacer sequence and a scaffold sequence. A spacer sequence is a short nucleic acid sequence used to target a nuclease (e.g., a Cas9 nuclease) to a specific nucleotide region of interest (e.g., a genomic DNA sequence to be cleaved). In embodiments, the spacer may be about 17-24 bases in length, such as about 20 bases in length. In embodiments, the spacer may be about 15, about 16, about 17, about 18, about 19, about 20, about 21, about 22, about 23, about 24, about 25, about 26, about 27, about 28, about 29, or about 30 bases in length. In embodiments, the spacer may be at least 15, at least 16, at least 17, at least 18, at least 19, at least 20, at least 21, at least 22, at least 23, at least 24, at least 25, at least 26, at least 27, at least 28, at least 29, or at least 30 bases in length. In embodiments, the spacer may be about 15, about 16, about 17, about 18, about 19, about 20, about 21, about 22, about 23, about 24, about 25, about 26, about 27, about 28, about 29, or about 30 bases in length. In embodiments, the spacer sequence has between about 40% to about 80% GC content.

In embodiments, the spacer binds to a target nucleotide sequence that immediately precedes a 5′ protospacer adjacent motif (PAM). The PAM sequence may be selected based on the desired nuclease. For example, the PAM sequence may be any one of the PAM sequences shown in Table 13 below, wherein N refers to any nucleic acid, R refers to A or G, Y refers to C or T, W refers to A or T, and V refers to A or C or G.

TABLE 13
Nucleases and PAM sequences

| PAM sequence | | |
|---|---|---|
| (5′ to 3′) | Nuclease | Isolated from |
| NGG | SpCas9 | Streptococcus pyogenes |
| NGRRT or | SaCas9 | Staphylococcus aureus |
| NGRRN | | |
| NNNNGATT | NmeCas9 | Neisseria meningitidis |
| NNNNRYAC | CjCas9 | Campylobacter jejuni |
| NNAGAAW | StCas9 | Streptococcus thermophiles |
| TTTV | LbCpf1 | Lachnospiraceae bacterium |
| TTTV | AsCpf1 | Acidaminococcus sp. |

In embodiments, a spacer binds to a target nucleotide sequence of a mammalian target transcript of a target gene, such as a human gene. In embodiments, the spacer may bind to a target nucleotide sequence of a target transcript. In embodiments, the spacer may bind to a target nucleotide sequence that includes at least a portion of a splice element (SE) and/or a splice regulatory element (SRE) of a target transcript or that is in sufficient proximity to a SE and/or a SRE of a target transcript to modulate splicing.

The scaffold sequence is the sequence within the sgRNA that is responsible for nuclease (e.g., Cas9) binding. The scaffold sequence does not include the spacer/targeting sequence. In embodiments, the scaffold may be about 1 to about 10, about 10 to about 20, about 20 to about 30, about 30 to about 10, about 40 to about 50, about 50 to about 60, about 60 to about 70, about 70 to about 80, about 80 to about 90, about 90 to about 100, about 100 to about 110, about 110 to about 120, or about 120 to about 130 nucleotides in length. In embodiments, the scaffold may be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, or about 125 nucleotides in length. In embodiments, the scaffold may be at least 10, at least 20, at least 30, at least 40, at least 50, at least 60, at least 70, at least 80, at least 90, at least 100, at least 110, at least 120, or at least 125 nucleotides in length.

In embodiments, the gRNA is a dual-molecule guide RNA, e.g, crRNA and tracrRNA. In embodiments, the gRNA may further include a poly(A) tail.

In embodiments, multiple gRNAs may be used a TMs in a single compound. In embodiments, the TM includes about 1, about 2, about 3, about 4, about 5, about 6, about 7, about 8, about 9, about 10, about 11, about 12, about 13, about, 14, about 15, about 16, about 17, about 18, about 19, or about 20 gRNAs. In embodiments, the gRNAs recognize the same target. In embodiments, the gRNAs recognize different targets. In embodiments, the nucleic acid that includes a gRNA includes a sequence encoding a promoter, wherein the promoter drives expression of the gRNA.

### Nuclease

In embodiments, the TM includes a nuclease. In embodiments, the nuclease is a Type II, Type V-A, Type V-B, Type VC, Type V-U, Type VI-B nuclease. In embodiments, the nuclease is a transcription, activator-like effector nuclease (TALEN), a meganuclease, or a zinc-finger nuclease. In embodiments, the nuclease is a Cas9, Cas12a (CF3), Cas12b, Cas12c, Tnp-B like, Cas13a (C2c2), Cas13b, or Cas14 nuclease. For example, In embodiments, the nuclease is a Cas9 nuclease or a Cpf1 nuclease.

In embodiments, the nuclease is a modified form or variant of a Cas9, Cas12a (Cpf1), Cas12b, Cas12c, Tnp-B like, Cas13a (C2c2), Cas13b, or Cas14 nuclease. In embodiments, the nuclease is a modified form or variant of a TAL nuclease, a meganuclease, or a zinc-finger nuclease. A “modified” or “variant” nuclease is one that is, for example, truncated, fused to another protein (such as another nuclease), catalytically inactivated, etc. In embodiments, the nuclease may have at least about 80%, at least about 85%, at least about 90%, at least about 95%, at least about 98%, at least about 99%, or about 100% sequence identity to a naturally occurring Cas9, Cas12a (Cpf1), Cas12b, Cas12c, Tnp-B like, Cas13a (C2c2), Cas13b, Cas14 nuclease, or a TALEN, meganuclease, or zinc-finger nuclease. In embodiments, the nuclease is a Cas9 nuclease derived from S. pyogenes (SpCas9). In embodiments, a nuclease has at least about 90%, at least about 95%, at least about 96%, at least about 97%, at least about 98%, or at least about 99% sequence identity to a Cas9 nuclease derived from S. pyogenes (SpCas9). In embodiments, the nuclease is a Cas9 derived from S. aureus (SaCas9). In embodiments, the nuclease has at least about 90%, at least about 95%, at least about 96%, at least about 97%, at least about 98%, or at least about 99% sequence identity to a Cas9 derived from S. aureus (SaCas9). In embodiments, the Cpf1 is a Cpf1 enzyme from Acidaminococcus (species BV3L6, UniProt Accession No. U2UMQ6). In embodiments, the nuclease has at least about 90%, at least about 95%, at least about 96%, at least about 97%, at least about 98%, or at least about 99% sequence identity to a Cpf1 enzyme from Acidaminococcus (species BV3L6, UniProt Accession No. U2UMQ6).

In embodiments, the Cpf1 is a Cpf1 enzyme from Lachnospiraceae (species ND2006, UniProt Accession No. A0A182DWE3). In embodiments, the nuclease has at least about 90%, at least about 95%, at least about 96%, at least about 97%, at least about 98%, or at least about 99% sequence identity to a Cpf1 enzyme from Lachnospiraceae. In embodiments, a sequence encoding the nuclease is codon optimized for expression in mammalian cells. In embodiments, the sequence encoding the nuclease is codon optimized for expression in human cells or mouse cells.

In embodiments, the nuclease is a soluble protein.

In embodiments, the TM is a nucleotide sequence that encodes a nuclease. In embodiments, the nucleic acid encoding a nuclease includes a sequence encoding a promoter, wherein the promoter drives expression of the nuclease.

gRNA and Nuclease Combinations

In embodiments, the compounds of the present disclosure include a gRNA and a nuclease or a nucleotide sequence encoding a nuclease as TMs. In embodiments, the nucleic acid encoding a nuclease and a gRNA includes a sequence encoding a promoter, wherein the promoter drives expression of the nuclease and the gRNA. In embodiments, the nucleic acid encoding a nuclease and a gRNA includes two promoters, wherein a first promoter controls expression of the nuclease and a second promoter controls expression of the gRNA. In embodiments, the nucleic acid encoding a gRNA and a nuclease encodes from about 1 to about 20 gRNA s, or from about 1, about 2, about 3, about 4, about 5, about 6, about 7, about 8, about 9, about 10, about 11, about 12, about 13, about 14, about 15, about 16, about 17, about 18, or about 19, and up to about 20 gRNAs. In embodiments, the gRNAs recognize different targets. In embodiments, the gRNAs recognize the same target.

In embodiments, compounds of the present disclosure include ribonucleoprotein (RNP) that includes a gRNA and a nuclease as a TM.

In embodiments, a composition that includes: (a) a first compound that includes a gRNA TM and (b) a second compound that is or includes a nuclease, are delivered to a cell. In embodiments, a composition that includes: (a) a first compound that includes a nuclease as a TM, CPP and (b) a second molecule that is or includes an gRNA are delivered to a cell. In embodiments, a composition that includes: (a) a first compound that includes a gRNA as a TM and (b) a second compound that includes a nuclease as a TM are delivered to a cell.

### Genetic Element of Interest

In embodiments, the compounds disclosed herein include a genetic element of interest as a TM. In embodiments, a genetic element of interest replaces a genomic DNA sequence cleaved by a nuclease. Non-limiting examples of genetic elements of interest include genes, a single nucleotide polymorphism, promoter, or terminators.

### Nuclease Inhibitors

In embodiments, the compounds disclosed herein include a nuclease inhibitor as a TM. A limitation of gene editing is potential off-target editing. The delivery of a nuclease inhibitor will limit off-target editing. In embodiments, the nuclease inhibitor is a polypeptide, polynucleotide, or small molecule. Nuclease inhibitors are described in U.S. Publication No. 2020/087354, International Publication No. 2018/085288, U.S. Publication No. 2018/0382741, International Publication No. 2019/089761, International Publication No. 2020/068304, International Publication No. 2020/041384, and International Publication No. 2019/076651, each of which is incorporated by reference herein in its entirety.

### Endosomal Escape Vehicles (EEVs)

An endosomal escape vehicle (EEV) can be used to transport a cargo across a cellular membrane, for example, to deliver the cargo to the cytosol or nucleus of a cell. Cargo can include a TM. The EEV can comprise a cell penetrating peptide (CPP), for example, a cyclic cell penetrating peptide (cCPP). In embodiments, the EEV comprises a cCPP, which is conjugated to an exocyclic peptide (EP). The EP can be referred to interchangeably as a modulatory peptide (MP). The EP can comprise a sequence of a nuclear localization signal (NLS). The EP can be coupled to the cargo. The EP can be coupled to the cCPP. The EP can be coupled to the cargo and the cCPP. Coupling between the EP, cargo, cCPP, or combinations thereof, may be non-covalent or covalent. The EP can be attached through a peptide bond to the N-terminus of the cCPP. The EP can be attached through a peptide bond to the C-terminus of the cCPP. The EP can be attached to the cCPP through a side chain of an amino acid in the cCPP. The EP can be attached to the cCPP through a side chain of a lysine which can be conjugated to the side chain of a glutamine in the cCPP. The EP can be conjugated to the 5′ or 3′ end of an oligonucleotide cargo. The EP can be coupled to a linker. The exocyclic peptide can be conjugated to an amino group of the linker. The EP can be coupled to a linker via the C-terminus of an EP and a cCPP through a side chain on the cCPP and/or EP. For example, an EP may comprise a terminal lysine which can then be coupled to a cCPP containing a glutamine through an amide bond. When the EP contains a terminal lysine, and the side chain of the lysine can be used to attach the cCPP, the C- or N-terminus may be attached to a linker on the cargo.

### Exocyclic Peptides

The exocyclic peptide (EP) can comprise from 2 to 10 amino acid residues e.g., 2, 3, 4, 5, 6, 7, 8, 9, or 10 amino acid residues, inclusive of all ranges and values therebetween. The EP can comprise 6 to 9 amino acid residues. The EP can comprise from 4 to 8 amino acid residues.

Each amino acid in the exocyclic peptide may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein. For example, the amino acids can be A, Ga, P, K, R, V, F, H, Nal, or citrulline.

The EP can comprise at least one positively charged amino acid residue, e.g., at least one lysine residue and/or at least one amine acid residue comprising a side chain comprising a guanidine group, or a protonated form thereof. The EP can comprise 1 or 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four or more lysine residues. The EP can comprise 2, 3, or 4 lysine residues. The amino group on the side chain of each lysine residue can be substituted with a protecting group, including, for example, trifluoroacetyl (COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF₃) group. The protecting group can be included to enable amide conjugation. The protecting group can be removed after the EP is conjugated to a cCPP.

The EP can comprise at least 2 amino acid residues with a hydrophobic side chain. The amino acid residue with a hydrophobic side chain can be selected from valine, proline, alanine, leucine, isoleucine, and methionine. The amino acid residue with a hydrophobic side chain can be valine or proline.

The EP can comprise at least one positively charged amino acid residue, e.g., at least one lysine residue and/or at least one arginine residue. The EP can comprise at least two, at least three or at least four or more lysine residues and/or arginine residues.

The EP can comprise KK, KR, RR, HH, HK, HR, RH, KKK, KGK, KBK, KBR, KRK, KRR, RKK, RRR, KKH, KHK, HKK, HRR, HRH, HHR, HBH, HHH, HHHH (SEQ ID NO:1), KHKK (SEQ ID NO:2), KKHK (SEQ ID NO: 3), KKKH (SEQ ID NO:4), KHKH (SEQ ID NO:5), HKHK (SEQ ID NO:6), KKKK (SEQ ID NO:7), KKRK (SEQ ID NO:8), KRKK (SEQ ID NO:9), KRRK (SEQ ID NO: 10), RKKR (SEQ ID NO: 11), RRRR (SEQ ID NO:12), KGKK (SEQ ID NO:13), KKGK (SEQ ID NO:14), HBHBH (SEQ ID NO: 15), HBKBH (SEQ ID NO: 16), RRRRR (SEQ ID NO: 17), KKKKK (SEQ ID NO:18), KKKRK (SEQ ID NO:19), RKKKK (SEQ ID NO:20), KRKKK (SEQ ID NO:21), KKRKK (SEQ ID NO:22), KKKKR (SEQ ID NO:23), KBKBK (SEQ ID NO:24), RKKKKG (SEQ ID NO: 25), KRKKKG (SEQ ID NO:26), KKRKKG (SEQ ID NO:27), KKKKRG (SEQ ID NO:28), RKKKKB (SEQ ID NO:29), KRKKKB (SEQ ID NO:30), KKRKKB (SEQ ID NO:31), KKKKRB (SEQ ID NO:32), KKKRKV (SEQ ID NO:33), RRRRRR (SEQ ID NO:34), HHHHHH (SEQ ID NO:35), RHRHRH (SEQ ID NO:36), HRHRHR (SEQ ID NO:37), KRKRKR (SEQ ID NO:38), RKRKRK (SEQ ID NO:39), RBRBRB (SEQ ID NO:40), KBKBKB (SEQ ID NO:41), PKKKRKV (SEQ ID NO:42), PGKKRKV (SEQ ID NO:43), PKGKRKV (SEQ ID NO:44), PKKGRKV (SEQ ID NO:45), PKKKGKV (SEQ ID NO:46), PKKKRGV (SEQ ID NO:47), or PKKKRKG (SEQ ID NO:48), wherein B is beta-alanine. The amino acids in the EP can have D or L stereochemistry.

The EP can comprise KK, KR, RR, KKK, KGK, KBK, KBR, KRK, KRR, RKK, RRR, KKKK (SEQ ID NO:7), KKRK (SEQ ID NO:8), KRKK (SEQ ID NO:9), KRRK (SEQ ID NO 10), RKKR (SEQ ID NO:11), RRRR (SEQ ID NO 12), KGKK (SEQ ID NO: 13), KKGK (SEQ ID NO: 14), KKKKK (SEQ ID NO: 15), KKKRK (SEQ ID NO:19), KBKBK (SEQ ID NO:24), KKKRKV (SEQ ID NO:33), PKKKRKV (SEQ ID NO:42), PGKKRKV (SEQ ID NO:43), PKGKRKV (SEQ ID NO:44), PKKGRKV (SEQ ID NO:45), PKKKGKV (SEQ ID NO:46), PKKKRGV (SEQ ID NO:47), or PKKKRKG (SEQ ID NO:48). The EP can comprise PKKKRKV (SEQ ID NO:42), RR, RRR, RHR, RBR, RBRBR (SEQ ID NO:49), RBHBR (SEQ ID NO:50), or HBRBH (SEQ ID NO:51), wherein B is beta-alanine. The amino acids in the EP can have D or L stereochemistry.

The EP can consist of KK, KR, RR, KKK, KGK, KBK, KBR, KRK, KRR, RKK, RRR, KKKK (SEQ ID NO:7), KKRK (SEQ ID NO:8), KRKK (SEQ ID NO:9), KRRK (SEQ ID NO:10), RKKR (SEQ ID NO: 11), RRRR (SEQ ID NO: 12), KGKK (SEQ ID NO:13), KKGK (SEQ ID NO: 14), KKKKK (SEQ ID NO: 18), KKKRK (SEQ ID NO: 19), KBKBK (SEQ ID NO:24), KKKRKV (SEQ ID NO:33), PKKKRKV (SEQ ID NO:42), PGKKRKV (SEQ ID NO:43), PKGKRKV (SEQ ID NO:44), PKKGRKV (SEQ ID NO:45), PKKKGKV (SEQ ID NO:46), PKKKRGV (SEQ ID NO:47), or PKKKRKG (SEQ ID NO:48). The EP can consist of PKKKRKV (SEQ ID NO:42), RR, RRR HR, RBR, RBRBR (SEQ ID NO:49), RBHBR (SEQ ID NO:50), or HBRBH (SEQ ID NO:51), wherein B is beta-alanine. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV (SEQ ID NO:42). The EP can consist of an NLS comprising the amino acid sequence PKKKRKV (SEQ ID NO:42). The EP can comprise an NLS comprising an amino acid sequence selected from NLSKRPAAIKKAGQAKKKK (SEQ ID NO:52), PAAKRVKLD (SEQ ID NO:53), RQRRNELKRSF (SEQ ID NO:54), RMRKFKNKGKDTAELRRRRVEVSVEER (SEQ ID NO:55), KAKKDEQILKRRNV (SEQ ID NO:56), VSRKIRPRP (SEQ ID NO:57), PPKKARED (SEQ ID NO:58), PQPKKKPL (SEQ ID NO:59), SAILKKKKKMAP (SEQ ID NO:60), DRLRR (SEQ ID NO:61), PKQKKRK (SEQ ID NO:62), RKLKKKIKKL (SEQ ID NO:63), REKKKFLKRR (SEQ ID NO:64), KRKGDEVDGVDEVAKKKSKK (SEQ ID NO:65), and RKCLQAGMNLEARKTKK (SEQ ID NO.66). The EP can consist of an NLS comprising an amino acid sequence selected from NLSKRPAAIKKAGQAKKKK (SEQ ID NO:52), PAAKRVKLD (SEQ ID NO:53), RQRRNELKRSF (SEQ ID NO:54), RMRKFKNKGKDTAELRRRRVEVSVELR (SEQ ID NO: 55), KAKKDEQILKRRNV (SEQ ID NO:56), VSRKRPRP (SEQ ID NO:57), PPKKARED (SEQ ID NO:58), PQPKKKPL (SEQ ID NO:59), SALIKKKKKMAP (SEQ ID NO:60), DRLRR (SEQ ID NO:61), PKQKKRK (SEQ ID NO:62), RKLKKKIKKL (SEQ ID NO:63), REKKKFLKRR (SEQ ID NO:64), KRKGDEVDGVDEVAKKKSKK (SEQ ID NO:65), and RKCLQAGMNLEARKTKK (SEQ ID NO:66).

All exocyclic sequences can also contain an N-terminal acetyl group. Hence, for example, the EP can have the structure: Ac-PKKKRKV (SEQ ID NO:42).

### Cell Penetrating Peptides (CPP)

The cell penetrating peptide (CPP) can comprise 6 to 20 amino acid residues. The cell penetrating peptide can be a cyclic cell penetrating peptide (cCPP). The cCPP is capable of penetrating a cell membrane. An exocyclic peptide (EP) can be conjugated to the cCPP, and the resulting construct can be referred to as an endosomal escape vehicle (EEV). The cCPP can direct a cargo (e.g., a therapeutic moiety (TM) such as an oligonucleotide, peptide or small molecule) to penetrate the membrane of a cell. The cCPP can deliver the cargo to the cytosol of the cell. The cCPP can deliver the cargo to a cellular location where a target (e.g., pre-mRNA) is located. To conjugate the cCPP to a cargo (e.g., peptide, oligonucleotide, or small molecule), at least one bond or lone pair of electrons on the cCPP can be replaced.

The total number of amino acid residues in the cCPP is in the range of from 6 to 20 amino acid residues, e.g., 6, 7, 8, 9, 10, i1, 12, 13, 14, 15, 16, 17, 18, 19, or 20 amino acid residues, inclusive of all ranges and subranges therebetween. The cCPP can comprise 6 to 13 amino acid residues. The cCPP disclosed herein can comprise 6 to 10 amino acids. By way of example, cCPP comprising 6-10 amino acid residues can have a structure according to any of Formula I-A to I-E:

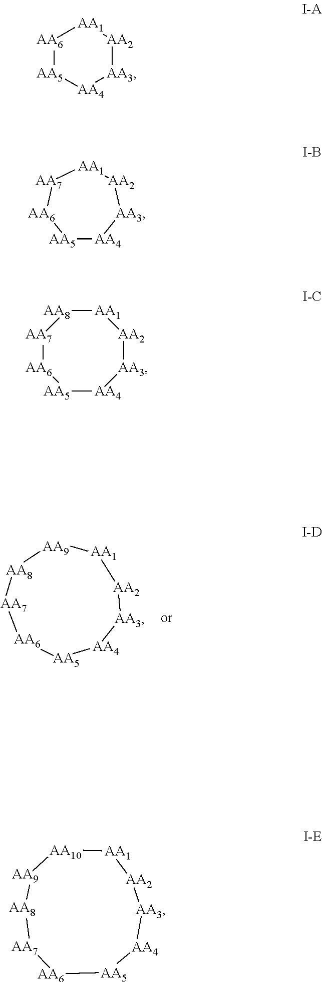

wherein AA₁, AA₂, AA₃, AA₄, AA₅, AA₆, AA₇, AA₈, AA₉, and AA₁₀ are amino acid residues.

The cCPP can comprise 6 to 8 amino acids. The cCPP can comprise 8 amino acids.

Each amino acid in the cCPP may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be a D-isomer of a natural amino acid. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein.

TABLE 1
Amino Acid Abbreviations

| | Abbreviations* | Abbreviations* |
|---|---|---|
| Amino Acid | L-amino acid | D-amino acid |
| 2-[2-[2-aminoethoxy]ethoxy]acetic acid | AEEA, miniPEG | NA |
| Alanine | Ala (A) | ala (a) |
| Allo-isoleucine | Aile | Aile |
| Arginine | Arg (R) | arg (r) |
| Asparagine | Asn (N) | asn (n) |
| aspartic acid | Asp (D) | asp (d) |
| Cysteine | Cys (C) | cys (c) |
| Citrulline | Cit | Cit |
| Cyclohexylalanine | Cha | cha |
| 2,3-diaminopropionic acid | Dap | dap |
| 4-fluorophenylalanine | Fpa (Σ) | pfa |
| glutamic acid | Glu (E) | glu (e) |
| glutamine | Gln (Q) | gln (q) |
| glycine | Gly (G) | gly (g) |
| histidine | His (H) | his (h) |
| Homoproline (aka pipecolic acid) | Pip (Θ) | pip (θ) |
| isoleucine | Ile (I) | ile (i) |
| leucine | Leu (L) | leu (l) |
| lysine | Lys (K) | lys (k) |
| methionine | Met (M) | met (m) |
| 3-(2-naphthyl)-alanine | Nal (Φ) | nal (ϕ) |
| 3-(1-naphthyl)-alanine | 1-Nal | 1-nal |
| norleucine | Nle (Ω) | nle |
| phenylalanine | Phe (f) | phe (f) |
| phenylglycine | Phg (Ψ) | phg |
| 4-(phosphonodifluoromethyl)phenylalanine | F₂Pmp (Λ) | f₂pmp |
| proline | Pro (P) | pro (p) |
| sarcosine | Sar (Ξ) | sar |
| selenocysteine | Sec (U) | sec (u) |
| serine | Ser (S) | ser (s) |
| threonine | Thr (T) | thr (y) |
| tyrosine | Tyr (Y) | tyr (y) |
| tryptophan | Trp (W) | trp (w) |
| valine | Val (V) | val (v) |
| Tert-butyl-alanine | Tle | tle |
| Penicillamine | Pen | Pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothienyl)-alanine | Bta | bta |

*single letter abbreviations: capital letters indicate the L-amino acid form, lower case letter indicate the D-amino acid form.

As used herein, “polyethylene glycol” and “PEG” are used interchangeably. “PEGm,” and “PEGₘ” are, or are derived from, a molecule of the formula HO(CO)—(CH₂)ₙ—(OCH₂CH₂)ₘ—NH₂ where n is any integer from 1 to 5 and m is any integer from 1 to 23. In embodiments, n is 1 or 2. In embodiments, n is 1. In embodiments, n is 2. In embodiments, n is 1 and m is 2. In embodiments, n is 2 and n is 2. In embodiments, n is 1 and n is 4. In embodiments, n is 2 and n is 4. In embodiments, n is 1 and m is 12. In embodiments, n is 2 and m is 12.

As used herein, “miniPEGm” or “miniPEGₘ” are, or are derived from, a molecule of the formula HO(CO)—(CH₂)ₙ—OC(CH₂)ₘ—NH₂ where n is 1 and m is any integer from 1 to 23. For example, “miniPEG2” or “miniPEG₂” is, or is derived from, (2-[2-[2-aminoethoxy]ethoxy]acetic acid), and “miniPEG4” or “miniPEG₄” is, or is derived from, HO(CO)—(CH₂)ₙ—(OCH₂CH₂)ₘ—NH₂ where n is 1 and m is 4.

The cCPP can comprise 4 to 20 amino acids, wherein: (i) at least one amino acid has a side chain comprising a guanidine group, or a protonated form thereof; (ii) at least one amino acid has no side chain or a side chain comprising

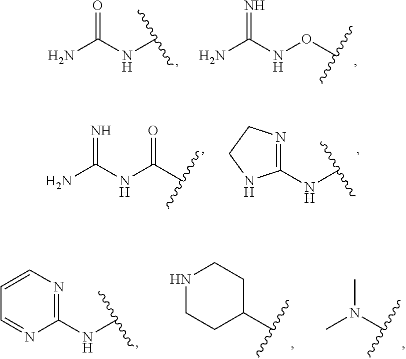

or protonated form thereof; and (iii) at least two amino acids independently have a side chain comprising an aromatic or heteroaromatic group.

At least two amino acids can have no side chain or a side chain comprising

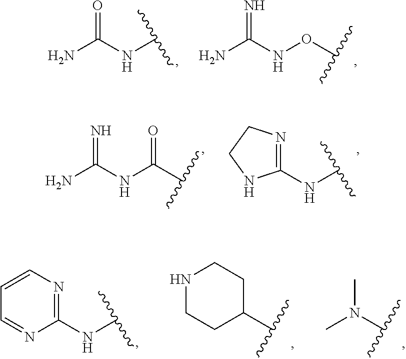

or a protonated form thereof. As used herein, when no side chain is present, the amino acid has two hydrogen atoms on the carbon atom(s) (e.g., —CH₂—) linking the amine and carboxylic acid.

The amino acid having no side chain can be glycine or β-alanine.

The cCPP can comprise from 6 to 20 amino acid residues which form the cCPP, wherein: (i) at least one amino acid can be glycine, β-alanine, or 4-aminobutyric acid residues; (ii) at least one amino acid can have a side chain comprising an aryl or heteroaryl group; and (iii) at least one amino acid has a side chain comprising a guanidine group,

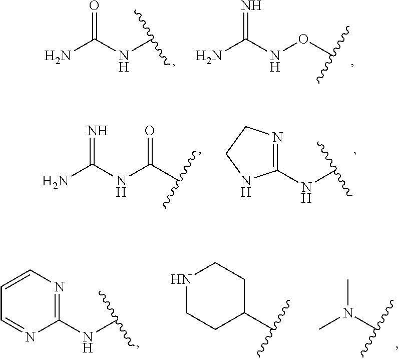

or a protonated form thereof.

The cCPP can comprise from 6 to 20 amino acid residues which form the cCPP, wherein: (i) at least two amino acid can independently beglycine, β-alanine, or 4-aminobutyric acid residues; (ii) at least one amino acid can have a side chain comprising an aryl or heteroaryl group; and (iii) at least one amino acid has a side chain comprising a guanidine group,

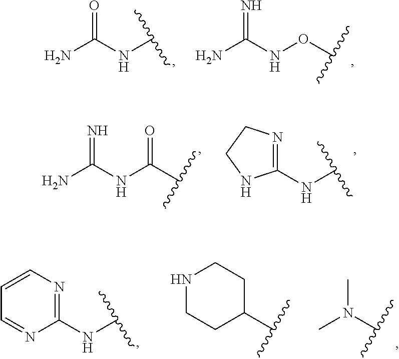

or a protonated form thereof.

The cCPP can comprise from 6 to 20 amino acid residues which form the cCPP, wherein: (i) at least three amino acids can independently be glycine, β-alanine, or 4-aminobutyric acid residues; (ii) at least one amino acid can have a side chain comprising an aromatic or heteroaromatic group; and (iii) at least one amino acid can have a side chain comprising a guanidine group,

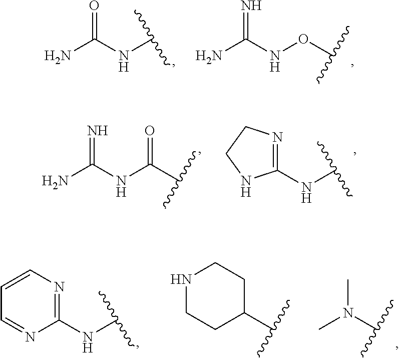

or a protonated form thereof.

### Glycine and Related Amino Acid Residues

The cCPP can comprise (i) 1, 2, 3, 4, 5, or 6 glycine, β-alanine, 4-aminobutyric acid residues, or combinations thereof. The cCPP can comprise (i) 2 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise (i) 1, 2, 3, 4, 5, or 6 glycine residues. The cCPP can comprise (i) 2 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues. The cCPP can comprise (i) 2 or 3 glycine residues. The cCPP can comprise (i) 1 or 2 glycine residues.

The cCPP can comprise (i) 3, 4, 5, or 6 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise at least three glycine residues. The cCPP can comprise (i) 3, 4, 5, or 6 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues

In embodiments, none of the glycine, β-alanine, or 4-aminobutyric acid residues in the cCPP are contiguous. Two or three glycine, β-alanine, 4- or aminobutyric acid residues can be contiguous. Two glycine, β-alanine, or 4-aminobutyric acid residues can be contiguous.

In embodiments, none of the glycine residues in the cCPP are contiguous. Each glycine residues in the cCPP can be separated by an amino acid residue that cannot be glycine. Two or three glycine residues can be contiguous. Two glycine residues can be contiguous

Amino Acid Side Chains with an Aromatic or Heteroaromatic Group

The cCPP can comprise (ii) 2, 3, 4, 5 or 6 amino acid residues independently having a side chain comprising an aromatic or heteroaromatic group. The cCPP can comprise (ii) 2 amino acid residues independently having a side chain comprising an aromatic or heteroaromatic group. The cCPP can comprise (ii) 3 amino acid residues independently having a side chain comprising an aromatic or heteroaromatic group. The cCPP can comprise (ii) 4 amino acid residues independently having a side chain comprising an aromatic or heteroaromatic group. The cCPP can comprise (ii) 5 amino acid residues independently having a side chain comprising an aromatic or heteroaromatic group. The cCPP can comprise (ii) 6 amino acid residues independently having a side chain comprising an aromatic or heteroaromatic group. The cCPP can comprise (ii) 2, 3, or 4 amino acid residues independently having a side chain comprising an aromatic or heteroaromatic group. The cCPP can comprise (ii) 2 or 3 amino acid residues independently having a side chain comprising an aromatic or heteroaromatic group.

The cCPP can comprise (ii) 2, 3, 4, 5 or 6 amino acid residues independently having a side chain comprising an aromatic group. The cCPP can comprise (ii) 2 amino acid residues independently having a side chain comprising an aromatic group. The cCPP can comprise (ii) 3 amino acid residues independently having a side chain comprising an aromatic group. The cCPP can comprise (ii) 4 amino acid residues independently having a side chain comprising an aromatic group. The cCPP can comprise (ii) 5 amino acid residues independently having a side chain comprising an aromatic group. The cCPP can comprise (ii) 6 amino acid residues independently having a side chain comprising an aromatic group. The cCPP can comprise (ii) 2, 3, or 4 amino acid residues independently having a side chain comprising an aromatic group. The cCPP can comprise (ii) 2 or 3 amino acid residues independently having a side chain comprising an aromatic group.

The aromatic group can be a 6- to 14-membered aryl. Aryl can be phenyl, naphthyl or anthracenyl, each of which is optionally substituted. Aryl can be phenyl or naphthyl, each of which is optionally substituted. The heteroaromatic group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aromatic or heteroaromatic group can each independently be bis(homonaphthylalanine), homonaphthylalanine, naphthylalanine, phenylglycine, bis(homophenylalanine), homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The amino acid having a side chain comprising an aromatic or heteroaromatic group can each independently be selected from:

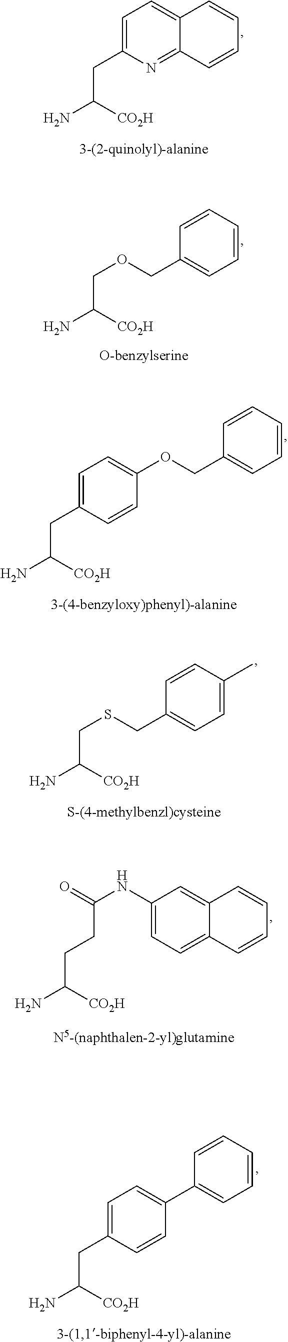 [SMILES: NC(Cc1ccc2ccccc2n1)C(=O)O ; NC(COCc1ccccc1)C(=O)O ; NC(Cc1ccc(OCc2ccccc2)cc1)C(=O)O ; Cc1ccc(CSCC(N)C(=O)O)cc1 ; NC(CCC(=O)Nc1ccc2ccccc2c1)C(=O)O ; NC(Cc1ccc(-c2ccccc2)cc1)C(=O)O]

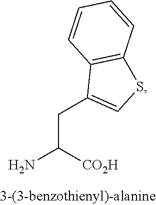 [SMILES: NC(Cc1csc2ccccc12)C(=O)O]

wherein the H on the N-terminus and/or the H on the C-terminus are replaced by a peptide bond.

The amino acid residue having a side chain comprising an aromatic or heteroaromatic group can each be independently a residue of phenylalanine, naphthylalanine, phenylglycine, homophenylalanine, homonaphthylalanine, bis(homophenylalanine), bis-(homonaphthylalanine), tryptophan, or tyrosine, each of which is optionally substituted with one or more substituents. The amino acid residue having a side chain comprising an aromatic group can each independently be a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine. The amino acid residue having a side chain comprising an aromatic group can each independently be a residue of phenylalanine, naphthylalanine, phenylglycine, homophenylalanine, or homonaphthylalanine, each of which is optionally substituted with one or more substituents. The amino acid residue having a side chain comprising an aromatic group can each be independently a residue of phenylalanine, naphthylalanine, homophenylalanine, homonaphthylalanine, bis(homonaphthylalanine), or bis(homonaphthylalanine), each of which is optionally substituted with one or more substituents. The amino acid residue having a side chain comprising an aromatic group can each be independently a residue of phenylalanine or naphthylalanine, each of which is optionally substituted with one or more substituents. At least one amino acid residue having a side chain comprising an aromatic group can be a residue of phenylalanine. At least two amino acid residues having a side chain comprising an aromatic group can be residues of phenylalanine. Each amino acid residue having a side chain comprising an aromatic group can be a residue of phenylalanine.

In embodiments, none of the amino acids having the side chain comprising the aromatic or heteroaromatic group are contiguous. Two amino acids having the side chain comprising the aromatic or heteroaromatic group can be contiguous. Two contiguous amino acids can have opposite stereochemistry. The two contiguous amino acids can have the same stereochemistry. Three amino acids having the side chain comprising the aromatic or heteroaromatic group can be contiguous. Three contiguous amino acids can have the same stereochemistry. Three contiguous amino acids can have alternating stereochemistry.

The amino acid residues comprising aromatic or heteroaromatic groups can be L-amino acids. The amino acid residues comprising aromatic or heteroaromatic groups can be D-amino acids. The amino acid residues comprising aromatic or heteroaromatic groups can be a mixture of D- and L-amino acids.

The optional substituent can be any atom or group which does not significantly reduce (e.g., by more than 50%) the cytosolic delivery efficiency of the cCPP, e.g., compared to an otherwise identical sequence which does not have the substituent. The optional substituent can be a hydrophobic substituent or a hydrophilic substituent. The optional substituent can be a hydrophobic substituent. The substituent can increase the solvent-accessible surface area (as defined herein) of the hydrophobic amino acid. The substituent can be halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

While not wishing to be bound by theory, it is believed that amino acids having an aromatic or heteroaromatic group having higher hydrophobicity values (i.e., amino acids having side chains comprising aromatic or heteroaromatic groups) can improve cytosolic delivery efficiency of a cCPP relative to amino acids having a lower hydrophobicity value. Each hydrophobic amino acid can independently have a hydrophobicity value greater than that of glycine. Each hydrophobic amino acid can independently be a hydrophobic amino acid having a hydrophobicity value greater than that of alanine. Each hydrophobic amino acid can independently have a hydrophobicity value greater or equal to phenylalanine. Hydrophobicity may be measured using hydrophobicity scales known in the art. Table 2 lists hydrophobicity values for various amino acids as reported by Eisenberg and Weiss (Proc. Natl. Acad. Sci. U.S.A. 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A. 1981; 78(6):3824-3828), and Janin (Nature. 1979; 277(5696):491-492), the entirety of each of which is herein incorporated by reference. Hydrophobicity can be measured using the hydrophobicity scale reported in Engleman, et al.

TABLE 2
Amino Acid Hydrophobicity

| | | | | Kyrie | | |
|---|---|---|---|---|---|---|
| | | Eisenberg | | and | Hoop | |
| Amino | | and | Engleman | Doo- | and | |
| Acid | Group | Weiss | et al. | little | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gln | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

The size of the aromatic or heteroaromatic groups may be selected to improve cytosolic delivery efficiency of the cCPP. While not wishing to be bound by theory, it is believed that a larger aromatic or heteroaromatic group on the side chain of amino acid may improve cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. The size of the hydrophobic amino acid can be measured in terms of the molecular weight of the hydrophobic amino acid, and the larger hydrophobic amino acid has a side chain with a molecular weight of at least about 90 g/mol, or at least about 130 g/mol, or at least about 141 g/mol. The size of the amino acid can be measured in terms of the SASA of the hydrophobic side chain. The hydrophobic amino acid can have a side chain with a SASA of greater than or equal to alanine, or greater than or equal to glycine. Larger hydrophobic amino acids can have a side chain with a SASA greater than alanine, or greater than glycine. The hydrophobic amino acid can have an aromatic or heteroaromatic group with a SASA greater than or equal to about piperidine-2-carboxylic acid, greater than or equal to about tryptophan, greater than or equal to about phenylalanine, or greater than or equal to about naphthylalanine. A first hydrophobic amino acid (AA_{H1}) can have a side chain with a SASA of at least about 200 Å², at least about 210 Å², at least about 220 Å², at least about 240 Å², at least about 250 Å², at least about 260 Å² at least about 270 Å, at least about 280 Å², at least about 290 Å² at least about 300 Å², at least about 310 Å², at least about 320 Å², or at least about 330 Å². A second hydrophobic amino acid (AA_{H2}) can have a side chain with a SASA of at least about 200 Å² at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². The side chains of AA_{H1} and AA_{H2} can have a combined SASA of at least about 350 Å², at least about 360 Å², at least about 370 Å², at least about 380 Å², at least about 390 Å² at least about 400 Å², at least about 410 Å², at least about 420 Å², at least about 430 Å², at least about 440 Å², at least about 450 Å², at least about 460 Å², at least about 470 Å², at least about 480 Å², at least about 490 Å2, greater than about 500 Å² at least about 510 Å², at least about 520 Å², at least about 530 Å² at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². AA_{H2} can be a hydrophobic amino acid residue with a side chain having a SASA that is less than or equal to the SASA of the hydrophobic side chain of AA_{H1}. By way of example, and not by limitation, a cCPP having a Nal-Arg motif may exhibit improved cytosolic delivery efficiency compared to an otherwise identical cCPP having a Phe-Arg motif; a cCPP having a Phe-Nal-Arg motif may exhibit improved cytosolic delivery efficiency compared to an otherwise identical cCPP having a Nal-Phe-Arg motif; and a phe-Nal-Arg motif may exhibit improved cytosolic delivery efficiency compared to an otherwise identical cCPP having a nal-Phe-Arg motif.

As used herein, “hydrophobic surface area” or “SASA” refers to the surface area (reported as square Ångstroms; Å²) of an amino acid side chain that is accessible to a solvent, SASA can be calculated using the ‘rolling ball’ algorithm developed by Shrake & Rupley (J Mol Biol. 79 (2): 351-71), which is herein incorporated by reference in its entirety for all purposes. This algorithm uses a “sphere” of solvent of a particular radius to probe the surface of the molecule. A typical value of the sphere is 1.4 Å, which approximates to the radius of a water molecule.

SASA values for certain side chains are shown below in Table 3. The SASA values described herein are based on the theoretical values listed in Table 3 below, as reported by Tien, et al. (PLOS ONE 8(11): e80635, available at doi.org/10.1371/journal.pone.0080635), which is herein incorporated by reference in its entirety for all purposes.

TABLE 3
Amino Acid SASA Values

| | | | Miller et al. | Rose et al. |
|---|---|---|---|---|
| Residue | Theoretical | Empirical | (1987) | (1985) |
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

### Amino Acid Residues Having a Side Chain Comprising a Guanidine Group, Guanidine Replacement Group, or Protonated Form Thereof

As used herein, guanidine refers to the structure:

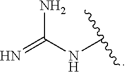 [SMILES: *NC(=N)N]

As used herein, a protonated form of guanidine refers to the structure:

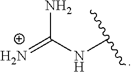 [SMILES: *NC(N)=[NH2+]]

Guanidine replacement groups refer to functional groups on the side chain of amino acids that will be positively charged at or above physiological pH or those that can recapitulate the hydrogen bond donating and accepting activity of guanidinium groups.

The guanidine replacement groups facilitate cell penetration and delivery of therapeutic agents while reducing toxicity associated with guanidine groups or protonated forms thereof. The cCPP can comprise at least one amino acid having a side chain comprising a guanidine or guanidinium replacement group. The cCPP can comprise at least two amino acids having a side chain comprising a guanidine or guanidinium replacement group. The cCPP can comprise at least three amino acids having a side chain comprising a guanidine or guanidinium replacement group

The guanidine or guanidinium group can be an isostere of guanidine or guanidinium. The guanidine or guanidinium replacement group can be less basic than guanidine.

As used herein, a guanidine replacement group refers to

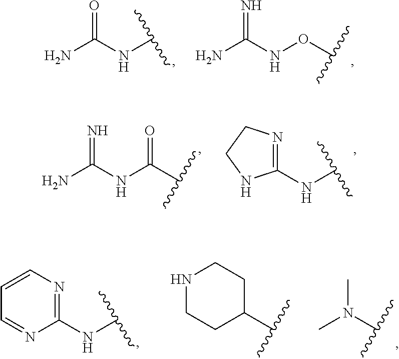

or a protonated form thereof.

The disclosure relates to a cCPP comprising from 4 to 20 amino acids residues, wherein: (i) at least one amino acid has a side chain comprising a guanidine group, or a protonated form thereof; (ii) at least one amino acid residue has no side chain or a side chain comprising

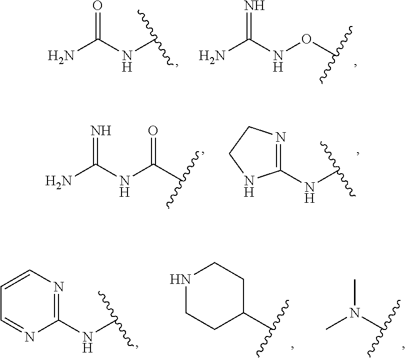

or a protonated form thereof; and (iii) at least two amino acids residues independently have a side chain comprising an aromatic or heteroaromatic group.

At least two amino acids residues can have no side chain or a side chain comprising

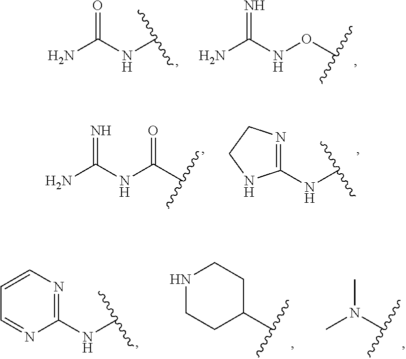

or a protonated form thereof. As used herein, when no side chain is present, the amino acid residue have two hydrogen atoms on the carbon atom(s) (e.g., —CH₂—) linking the amine and carboxylic acid.

The cCPP can comprise at least one amino acid having a side chain comprising one of the following moieties:

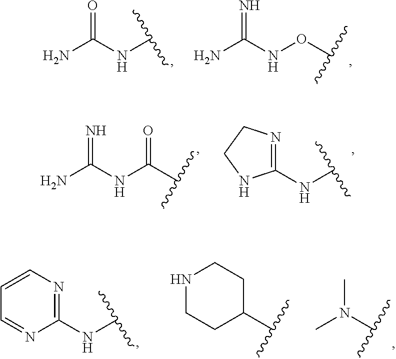

or a protonated form thereof.

The cCPP can comprise at least two amino acids each independently having one of the following moieties

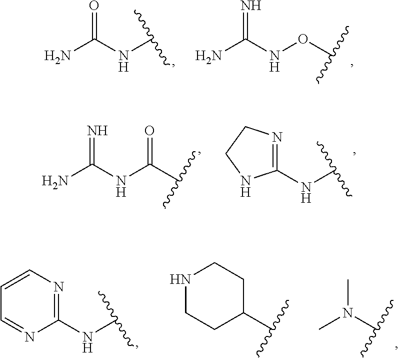

or a protonated form thereof. At least two amino acids can have a side chain comprising the same moiety selected from:

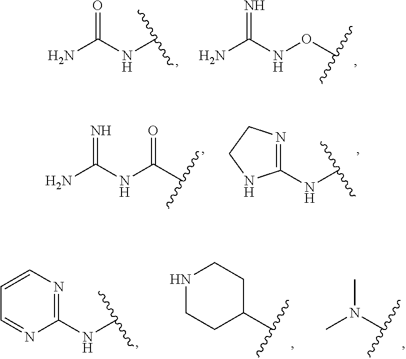

or a protonated form thereof. At least one amino acid can have a side chain comprising

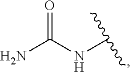 [SMILES: *NC(N)=O]

or a protonated form thereof. At least two amino acids can have a side chain comprising

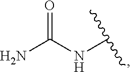 [SMILES: *NC(N)=O]

or a protonated form thereof. One, two, three, or four amino acids can have a side chain comprising

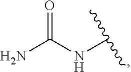 [SMILES: *NC(N)=O]

or a protonated form thereof. One amino acid can have a side chain comprising

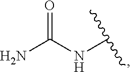 [SMILES: *NC(N)=O]

or a protonated form thereof. Two amino acids can have a side chain comprising

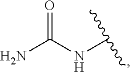 [SMILES: *NC(N)=O]

or a protonated form thereof.

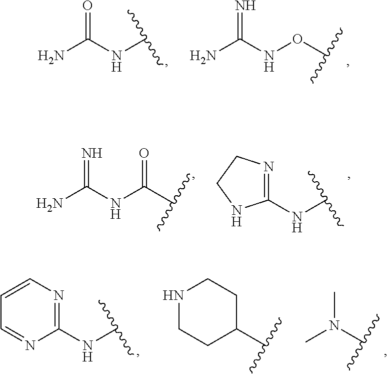

or a protonated form thereof, can be attached to the terminus of the amino acid side chain.

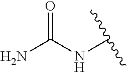 [SMILES: *NC(N)=O]

pan be attached to the terminus of the amino acid side chain.

The cCPP can comprise (iii) 2, 3, 4, 5 or 6 amino acid residues independently having a side chain comprising a guanidine group, guanidine replacement group, or a protonated form thereof. The cCPP can comprise (iii) 2 amino acid residues independently having a side chain comprising a guanidine group, guanidine replacement group, or a protonated form thereof. The cCPP can comprise (iii) 3 amino acid residues independently having a side chain comprising a guanidine group, guanidine replacement group, or a protonated form thereof. The cCPP can comprise (iii) 4 amino acid residues independently having a side chain comprising a guanidine group, guanidine replacement group, or a protonated form thereof. The cCPP can comprise (iii) 5 amino acid residues independently having a side chain comprising a guanidine group, guanidine replacement group, or a protonated form thereof. The cCPP can comprise (iii) 6 amino acid residues independently having a side chain comprising a guanidine group, guanidine replacement group, or a protonated form thereof. The cCPP can comprise (iii) 2, 3, 4, or 5 amino acid residues independently having a side chain comprising a guanidine group, guanidine replacement group, or a protonated form thereof. The cCPP can comprise (iii) 2, 3, or 4 amino acid residues independently having a side chain comprising a guanidine group, guanidine replacement group, or a protonated form thereof. The cCPP can comprise (iii) 2 or 3 amino acid residues independently having a side chain comprising a guanidine group, guanidine replacement group, or a protonated form thereof. The cCPP can comprise (iii) at least one amino acid residue having a side chain comprising a guanidine group or protonated form thereof. The cCPP can comprise (iii) two amino acid residues having a side chain comprising a guanidine group or protonated form thereof. The cCPP can comprise (iii) three amino acid residues having a side chain comprising a guanidine group or protonated form thereof.

The amino acid residues can independently have the side chain comprising the guanidine group, guanidine replacement group, or the protonated form thereof that are not contiguous. Two amino acid residues can independently have the side chain comprising the guanidine group, guanidine replacement group, or the protonated form thereof can be contiguous. Three amino acid residues can independently have the side chain comprising the guanidine group, guanidine replacement group, or the protonated form thereof can be contiguous. Four amino acid residues can independently have the side chain comprising the guanidine group, guanidine replacement group, or the protonated form thereof can be contiguous. The contiguous amino acid residues can have the same stereochemistry. The contiguous amino acids can have alternating stereochemistry.

The amino acid residues independently having the side chain comprising the guanidine group, guanidine replacement group, or the protonated form thereof, can be L-amino acids. The amino acid residues independently having the side chain comprising the guanidine group, guanidine replacement group, or the protonated form thereof, can be D-amino acids. The amino acid residues independently having the side chain comprising the guanidine group, guanidine replacement group, or the protonated form thereof, can be a mixture of L- or D-amino acids.

Each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. Each amino acid residue having the side chain comprising the guanidine group, or the protonated form thereof, can independently be a residue of arginine or a protonated form thereof.

Each amino acid having the side chain comprising a guanidine replacement group, or protonated form thereof, can independently be

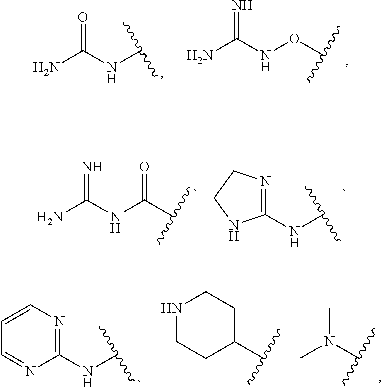

or a protonated form thereof.

Without being bound by theory, it is hypothesized that guanidine replacement groups have reduced basicity, relative to arginine and in some cases are uncharged at physiological pH (e.g., a —N(H)C(O)), and are capable of maintaining the bidentate hydrogen bonding interactions with phospholipids on the plasma membrane that is believed to facilitate effective membrane association and subsequent internalization. The removal of positive charge is also believed to reduce toxicity of the cCPP.

Those skilled in the art will appreciate that the N- and/or C-termini of the above non-natural aromatic hydrophobic amino acids, upon incorporation into the peptides disclosed herein, form amide bonds.

The cCPP can comprise a first amino acid having a side chain comprising an aromatic or heteroaromatic group and a second amino acid having a side chain comprising an aromatic or heteroaromatic group, wherein an N-terminus of a first glycine forms a peptide bond with the first amino acid having the side chain comprising the aromatic or heteroaromatic group, and a C-terminus of the first glycine forms a peptide bond with the second amino acid having the side chain comprising the aromatic or heteroaromatic group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may refer to an amino acid located at the N-terminus of the peptide sequence.

The cCPP can comprise an N-terminus of a second glycine forms a peptide bond with an amino acid having a side chain comprising an aromatic or heteroaromatic group, and a C-terminus of the second glycine forms a peptide bond with an amino acid having a side chain comprising a guanidine group, or a protonated form thereof.

The cCPP can comprise a first amino acid having a side chain comprising a guanidine group, or a protonated form thereof, and a second amino acid having a side chain comprising a guanidine group, or a protonated form thereof, wherein an N-terminus of a third glycine forms a peptide bond with a first amino acid having a side chain comprising a guanidine group, or a protonated form thereof, and a C-terminus of the third glycine forms a peptide bond with a second amino acid having a side chain comprising a guanidine group, or a protonated form thereof.

The cCPP can comprise a residue of asparagine, aspartic acid, glutamine, glutamic acid, or homoglutamine. The cCPP can comprise a residue of asparagine. The cCPP can comprise a residue of glutamine.

The cCPP can comprise a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

While not wishing to be bound by theory, it is believed that the chirality of the amino acids in the cCPPs may impact cytosolic uptake efficiency. The cCPP can comprise at least one D amino acid. The cCPP can comprise one to fifteen D amino acids. The cCPP can comprise one to ten D amino acids. The cCPP can comprise 1, 2, 3, or 4 D amino acids. The cCPP can comprise 2, 3, 4, 5, 6, 7, or 8 contiguous amino acids having alternating D and L chirality. The cCPP can comprise three contiguous amino acids having the same chirality. The cCPP can comprise two contiguous amino acids having the same chirality. At least two of the amino acids can have the opposite chirality. The at least two amino acids having the opposite chirality can be adjacent to each other. At least three amino acids can have alternating stereochemistry relative to each other. The at least three amino acids having the alternating chirality relative to each other can be adjacent to each other. At least four amino acids have alternating stereochemistry relative to each other. The at least four amino acids having the alternating chirality relative to each other can be adjacent to each other. At least two of the amino acids can have the same chirality. At least two amino acids having the same chirality can be adjacent to each other. At least two amino acids have the same chirality and at least two amino acids have the opposite chirality. The at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. The amino acid residues that form the cCPP can all be L-amino acids. The amino acid residues that form the cCPP can all be D-amino acids.

At least two of the amino acids can have a different chirality. At least two amino acids having a different chirality can be adjacent to each other. At least three amino acids can have different chirality relative to an adjacent amino acid. At least four amino acids can have different chirality relative to an adjacent amino acid. At least two amino acids have the same chirality and at least two amino acids have a different chirality. One or more amino acid residues that form the cCPP can be achiral. The cCPP can comprise a motif of 3, 4, or 5 amino acids, wherein two amino acids having the same chirality can be separated by an achiral amino acid. The cCPPs can comprise the following sequences: D-X-D; D-X-D-X; D-X-D-X-D; L-X-L; L-X-L-X; or L-X-L-X-L, wherein X is an achiral amino acid. The achiral amino acid can be glycine.

An amino acid having a side chain comprising:

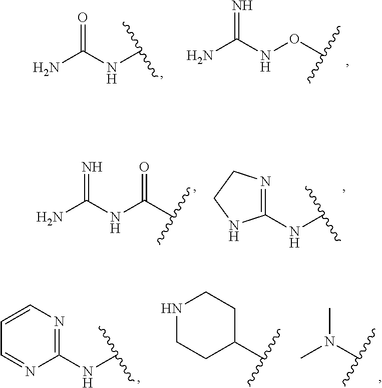

or a protonated form thereof, can be adjacent to an amino acid having a side chain comprising an aromatic or heteroaromatic group. An amino acid having a side chain comprising:

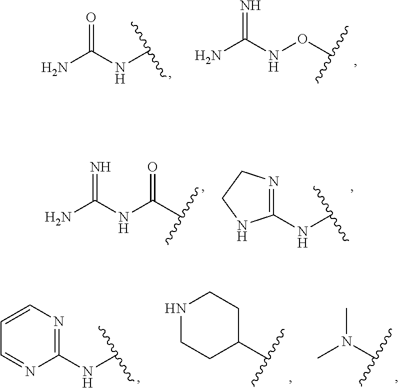

or a protonated form thereof, can be adjacent to at least one amino acid having a side chain comprising a guanidine or protonated form thereof. An amino acid having a side chain comprising a guanidine or protonated form thereof can be adjacent to an amino acid having a side chain comprising an aromatic or heteroaromatic group. Two amino acids having a side chain comprising:

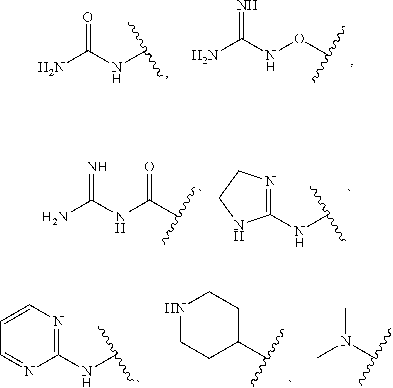

or protonated forms thereof can be adjacent to each other. Two amino acids having a side chain comprising a guanidine or protonated form thereof are adjacent to each other. The cCPPs can comprise at least two contiguous amino acids having a side chain can comprise an aromatic or heteroaromatic group and at least two non-adjacent amino acids having a side chain comprising:

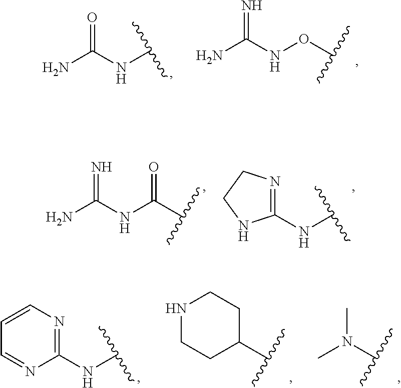

or a protonated form thereof. The cCPPs can comprise at least two contiguous amino acids having a side chain comprising an aromatic or heteroaromatic group and at least two non-adjacent amino acids having a side chain comprising

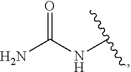 [SMILES: *NC(N)=O]

or a protonated form thereof. The adjacent amino acids can have the same chirality. The adjacent amino acids can have the opposite chirality. Other combinations of amino acids can have any arrangement of D and L amino acids, e.g., any of the sequences described in the preceding paragraph.

At least two amino acids having a side chain comprising:

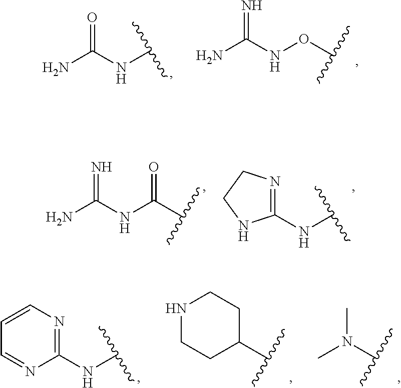

or a protonated form thereof, are alternating with at least two amino acids having a side chain comprising a guanidine group or protonated form thereof.

The cCPP can comprise the structure of Formula (A):

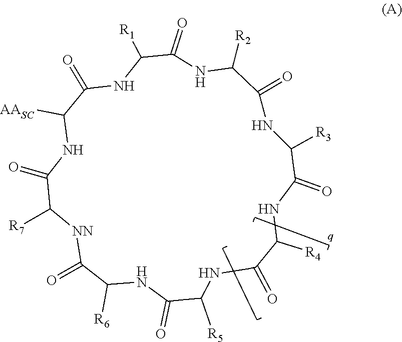

or a protonated form thereof,
wherein:
   R₁, R₂ and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently 1-1 or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine;
AA_{SC} is an amino acid side chain; and
q is 1, 2, 3 or 4.

In embodiments, the cyclic peptide of Formula (A) is not FfΦRrRrQ (SEQ ID NO:67). In embodiments, the cyclic peptide of Formula (A) is FfΦRrRrQ (SEQ ID NO:67).

The cCPP can comprise the structure of Formula (I):

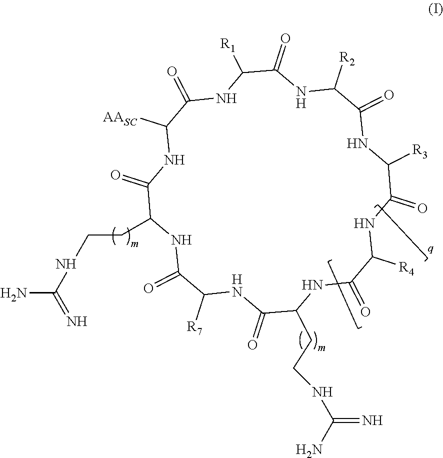

or a protonated form thereof,
wherein:
   R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₇ are independently H or an amino acid side chain;
AA_{SC} is an amino acid side chain;
q is 1, 2, 3 or 4; and
each m is independently an integer of 0, 1, 2, or 3.

R₁, R₂, and R₃ can each independently be H, -alkylene-aryl, or -alkylene-heteroaryl. R₁, R₂, and R₃ can each independently be H, —C₁₋₃alkylene-aryl, or —C₁₋₃alkylene-heteroaryl. R₁, R₂, and R₃ can each independently be H or -alkylene-aryl. R₁, R₂, and R₃ can each independently be H or —C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, —C₁₋₃alkylene-Ph or —C₁₋₃alkylene-Naphthyl. R₁, R₂, and R₃ can each independently be H, —CH₂Ph, or —CH₂Naphthyl. R₁, R₂, and R₃ can each independently be H or —CH₂Ph.

R₁, R₂, and R₃ can each independently be the side chain of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

R₁ can be the side chain of tyrosine. R₁ can be the side chain of phenylalanine. R₁ can be the side chain of 1-naphthylalanine. R₁ can be the side chain of 2-naphthylalanine. R₁ can be the side chain of tryptophan. R₁ can be the side chain of 3-benzothienylalanine. R₁ can be the side chain of 4-phenylphenylalanine. R₁ can be the side chain of 3,4-difluorophenylalanine. R₁ can be the side chain of 4-trifluoromethylphenylalanine. R₁ can be the side chain of 2,3,4,5,6-pentafluorophenylalanine. R₁ can be the side chain of homophenylalanine. R₁ can be the side chain of β-homophenylalanine. R₁ can be the side chain of 4-tert-butyl-phenylalanine. R₁ can be the side chain of 4-pyridinylalanine. R₁ can be the side chain of 3-pyridinylalanine. R₁ can be the side chain of 4-methylphenylalanine. R₁ can be the side chain of 4-fluorophenylalanine. R₁ can be the side chain of 4-chlorophenylalanine. R₁ can be the side chain of 3-(9-anthryl)-alanine.

R₂ can be the side chain of tyrosine. R₂ can be the side chain of phenylalanine. R₂ can be the side chain of 1-naphthylalanine. R₁ can be the side chain of 2-naphthylalanine. R₂ can be the side chain of tryptophan. R₂ can be the side chain of 3-benzothienylalanine. R₂ can be the side chain of 4-phenylphenylalanine. R₂ can be the side chain of 3,4-difluorophenylalanine. R₂ can be the side chain of 4-trifluoromethylphenylalanine. R₂ can be the side chain of 2,3,4,5,6-pentafluorophenylalanine. R₂ can be the side chain of homophenylalanine. R₂ can be the side chain of β-homophenylalanine. R₂ can be the side chain of 4-tert-butyl-phenylalanine. R₂ can be the side chain of 4-pyridinylalanine. R₂ can be the side chain of 3-pyridinylalanine. R₂ can be the side chain of 4-methylphenylalanine. R₂ can be the side chain of 4-fluorophenylalanine. R₂ can be the side chain of 4-chlorophenylalanine. R₂ can be the side chain of 3-(9-anthryl)-alanine.

R₃ can be the side chain of tyrosine. R₃ can be the side chain of phenylalanine. R₃ can be the side chain of 1-naphthylalanine. R₃ can be the side chain of 2-naphthylalanine. R₃ can be the side chain of tryptophan. R₃ can be the side chain of 3-benzothienylalanine. R₃ can be the side chain of 4-phenylphenylalanine. R₃ can be the side chain of 3,4-difluorophenylalanine. R₃ can be the side chain of 4-trifluoromethylphenylalanine. R₃ can be the side chain of 2,3,4,5,6-pentafluorophenylalanine. R₃ can be the side chain of homophenylalanine. R₃ can be the side chain of β-homophenylalanine. R₃ can be the side chain of 4-tert-butyl-phenylalanine. R₃ can be the side chain of 4-pyridinylalanine. R₃ can be the side chain of 3-pyridinylalanine. R₃ can be the side chain of 4-methylphenylalanine. R₃ can be the side chain of 4-fluorophenylalanine. R₃ can be the side chain of 4-chlorophenylalanine. R₃ can be the side chain of 3-(9-anthryl)-alanine.

R₄ can be H, -alkylene-aryl, -alkylene-heteroaryl. R₄ can be H, —C₁₋₃alkylene-aryl, or —C₁₋₃alkylene-heteroaryl. R₄ can be H or -alkylene-aryl. 14 can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₄ can be H, —C₁₋₃alkylene-Ph or —C₁₋₃alkylene-Naphthyl. R₁ can be H or the side chain of an amino acid in Table 1 or Table 3. R₄ can be H or an amino acid residue having a side chain comprising an aromatic group. R₄ can be H, —CH₂Ph, or —CH₂Naphthyl. R₄ can be H or —CH₂Ph.

R₅ can be H, -alkylene-aryl, -alkylene-heteroaryl. R₅ can be H, —C₁₋₃alkylene-aryl, or —C₁₋₃alkylene-heteroaryl. R₅ can be H or -alkylene-aryl. R₅ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₅ can be H, —C₁₋₃alkylene-Ph or —C₁₋₃alkylene-Naphthyl. R₅ can be H or the side chain of an amino acid in Table 1 or Table 3. R₄ can be H or an amino acid residue having a side chain comprising an aromatic group. R₅ can be H, —CH₂Ph, or —CH₂Naphthyl. R₄ can be H or —CH₂Ph.

R₆ can be H, -alkylene-aryl, -alkylene-heteroaryl. R₆ can be H, —C₁₋₃alkylene-aryl, or —C₁₋₃alkylene-heteroaryl. R₆ can be H or -alkylene-aryl. R₆ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl Aryl can be phenyl or naphthyl Aryl can phenyl. Heteroaryl can be pyridyl, quinolyl, and isoquinolyl. R₆ can be H, —C₁₋₃alkylene-Ph or —C₁₋₃alkylene-Naphthyl. R₆ can be H or the side chain of an amino acid in Table 1 or Table 3. R₆ can be H or an amino acid residue having a side chain comprising an aromatic group. R₆ can be H, —CH₂Ph, or —CH₂Naphthyl. R₆ can be H or —CH₂Ph.

R₇ can be H, -alkylene-aryl, -alkylene-heteroaryl. R₇ can be H, —C₁₋₃alkylene-aryl, or —C₁₋₃alkylene-heteroaryl. R₇ can be H or -alkylene-aryl. R₇ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl, Aryl can be phenyl or naphthyl Aryl can phenyl. Heteroaryl can be pyridyl, quinolyl, and isoquinolyl. R₇ can be H, —C₁₋₃alkylene-Ph or —C₁₋₃alkylene-Naphthyl. R₇ can be H or the side chain of an amino acid in Table 1 or Table 3. R₇ can be H or an amino acid residue having a side chain comprising an aromatic group. R₇ can be H, —CH₂Ph, or —CH₂Naphthyl. R₇ can be H or —CH₂Ph.

One, two or three of R₁, R₂, R₃, R₄, R₅, R₆, and R₇ can be —CH₂Ph. One of R₁, R₂, R₃, R₄, R₅, R₆, and R₇ can be —CH₂Ph. Two of R₁, R₂, R₃, R₄, R₅, R₆, and R₇ can be —CH₂Ph. Three of R₁, R₂, R₃, R₄, R₅, R₆, and R₇ can be —CH₂Ph. At least one of R₁, R₂, R₃, R₄, R₅, R₆, and R₇, can be —CH₂Ph. No more than four of R₁, R₂, R₃, R₄, R₅, R₆, and R₇ can be —CH₂Ph.

One, two or three of R₁, R₂, R₃, and R₄ are —CH₂Ph. One of R₁, R₂, R₃, and R₄ is —CH₂Ph. Two of R₁, R₂, R₃, and R₄ are —CH₂Ph. Three of R₁, R₂, R₃, and R₄ are —CH₂Ph. At least one of R₁, R₂, R₃, and R₄ is —CH₂Ph.

One, two or three of R₁, R₂, R₃, R₄, R₅, R₆, and R₇ can be H. One of R₁, R₂, R₃, R₄, R₅, R₆, and R₇ can be H. Two of R₁, R₂, R₃, R₄, R₅, R₆, and R₇ are H. Three of R₁, R₂, R₃, R₄, R₅, R₆, and R₇ can be H. At least one of R₁, R₂, R₃, R₄, R₅, R₆, and R₇ can be H. No more than three of R₁, R₂, R₃, R₄, R₅, R₆, and R₇ can be —CH₂Ph.

One, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

At least one of R₄, R₅, R₆, and R₇ can be side chain of 3-guanidino-2-aminopropionic acid. At least one of R₄, R₅, R₆, and R₇ can be side chain of 4-guanidino-2-aminobutanoic acid. At least one of R₄, R₅, R₆, and R₇, can be side chain of arginine. At least one of R₄, R₅, R₆, and R₇, can be side chain of homoarginine. At least one of R₄, R₅, R₆, and R₇ can be side chain of N-methylarginine. At least one of R₄, R₅, R₆, and R₇ can be side chain of N,N-dimethylarginine. At least one of R₄, R₅, R₆, and R₇ can be side chain of 2,3-diaminopropionic acid. At least one of R₄, R₅, R₆, and R₇ can be side chain of 2,4-diaminobutanoic acid, lysine. At least one of R₄, R₅, R₆, and R₇ can be side chain of N-methyllysine. At least one of R₄, R₅, R₆, and R₇ can be side chain of N,N-dimethyllysine. At least one of R₄, R₅, R₆, and R₇ can be side chain of N-ethyllysine. At least one of R₄, R₅, R₆, and R₇ can be side chain of N,N,N-trimethyllysine, 4-guanidinophenylalanine. At least one of R₄, R₅, R₆, and R₇ can be side chain of citrulline. At least one of R₄, R₅, R₆, and R can be side chain of N,N-dimethyllysine, β-homoarginine. At least one of R₄, R₅, R₆, and R₇ can be side chain of 3-(1-piperidinyl)alanine.

At least two of R₄, R₅, R₆, and R₇ can be side chain of 3-guanidino-2-aminopropionic acid. At least two of R₄, R₅, R₆, and R₇ can be side chain of 4-guanidino-2-aminobutanoic acid. At least two of R₄, R₅, R₆, and R₇ can be side chain of arginine. At least two of R₄, R₅, R₆, and R₇ can be side chain of homoarginine. At least two of R₄, R₅, R₆, and R₇ can be side chain of N-methylarginine. At least two of R₄, R₅, R₆, and R₇ can be side chain of N,N-dimethylarginine. At least two of R₄, R₅, R₆, and R₇ can be side chain of 2,3-diaminopropionic acid. At least two of R₄, R₅, R₆, and R₇ can be side chain of 2,4-diaminobutanoic acid, lysine. At least two of R₄, R₅, R₆, and R₇ can be side chain of N-methyllysine. At least two of R₄, R₅, R₆, and R₇ can be side chain of N,N-dimethyllysine. At least two of R₄, R₅, R₆, and R₇ can be side chain of N-ethyllysine. At least two of R₄, R₅, R₆, and R₇ can be side chain of N,N,N-trimethyllysine, 4-guanidinophenylalanine. At least two of R₄, R₅, R₆, and R₇ can be side chain of citrulline. At least two of R₄, R₅, R₆, and R₇ can be side chain of N,N-dimethyllysine, β-homoarginine. At least two of R₄, R₅, R₆, and R₇ can be side chain of 3-(1-piperidinyl)alanine.

At least three of R₄, R₅, R₆, and R₇ can be side chain of 3-guanidino-2-aminopropionic acid. At least three of R₄, R₅, R₆, and R₇ can be side chain of 4-guanidino-2-aminobutanoic acid. At least three of R₄, R₅, R₆, and R₇ can be side chain of arginine. At least three of R₄, R₅, R₆, and R₇ can be side chain of homoarginine. At least three of R₄, R₅, R₆, and R₇ can be side chain of N-methylarginine. At least three of R₄, R₅, R₆, and R₇ can be side chain of N,N-dimethylarginine. At least three of R₄, R₅, R₆, and R₇ can be side chain of 2,3-diaminopropionic acid. At least three of R₄, R₅, R₆, and R₇ can be side chain of 2,4-diaminobutanoic acid, lysine. At least three of R₄, R₅, R₆, and R₇ can be side chain of N-methyllysine. At least three of R₄, R₅, R₆, and R₇ can be side chain of N,N-dimethyllysine. At least three of R₄, R₅, R₆, and R₇ can be side chain of N-ethyllysine. At least three of R₄, R₅, R₆, and R₇ can be side chain of N,N,N-trimethyllysine, 4-guanidinophenylalanine. At least three of R₄, R₅, R₆, and R₇ can be side chain of citrulline. At least three of R₄, R₅, R₆, and R₇ can be side chain of N, N-dimethyllysine, -homoarginine. At least three of R₄, R₅, R₆, and R₇ can be side chain of 3-(1-piperidinyl)alanine.

AA_{SC} can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to the glutamine residue.

q can be 1, 2, or 3. q can 1 or 2. q can be 1. q can be 2. q can be 3. q can be 4.

m can be 1-3. m can be 1 or 2. m can be 0. m can be 1. m can be 2. m can be 3.

The cCPP of Formula (A) can comprise the structure of Formula (I)

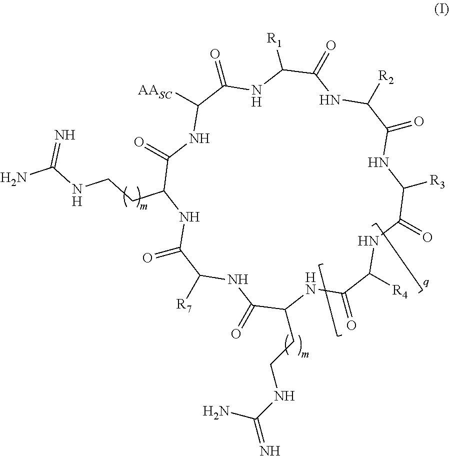

or protonated form thereof, wherein AA_{SC}, R₁, R₂, R₃, R₄, R₇, m, and q are as defined herein.

The cCPP of Formula (A) can comprise the structure of Formula (I-a) or Formula (I-b):

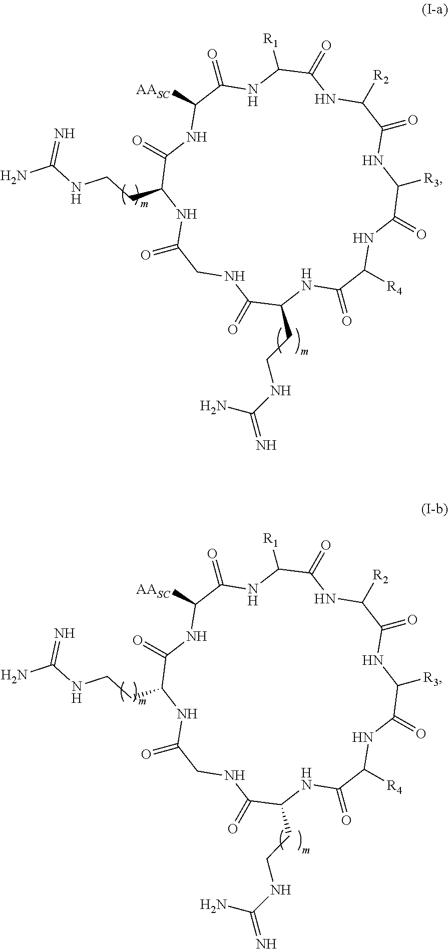

or protonated form thereof, wherein AA_{SC}, R₁, R₂, R₃, R₄, and in areas defined herein.

The cCPP of Formula (A) can comprise the structure of Formula (I-1), (I-2), (I-3), or (I-4):

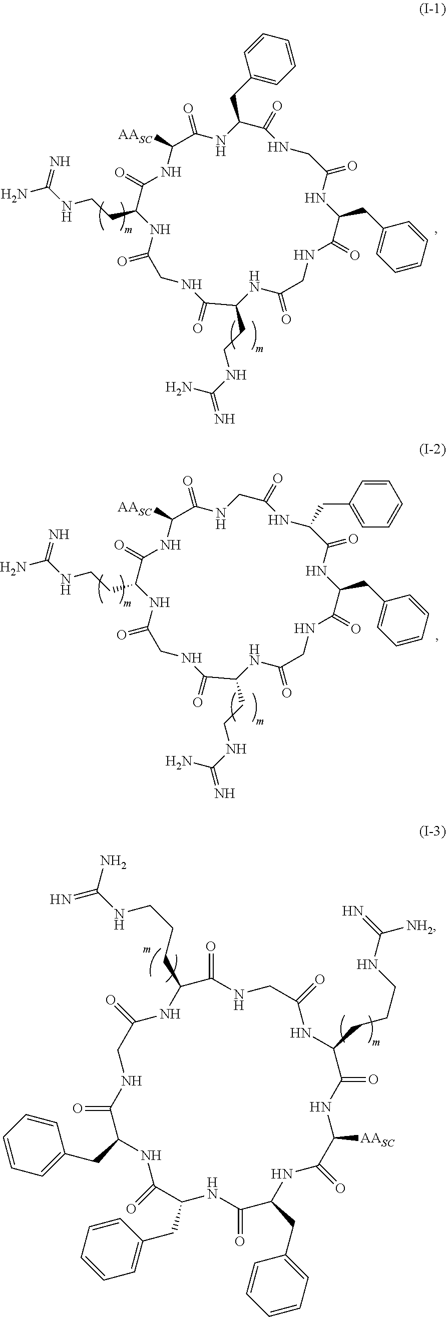

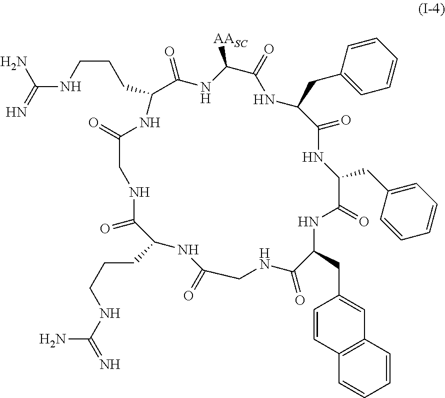

or protonated form thereof, wherein AA_{SC} and m are as defined herein.

The cCPP of Formula (A) can comprise the structure of Formula (I-5) or (I-6):

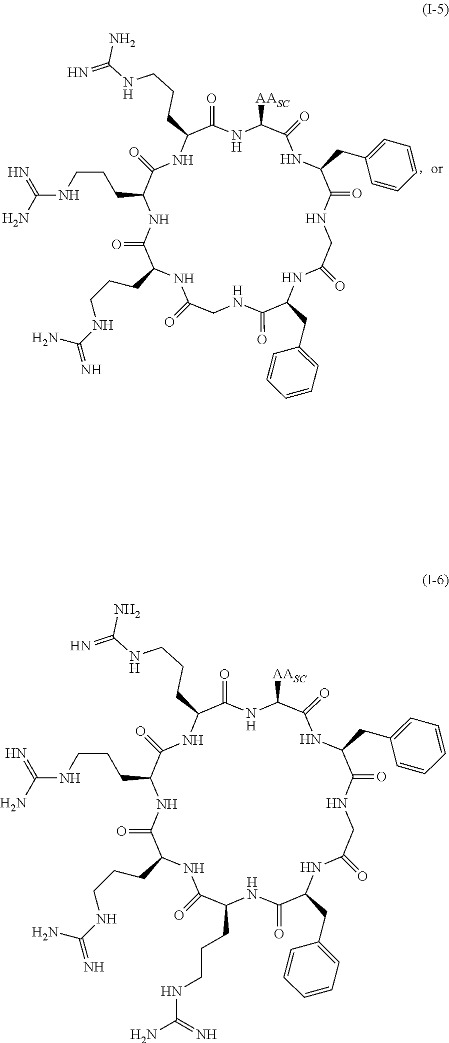

or protonated form thereof, wherein AA_{SC} is as defined herein.

The cCPP of Formula (A) can comprise the structure of Formula (I-1):

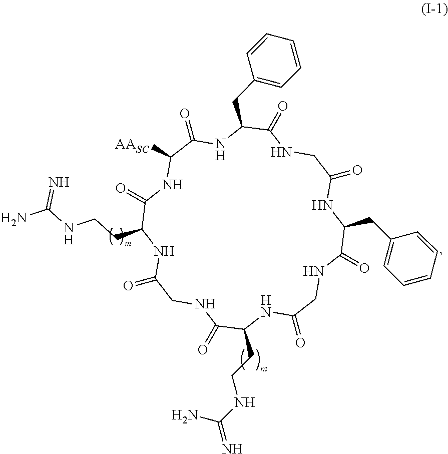

or a protonated form thereof,
wherein AA_{SC} and m are as defined herein.

The cCPP of Formula (A) can comprise the structure of Formula (I-2):

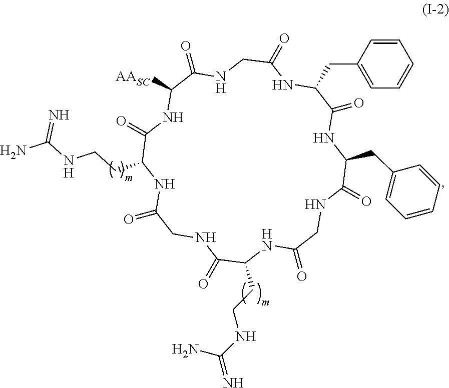

or a protonated form thereof,
wherein AA_{SC} and m are as defined herein.

The cCPP of Formula (A) can comprise the structure of Formula (I-3):

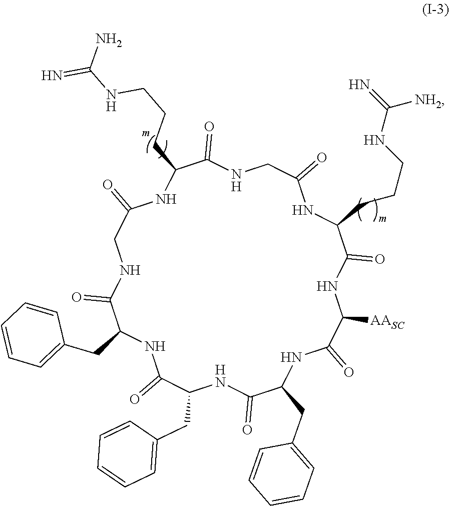

or a protonated form thereof,
wherein AA_{SC} and n are as defined herein.

The cCPP of Formula (A) can comprise the structure of Formula (I-4):

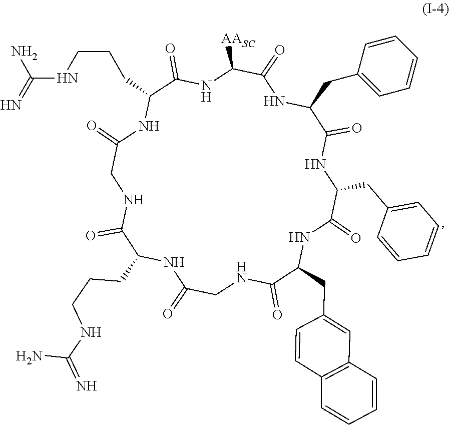

or a protonated form thereof,
wherein AA_{SC} and m are as defined herein.

The cCPP of Formula (A) can comprise the structure of Formula (I-5):

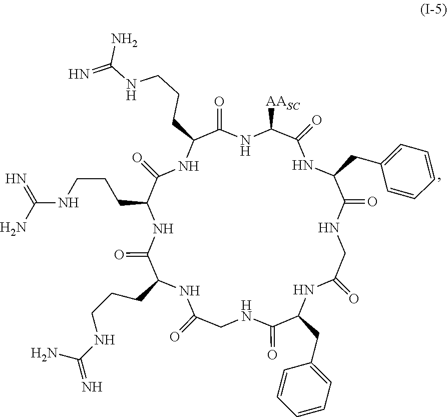

or a protonated form thereof,
wherein AA_{SC} and m are as defined herein.

The cCPP of Formula (A) can comprise the structure of Formula (I-6):

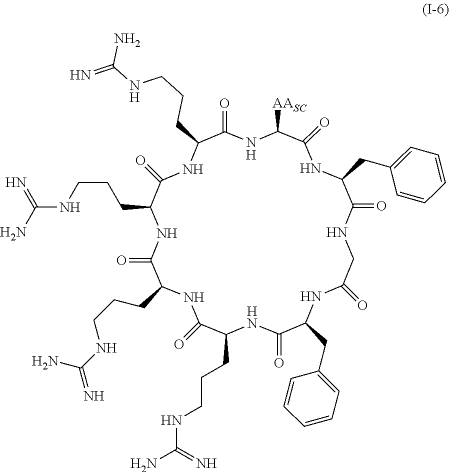

or a protonated form thereof, wherein AA_{SC} and n are as defined herein.

The cCPP can comprise one of the following sequences: FGFGRGR (SEQ ID NO:68); GfFGrGr (SEQ ID NO:69), FfΦGRGR (SEQ ID NO:70); FfFGRGR (SEQ ID NO:71); or FfΦGrGr (SEQ ID NO:72). The cCPP can have one of the following sequences: FGFΦ (SEQ ID NO:73); GfFGrGrQ (SEQ ID NO:74), FfΦGRGRQ (SEQ ID NO:75); FfFGRGRQ (SEQ ID NO:76); or FfΦGrGrQ (SEQ ID NO:77).

The disclosure also relates to a cCPP having the structure of Formula (II):

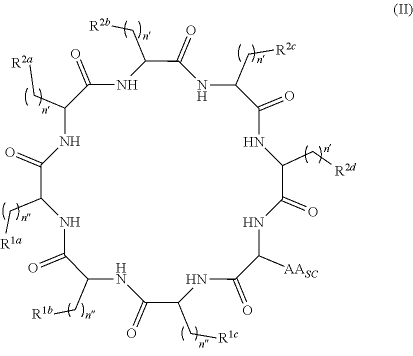

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is

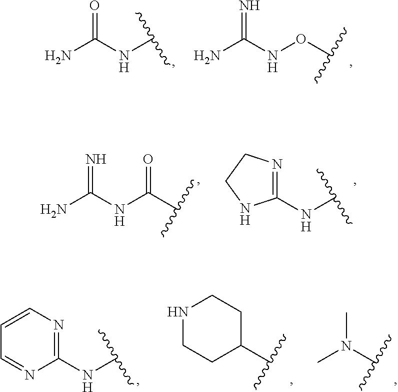

or a protonated form thereof;
at least one of R^{2a}, R^{2b}, R^{2c} and R^{2d} is guanidine or a protonated form thereof;
each n″ is independently an integer 0, 1, 2, 3, 4, or 5;
each n′ is independently an integer from 0, 1, 2, or 3, and
if n′ is 0 then R^{2a}, R^{2b}, R^{2c} and R^{2d} is absent.

At least two of R^{2a}, R^{2b}, R^{2c} and R^{2d} can be

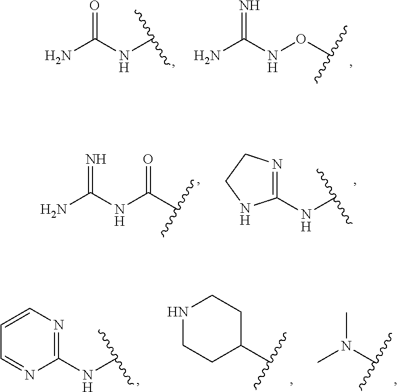

or a protonated form thereof. Two or three of R^{2a}, R^{2b}, R^{2c} and R^{2d} can be

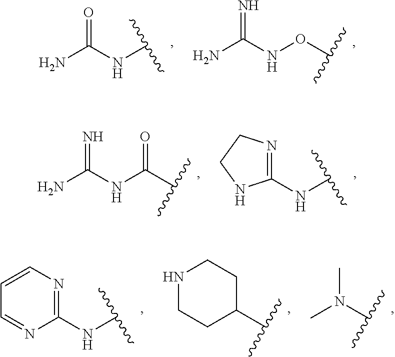

or a protonated form thereof. One of R^{2a}, R^{2b}, R^{2c} and R^{2d} can be

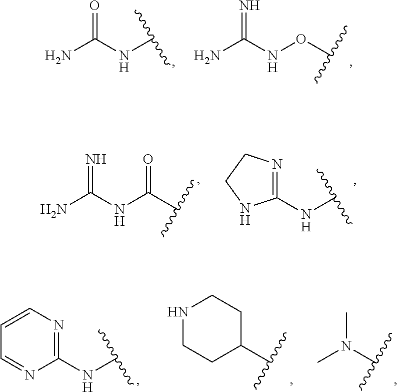

or protonated form thereof. At least one of R^{2a}, R^{2b}, R^{2c} and R^{2d} can be

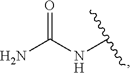 [SMILES: *NC(N)=O]

or a protonated form thereof, and the remaining of R^{2a}, R^{2b}, R^{2c} and R^{2d} can be guanidine or a protonated form thereof. At least two of R^{2a}, R^{2b}, R^{2c} and R^{2d} can be

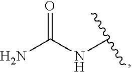 [SMILES: *NC(N)=O]

or a protonated form thereof, and the remaining of R^{2a}, R^{2b}, R^{2c} and R^{2d} can be guanidine, or a protonated form thereof.

All of R^{2a}, R^{2b}, R^{2c} and R^{2d} can be

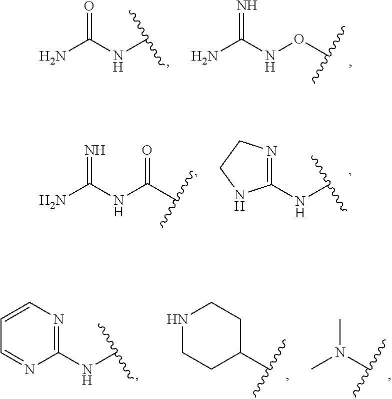

or a protonated form thereof. At least of R^{2a}, R^{2b}, R^{2c} and R^{2d} can be

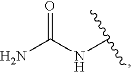 [SMILES: *NC(N)=O]

or a protonated form thereof, and the remaining of R^{2a}, R^{2b}, R^{2c} and R^{2d} can be guaninide or a protonated form thereof. At least two R^{2a}, R^{2b}, R^{2c} and R^{2d} groups can be

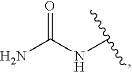 [SMILES: *NC(N)=O]

or a protonated form thereof, and the remaining of R^{2a}, R^{2b}, R^{2c} and R^{2d} are guanidine, or a protonated form thereof.

Each of R^{2a}, R^{2b}, R^{2c} and R^{2d} can independently be 2,3-diaminopropionic acid, 2,4-diaminobutyric acid, the side chains of ornithine, lysine, methyllysine, dimethyllysine, trimethyllysine, homo-lysine, serine, homo-serine, threonine, allo-threonine, histidine, 1-methylhistidine, 2-aminobutanedioic acid, aspartic acid, glutamic acid, or homo-glutamic acid.

AA_{SC} can be

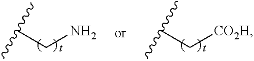

wherein t can be an integer from 0 to 5. AA_{SC} can be

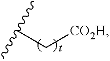

wherein t can be an integer from 0 to 5. t can be 1 to 5. t is 2 or 3. t can be 2. t can be 3.

R^{1a}, R^{1b}, and R^{1c} can each independently be 6- to 14-membered aryl. R^{1a}, R^{1b}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1b}, and R^{1c} can each be independently selected pyridyl, quinolyl, or isoquinolyl.

Each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

Each n″ can independently be an integer from 1 to 3. Each n″ can independently be 2 or 3. Each n″ can be 2. Each n″ can be 3. At least one n″ can be 0. At least one n″ can be 1. At least one n″ can be 2. At least one n″ can be 3.

Each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′ is 3.

The cCPP of Formula (II) can have the structure of Formula (II-1):

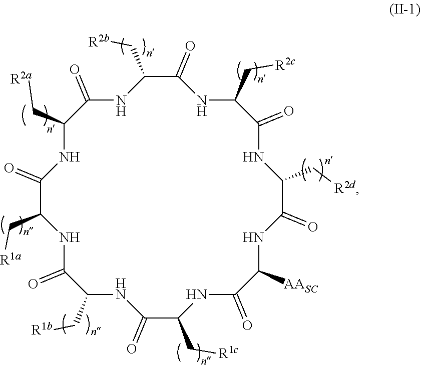

wherein R^{1a}, R^{1b}, R^{1c}, R^{2a}, R^{2b}, R^{2c}, R^{2d}, AA_{SC}, n′ and n″ are as defined herein.

The cCPP of Formula (II) can have the structure of Formula (IIa):

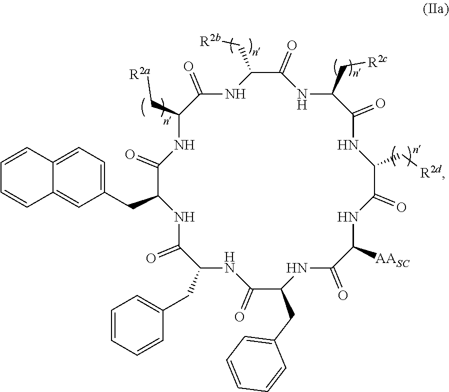

wherein R^{1a}, R^{1b}, R^{1c}, R^{2a}, R^{2b}, R^{2c}, R^{2d}, AA_{SC} and n′ are as defined herein.

The cCPP of formula (II) can have the structure of Formula (IIb):

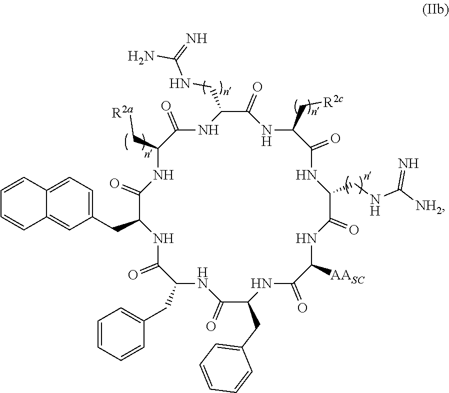

wherein R^{2a}, R^{2b}, AA_{SC}, and n′ are as defined herein.

The cCPP can have the structure of Formula (IIc):

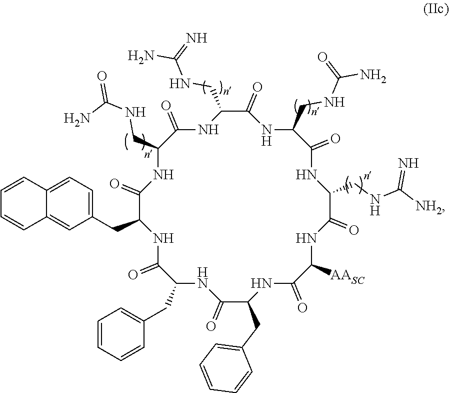

or a protonated form thereof,
wherein:
AA_{SC} and n′ are as defined herein.

The cCPP of Formula (IIa) has one of the following structures:

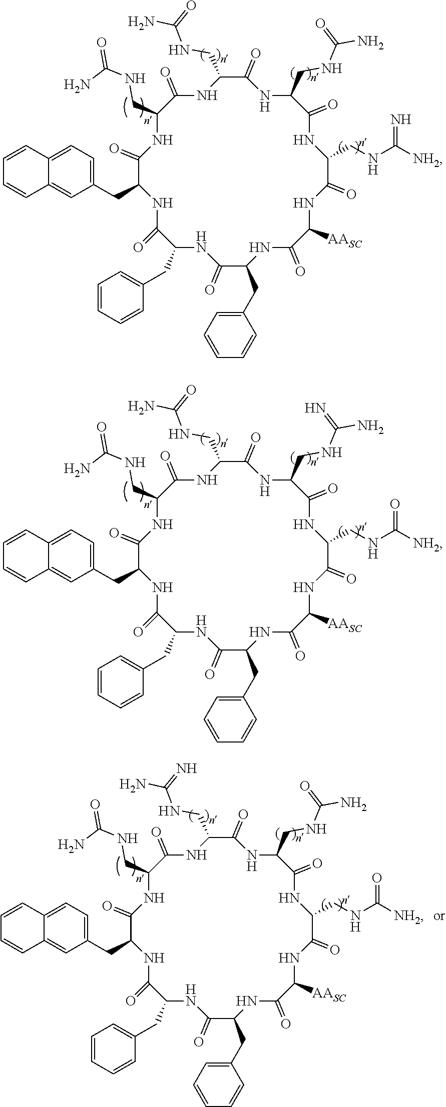

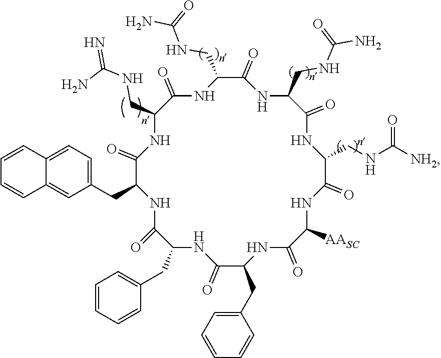

wherein AA_{SC} and n are as defined herein.

The cCPP of Formula (IIa) has one of the following structures:

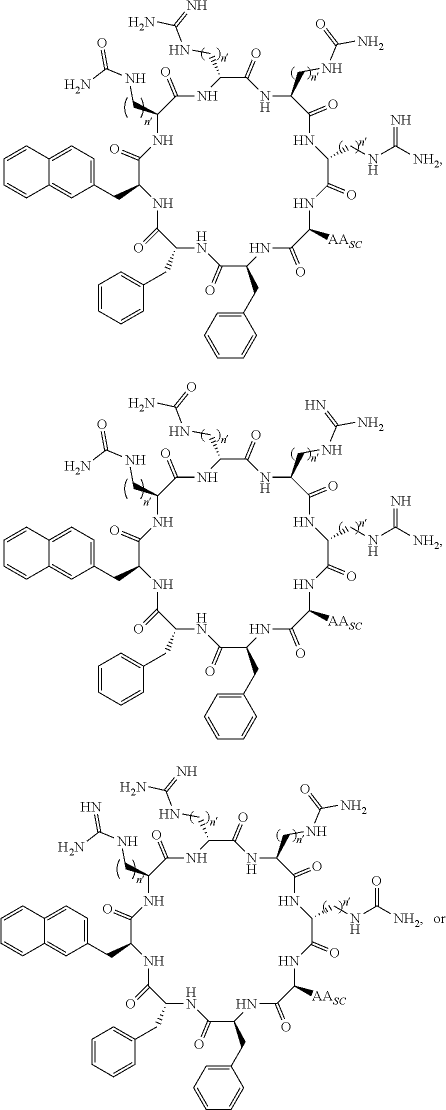

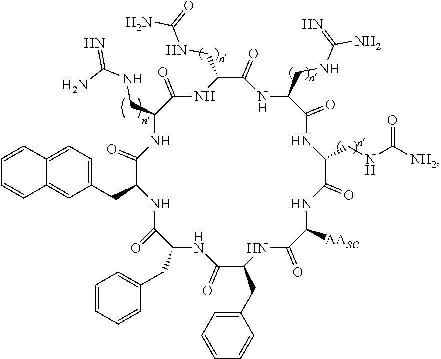

wherein AA_{SC} and n are as defined herein

The cCPP of Formula (IIa) has one of the following structures:

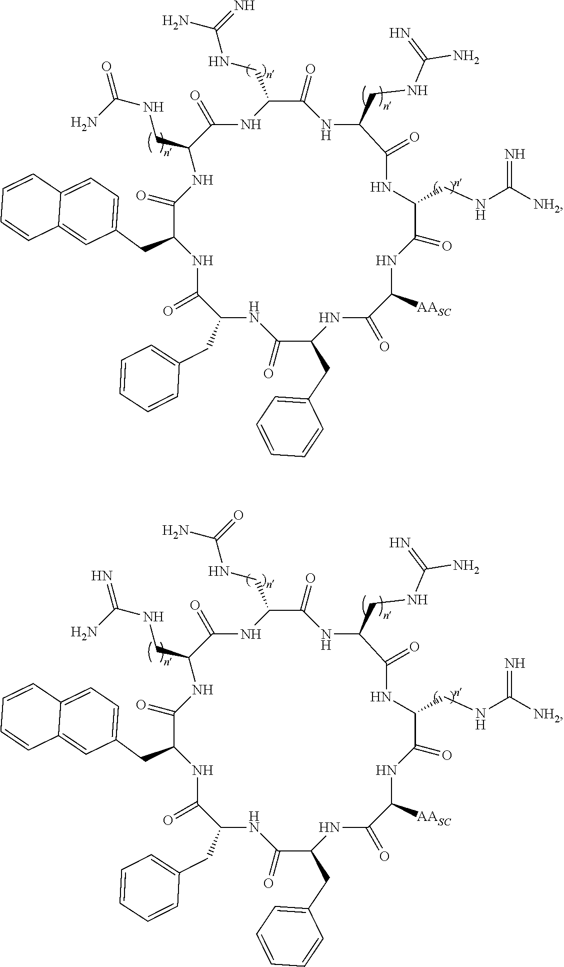

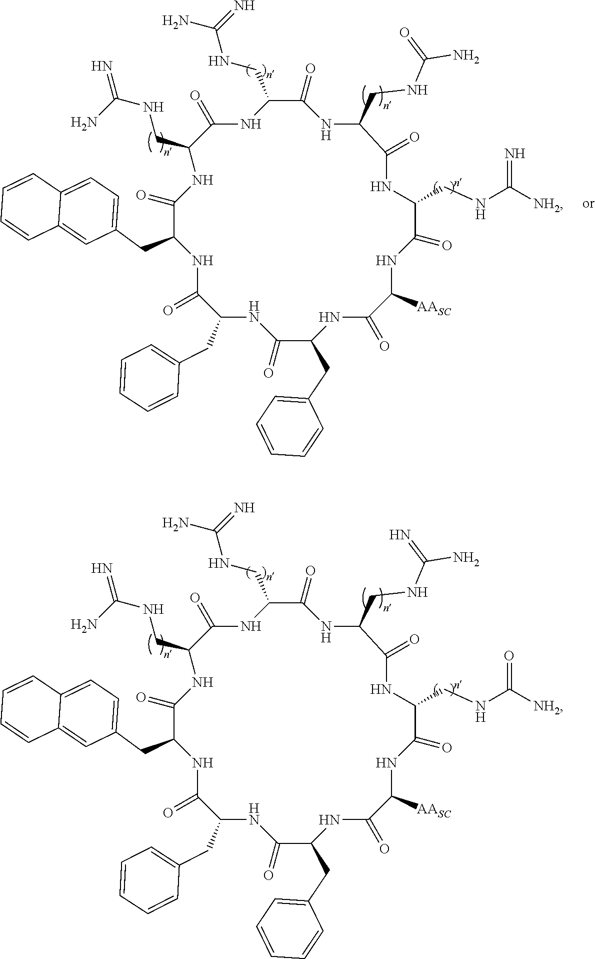

wherein AA_{SC} and n are as defined herein.

The cCPP of Formula (II) has one of the following structures:

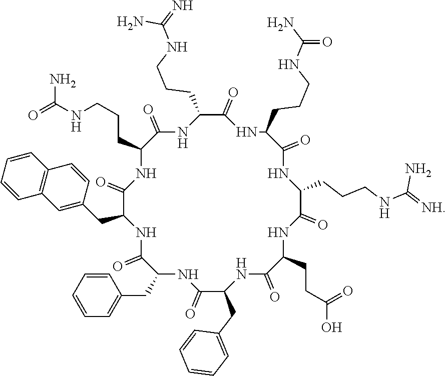 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP of Formula (II) can have the structure:

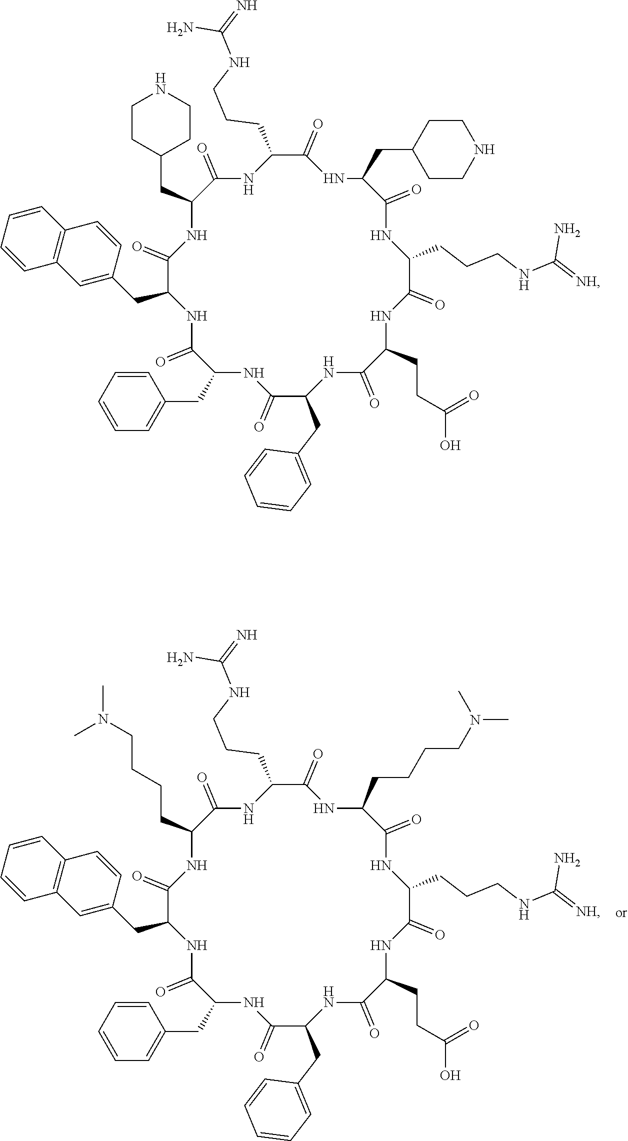 [SMILES: N=C(N)NCCC[C@H]1NC(=O)[C@H](CC2CCNCC2)NC(=O)[C@@H](CCCNC(=N)N)NC(=O)[C@H](CC2CCNCC2)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O ; CN(C)CCCC[C@@H]1NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)[C@H](CCCCN(C)C)NC(=O)[C@@H](CCCNC(=N)N)NC1=O]

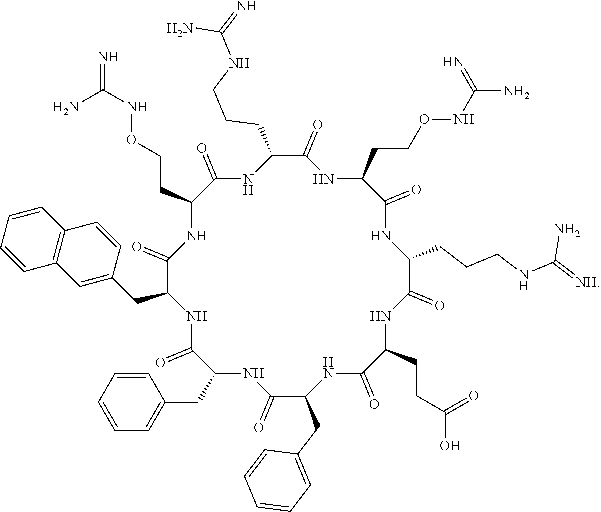 [SMILES: N=C(N)NCCC[C@H]1NC(=O)[C@H](CCONC(=N)N)NC(=O)[C@@H](CCCNC(=N)N)NC(=O)[C@H](CCONC(=N)N)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP can have the structure of Formula (III):

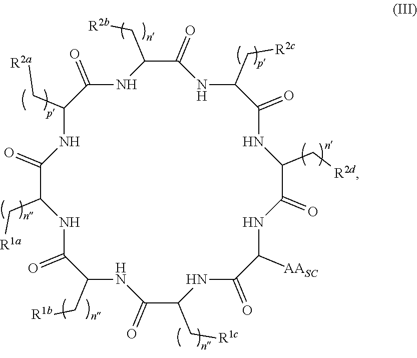

wherein:
AA_{SC} is an amino acid side chain;
R^{1a}, R^{1b}, and R^{1c} are each independently a 6- to 14-membered aryl or a 6- to 14-membered heteroaryl;
R^{2a} and R^{2c} are each independently H,

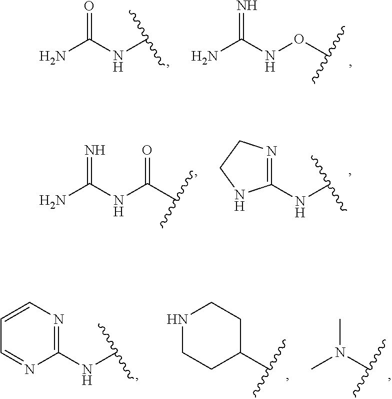

or a protonated form thereof;
R^{2b} and R^{2d} are each independently guanidine or a protonated form thereof;
each n″ is independently an integer from 1 to 3;
each n′ is independently an integer from 1 to 5; and
each p′ is independently an integer from 0 to 5.

The cCPP of Formula (III) can have the structure of Formula (III-1):

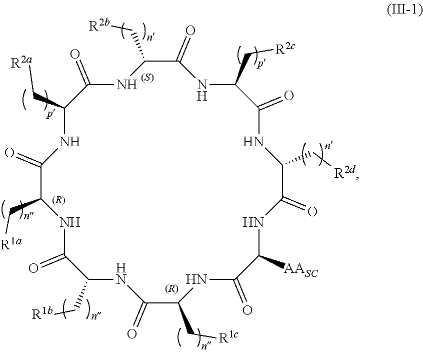

wherein:
AA_{SC}, R^{1a}, R^{1b}, R^{1c}, R^{2a}, R^{2c}, R^{2b}, R^{2d}, n′, n″, and p′ are as defined herein.

The cCPP of Formula (III) can have the structure of Formula (IIIa):

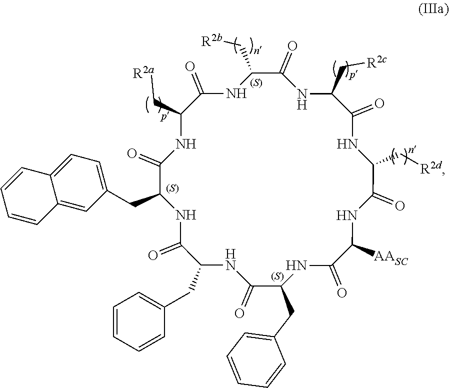

wherein:
AA_{SC}, R^{2a}, R^{2c}, R^{2b}, R^{2d}, n′, n″, and p′ are as defined herein.

In Formulas (III), (III-1), and (IIIa), R^{a} and R^{c} can be H. R^{a} and R^{c} can be H and R^{b} and R^{d} can each independently be guanidine or protonated form thereof. R^{a} can be H. R^{b} can be H. p′ can be 0. R^{a} and R^{c} can be H and each p′ can be 0.

In Formulas (III), (III-1), and (IIIa), R^{a} and R^{c} can be H, R^{b} and R^{d} can each independently be guanidine or protonated form thereof, n″ can be 2 or 3, and each p′ can be 0.

p′ can 0. p′ can 1. p′ can 2. p′ can 3. p′ can 4. p′ can be 5.

The cCPP can have the structure:

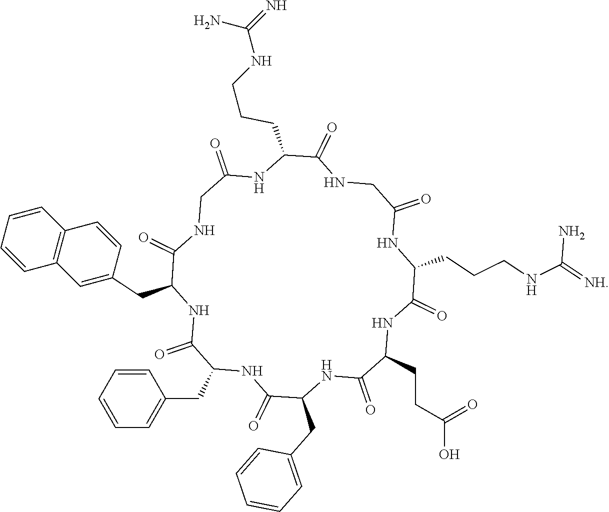 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]

The cCPP of Formula (A) can be selected from:

| CPP Sequence | SEQ ID NO: |
|---|---|
| (FfΦRrRrQ) | 78 |
| (FfΦCit-r- | 79 |
| Cit-rQ) | |
| (FfΦGrGrQ) | 80 |
| (FfFGRGRQ) | 81 |
| (FGFGRGRQ) | 82 |
| (GfFGrGrQ) | 83 |
| (FGFGRRRQ) | 84 |
| (FGFRRRRQ) | 85 |

The cCPP of Formula (A) can be selected from:

| CPP Sequence | SEQ ID NO: |
|---|---|
| FΦRRRRQ | 86 |
| fΦRrRrQ | 87 |
| FfΦRrRrQ | 78 |
| FfΦCit-r- | 79 |
| Cit-rQ | |
| FfΦGrGrQ | 80 |
| FfΦRGRGQ | 88 |
| FfFGRGRQ | 81 |
| FGFGRGRQ | 82 |
| GfFGrGrQ | 83 |
| FGFGRRRQ | 84 |
| FGFRRRRQ | 85 |

In embodiments, the cCPP is not selected from:

CPP sequences and SEQ ID NOs:

| | |
|---|---|
| FΦRRRQ | 89 |
| FΦRRRC | 90 |
| FΦRRRU | 91 |
| RRRΦFQ | 92 |
| RRRRΦF | 93 |
| FΦRRRR | 94 |
| FϕrRrRq | 95 |
| FϕrRrRQ | 96 |
| FΦRRRRQ | 97 |
| fΦRrRrQ | 98 |
| RRFRΦRQ | 99 |
| FRRRRΦQ | 100 |
| FRFRΦRQ | 101 |
| RRΦFRRQ | 102 |
| CRRRRFWQ | 103 |
| FfΦRrRrQ | 104 |
| FFΦRRRRQ | 105 |
| RFRFRΦRQ | 106 |
| URRRRFWQ | 107 |
| CRRRRFWQ | 108 |
| FΦRRRRQK | 109 |
| FΦRRRRQC | 110 |
| fΦRrRrRQ | 111 |
| FΦRRRRRQ | 112 |
| RRRRΦFDΩC | 113 |
| FΦRRR | 114 |
| FWRRR | 115 |
| RRRΦF | 116 |
| RRRWF | 117 |

Where Φ = L-naphthylalanine; ϕ = D-naphthylalanine; Ω = L-norleucine

In embodiments, the cCPP is not selected from:

CPP sequences and SEQ ID NOs:

| | |
|---|---|
| FΦRRRQ | 89 |
| FΦRRRC | 90 |
| FΦRRRU | 91 |
| RRRΦFQ | 92 |
| RRRRΦF | 93 |
| FΦRRRR | 94 |
| FϕrRrRq | 95 |
| FϕrRrRQ | 96 |
| FΦRRRRQ | 97 |
| fΦRrRrQ | 98 |
| RRFRΦRQ | 99 |
| FRRRRΦQ | 100 |
| rRFRΦRQ | 101 |
| RRΦFRRQ | 102 |
| CRRRRFWQ | 103 |
| FfΦRrRrQ | 104 |
| FFΦRRRRQ | 105 |
| RFRFRΦRQ | 106 |
| URRRRFWQ | 107 |
| CRRRRFWQ | 108 |
| FΦRRRRQK | 109 |
| FΦRRRRQC | 110 |
| fΦRrRrRQ | 111 |
| FΦRRRRRQ | 112 |
| RRRRΦFDΩC | 113 |
| FΦRRR | 114 |
| FWRRR | 115 |
| RRRΦF | 116 |
| RRRWF | 117 |

Where Φ = L-naphthylalanine; ϕ = D-naphthylalanine; Ω = L-norleucine

AA_{SC} can be conjugated to a linker.

### Linker

The cCPP of the disclosure can be conjugated to a linker. The linker can link a cargo to the cCPP. The linker can be attached to the side chain of an amino acid of the cCPP, and the cargo can be attached at a suitable position on linker.

The linker can be any appropriate moiety which can conjugate a cCPP to one or more additional moieties, e.g., an exocyclic peptide (EP) and/or a cargo. Prior to conjugation to the cCPP and one or more additional moieties, the linker has two or more functional groups, each of which are independently capable of forming a covalent bond to the cCPP and one or more additional moieties. If the cargo is an oligonucleotide, the linker can be covalently bound to the 5′ end of the cargo or the 3′ end of the cargo. The linker can be covalently bound to the 5′ end of the cargo. The linker can be covalently bound to the 3′ end of the cargo. If the cargo is a peptide, the linker can be covalently bound to the N-terminus or the C-terminus of the cargo. The linker can be covalently bound to the backbone of the oligonucleotide or peptide cargo. The linker can be any appropriate moiety which conjugates a cCPP described herein to a cargo such as an oligonucleotide, peptide or small molecule.

The linker can comprise hydrocarbon linker.

The linker can comprise a cleavage site. The cleavage site can be a disulfide, or caspase-cleavage site (e.g, Val-Cit-PABC).

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more —(R¹-J-R²)z″— subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R³ is independently selected from H, alkyl, alkenyl, alkynyl, carbocyclyl, and heterocyclyl, each of which is optionally substituted, and z″ is an integer from 1 to 50; (viii) —(R¹-J)z″- or -(J-R¹)z″—, wherein each of R¹, at each instance, is independently alkylene, alkenylene, alkynylene, carbocyclyl, or heterocyclyl, each J is independently C, NR³, —NRC(O)—, S, or O, wherein R³ is H, alkyl, alkenyl, alkynyl, carbocyclyl, or heterocyclyl, each of which is optionally substituted, and z″ is an integer from 1 to 50; or (ix) the linker can comprise one or more of (i) through (x).

The linker can comprise one or more D or L amino acids and/or —(R¹-J-R²)z″—, wherein each of R¹ and R², at each instance, are independently alkylene, each J is independently C, NR³, —N³C(O)—, S, and O, wherein R⁴ is independently selected from H and alkyl, and z″ is an integer from 1 to 50; or combinations thereof.

The linker can comprise a —(OCH₂CH₂)_{z′}— (e.g., as a spacer), wherein z′ is an integer from 1 to 23, e.g., 2, 3, 4, 5, 6, 7, 8, 9, 10, 11, 12, 13, 14, 15, 16, 17, 18, 19, 20, 21, 22, or 23. “—(OCH₂CH₂) z′ can also be referred to as polyethylene glycol (PEG).

The linker can comprise one or more amino acids. The linker can comprise a peptide. The linker can comprise a —(OCH₂CH₂)_{z′}, wherein z′ is an integer from 1 to 23, and a peptide. The peptide can comprise from 2 to 10 amino acids. The linker can further comprise a functional group (FG) capable of reacting through click chemistry. FG can be an azide or alkyne, and a triazole is formed when the cargo is conjugated to the linker.

The linker can comprises (i) a β alanine residue and lysine residue; (ii) -(J-R¹)z″; or (iii) a combination thereof. Each R can independently be alkylene, alkenylene, alkynylene, carbocyclyl, or heterocyclyl, each J is independently C, NR³, —NR³C(O)—, S, or O, wherein R₃ is H, alkyl, alkenyl, alkynyl, carbocyclyl, or heterocyclyl, each of which is optionally substituted, and z″ can be an integer from 1 to 50. Each R¹ can be alkylene and each J can be O.

The linker can comprise (i) residues of f-alanine, glycine, lysine, 4-aminobutyric acid, 5-aminopentanoic acid, 6-aminohexanoic acid or combinations thereof; and (ii) —(R¹-J)z″- or -(J-R¹)z″. Each R¹ can independently be alkylene, alkenylene, alkynylene, carbocyclyl, or heterocyclyl, each J is independently C, NR³, —NRC(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be 0. The linker can comprise glycine, beta-alanine, 4-aminobutyric acid, 5-aminopentanoic acid, 6-aminohexanoic acid, or a combination thereof.

The linker can be a trivalent linker. The linker can have the structure:

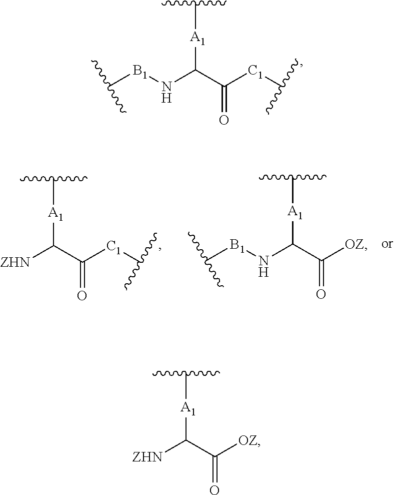

wherein A₁, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂—(CH₂O)ₙ—S—S—(CH₂O)ₙ—COOH], or caspase-cleavage site (Val-Cit-PABC).

The hydrocarbon can be a residue of glycine or beta-alanine.

The linker can be bivalent and link the cCPP to a cargo. The linker can be bivalent and link the cCPP to an exocyclic peptide (EP).

The linker can be trivalent and link the cCPP to a cargo and to an EP.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)—, or a combination thereof.

The linker can have the structure:

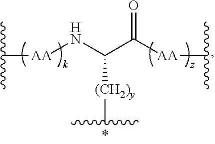

Wherein: each AA is independently an amino acid residue; * is the point of attachment to the AA_{SC}, and AA_{SC} is side chain of an amino acid residue of the cCPP; x is an integer from 1-10; y is an integer from 1-5; and z is an integer from 1-10. X can be an integer from 1-5. X can be an integer from 1-3. X can be 1. Y can be an integer from 2-4. Y can be 4. Z can be an integer from 1-5. Z can be an integer from 1-3. Z can be 1. Each AA can independently be selected from glycine, β-alanine, 4-aminobutyric acid, 5-aminopentanoic acid, and 6-aminohexanoic acid.

The cCPP can be attached to the cargo through a linker (“L”). The linker can be conjugated to the cargo through a bonding group (“M”).

The linker can have the structure:

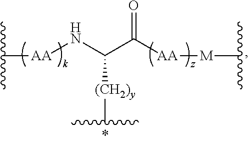

wherein: x is an integer from 1-10; y is an integer from 1-5; z is an integer from 1-10; each AA is independently an amino acid residue; * is the point of attachment to the AA_{SC}, and AA_{SC} is side chain of an amino acid residue of the cCPP; and M is a bonding group defined herein.

The linker can have the structure:

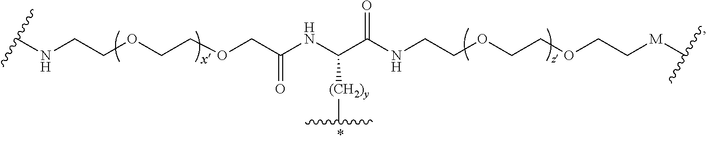

wherein: x′ is an integer from 1-23; y is an integer from 1-5; Z is an integer from 1-23; * is the point of attachment to the AA_{SC}, and AA_{SC} is a side chain of an amino acid residue of the cCPP; and M is a bonding group defined herein.

The linker can have the structure:

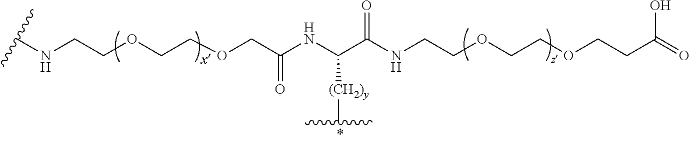

wherein: x′ is an integer from 1-23; y is an integer from 1-5; and z′ is an integer from 1-23; * is the point of attachment to the AA_{SC}, and AA_{SC} is a side chain of an amino acid residue of the cCPP.

x can be an integer from 1-10, e.g., 1, 2, 3, 4, 5, 6, 7, 8, 9, or 10, inclusive of all ranges and subranges therebetween.

x′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. X′ can be an integer from 5-15. X′ can be an integer from 9-13. V can be an integer from 1-5. X′ can be 1.

y can be an integer from 1-5, e.g., 1, 2, 3, 4, or 5, inclusive of all ranges and subranges therebetween. Y can be an integer from 2-5. Y can be an integer from 3-5. Y can be 3 or 4. Y can be 4 or 5. Y can be 3. Y can be 4. Y can be 5.

z can be an integer from 1-10, e.g., 1, 2, 3, 4, 5, 6, 7, 8, 9, or 10, inclusive of all ranges and subranges therebetween.

z′ can be an integer from 1-23, e.g., 1, 2, 3, 4, 5, 6, 7, 8, 9, 10, i1, 12, 13, 14, 15, 16, 17, 18, 19, 20, 21, 22, or 23, inclusive of all ranges and subranges therebetween. Z′ can be an integer from 5-15 Z′ can be an integer from 9-13. Z′ can be 11.

As discussed above, the linker or M (wherein M is part of the linker) can be covalently bound to cargo at any suitable location on the cargo. The linker or M (wherein M is part of the linker) can be covalently bound to the 3′ end of oligonucleotide cargo or the 5′ end of an oligonucleotide cargo. The linker or M (wherein M is part of the linker) can be covalently bound to the N-terminus or the C-terminus of a peptide cargo. The linker or M (wherein M is pail of the linker) can be covalently bound to the backbone of an oligonucleotide or a peptide cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can be bound to the side chain of aspartic acid, glutamic acid, glutamine, asparagine, or lysine, or a modified side chain of glutamine or asparagine (e.g., a reduced side chain having an amino group), on a peptide cargo. The linker can be bound to the side chain of lysine on the peptide cargo.

The linker can have a structure:

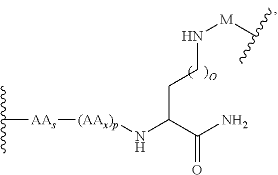

wherein
M is a group that conjugates L to a cargo, for example, an oligonucleotide;
AAₛ is a side chain or terminus of an amino acid on the cCPP;
each AAₓ is independently an amino acid residue;
is an integer from 0 to 10; and
p is an integer from 0 to 5.

The linker can have a structure:

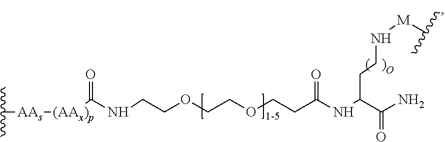

wherein
M is a group that conjugates L to a cargo, for example an oligonucleotide;
AAₛ is a side chain or terminus of an amino acid on the cCPP;
each AAₓ is independently an amino acid residue;
is an integer from 0 to 10; and
p is an integer from 0 to 5.

M can comprise an alkylene, alkenylene, alkynylene, carbocyclyl, or heterocyclyl, each of which is optionally substituted. M can be selected from:

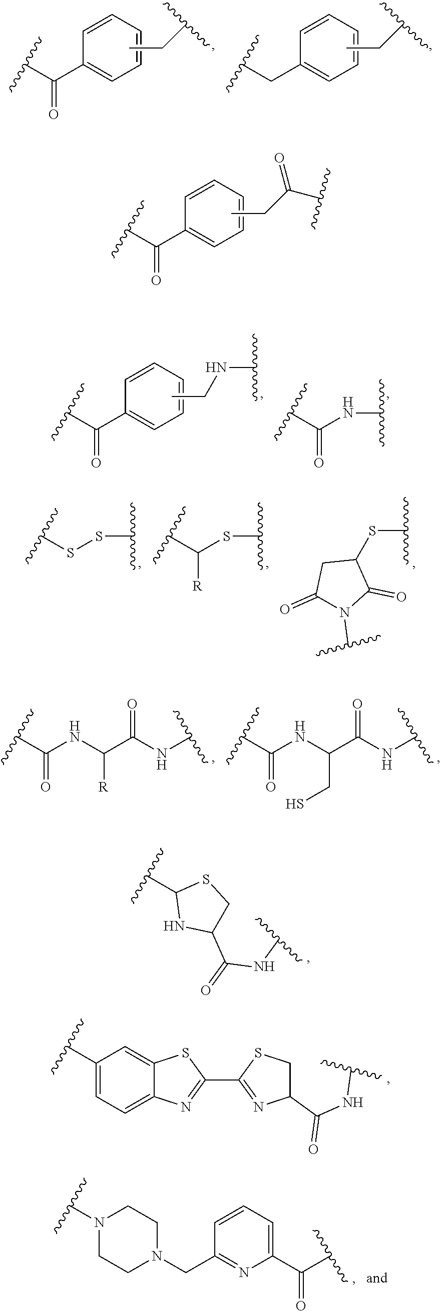

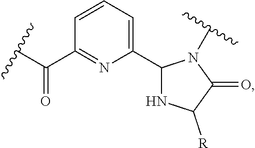

wherein R is alkyl, alkenyl, alkynyl, carbocyclyl, or heterocyclyl.

M can be selected from:

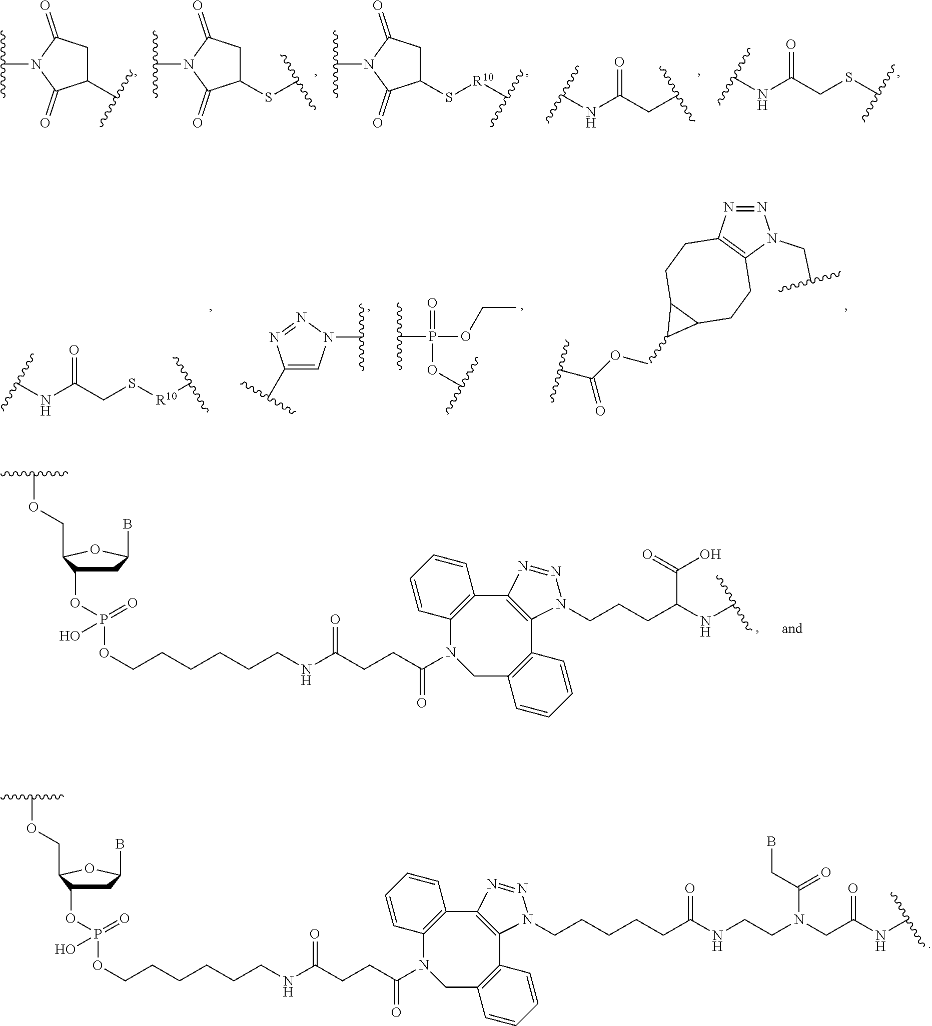

Wherein: R¹⁰ is alkylene, cycloalkyl, or

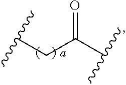

wherein a is 0 to 10.

M can be

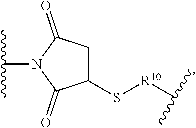

### R¹⁰ can be

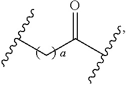

### and a is 0 to 10. M can be

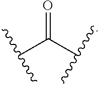 [SMILES: *C(*)=O]

M can be a heterobifunctional crosslinker, e.g.,

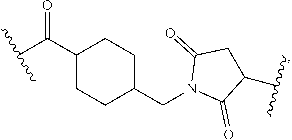 [SMILES: *C(=O)C1CCC(CN2C(=O)CC(*)C2=O)CC1]

which is disclosed in Williams et al. Curr. Protoc Nucleic Acid Chem. 2010, 42, 4.41.1-4.41.20, incorporated herein by reference its entirety.

M can be —C(O)—.

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be an AA_{SC} as defined herein.

Each AAₓ is independently a natural or non-natural amino acid. One or more AAₓ can be a natural amino acid. One or more AAₓ can be a non-natural amino acid. One or more AAₓ can be a β-amino acid. The β-amino acid can be β-alanine.

o can be an integer from 0 to 10, e.g., 0, 1, 2, 3, 4, 5, 6, 7, 8, 9, and 10. O can be 0, 1, 2, or 3. O can be 0. O can be 1. O can be 2. O can be 3.

p can be 0 to 5, e.g., 0, 1, 2, 3, 4, or 5. P can be 0. P can be 1. P can be 2. P can be 3. P can be 4. P can be 5.

The linker can have the structure:

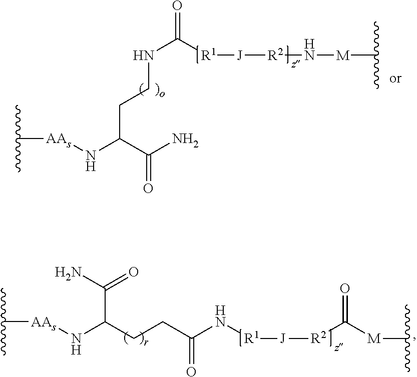

wherein M, AAₛ, each —(R¹-J-R²)z″—, o and z″ are defined herein; r can be 0 or 1.

r can be 0. R can be 1.

The linker can have the structure:

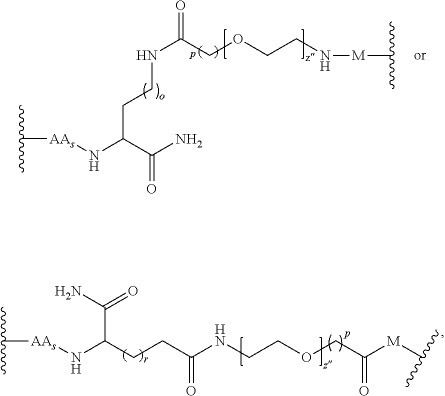

wherein each of M, AAₛ, o, p, q, r and z″ can be as defined herein.

z″ can be an integer from 1 to 50, e.g., 1, 2, 3, 4, 5, 6, 7, 8, 9, 10, 11, 12, 13, 14, 15, 16, 17, 18, 19, 20, 21, 22, 23, 24, 25, 26, 2⁷, 28, 29, 30, 31, 32, 33, 34, 35, 36, 3738, 39, 40, 41, 42, 43, 44, 45, 46, 47, 48, 49, and 50, inclusive of all ranges and values therebetween. Z″ can be an integer from 5-20. Z″ can be an integer from 10-1.

The linker can have the structure:

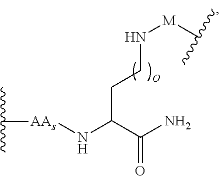

wherein:
M, AAₛ, and o are as defined herein.

Other non-limiting examples of suitable linkers include:

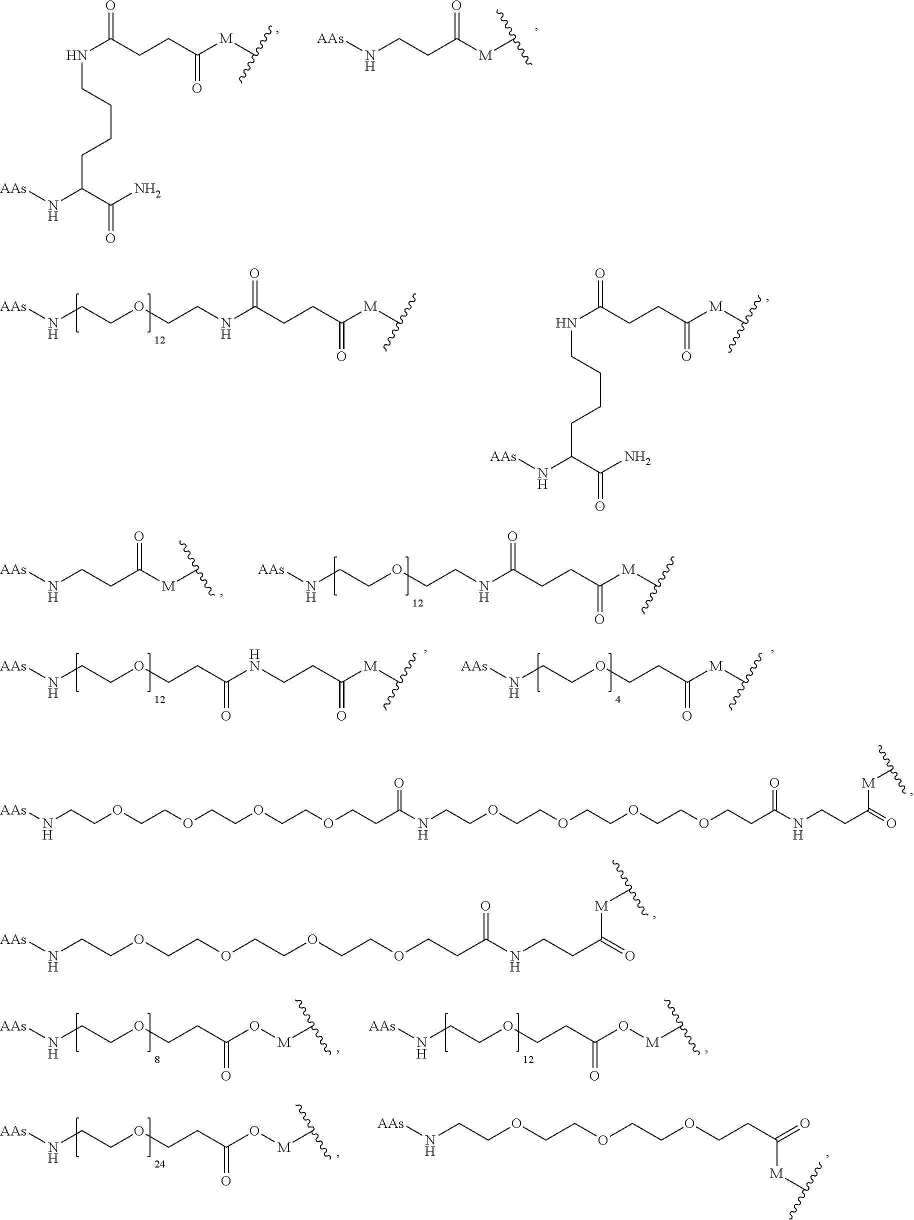

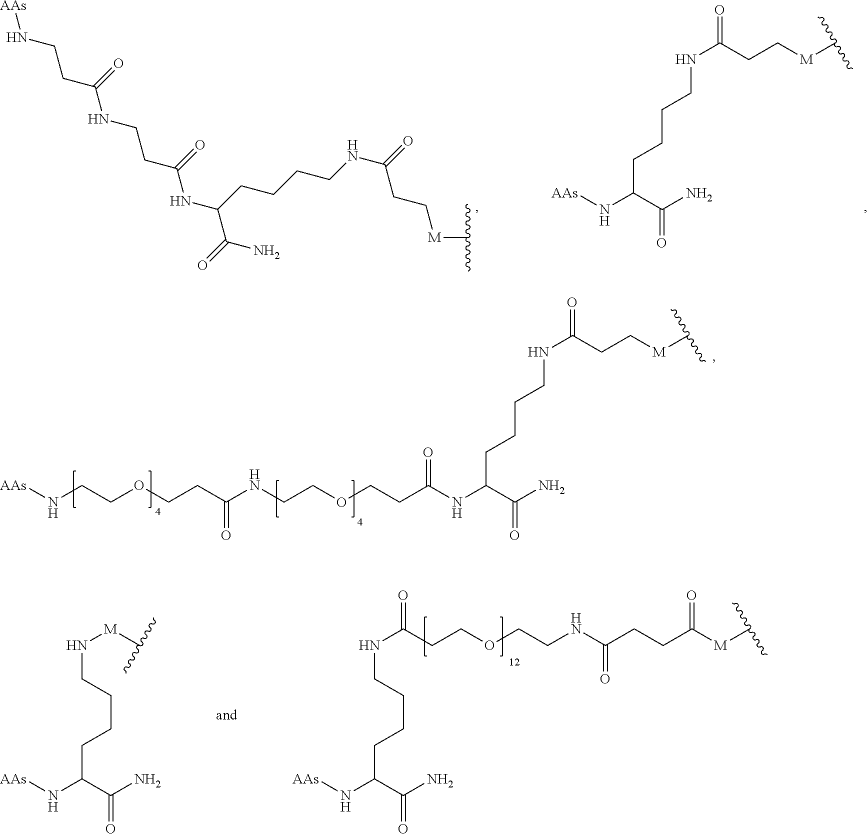

wherein M and AAₛ are as defined herein.

Provided herein is a compound comprising a cCPP and an AC that is complementary to a target in a pre-mRNA sequence further comprising L, wherein the linker is conjugated to the AC through a bonding group (M), wherein M is

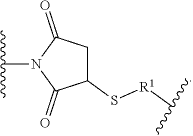

Provided herein is a compound comprising a cCPP and a cargo that comprises an antisense compound (AC), for example, an antisense oligonucleotide, that is complementary to a target in a pre-mRNA sequence, wherein the compound further comprises L, wherein the linker is conjugated to the AC through a bonding group (M), wherein M is selected from:

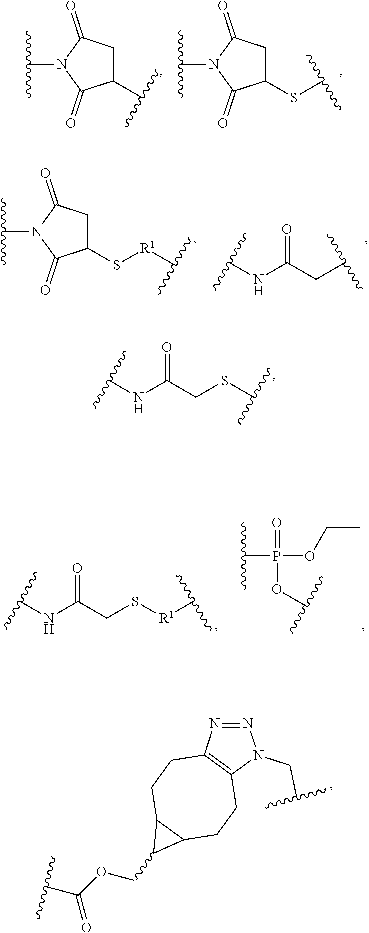

wherein: R¹ is alkylene, cycloalkyl, or

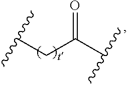

wherein t′ is 0 to 10 wherein each R is independently an alkyl, alkenyl, alkynyl, carbocyclyl, or
heterocyclyl, wherein R¹ is

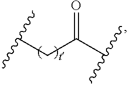

and t′ is 2.

The linker can have the structure:

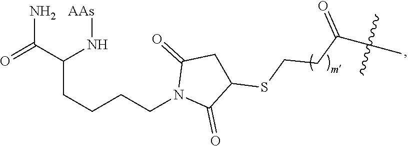

wherein AAₛ is as defined herein, and n′ is 0-10.

The linker can be of the formula:

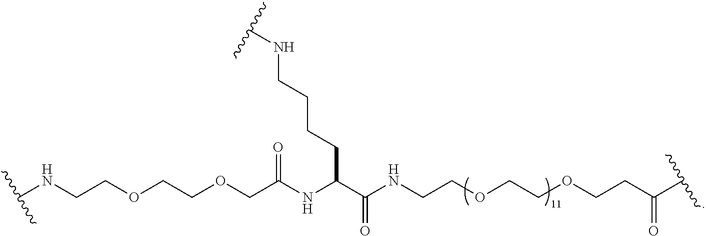

The linker can be of the formula:

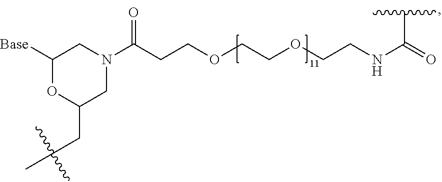

wherein “base” is a nucleobase at the 3′ end of a cargo phosphorodiamidate morpholino oligomer.

The linker can be of the formula:

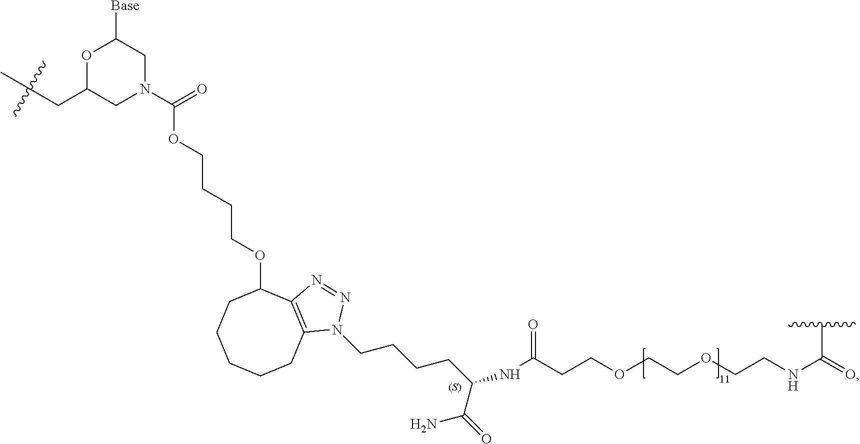

wherein “base” corresponds to a nucleobase at the 3′ end of a cargo phosphorodiamidate morpholino oligomer.

The linker can be of the formula:

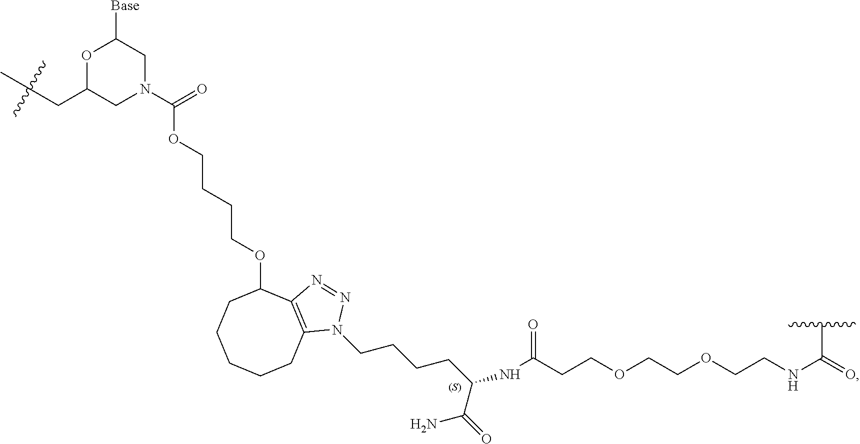

wherein “base” is a nucleobase at the 3′ end of a cargo phosphorodiamidate morpholino oligomer.

The linker can be of the formula:

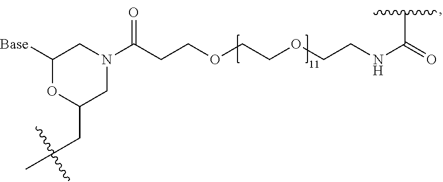

wherein “base” is a nucleobase at the 3′ end of a cargo phosphorodiamidate morpholino oligomer.

The linker can be of the formula:

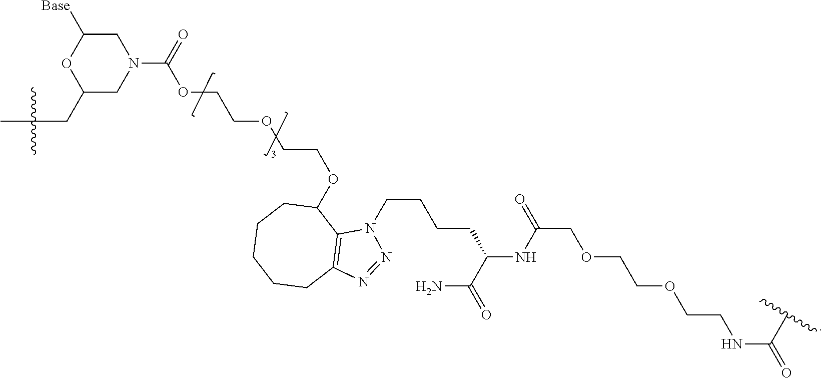

The linker can be covalently bound to a cargo at any suitable location on the cargo. The linker is covalently bound to the 3′ end of cargo or the 5′ end of an oligonucleotide cargo The linker can be covalently bound to the backbone of a cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

cCPP-Linker Conjugates

The cCPP can be conjugated to a linker defined herein. The linker can be conjugated to an AA_{SC} of the cCPP as defined herein.

The linker can comprise a —(OCH₂CH₂)_{z′}— subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂)_{z′ is also referred to as PEG The cCPP-linker conjugate can have a structure selected from Table} 4:

TABLE 4
cCPP-linker conjugates and SEQ ID NOS

| | |
|---|---|
| cyclo(FfΦ-4gp-r-4gp-rQ)-PEG₄-K-NH₂ | cyclo(SEQ ID NO: 118)-PEG₄-K-NH₂ |
| cyclo(FfΦ-Cit-r-Cit-rQ)-PEG₄-K-NH₂ | cyclo(SEQ ID NO: 119)-PEG₄-K-NH₂ |
| cyclo(FfΦ-Pia-r-Pia-rQ)-PEG₄-K-NH₂ | cyclo(SEQ ID NO: 120)-PEG₄-K-NH₂ |
| cyclo(FfΦ-Dml-r-Dml -rQ)-PEG₄-K-NH₂ | cyclo(SEQ ID NO: 121)-PEG₄-K-NH₂ |
| cyclo(FfΦ-Cit-r-Cit-rQ)-PEG₁₂-OH | cyclo(SEQ ID NO: 122)-PEG₁₂-OH |
| cyclo(fΦR-Cit-R-Cit-Q)-PEG₁₂-OH | cyclo(SEQ ID NO: 123)-PEG₁₂-OH |
| cyclo(fΦ-Can-r-Can-r-Q)-PEG₁₂-OH | cyclo(SEQ ID NO: 124)-PEG₁₂-OH |
| cyclo(FfΦ-Can-r-Can-r-Q)-PEG₁₂-OH | cyclo(SEQ ID NO: 125)-PEG₁₂-OH |

linker can comprise a —(OCH₂CH₂)_{z′}— subunit, wherein z′ is an integer from 1 to 23, and a peptide subunit. The peptide subunit can comprise from 2 to 10 amino acids. The cCPP-linker conjugate can have a structure selected from Table 5:

TABLE 5
cCPP-linker conjugate and SEQ ID NOs

| | |
|---|---|
| Ac-PKKK-Can-KV-PEG₂-K(cyclo(FfΦ-Can-r- | Ac-SEQ ID NO: 126-PEG₂-K(cyclo(SEQ ID |
| Can-r-Q)-PEG₁₂-K(N₃)-NH₂ | NO: 125)-PEG₁₂-K(N₃)-NH₂ |
| Ac-PKKKRKV-Lys(cyclo[FfΦ-R-r-Cit-rQ])- | Ac-SEQ ID NO: 42-Lys(cyclo[SEQ ID |
| PEG₁₂-K(N₃)-NH₂ | NO: 127])-PEG₁₂-K(N₃)-NH₂ |
| Ac-PKKKRKV-Lys(cyclo[FfΦ-Cit-r-R-rQ])- | Ac-SEQ ID NO: 42-Lys(cyclo[SEQ ID |
| PEG₁₂-K(N₃)-NH₂ | NO: 128])-PEG₁₂-K(N₃)-NH₂ |
| Ac-PKKKRKV-K(cyclo(FfΦR-cit-R-cit-Q))- | Ac-SEQ ID NO: 42-K(cyclo(SEQ ID |
| PEG₁₂-K(N₃)-NH₂ | NO: 129))-PEG₁₂-K(N₃)-NH₂ |
| Ac-PKKKRKV-PEG₂-Lys(cyclo[FfΦ-Cit-r-Cit- | Ac-SEQ ID NO: 42-PEG₂-Lys(cyclo[SEQ |
| rQ])-B-k(N₃)-NH₂ | ID NO: 130])-B-k(N₃)-NH₂ |
| Ac-PKKKRKV-PEG₂-Lys(cyclo[FfΦ-Cit-r-Cit- | Ac-SEQ ID NO: 42-PEG₂-Lys(cyclo[SEQ |
| rQ])-PEG₂-k(N₃)-NH₂ | ID NO: 130])-PEG₂-k(N₃)-NH₂ |
| Ac-PKKKRKV-PEG₂-Lys(cyclo[FfΦ-Cit-r-Cit- | Ac-SEQ ID NO: 42-PEG₂-Lys(cyclo[SEQ |
| rQ])-PEG4-k(N₃)-NH₂ | ID NO: 130])-PEG4-k(N₃)-NH₂ |
| Ac-PKKKRKV-Lys(cyclo[FfΦ-Cit-r-Cit-rQ])- | Ac-SEQ ID NO: 42-Lys(cyclo[SEQ ID |
| PEG₁₂-k(N₃)-NH₂ | NO: 130])-PEG₁₂-k(N₃)-NH₂ |
| Ac-pkkkrkv-PEG₂-Lys(cyclo[FfΦ-Cit-r-Cit- | Ac-SEQ ID NO: 131-PEG₂-Lys(cyclo[SEQ |
| rQ])-PEG₁₂-k(N₃)-NH₂ | ID NO: 130])-PEG₁₂-k(N₃)-NH₂ |
| Ac-rrv-PEG₂-Lys(cyclo[FfΦ-Cit-r-Cit-rQ])- | Ac-rrv-PEG₂-Lys(cyclo[SEQ ID NO: 130])- |
| PEG₁₂-OH | PEG₁₂-OH |
| Ac-PKKKRKV-PEG₂-Lys(cyclo[FfΦ-Cit-r-Cit- | Ac-SEQ ID NO: 42-PEG₂-Lys(cyclo[SEQ |
| r-Q])-PEG₁₂-k(N₃)-NH₂ | ID NO: 130])-PEG₁₂-k(N₃)-NH₂ |
| Ac-PKKK-Cit-KV-PEG₂-Lys(cyclo[FfΦ-Cit-r- | Ac-SEQ ID NO: 126-PEG₂-Lys(cyclo[SEQ |
| Cit-r-Q])-PEG₁₂-k(N₃)-NH₂ | ID NO: 130])-PEG₁₂-k(N₃)-NH₂ |
| Ac-PKKKRKV-PEG₂-Lys(cyclo[FfΦ-Cit-r-Cit- | Ac-SEQ ID NO: 42-PEG₂-Lys(cyclo[SEQ |
| r-Q]-PEG₁₂-K(N₃)-NH₂ | ID NO: 130]-PEG₁₂-K(N₃)-NH₂ |

EEVs comprising a cyclic cell penetrating peptide (cCPP), linker and exocyclic peptide (EP) are provided. An EEV can comprise the structure of Formula (B):

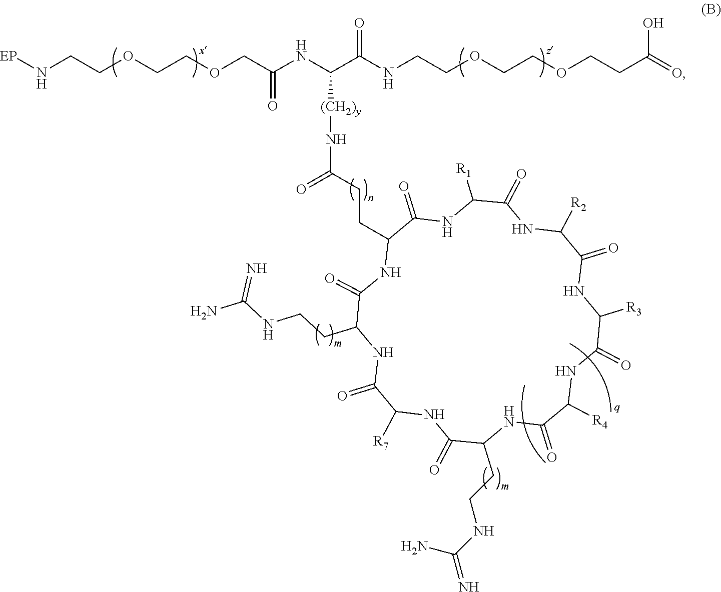

or a protonated form thereof,
wherein:
R₁, R₂, and R₃ are each independently H or an aromatic or heteroaromatic side chain of an amino acid;
R₄ and R₇ are independently H or an amino acid side chain;
EP is an exocyclic peptide as defined herein;
each m is independently an integer from 0-3;
n is an integer from 0-2;
x′ is an integer from 1-20;
y is an integer from 1-5;
q is 1-4; and
   z′ is an integer from 1-23.

R₁, R₂, R₃, R₄, R₇, EP, in, q, y, x′, z′ are as described herein.

n can be 0. n can be 1. n can be 2.

The EEV can comprise the structure of Formula (B-a) or (B-b):

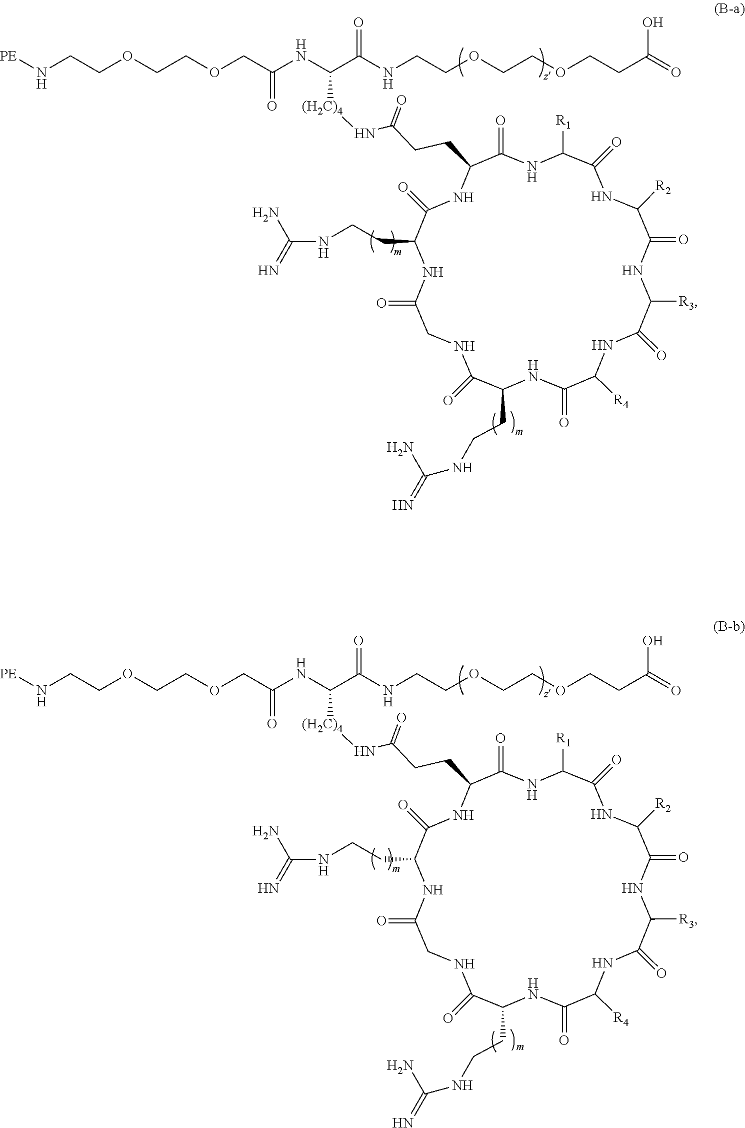

or a protonated form thereof, wherein EP (shown as “PE”), R¹, R², R³, R⁴, m and z′ are as defined above in Formula (B).

The EEV can comprises the structure of Formula (B-c):

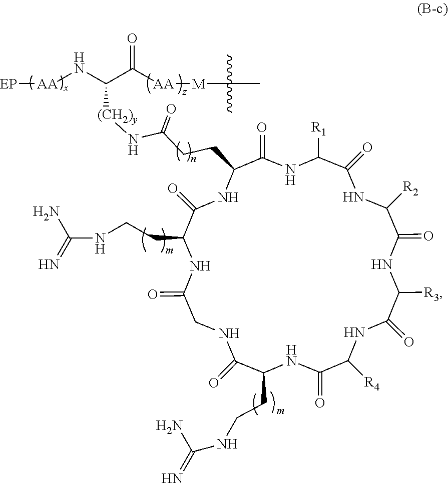

or a protonated form thereof, wherein EP, R¹, R², R³, R⁴, and m are as defined above in Formula (B); AA is an amino acid as defined herein; M is as defined herein; n is an integer from 0-2; x is an integer from 1-10; y is an integer from 1-5; and z is an integer from 1-10.

The EEV can have the structure of Formula (B-1) (B-2), (B-3), or (B-4):

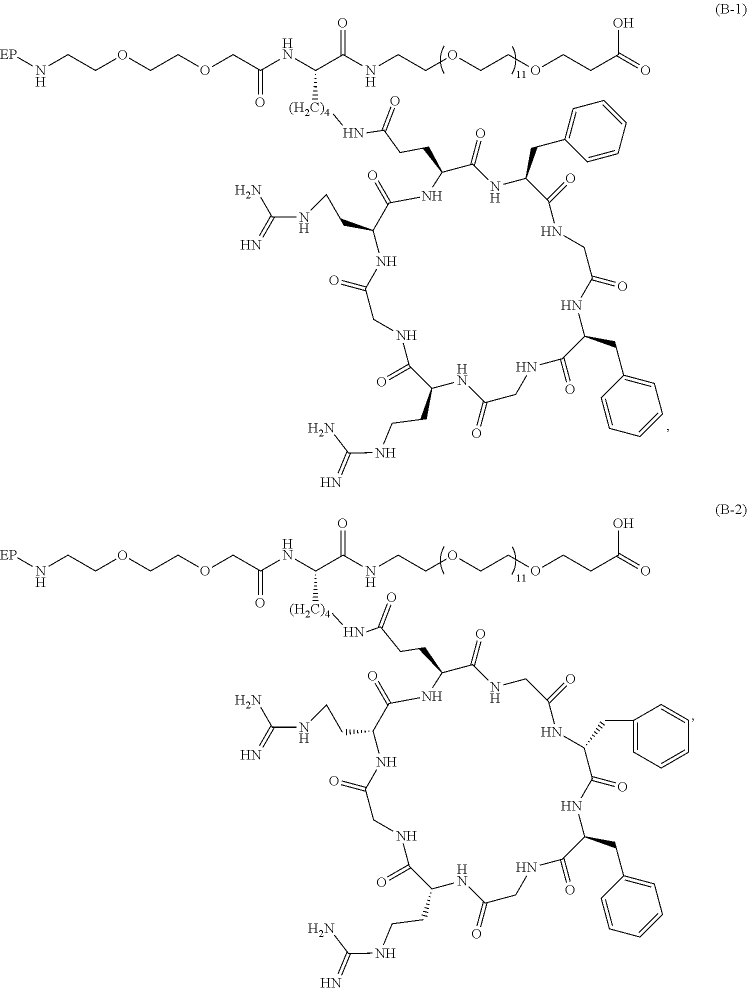

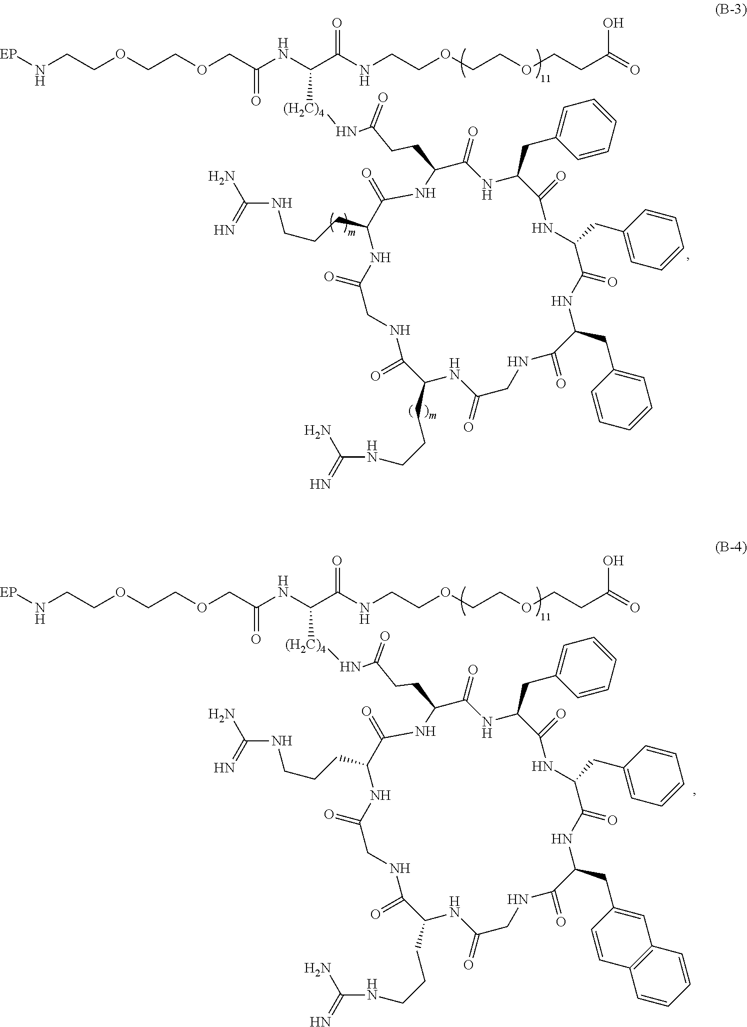

or a protonated form thereof, wherein EP is as defined above in Formula (B).

The EEV can comprise Formula (B) and can have the structure: Ac-PKKKRKVAEEA-K(cyclo[FGFGRGRQ])-PEG₁₂-OH (Ac-SEQ ID NO: 132—K(cyclo[SEQ ID NO:82])-PEG₁₂-OH) or Ac-PK—KKR—KV-AEEA-K(cyclo[GfFGrGrQ])-PEG₁₂-OH (Ac-SEQ ID NO:133-K(cyclo[SEQ ID NO:83])-PEG₁₂-OH).

The EEV can comprise a cCPP of formula:

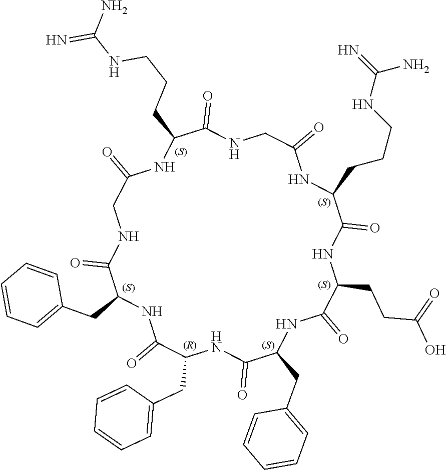

The EEV can comprise formula: Ac-PKKKRKV-miniPEG2-Lys(cyclo(FfFGRGRQ)-mini PEG2-K(N3) (Ac-SEQ ID NO:42-PEG₂-Lys(cyclo(SEQ ID NO:81)-PEG₂-K(N₃)).

The EEV can be:

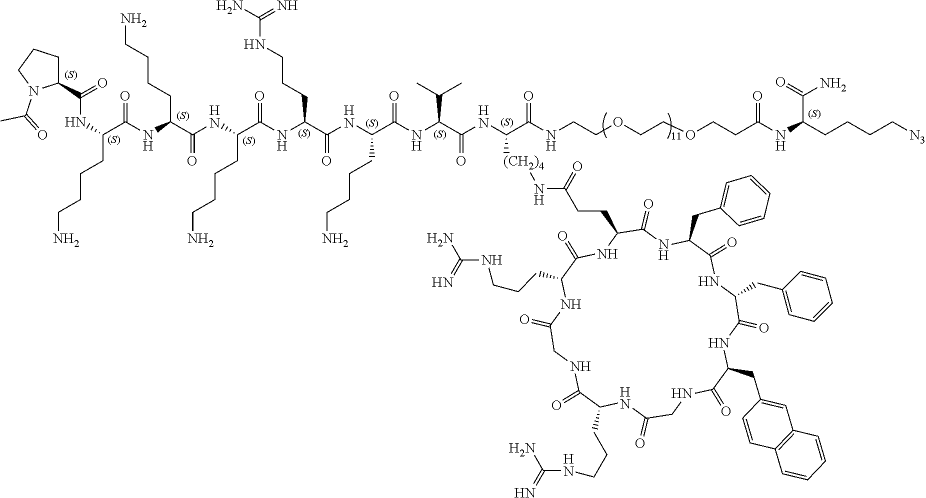

The EEV can be

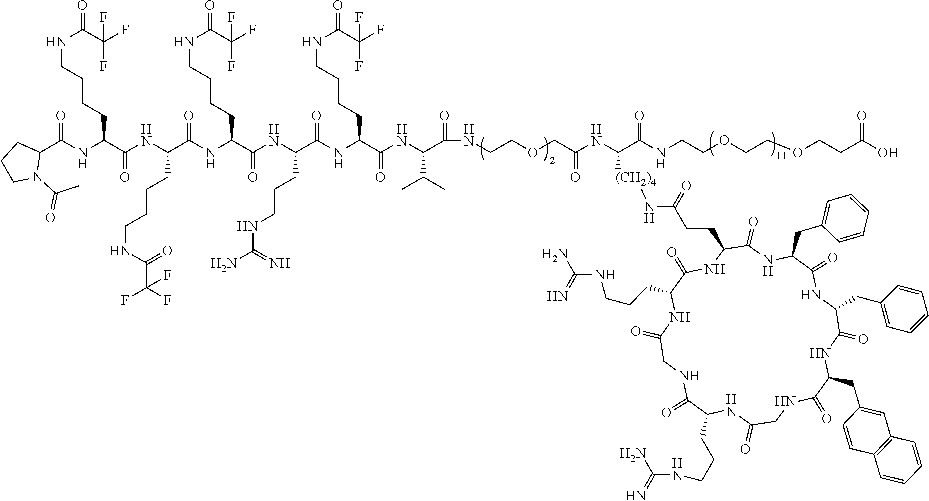

The EEV can be Ac-P—K(Tfa)-K(Tfa)-K(Tfa)-R—K(Tfa)-V-miniPEG₂-K(cyclo(Ff-Nal-GrGrQ)-PEG₁₂-OH (Ac-SEQ ID NO:134-miniPEG₂-K(cyclo(SEQ ID NO:135)-PEG₁₂-OH).

The EEV can be

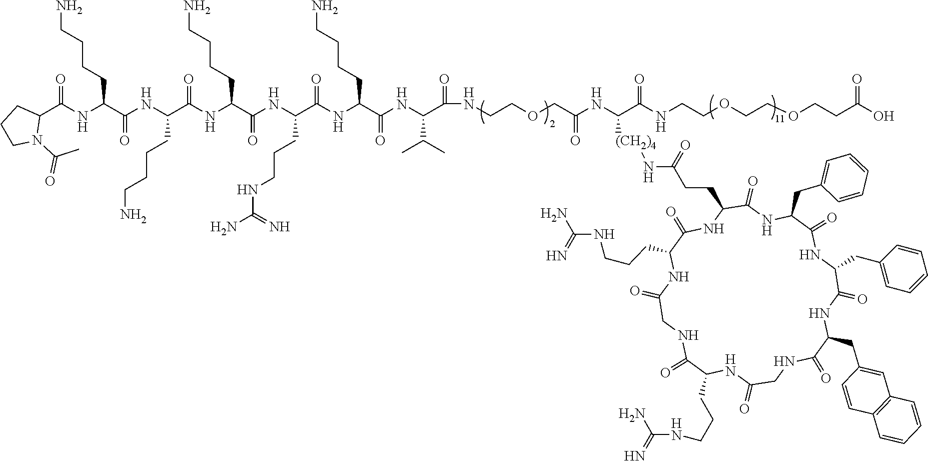

The EEV can be Ac-P—K—K—K—R—K—V-miniPEG₂-K(cyclo(Ff-Nal-GrGrQ)-PEG₂-OH (Ac-SEQ ID NO:42-PEG₂-K(cyclo(SEQ ID NO 135)-PEG₁₂-OH).

The EEV can be

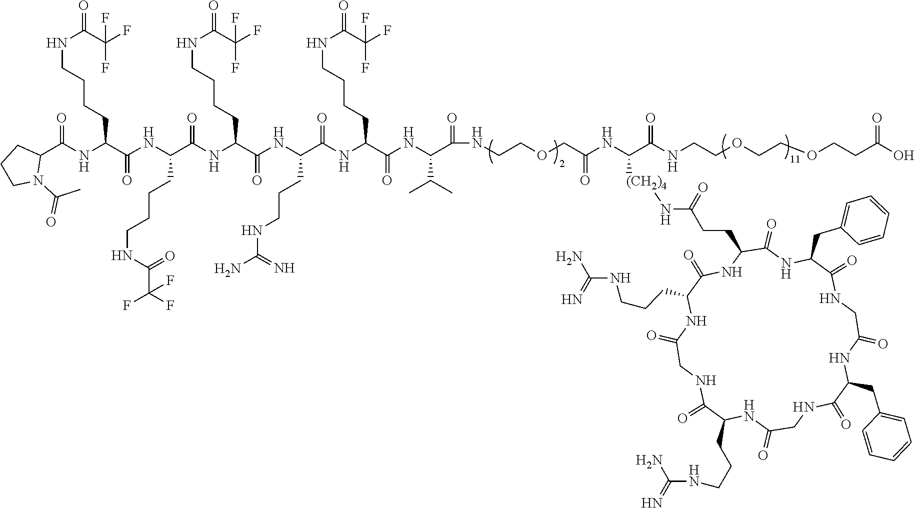

The EEV can be

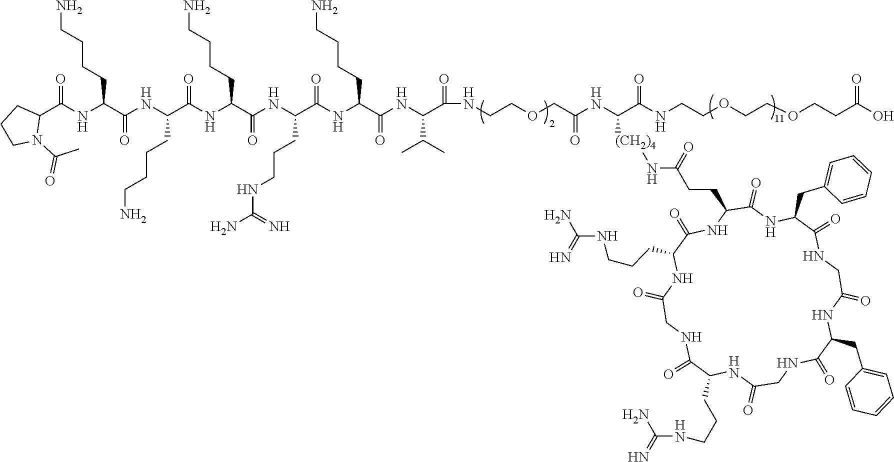

### The EEV can be

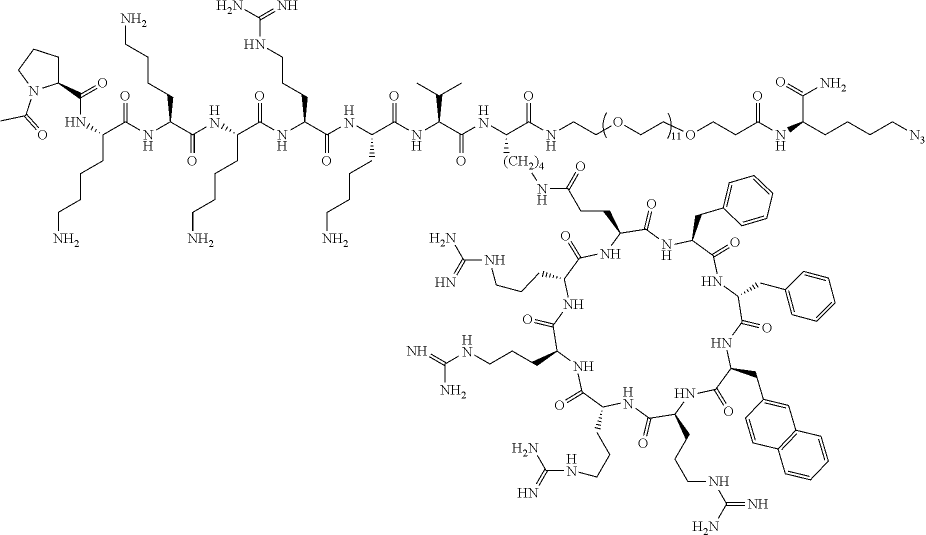

The EEV can be

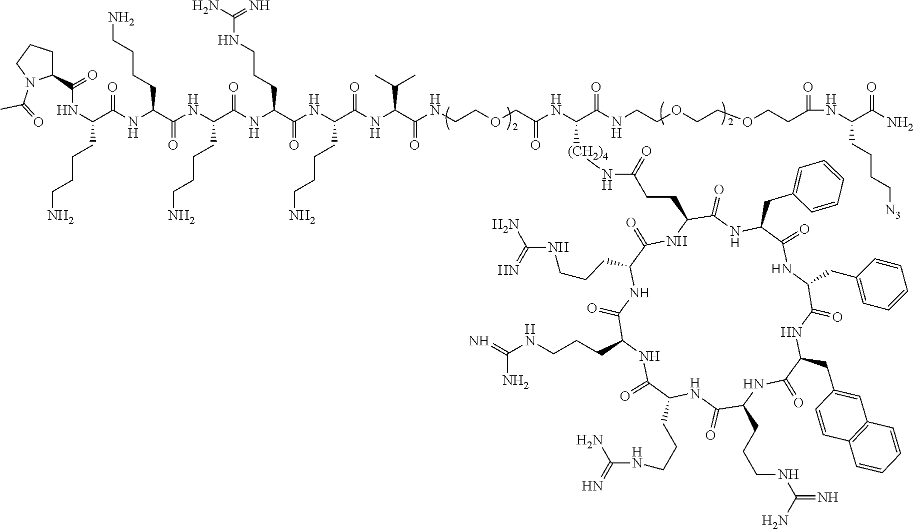

The EEV can be

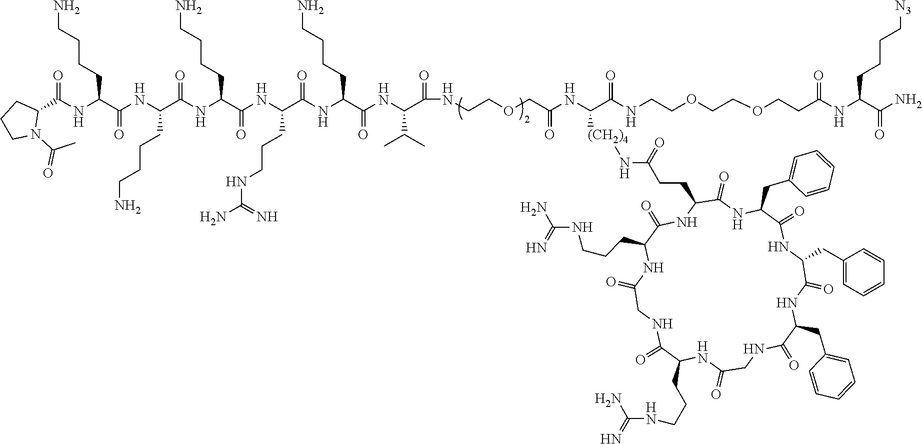

The EEV can be

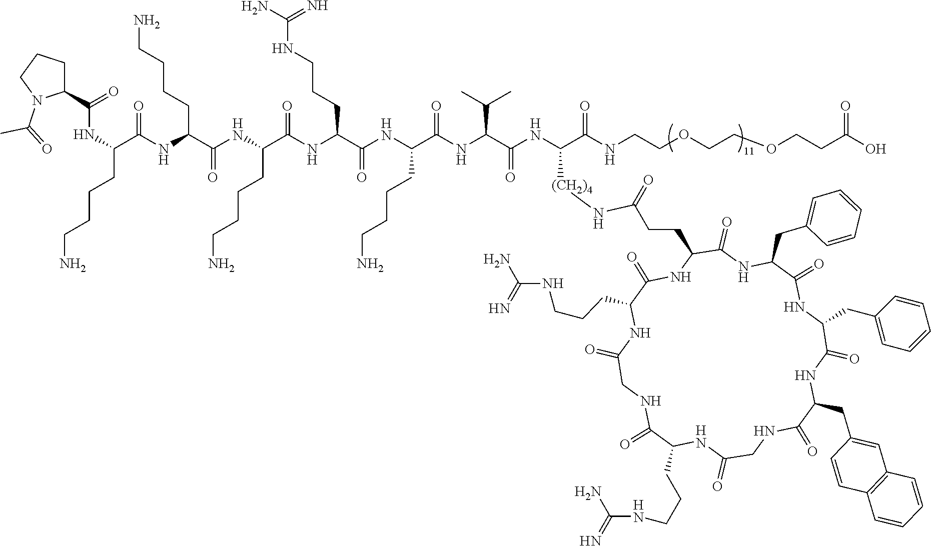

The EEV can be:

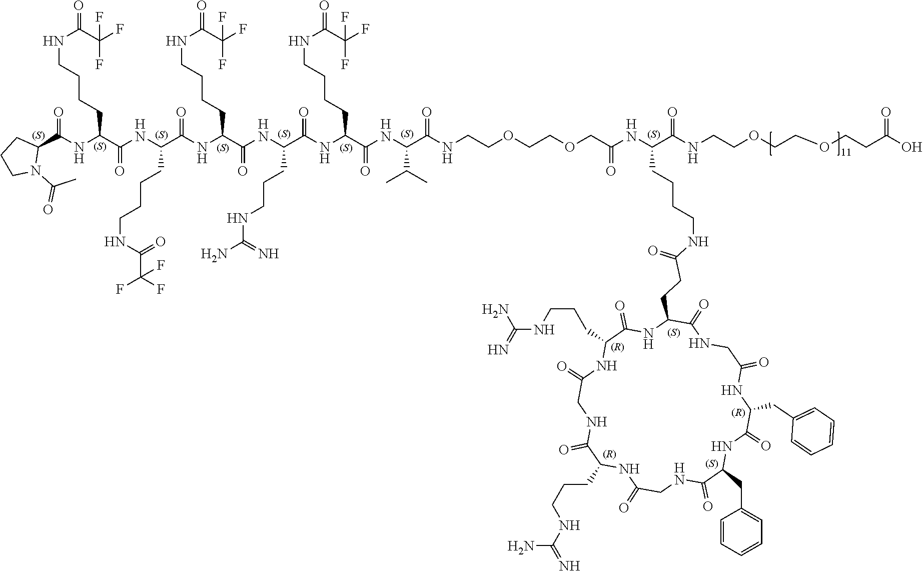

The EEV can be

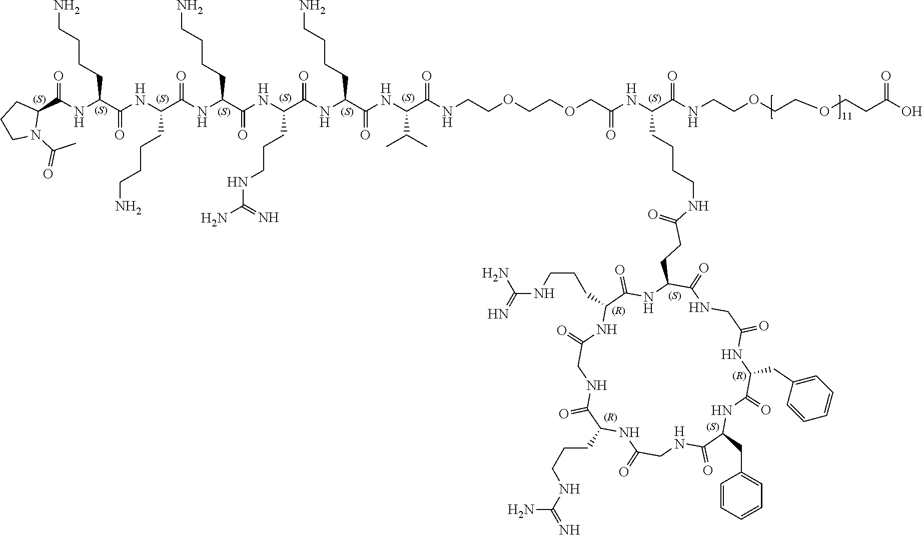

The EEV can be

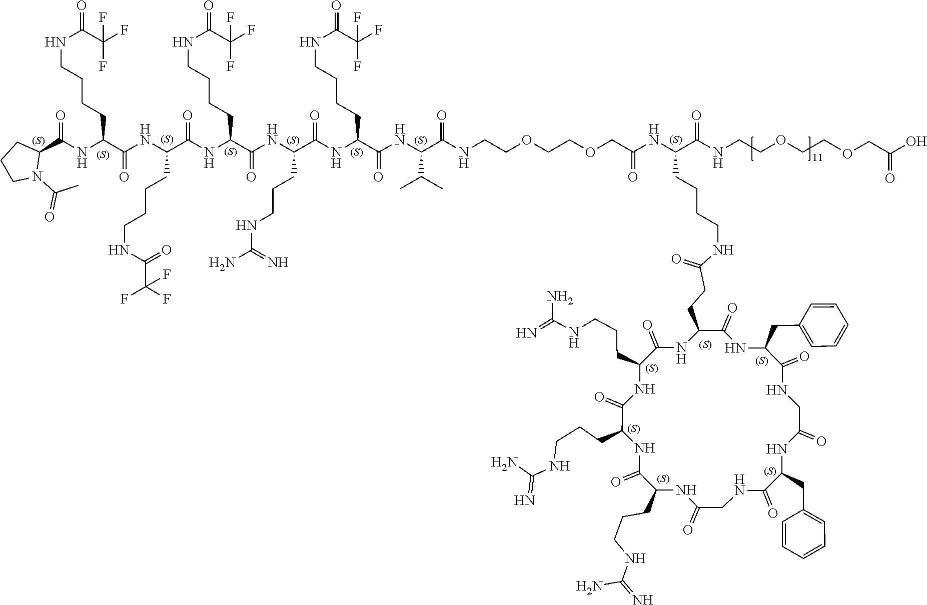

The EEV can be

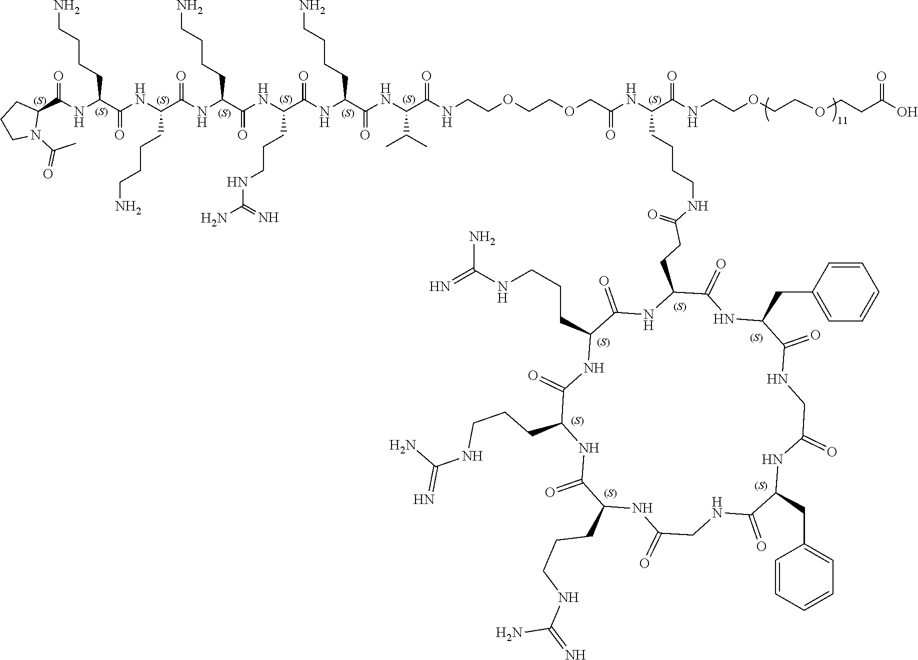

The EEV can be

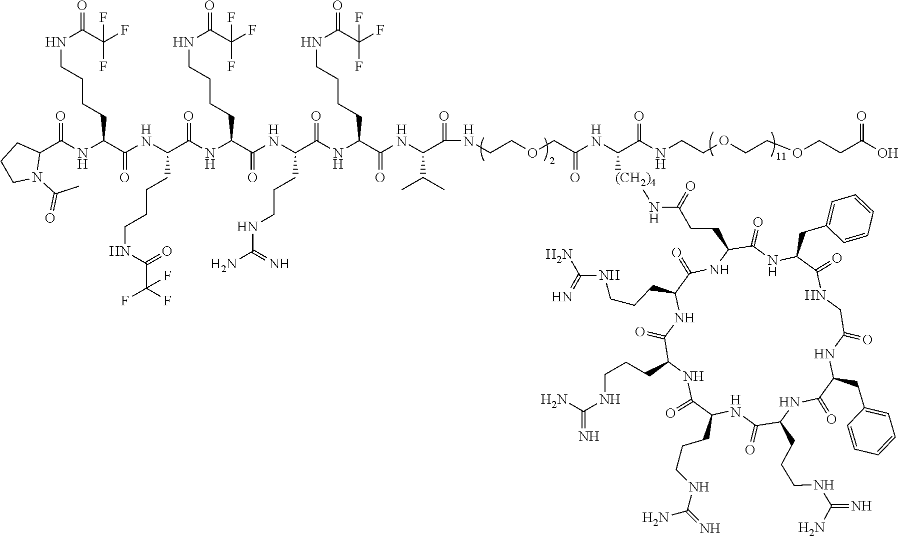

The EEV can be selected from

| | |
|---|---|
| Ac-rr-miniPBG₂-Dap(cyclo[FfΦ-Cit-r-Cit-rQ])- | Ac-rr-miniPEG₂-Dap(cyclo[SEQ |
| PEG₁₂-OH | ID NO: 136])-PEG₁₂-OH |
| Ac-frr-PEG₂-Dap(cyclo[FfΦ-Cit-r-Cit-rQ])- | Ac-frr-PEG₂-Dap(cyclo[SEQ ID |
| PEG₁₂-OH | NO: 136])-PEG₁₂-OH |
| Ac-rfr-PEG₂-Dap(cyclo[FfΦ-Cit-r-Cit-rQ])- | Ac-rfr-PEG₂-Dap(cyclo[SEQ ID |
| PEG₁₂-OH | NO: 136])-PEG₁₂-OH |
| Ac-rbfbr-PEG₂-Dap(cyclo[FfΦ-Cit-r-Cit-rQ])- | Ac-SEQ ID NO: 137-PEG₂- |
| PEG₁₂-OH | Dap(cyclo[SEQ ID NO: 136])- |
| | PEG₁₂-OH |
| Ac-rrr-PEG₂-Dap(cyclo[FfΦ-Cit-r-Cit-rQ])- | Ac-rrr-PEG₂-Dap(cyclo[SEQ ID |
| PEG₁₂-OH | NO: 136])-PEG₁₂-OH |
| Ac-rbr-PEG₂-Dap(cyclo[FfΦ-Cit-r-Cit-rQ])- | Ac-rbr-PEG₂-Dap(cyclo[SEQ ID |
| PEG₁₂-OH | NO: 1361)-PEG₁₂-OH |
| Ac-rbrbr-PEG₂-Dap(cyclo[FfΦ-Cit-r-Cit-rQ])- | Ac-SEQ ID NO: 138-PEG₂- |
| PEG₁₂-OH | Dap(cyclo[SEQ ID NO: 136])- |
| | PEG₁₂-OH |
| Ac-hh-PEG₂-Dap(cyclo[FfΦ-Cit-r-Cit-rQ])- | Ac-hh-PEG₂-Dap(cyclo[SEQ ID |
| PEG₁₂-OH | NO: 136])-PEG₁₂-OH |
| Ac-hbh-PEG₂-Dap(cyclo[FfΦ-Cit-r-Cit-rQ])- | Ac-hbh-PEG₂-Dap(cyclo[SEQ ID |
| PEG₁₂-OH | NO: 136])-PEG₁₂-OH |
| Ac-hbhbh-PEG₂-Dap(cyclo[FfΦ-Cit-r-Cit-rQ])- | Ac-SEQ IN NO: 139-PEG₂- |
| PEG₁₂-OH | Dap(cyclo[SEQ ID NO: 136])- |
| | PEG₁₂-OH |
| Ac-rbhbh-PEG₂-Dap(cyclo[FfΦ-Cit-r-Cit-rQ])- | Ac-SEQ ID NO: 140-PEG₂- |
| PEG₁₂-OH | Dap(cyclo[SEQ ID NO: 136])- |
| | PEG₁₂-OH |
| Ac-hbrbh-PEG₂-Dap(cyclo[FfΦ-Cit-r-Cit-rQ])- | Ac-SEQ ID NO: 141-PEG₂- |
| PEG₁₂-OH | Dap(cyclo[SEQ ID NO: 136])- |
| | PEG₁₂-OH |
| Ac-rr-Dap(cyclo[FfΦ-Cit-r-Cit-rQ])-b-OH | Ac-rr-Dap(cyclo[SEQ ID |
| | NO: 1361-b-OH |
| Ac-frr-Dap(cyclo[FfΦ-Cit-r-Cit-rQD)-b-OH | Ac-frr-Dap(cyclo[SEQ ID |
| | NO: 136]-b-OH |
| Ac-rfr-Dap(cyclo[FfΦ-Cit-r-Cit-rQ])-b-OH | Ac-rfr-Dap(cyclo[SEQ ID |
| | NO: 136])-b-OH |
| Ac-rbfbr-Dap(cyclo[FfΦ-Cit-r-Cit-rQ])-b-OH | Ac-SEQ ID NO: 137- |
| | Dap(cyclo[SEQ ID NO: 136])-b- |
| | OH |
| Ac-rrr-Dap(cyclo[FfΦ-Cit-r-Cit-rQ1)-b-OH | Ac-rrr-Dap(cyclo[SEQ ID |
| | NO: 136])-b-OH |
| Ac-rbr-Dap(cyclo[FfΦ-Cit-r-Cit-rQ])-b-OH | Ac-rbr-Dap(cyclo[SEQ ID |
| | NO: 136])-b-OH |
| Ac-rbrbr-Dap(cyclo[FfΦ-Cit-r-Cit-rQ])-b-OH | Ac-SEQ ID NO: 138- |
| | Dap(cyclo[SEQ ID NO: 136])-b- |
| | OH |
| Ac-hh-Dap(cyclo[FfΦ-Cit-r-Cit-rQ])-b-OH | Ac-hh-Dap(cyclo[SEQ ID |
| | NO: 1361)-b-OH |
| Ac-hbh-Dap(cyclo[FfΦ-Cit-r-Cit-rQ])-b-OH | Ac-hbh-Dap(cyclo[SEQ ID |
| | NO: 1361)-b-OH |
| Ac-hbhbh-Dap(cyclo[FfΦ-Cit-r-Cit-rQ])-b-OH | Ac-SEQ IN NO: 139- |
| | Dap(cyclo[SEQ ID NO: 136])-b- |
| | OH |
| Ac-rbhbh-Dap(cyclo[FfΦ-Cit-r-Cit-rQ])-b-OH | Ac-SEQ ID NO: 140- |
| | Dap(cyclo[SEQ ID NO: 136])-b- |
| | OH |
| Ac-hbrbh-Dap(cyclo[FfΦ-Cit-r-Cit-rQ])-b-OH | Ac-SEQ ID NO: 141- |
| | Dap(cyclo[SEQ ID NO: 136])-b- |
| | OH |
| Ac-KKKK-miniPEG₂-Lys(cyclo[FfΦGrGrQ])-miniPEG₂- | Ac-SEQ ID NO: 7-miniPEG₂- |
| K(N₃)-NH₂ | Lys(cyclo[SEQ ID NO: Z80])- |
| | miniPEG₂-K(N₃)-NH₂ |
| Ac-KGKK-miniPEG₂-Lys(cyclo[FfΦGrGrQ])-miniPEG₂- | Ac-SEQ ID NO: 13-PEG₂- |
| K(N₃)-NH₂ | Lys(cyclo[SEQ ID NO: Z80]- |
| | PEG₂-K(N₃)-NH₂ |
| Ac-KKGK-miniPEG₂-Lys(cyclo[FfΦGrGrQ])-miniPEG₂- | Ac-SEQ ID NO: 14-miniPEG₂- |
| K(N₃)-NH₂ | Lys(cyclo[SEQ ID NO: Z80]- |
| | miniPEG₂-K(N₃)-NH₂ |
| Ac-KKK-miniPEG₂-Lys(cyclo[FfΦGrGrQ])-miniPEG₂- | Ac-KKK-miniPEG₂- |
| K(N₃)-NH₂ | Lys(cyclo[SEQ ID NO: 80])- |
| | miniPEG₂-K(N₃)-NH₂ |
| Ac-KK-miniPEG₂-Lys(cyclo[FfΦGrGrQ])-miniPEG₂- | Ac-KK-miniPEG₂-Lys(cyclo[SEQ |
| K(N₃)-NH₂ | ID NO: 801)-miniPEG₂-K(N₃)-NH₂ |
| Ac-KGK-miniPEG₂-Lys(cyclo[FfΦGrGrQ])-miniPEG₂- | Ac-KGK-miniPEG₂- |
| K(N₃)-NH₂ | Lys(cyclo[SEQ ID NO: 80])- |
| | miniPEG₂-K(N₃)-NH₂ |
| Ac-KBK-miniPEG₂-Lys(cyclo[FfΦGrGrQ])-miniPEG₂- | Ac-KBK-miniPEG₂- |
| K(N₃)-NH₂ | Lys(cyclo[SEQ ID NO: 80])- |
| | miniPEG₂-K(N₃)-NH₂ |
| Ac-KBKBK-miniPEG₂-Lys(cyclo[FfΦGrGrQ])-miniPEG₂- | Ac-SEQ ID NO: 24-miniPEG₂- |
| K(N₃)-NH₂ | Lys(cyclo[SEQ ID NO: 80])- |
| | miniPEG₂-K(N₃)-NH₂ |
| Ac-KR-miniPEG₂-Lys(cyclo[FfΦGrGrQ])-miniPEG₂- | Ac-KR-miniPEG₂-Lys(cyclo[SEQ |
| K(N₃)-NH₂ | ID NO: 80])-miniPEG₂-K(N₃)-NH₂ |
| Ac-KBR-miniPEG₂-Lys(cyclo[FfΦGrGrQ])-miniPEG₂- | Ac-KBR-miniPEG₂- |
| K(N₃)-NH₂ | Lys(cyclo[SEQ ID NO: 80])- |
| | miniPEG₂-K(N₃)-NH₂ |
| Ac-PKKKRKV-miniPEG₂-Lys(cyclo[FfΦGrGrQ])- | Ac-SEQ ID NO: 42-miniPEG₂- |
| miniPEG₂-K(N₃)-NH₂ | Lys(cyclo[SEQ ID NO: 80]- |
| | miniPEG₂-K(N₃)-NH₂ |
| Ac-PKKKRKV-miniPEG₂-Lys(cyclo[FfΦGrGrQ])- | Ac-SEQ ID NO: 42-miniPEG₂- |
| miniPEG₂-K(N₃)-NH₂ | Lys(cyclo[SEQ ID NO: 80])- |
| | miniPEG₂-K(N₃)-NH₂ |
| Ac-PGKKRKV-miniPEG₂-Lys(cyclo[FfΦGrGrQ])- | Ac-SEQ ID NO: 43-miniPEG₂- |
| miniPEG₂-K(N₃)-NH₂ | Lys(cyclo[SEQ ID NO: 80])- |
| | miniPEG₂-K(N₃)-NH₂ |
| Ac-PKGKRKV-miniPEG₂-Lys(cyclo[FfΦGrGrQ])- | Ac-SEQ ID NO: 44-miniPEG₂- |
| miniPEG₂-K(N₃)-NH₂ | Lys(cyclo[SEQ ID NO: 80]- |
| | miniPEG₂-K(N₃)-NH₂ |
| Ac-PKKGRKV-miniPEG₂-Lys(cyclo[FfΦGrGrQ])- | Ac-SEQ ID NO: 45-miniPEG₂- |
| miniPEG₂-K(N₃)-NH₂ | Lys(cyclo[SEQ ID NO: 80])- |
| | miniPEG₂-K(N₃)-NH₂ |
| Ac-PKKKGKV-miniPEG₂-Lys(cyclo[FfΦGrGrQ])- | Ac-SEQ ID NO: 46-miniPEG₂- |
| miniPEG₂-K(N₃)-NH₂ | Lys(cyclo[SEQ ID NO: 80])- |
| | miniPEG₂-K(N₃)-NH₂ |
| Ac-PKKKRGV-miniPEG₂-Lys(cyclo[FfΦGrGrQ])- | Ac-SEQ ID NO: 47-miniPEG₂- |
| miniPEG₂-K(N₃)-NH₂ | Lys(cyclo[SEQ ID NO: 80])- |
| | miniPEG₂-K(N₃)-NH₂ |
| Ac-PKKKRKG-miniPEG₂-Lys(cyclo[FfΦGrGrQ])- | Ac-SEQ ID NO: 48-miniPEG₂- |
| miniPEG₂-K(N₃)-NH₂ | Lys(cyclo[SEQ ID NO: 80])- |
| | miniPEG₂-K(N₃)-NH₂ |
| Ac-KKKRK-miniPEG₂-Lys(cyclo[FfΦGrGrQ])- | Ac-SEQ ID NO: 19-miniPEG₂- |
| miniPEG₂-K(N₃)-NH₂ | Lys(cyclo[SEQ ID NO: 80])- |
| | miniPEG₂-K(N₃)-NH₂ |
| Ac-KKRK-miniPEG₂-Lys(cyclo[FfΦGrGrQ])-miniPEG₂- | Ac-SEQ ID NO: 8-miniPEG₂- |
| K(N₃)-NH₂ and | Lys(cyclo[SEQ ID NO: 80])- |
| | miniPEG₂-K(N₃)-NH₂ and |
| Ac-KRK-miniPEG₂-Lys(cyclo[FfΦGrGrQ])-miniPEG₂- | Ac-KRK-miniPEG₂- |
| K(N₃)-NH₂. | Lys(cyclo[SEQ ID NO: 80])- |
| | miniPEG₂-K(N₃)-NH₂. |

The EEV can be selected from:

Ac-PKKKRKV-Lys(cyclo[FfΦGrGrQ])-PEG₁₂-K(N₃)-NH₂
(Ac-SEQ ID NO: 42-Lys(cyclo[SEQ ID NO: 80])-PEG₁₂-K(N₃)-NH₂)
Ac-PKKKRKV-miniPEG₂-Lys(cyclo[FfΦGrGrQ])-miniPEG₂-K(N₃)-NH₂
(Ac-SEQ ID NO: 42-miniPEG₂-Lys(cyclo[SEQ ID NO: 80])-miniPEG₂-K(N₃)-NH₂)
Ac-PKKKRKV-miniPEG₂-Lys(cyclo[FGFGRGRQ])-miniPEG₂-K(N₃)-NH₂
(Ac-SEQ ID NO: 42-miniPEG₂-Lys(cyclo[SEQ ID NO: 82])-miniPEG₂-K(N₃)-NH₂)
Ac-KR-PEG₂-K(cyclo[FGFGRGRQ])-PEG₂-K(N₃)-NH₂
(Ac-KR-PEG₂-K(cyclo[SEQ ID NO: 82])-PEG₂-K(N₃)-NH₂)
Ac-PKKKGKV-PEG₂-K(cyclo[FGFGRGRQ])-PEG₂-K(N₃)-NH₂
(Ac-SEQ ID NO: 46-PEG₂-K(cyclo[SEQ ID NO: 82])-PEG₂-K(N₃)-NH₂)
Ac-PKKKRKG-PEG₂-K(cyclo[FGFGRGRQ])-PEG₂-K(N₃)-NH₂
(Ac-SEQ ID NO: 48-PEG₂-K(cyclo[SEQ ID NO: 82])-PEG₂-K(N₃)-NH₂)
Ac-KKKRK-PEG₂-K(cyclo[FGFGRGRQ])-PEG₂-K(N₃)-NH₂
(Ac-SEQ ID NO: 19-PEG₂-K(cyclo[SEQ ID NO: 82])-PEG₂-K(N₃)-NH₂)
Ac-PKKKRKV-miniPEG₂-Lys(cyclo[FFΦGRGRQ])-miniPEG₂-K(N₃)-NH₂
(Ac-SEQ ID NO: 42-mini-PEG₂-Lys(cyclo[SEQ ID NO: 80])-miniPEG₂-K(N₃)-NH₂)
Ac-PKKKRKV-miniPEG₂-Lys(cyclo[BhFfΦGrGrQ])-miniPEG₂-K(N₃)-NH₂
(Ac-SEQ ID NO: 42-miniPEG₂-Lys(cyclo[SEQ ID NO: 142])-miniPEG₂-K(N₃)-NH₂)
Ac-PKKKRKV-miniPEG₂-Lys(cyclo[FfΦSrSrQ])-miniPEG₂-K(N₃)-NH₂
(Ac-SEQ ID NO: 42-miniPEG₂-Lys(cyclo[SEQ ID NO: 143])-miniPEG₂-K(N₃)-NH₂).

The EEV can be selected from:

Ac-PKKKRKV-miniPEG₂-Lys(cyclo(GfFGrGrQ])-PEG₁₂-OH
(Ac-SEQ ID NO: 42-miniPEG₂-Lys(cyclo(SEQ ID NO: 133])-PEG₁₂-OH)
Ac-PKKKRKV-miniPEG₂-Lys(cyclo[FGFKRKRQ])-PEG₁₂-OH
(Ac-SEQ ID NO: 42-miniPEG₂-Lys(cyclo[SEQ ID NO: 144])-PEG₁₂-OH)
Ac-PKKKRKV-miniPEG₂-Lys(cyclo[FGFRGRGQ])-PEG₁₂-OH
(Ac-SEQ ID NO: 42-miniPEG₂-Lys(cyclo[SEQ ID NO: 145])-PEG₁₂-OH)
Ac-PKKKRKV-miniPEG₂-Lys(cyclo[FGFGRGRGRQ])-PEG₁₂-OH
(Ac-SEQ ID NO: 42-miniPEG₂-Lys(cyclo[SEQ ID NO: 146])-PEG₁₂-OH)
Ac-PKKKRKV-miniPEG₂-Lys(cyclo[FGFGRrRQ])-PEG₁₂-OH
(Ac-SEQ ID NO: 42-miniPEG₂-Lys(cyclo[SEQ ID NO: 147])-PEG₁₂-OH)
Ac-PKKKRKV-miniPEG₂-Lys(cyclo[FGFGRRRQ])-PEG₁₂-OH
(Ac-SEQ ID NO: 42-miniPEG₂-Lys(cyclo[SEQ ID NO: 84])-PEG₁₂-OH) and
Ac-PKKKRKV-miniPEG₂-Lys(cyclo[FGFRRRRQ])-PEG₁₂-OH
(Ac-SEQ ID NO: 42-miniPEG₂-Lys(cyclo[SEQ ID NO: 85])-PEG₁₂-OH).

The EEV can be selected from:

Ac-K-K-K-R-K-G-miniPEG₂-K(cyclo[FGFGRGRQ])-PEG₁₂-OH
(Ac-SEQ ID NO: 148-miniPEG₂-K(cyclo[SEQ ID NO: 82])-PEG₁₂-OH)
Ac-K-K-K-R-K-miniPEG₂-K(cyclo[FGFGRGRQ])-PEG₁₂-OH
(Ac-SEQ ID NO: 19-miniPEG₂-K(cyclo[SEQ ID NO: 82])-PEG₁₂-OH)
Ac-K-K-R-K-K-PEG₄-K(cyclo[FGFGRGRQ])-PEG₁₂-OH
(Ac-SEQ ID NO: 22-PEG₄-K(cyclo[SEQ ID NO: 82])-PEG₁₂-OH)
Ac-K-R-K-K-K-PEG₄-K(cyclo[FGFGRGRQ])-PEG₁₂-OH
(Ac-SEQ ID NO: 21-PEG₄-K(cyclo[SEQ ID NO: 82])-PEG₁₂-OH)
Ac-K-K-K-K-R-PEG₄-K(cyclo[FGFGRGRQ])-PEG₁₂-OH
(Ac-SEQ ID NO: 23-PEG₄-K(cyclo[SEQ ID NO: 82])-PEG₁₂-OH)
Ac-R-K-K-K-K-PEG₄-K(cyclo[FGFGRGRQ])-PEG₁₂-OH
(Ac-SEQ ID NO: 20-PEG₄-K(cyclo[SEQ ID NO: 82])-PEG₁₂-OH) and
Ac-K-K-K-R-K-PEG₄-K(cyclo[FGFGRGRQ])-PEG₁₂-OH
(Ac-SEQ ID NO: 19-PEG₄-K(cyclo[SEQ ID NO: 82])-PEG₁₂-OH).

The EEV can be selected from:

Ac-PKKKRKV-PEG₂-K(cyclo[FGFGRGRQ])-PEG₂-K(N₃)-NH₂
(Ac-SEQ ID NO: 42-PEG₂-K(cyclo[SEQ ID NO: 82])-PEG₂-K(N₃)-NH₂)
Ac-PKKKRKV~PEG₂-K(cyclo[FGFGRGRQ])-PEG₁₂-OH
(Ac-SEQ ID NO: 42-PEG₂-K(cyclo[SEQ ID NO: 82])-PEG₁₂-OH)
Ac-PKKKRKV-PEG₂-K(cyclo[GfFGrGrQ])-PEG₂-K(N₃)-NH₂
(Ac-SEQ ID NO: 42-PEG₂-K(cyclo[SEQ ID NO: 133])-PEG₂-K(N₃)-NH₂) and
Ac-PKKKRKV-PEG₂-K(cyclo[GfFGrGrQ])-PEG₁₂-OH
(Ac-SEQ ID NO: 42-PEG₂-K(cyclo[SEQ ID NO: 133])-PEG₁₂-OH).

The cargo can be a protein and the EEV can be selected from:

Ac-PKKKRKV-PEG₂-K(cyclo[FfΦGrGrQ])-PEG₁₂-OH
(Ac-SEQ ID NO: 42-PEG₂-K(cyclo[SEQ ID NO: 80])-
PEG₁₂-OH)
Ac-PKKKRKV-PEG₂-K(cyclo[FfΦCit-r-Cit-rQ])-PEG₁₂-OH
(Ac-SEQ ID NO: 42-PEG₂-K(cyclo[SEQ ID NO: 79])-
PEG₁₂-OH)
Ac-PKKKRKV-PEG₂-K(cyclo[FfFGRGRQ])-PEG1-OH
(Ac-SEQ ID NO: 42-PEG₂-K(cyclo[SEQ ID NO: 81])-
PEG₁₂-OH)
Ac-PKKKRKV-PEG₂-K(cyclo[FGFGRGRQ])-PEG₁₂-OH
(Ac-SEQ ID NO: 42-PEG₂-K(cyclo[SEQ ID NO: 82])-
PEG₁₂-OH)
Ac-PKKKRKV-PEG₂-K(cyclo[GfFGrGrQ])-PEG₁₂-OH
(Ac-SEQ ID NO: 42-PEG₂-K(cyclo[SEQ ID NO: 133])-
PEG₁₂-OH)
Ac-PKKKRKV-PEG₂-K(cyclo[FGFGRRRQ])-PEG₁₂-OH
(Ac-SEQ ID NO: 42-PEG₂-K(cyclo[SEQ ID NO: 84])-
PEG₁₂-OH)
Ac-PKKKRKV-PEG₂-K(cyclo[FGFRRRRQ])-PEG₁₂-OH
(Ac-SEQ ID NO: 42-PEG₂-K(cyclo[SEQ ID NO: 85])-
PEG₁₂-OH)
Ac-rr-PEG₂-K(cyclo[FfΦGrGrQ])-PEG₁₂-OH
(Ac-rr-PEG₂-K(cyclo[SEQ ID NO: 80])-PEG₁₂-OH)
Ac-rr-PEG₂-K(cyclo[FfΦCit-r-Cit-rQ])-PEG₁₂-OH
(Ac-rr-PEG₂-K(cyclo[SEQ ID NO: 79])-PEG₁₂-OH)
Ac-rr-PEG₂-K(cyclo[FfF-GRGRQ])-PEGE-OH
(Ac-rr-PEG₂-K(cyclo[SEQ ID NO: 81])-PEG₁₂-OH)
Ac-rr-PEG₂-K(cyclo[FGFGRGRQ])-PEG₁₂-OH
(Ac-rr-PEG₂-K(cyclo[SEQ ID NO: 82])-PEG₁₂-OH)
Ac-rr-PEG₂-K(cyclo[GfFGrGrQ])-PEG₁₂-OH
(Ac-rr-PEG₂-K(cyclo[SEQ ID NO: 133])-PEG₁₂-OH)
Ac-rr-PEG₂-K(cyclo[FGFGRRRQ])-PEG₁₂-OH
(Ac-rr-PEG₂-K(cyclo[SEQ ID NO: 84])-PEG₁₂-OH)
Ac-rr-PEG₂-K(cyclo[FGFRRRRQ])-PEG₁₂-OH
(Ac-rr-PEG₂-K(cyclo[SEQ ID NO: 85])-PEG₁₂-OH)
Ac-rrr-PEG₂-K(cyclo[FfΦGrGrQ])-PEG₁₂-OH
(Ac-rrr-PEG₂-K(cyclo[SEQ ID NO: 80])-PEG₁₂-OH)
Ac-rrr-PEG₂-K(cyclo[FfΦCit-r-Cit-rQ])-PEG₁₂-OH
(Ac-rrr-PEG₂-K(cyclo[SEQ ID NO: 79])-PEG₁₂-OH)
Ac-rrr-PEG₂-K(cyclo[FfFGRGRQ])-PEG₁₂-OH
(Ac-rrr-PEG₂-K(cyclo[SEQ ID NO: 81])-PEG₁₂-OH)
Ac-rrr-PEG₂-K(cyclo[FGFGRGRQ])-PEG₁₂-OH
(Ac-rrr-PEG₂-K(cyclo[SEQ ID NO: 82])-PEG₁₂-OH)
Ac-rrr-PEG₂-K(cyclo[GfFGrGrQ])-PEG₁₂-OH
(Ac-rrr-PEG₂-K(cyclo[SEQ ID NO: 133])-PEG₁₂-OH)
Ac-rrr-PEG₂-K(cyclo[FGFGRRRQ])-PEG₁₂-OH
(Ac-rrr-PEG₂-K(cyclo[SEQ ID NO: 84])-PEG₁₂-OH)
Ac-rrr-PEG₂-K(cyclo[FGFRRRRQ])-PEG₁₂-OH
(Ac-rrr-PEG₂-K(cyclo[SEQ ID NO: 85])-PEG 1-OH)
Ac-rhr-PEG₂-K(cyclo[FfΦGrGrQ])-PEG₁₂-OH
(Ac-rhr-PEG₂-K(cyclo[SEQ ID NO: 80])-PEG₁₂-OH)
Ac-rhr-PEG₂-K(cyclo[FfΦCit-r-Cit-rQ])-PEG₁₂-OH
(Ac-rhr-PEG₂-K(cyclo[SEQ ID NO: 79])-PEG₁₂-OH)
Ac-rhr-PEG₂-K(cyclo[FfFGRGRQ])-PEG₁₂-OH
(Ac-rhr-PEG₂-K(cyclo[SEQ ID NO: 81])-PEG₁₂-OH)
Ac-rhr-PEG₂-K(cyclo[FGFGRGRQ])-PEG₁₂-OH
(Ac-rhr-PEG₂-K(cyclo[SEQ ID NO: 82])-PEG ₁₂-OH)
Ac-rhr-PEG₂-K(cyclo[GfFGrGrQ1)-PEG₁₂-OH
(Ac-rbr-PEG₂-K(cyclo[SEQ ID NO: 133])-PEG₁₂-OH)
Ac-rhr-PEG₂-K(cyclo[FGFGRRRQ])-PEG₁₂-OH
(Ac-rhr-PEG₂-K(cyclo[SEQ ID NO: 84])-PEG ₁₂-OH)
Ac-rhr-PEG₂-K(cyclo[FGFRRRRQ])-PEG₁₂-OH
(Ac-rhr-PEG₂-K(cyclo[SEQ ID NO: 85])-PEG₁₂-OH)
Ac-rbr-PEG₂-K(cyclo[FfΦGrGrQ])-PEG₁₂-OH
(Ac-rbr-PEG₂-K(cyclo[SEQ ID NO: 80])-PEG₁₂-OH)
Ac-rbr-PEG₂-K(cyclo[FfΦCit-r-Cit-rQ])-PEG₁₂-OH
(Ac-rbr-PEG₂-K(cyclo[SEQ ID NO: 79])-PEG₁₂-OH)
Ac-rbr-PEG₂-K(cyclo[FfFGRGRQ])-PEG₁₂-OH
(Ac-rbr-PEG₂-K(cyclo[SEQ ID NO: 81])-PEG₁₂-OH)
Ac-rbr-PEG₂-K(cyclo[FGFGRGRQ])-PEG₁₂-OH
(Ac-rbr-PEG₂-K(cyclo[SEQ ID NO: 82])-PEG₁₂-OH)
Ac-rbr-PEG₂-K(cyclo[GfFGrGrQ])-PEG₁₂-OH
(Ac-rbr-PEG₂-K(cyclo[SEQ ID NO: 133])-PEG ₁₂-OH)
Ac-rbr-PEG₂-K(cyclo[FGFGRRRQ])-PEG₁₂-OH
(Ac-rbr-PEG₂-K(cyclo[SEQ ID NO: 84])-PEG₁₂-OH)
Ac-rbr-PEG₂-K(cyclo[FGFRRRRQ])-PEG₁₂-OH
(Ac-rbr-PEG₂-K(cyclo[SEQ ID NO: 85])-PEG₁₂-OH)
Ac-rbrbr-PEG₂-K(cyclo[FfΦGrGrQ])-PEG₁₂-OH
(Ac-SEQ ID NO: 138-PEG₂-K(cyclo[SEQ ID NO: 80])-
PEG₁₂-OH)
Ac-rbrbr-PEG₂-K(cyclo[FfΦCit-r-Cit-rQ])-PEG₁₂-OH
(Ac-SEQ ID NO: 138-PEG₂-K(cyclo[SEQ ID NO: 79])-
PEG₁₂-OH)
Ac-rbrbr-PEG₂-K(cyclo[FfFGRGRQ])-PEG₁₂-OH
(Ac-SEQ ID NO: 138-PEG₂-K(cyclo[SEQ ID NO: 81])-
PEG₁₂-OH)
Ac-rbrbr-PEG₂-K(cyclo[FGFGRGRQ])-PEG₁₂-OH
(Ac-SEQ ID NO: 138-PEG₂-K(cyclo[SEQ ID NO: 82])-
PEG₁₂-OH)
Ac-rbrbr-PEG₂-K(cyclo[GfFGrGrQ])-PEG₁₂-OH
(Ac-SEQ ID NO: 138-PEG₂-K(cyclo[SEQ ID NO: 133])-
PEG₁₂-OH)
Ac-rbrbr-PEG₂-K(cyclo[FGFGRRRQ])-PEG₁₂-OH
(Ac-SEQ ID NO:138-PEG₂-K(cyclo[SEQ ID NO: 84])-
PEG₁₂-OH)
Ac-rbrbr-PEG₂-K(cyclo[FGFRRRRQ])-PEG₁₂-OH
(Ac-SEQ ID NO: 138-PEG₂-K(cyclo[SEQ ID NO: 85])-
PEG₁₂-OH)
Ac-rbhbr-PEG₂-K(cyclo[FfΦGrGrQ])-PEG1-OH
(Ac-SEQ ID NO:149-PEG₂-K(cyclo[SEQ ID NO: 80])-
PEG₁₂-OH)
Ac-rbhbr-PEG₂-K(cyclo[FfΦCit-r-Cit-rQ])-PEG₁₂-OH
(Ac-SEQ ID NO: 149-PEG₂-K(cyclo[SEQ ID NO: 79])-
PEG₁₂-OH)
Ac-rbhbr-PEG₂-K(cyclo[FfFGRGRQ])-PEG₁₂-OH
(Ac-SEQ ID NO:149-PEG₂-K(cyclo[SEQ ID NO: 81])-
PEG₁₂-OH)
Ac-rbhbr-PEG₂-K(cyclo[FGFGRGRQ])-PEG₁₂-OH
(Ac-SEQ ID NO: 149-PEG₂-K(cyclo[SEQ ID NO: 82])-
PEG₁₂-OH)
Ac-rbhbr-PEG₂-K(cyclo[GfFGrGrQ])-PEG₁₂-OH
(Ac-SEQ ID NO: 149-PEG₂-K(cyclo[SEQ ID NO: 133])-
PEG₁₂-OH)
Ac-rbhbr-PEG₂-K(cyclo[FGFGRRRQ])-PEG₁₂-OH
(Ac-SEQ ID NO:149-PEG₂-K(cyclo[SEQ ID NO: 84])-
PEG₁₂-OH)
Ac-rbhbr-PEG₂-K(cyclo[FGFRRRRQ])-PEG₁₂-OH
(Ac-SEQ ID NO: 149-PEG₂-K(cyclo[SEQ ID NO: 85])-
PEG₁₂-OH)
Ac-hbrbh-PEG₂-K(cyclo[FfΦGrGrQ])-PEG₁₂-OH
(Ac-SEQ ID NO: 141-PEG₂-K(cyclo[SEQ ID NO: 80])-
PEG₁₂-OH)
Ac-hbrbh-PEG₂-K(cyclo[FfΦCit-r-Cit-rQ])-PEG₁₂-OH
(Ac-SEQ ID NO:141-PEG₂-K(cyclo[SEQ ID NO: 79])-
PEG₁₂-OH)
Ac-hbrbh-PEG₂-K(cyclo[FfFGRGRQ])-PEG₁₂-OH
(Ac-SEQ ID NO: 141-PEG₂-K(cyclo[SEQ ID NO: 81])-
PEG₁₂-OH)
Ac-hbrbh-PEG₂-K(cyclo[FGFGRGRQ])-PEG₁₂-OH
(Ac-SEQ ID NO:141-PEG₂-K(cyclo[SEQ ID NO: 82])-
PEG₁₂-OH)
Ac-hbrbh-PEG₂-K(cyclo[GfFGrGrQ])-PEG₁₂-OH
(Ac-SEQ ID NO: 141-PEG₂-K(cyclo[SEQ ID NO: 133])-
PEG₁₂-OH)
Ac-hbrbh-PEG₂-K(cyclo[FGFGRRRQ])-PEG₁₂-OH
(Ac-SEQ ID NO: 141-PEG₂-K(cyclo[SEQ ID NO: 84])-
PEG₁₂-OH)
Ac-hbrbh-PEG₂-K(cyclo[FGFRRRRQ])-PEG₁₂-OH
(Ac-SEQ ID NO: 141-PEG₂-K(cyclo[SEQ ID NO: 85])-
PEG₁₂-OH).

wherein b is beta-alanine, and the exocyclic sequence can be D or L stereochemistry.

### Cargo

The cell penetrating peptide (CPP), such as a cyclic cell penetrating peptide (e.g., cCPP), can be conjugated to a cargo. As used herein, “cargo” is a compound or moiety for which delivery into a cell is desired. The cargo can be conjugated to a terminal carbonyl group of a linker. At least one atom of the cyclic peptide can be replaced by a cargo or at least one lone pair can form a bond to a cargo. The cargo can be conjugated to the cCPP by a linker. The cargo can be conjugated to an AA_{SC} by a linker. At least one atom of the cCPP can be replaced by a therapeutic moiety or at least one lone pair of the cCPP forms a bond to a therapeutic moiety. A hydroxyl group on an amino acid side chain of the cCPP can be replaced by a bond to the cargo. A hydroxyl group on a glutamine side chain of the cCPP can be replaced by a bond to the cargo. The cargo can be conjugated to the cCPP by a linker. The cargo can be conjugated to an AA_{SC} by a linker.

In embodiments, the amino acid side chain comprises a chemically reactive group to which the linker or cargo is conjugated comprises. The chemically reactive group can comprise an amine group, a carboxylic acid, an amide, a hydroxyl group, a sulfhydryl group, a guanidinyl group, a phenolic group, a thioether group, an imidazolyl group, or an indolyl group. in embodiments, the amino acid of the cCPP to which the cargo is conjugated comprises lysine, arginine, aspartic acid, glutamic acid, asparagine, glutamine, homoglutamine, serine, threonine, tyrosine, cysteine, arginine, tyrosine, methionine, histidine or tryptophan.

The cargo can comprise one or more detectable moieties, one or more therapeutic moieties (TMs), one or more targeting moieties, or any combination thereof. In embodiments, the cargo comprises a TM. In embodiments, the TM comprises an antisense compound (AC). In embodiments, the AC binds to at least a portion of splice element (SE) of a target gene transcript or in sufficient proximity to the SE of the target gene transcript to modulate splicing of the target gene transcript. In embodiments, the AC binds to at least a portion of a SE of a target IRF-5, DPMK, or DUX4 gene transcript. In embodiments, the AC binds in sufficient proximity to a SE of a target IRF-5, DPMK, or DUX4 gene transcript to modulate splicing of the target IRF-5, DPMK, or DUX4 gene transcript.

Cyclic Cell Penetrating Peptides (cCPPs) Conjugated to a Cargo Moiety

The cyclic cell penetrating peptide (cCPP) can be conjugated to a cargo moiety.

The cargo moiety can be conjugated to the linker at the terminal carbonyl group to provide the following structure:

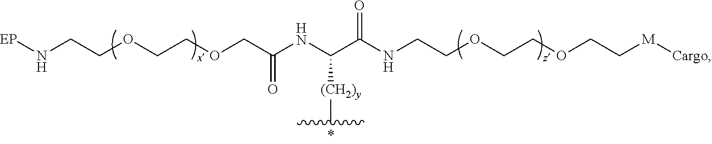

wherein:
EP is an exocyclic peptide and M, AA_{SC}, Cargo, x′, y, and z′ are as defined above, * is the point of attachment to the AA_{SC}. x′ can be 1. y can be 4. z′ can be 11. —(OCH₂CH₂)_{x′}— and/or —(OCH₂CH₂)_{z′}— can be independently replaced with one or more amino acids, including, for example, glycine, beta-alanine, 4-aminobutyric acid, 5-aminopentanoic acid, 6-aminohexanoic acid, or combinations thereof.

An endosomal escape vehicle (EEV) can comprise a cyclic cell penetrating peptide (cCPP), an exocyclic peptide (EP) and linker, and can be conjugated to a cargo to form an EEV-conjugate comprising the structure of Formula (C):

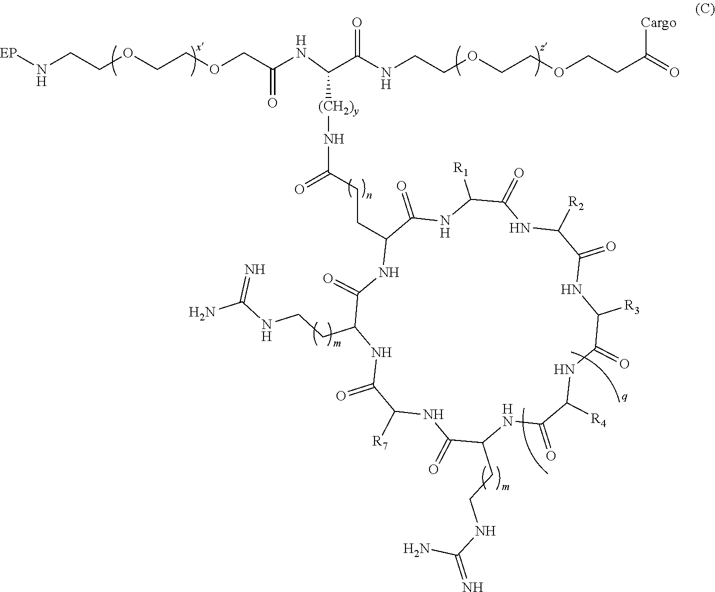

or a protonated form thereof,
wherein:
   R₁, R₂, and R₃ can each independently be H or an amino acid residue having a side chain comprising an aromatic group;
R₄ is H or an amino acid side chain;
EP is an exocyclic peptide as defined herein;
Cargo is a, moiety as defined herein;
each mi is independently an integer from 0-3;
n is an integer from 0-2;
x′ is an integer from 2-20;
y is an integer from 1-5;
q is an integer from 1-4; and
z′ is an integer from 2-20.

R₁, R₂, R₃, R₄, EP, cargo, m, n, x′, y, q, and z′ are as defined herein.

The EEV can be conjugated to a cargo and the EEV-conjugate can comprise the structure of Formula (C-a) or (C-b):

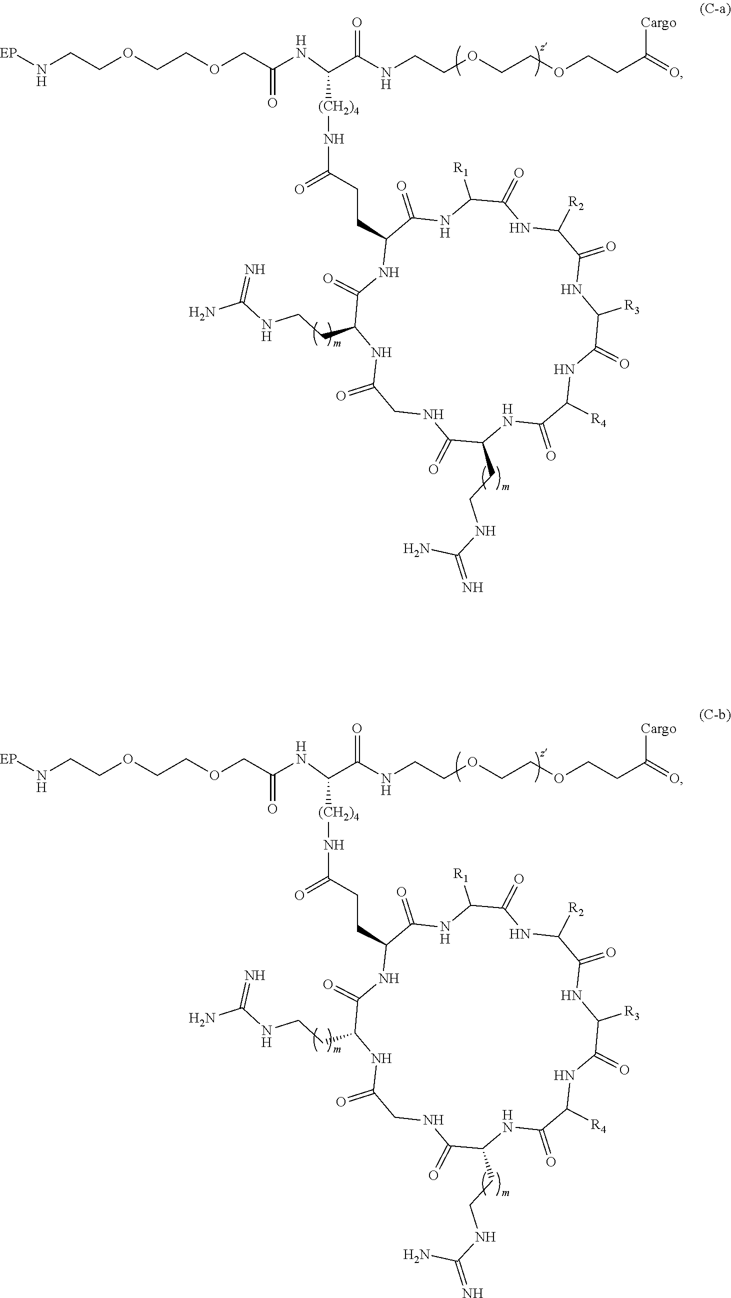

or a protonated form thereof, wherein EP, m and z are as defined above in formula (C).

The EEV can be conjugated to a cargo and the EEV-conjugate can comprise the structure of Formula (C-c):

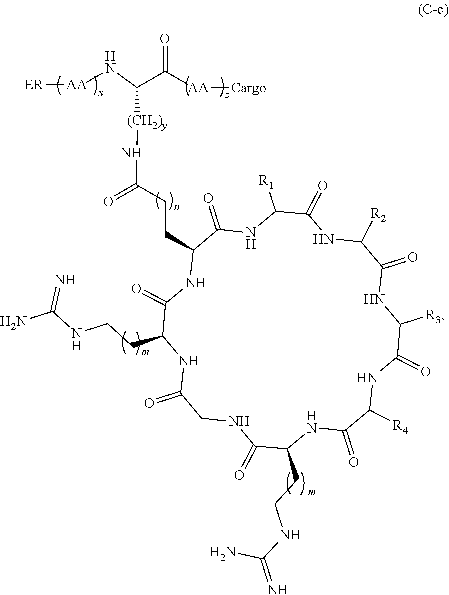

or a protonated form thereof, wherein EP, R¹, R², R³, R⁴, and m are as defined above in Formula (III); AA can be an amino acid as defined herein; n can be an integer from 0-2; x can be an integer from 1-10; y can be an integer from 1-5; and z can be an integer from 1-10.

The EEV can be conjugated to an oligonucleotide cargo and the EEV-oligonucleotide conjugate can comprises a structure of Formula (C-1), (C-2), (C-3), or (C-4):

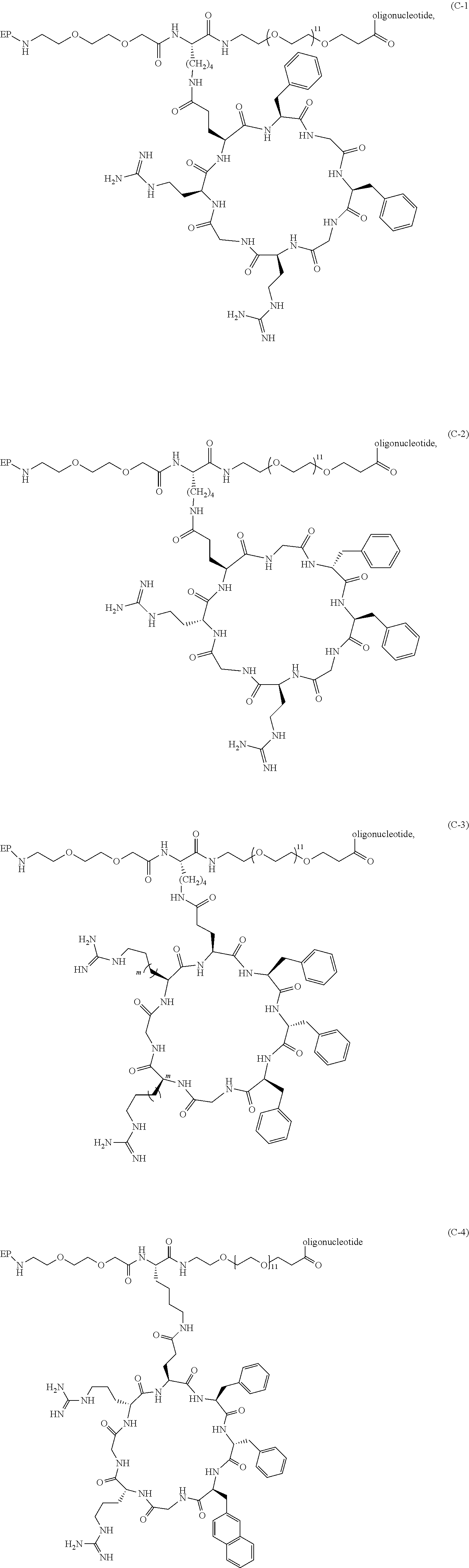

The EEV can be conjugated to an oligonucleotide cargo and the EEV-conjugate can comprise the structure:

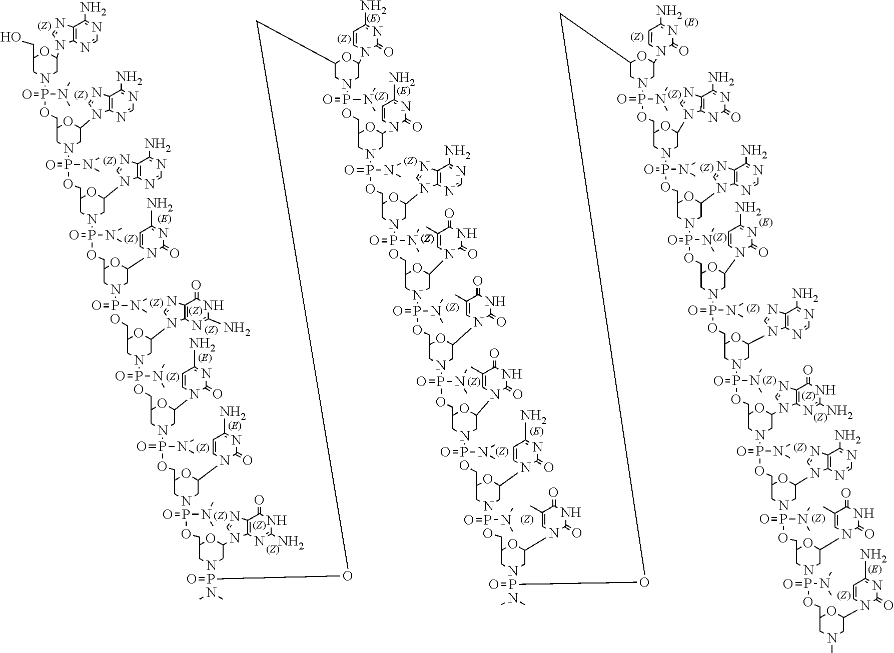

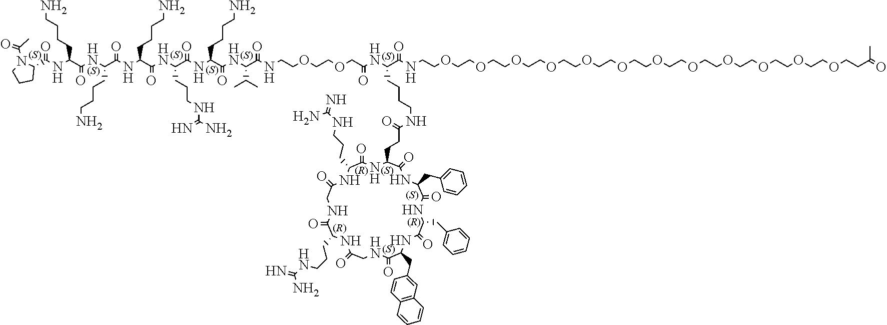

### Cytosolic Delivery Efficiency

Modifications to a cyclic cell penetrating peptide (cCPP) may improve cytosolic delivery efficiency. Improved cytosolic uptake efficiency can be measured by comparing the cytosolic delivery efficiency of a cCPP having a modified sequence to a control sequence. The control sequence does not include a. particular replacement amino acid residue in the modified sequence (including, but not limited to arginine, phenylalanine, and/or glycine), but is otherwise identical.

As used herein cytosolic delivery efficiency refers to the ability of a cCPP to traverse a cell membrane and enter the cytosol of a cell. Cytosolic: delivery efficiency of the cCPP is not necessarily dependent on a receptor or a cell type. Cytosolic delivery efficiency can refer to absolute cytosolic delivery efficiency or relative cytosolic delivery efficiency.

Absolute cytosolic delivery efficiency is the ratio of cytosolic concentration of a cCPP (or a cCPP-cargo conjugate) over the concentration of the cCPP (or the cCPP-cargo conjugate) in the growth medium. Relative cytosolic delivery efficiency refers to the concentration of a cCPP in the cytosol compared to the concentration of a control cCPP in the cytosol. Quantification can be achieved by fluorescently labeling the cCPP (e.g., with a FITC dye) and measuring the fluorescence intensity using techniques well-known in the art.

Relative cytosolic delivery efficiency is determined by comparing (i) the amount of a cCPP of the invention internalized by a cell type (e.g., HeLa cells) to (ii) the amount of a control cCPP internalized by the same cell type. To measure relative cytosolic delivery efficiency, the cell type may be incubated in the presence of a cCPP for a specified period of time (e.g., 30 minutes, 1 hour, 2 hours, etc.) after which the amount of the cCPP internalized by the cell is quantified using methods known in the art, e.g., fluorescence microscopy. Separately, the same concentration of the control cCPP is incubated in the presence of the cell type over the same period of time, and the amount of the control cCPP internalized by the cell is quantified.

Relative cytosolic delivery efficiency can be determined by measuring the IC₅₀ of a cCPP having a modified sequence for an intracellular target and comparing the IC₅₀ of the cCPP having the modified sequence to a control sequence (as described herein).

The relative cytosolic delivery efficiency of the cCPPs can be in the range of from about 50% to about 450% compared to cyclo(FfΦRrRrQ, SEQ ID NO:150), e.g., about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 510%, about 520%, about 530%, about 540%, about 550%, about 560%, about 570%, about 580%, or about 590%. inclusive of all values and subranges therebetween. The relative cytosolic delivery efficiency of the cCPPs can be improved by greater than about 600% compared to a cyclic peptide comprising cyclo(FfΦRrRrQ, SEQ ID NO: 150).

The absolute cytosolic delivery efficacy of from about 40% to about 100%, e.g., about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, inclusive of all values and subranges therebetween.

The cCPPs of the present disclosure can improve the cytosolic delivery efficiency by about 1.1 fold to about 30 fold, compared to an otherwise identical sequence, e.g., about 1.2, about 13, about 1.4, about 1.5, about 16, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween.

### Detectable Moiety

In embodiments, the compound disclosed herein includes a detectable moiety. In embodiments, the detectible moiety is attached to the cell penetrating peptide at the amino group, the carboxylate group, or the side chain of any of the amino acids of the cell penetrating peptide moiety (e.g., at the amino group, the carboxylate group, or the side chain of any amino acid in the CPP). In embodiments, the therapeutic moiety includes a detectable moiety. The detectable moiety can include any detectable label. Examples of suitable detectable labels include, but are not limited to, a UV-Vis label, a near-infrared label, a luminescent group, a phosphorescent group, a magnetic spin resonance label, a photosensitizer, a photocleavable moiety, a chelating center, a heavy atom, a radioactive isotope, an isotope detectable spin resonance label, a paramagnetic moiety, a chromophore, or any combination thereof. In embodiments, the label is detectable without the addition of further reagents.

In embodiments, the detectable moiety is a biocompatible detectable moiety, such that the compounds can be suitable for use in a variety of biological applications. “Biocompatible” and “biologically compatible”, as used herein, generally refer to compounds that are, along with any metabolites or degradation products thereof, generally non-toxic to cells and tissues, and which do not cause any significant adverse effects to cells and tissues when cells and tissues are incubated (e.g., cultured) in their presence.

The detectable moiety can contain a luminophore such as a fluorescent label or near-infrared label. Examples of suitable luminophores include, but are not limited to, metal porphyrins; benzoporphyrins; azabenzoporphyrine; napthoporphyrin; phthalocyanine; polycyclic aromatic hydrocarbons such as perylene diimine, pyrenes; azo dyes; xanthene dyes; boron dipyoromethene, aza-boron dipyoromethene, cyanine dyes, metal-ligand complex such as bipyridine, bipyridyls, phenanthroline, coumarin, and acetylacetonates of ruthenium and iridium; acridine, oxazine derivatives such as benzophenoxazine; aza-annulene, squaraine; 8-hydroxyquinoline, polymethines, luminescent producing nanoparticle, such as quantum dots, nanocrystals; carbostyril; terbium complex; inorganic phosphor; ionophore such as crown ethers affiliated or derivatized dyes; or combinations thereof. Specific examples of suitable luminophores include, but are not limited to, Pd(II) octaethylporphyrin; Pt(II)-octaethylporphyrin; Pd(II) tetraphenylporphyrin; Pt(II) tetraphenylporphyrin; Pd(II) meso-tetraphenylporphyrin tetrabenzoporphine; Pt(II) meso-tetraphenyl metrylbenzoporphyrin; Pd(II) octaethylporphyrin ketone; Pt(II) octaethylporphyrin ketone; Pd(II) meso-tetra(pentafluorophenyl)porphyrin; Pt(II) meso-tetra (pentafluorophenyl) porphyrin; Ru(II) tris(4,7-diphenyl-1,10-phenanthroline) (Ru (dpp)₃); Ru(II) tris(1,10-phenanthroline) (Ru(phen)₃), tris(2,2′-bipyridine)ruthenium (II) chloride hexahydrate (Ru(bpy)₃); erythrosine B; fluorescein; fluorescein isothiocyanate (FITC); eosin; iridium (III) ((N-methyl-benzimidazol-2-yl)-7-(diethylamino)-coumarin)); 152enzothiazole) ((benzothiazol-2-yl)-7-(diethylamino)-coumarin))-2-(acetylacetonate); Lumogen dyes; Macroflex fluorescent red; Macrolex fluorescent yellow; Texas Red; rhodamine B; rhodamine 6G; sulfur rhodamine; m-cresol; thymol blue; xylenol blue; cresol red; chlorophenol blue; bromocresol green; bromocresol red; bromothymol blue; Cy2; a Cy3; a Cy5; a Cy5.5; Cy7; 4-nitirophenol; alizarin; phenolphthalein; o-cresolphthalein; chlorophenol red; calmagite; bromo-xylenol; phenol red; neutral red: nitrazine; 3,4,5,6-tetrabromphenolphtalein; congo red; fluor‘sc’in; eosin; 2,7-dichlorofluorescein; 5(6)-carboxy-fluoreesein; carboxynaphthofluorescein; 8-hydroxypyrene-1,3,6-trisulfonic acid; semi-naphthorhodafluor; semi-naphthofluorescein; tris (4,7-diphenyl-1,10-phenanthroline) ruthenium (II) dichloride; (4,7-diphenyl-1,10-phenanthroline) ruthenium (II) tetraphenylboron; platinum (II) octaethylporphyin; dialkylcarbocyanine; dioctadecylcycloxacarbocyanine; fluorenylmethyloxycarbonyl chloride; 7-amino-4-methylcourmarin (Amc); green fluorescent protein (GFP); and derivatives or combinations thereof.

In some examples, the detectable moiety can include Rhodamine B (Rho), fluorescein isothiocyanate (FITC), 7-amino-4-methylcourmarin (Amc), green fluorescent protein (GFP), or derivatives or combinations thereof.

### Methods of Making

The compounds described herein can be prepared in a variety of ways known to one skilled in the art of organic synthesis or variations thereon as appreciated by those skilled in the art. The compounds described herein can be prepared from readily available starting materials, Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, e.g., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high-performance liquid chromatography (HPLC) or thin layer chromatography.

The disclosed compounds can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for aspartic acid and glutamic acid, benzyl and t-butyl and for cysteine, triphenylmethyl (trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif.). The α-C-terminal amino acid is coupled to the resin by means of N,N-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxy-acetamidoethyl resin, the Fmoc group is cleaved with a secondary amine, preferably piperidine, prior to coupling with the α-C-terminal amino acid as described above. One method for coupling to the deprotected 4 (2′,4dimethoxyphenyl-Fmoc-aminomethyl)phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

The above polymers, such as PEG groups, can be attached to an oligonucleotide, such as an AC, under any suitable conditions. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the AC (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., α-iodo acetic acid, α-bromoacetic acid, α-chloroacetic acid). If attached to the AC by reductive alkylation, the polymer selected should have a single reactive aldehyde so that the degree of polymerization is controlled. See, for example, Kinstler et al., Adv, Drug. Delivery Rev. (2002), 54: 477-485; Roberts et al., Adv. Drug Delivery Rev. (2002), 54: 459-476; and Zalipsky et al., Adv. Drug Delivery Rev. (1995), 16: 157-182.

In order to direct covalently link the AC or linker to the CPP, appropriate amino acid residues of the CPP may be reacted with an organic derivatizing agent that is capable of reacting with a selected side chain or the N- or C-termini of an amino acids. Reactive groups on the peptide or conjugate moiety include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art.

Methods of making AC and conjugating AC to linear CPP are generally described in US Pub. No. 2018/0298383, which is herein incorporated by reference for all purposes. The methods may be applied to the cyclic CPPs disclosed herein.

Synthetic schemes are provided in FIGS. 5A-5D and FIG. 6.

Non-limiting examples of compounds that include a CPPs and a reactive group useful for conjugation to an AC are shown in Table 6. Example linker groups are also shown. Example reactive groups include tetrafluorophenyl ester (TFP), free carboxylic acid (COOH), and azide (N₃). In Table 6, n is an integer from 0 to 20; Pipa6 is AcRXRRBRRXRYQFLIRXRBRXRB wherein B is β-Alanine and X is aminohexanoic acid; Dap is 2,3-diaminopropionic acid; NLS is a nuclear localization sequence; βA is beta alanine; -ss- is a disulfide; PABC is poly(A) binding protein C-terminal domain; Cₓ where x is a number is an alkyl chain of length x; and BCN is bicyclo[6.1.0]nonyne.

TABLE 6

Compounds that include a CPPs and a reactive group
TFP-PEGₙ-K(CPP)
TFP-PEGₙ-K(CPP)-PEGₙ-Dap(palmitoyl)
TFP-PEGₙ-K(CPP)-PEGₙ-Dap(CPP)
TFP-Pip6a
CPP-PEGₙ-TFP
CPP-PEGₙ-K(CPP)-PEGₙ-TFP
CPP-PEGₙ-Lys(N₃)
CPP-K(CPP)-PEGₙ-K(N₃)
CPP-PEGₙ-K(PEGₙ-CPP)-PEGₙ-K(N₃)
CPP-PEGₙ-K(PEGₙ-CPP)-PEGₙ-K(N₃)
CPP-K(CPP)-K(CPP)-PEGₙ-K(N₃)
CPP-PEGₙ-K(PEGₙ-CPP)-K(PEGₙ-CPP)-PEGₙ-K(N₃)
CPP-PEGₙ-K(PEGₙ-CPP)-K(PEGₙ-CPP)-PEGₙ-K(N₃)
Ac-NLS-Lys(CPP)-PEGₙ-K(N₃)
K(N₃)-PEGₙ-NLS-ss-PEGₙ-CPP
BCN-NLS-ss-CPP
CPP-PEGₙ-Val-Cit-PABC-K(N₃)
CPP-PEGₙ-Cys-ss-Cys-K(N₃)
CPP-PEGₙ-Cys-ss-Cys-K(N₃)
CPP-PEGₙ-TFP
CPP-PEGₙ-Lys(N₃)
CPP-PEGₙ-Cys-prodisulfide-K(N₃)
CPP-PEGₙ-K(N₃)
CPP-K(CPP)-PEGₙ-K(N₃)
CPP-PEGₙ-K(CPP)-PEGₙ-TFP
CPP-C₆-TFP
CPP-PEGₙ-K(PEGₙ-CPP)-PEGₙ-K(N₃)
Ac-T9-PEGₙ-Lys(CPP-PEGₙ)-K(N₃)
Ac-MSP-PEGₙ-K(CPP-PEGₙ)-K(N₃)
CPP-PEGₙ-TFP (ENTRD 802)
CPP-C₆-TFP (ENTRD 696)
CPP-PEGₙ-K(CPP)-PEGₙ-TFP
CPP-PEGₙ-COOH
CPP-C₁₂-TFP
palmitoyl-PEGₙ-K(CPP)-PEGₙ-TFP
CPP-PEGₙ-K(N₃)
Ac-T9-PEGₙ-K(CPP)-K(N₃)
Ac-MSP-PEGₙ-K(CPP-PEGₙ)-K(N₃)
Ac-NLS-K(CPP)-PEGₙ-K(N₃
K(N3)-PEGₙ-NLS-ss-PEGₙ-CPP
K(N3)-PEGₙ-NLS-K-βA-βA-CPP

In embodiments, the CPPs have free carboxylic acid groups that may be utilized for conjugation to an AC. In embodiments, the EEVs have free carboxylic acid groups that may be utilized for conjugation to an AC.

The structure below is a 3 cyclooctyne modified PMO used for a click reaction with a compound that includes an azide:

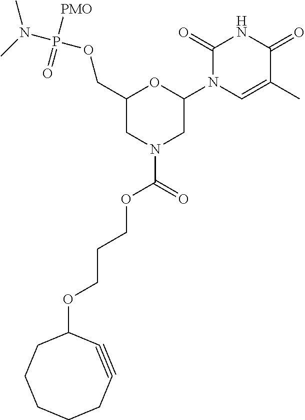

An example scheme of conjugation of a CPP and linker to the 3′ end of an AC via an amide bond is shown below.

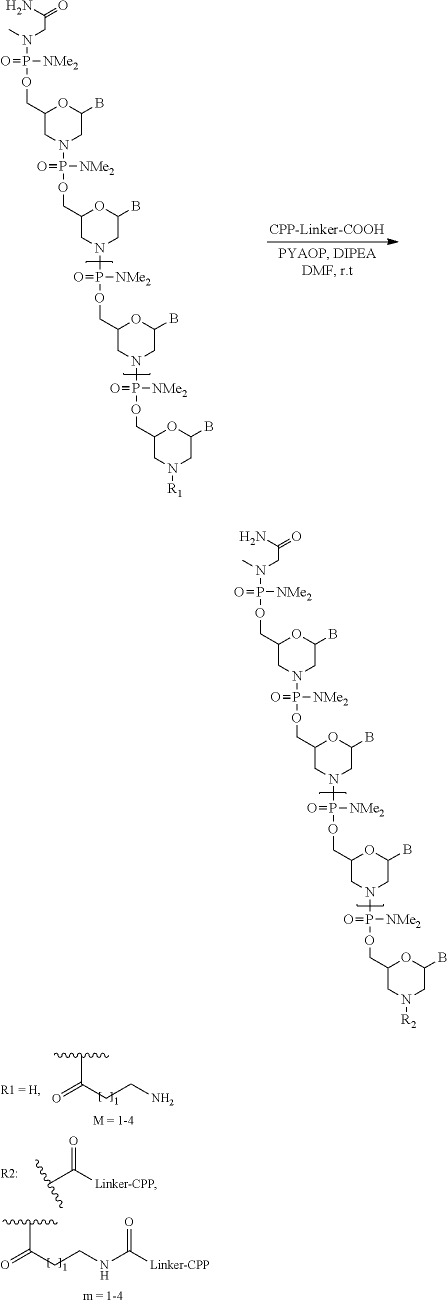

An example scheme of conjugation of a CPP and linker to a 3′-cyclooctyne modified PMO via strain-promoted azide-alkyne cycloaddition is shown below:

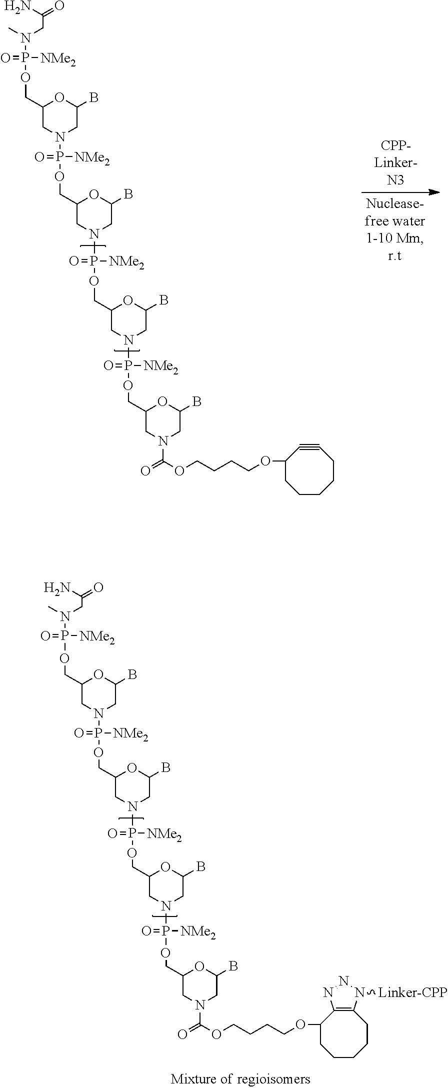

An example of the conjugation chemistry used to connect an AC and CPP with an additional linker containing a polyethylene glycol moiety is shown below:

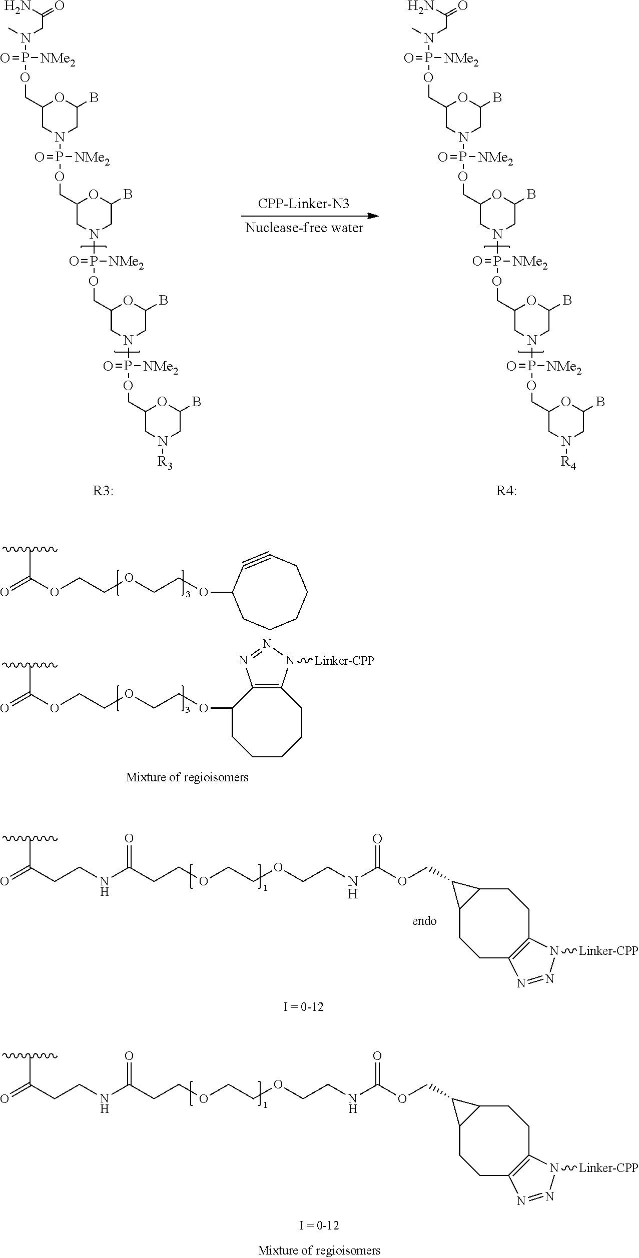

An example of conjugation of a CPP-linker to a 5′-cyclooctyne modified PMO via strain-promoted azide-alkyne cycloaddition (click chemistry) is shown below:

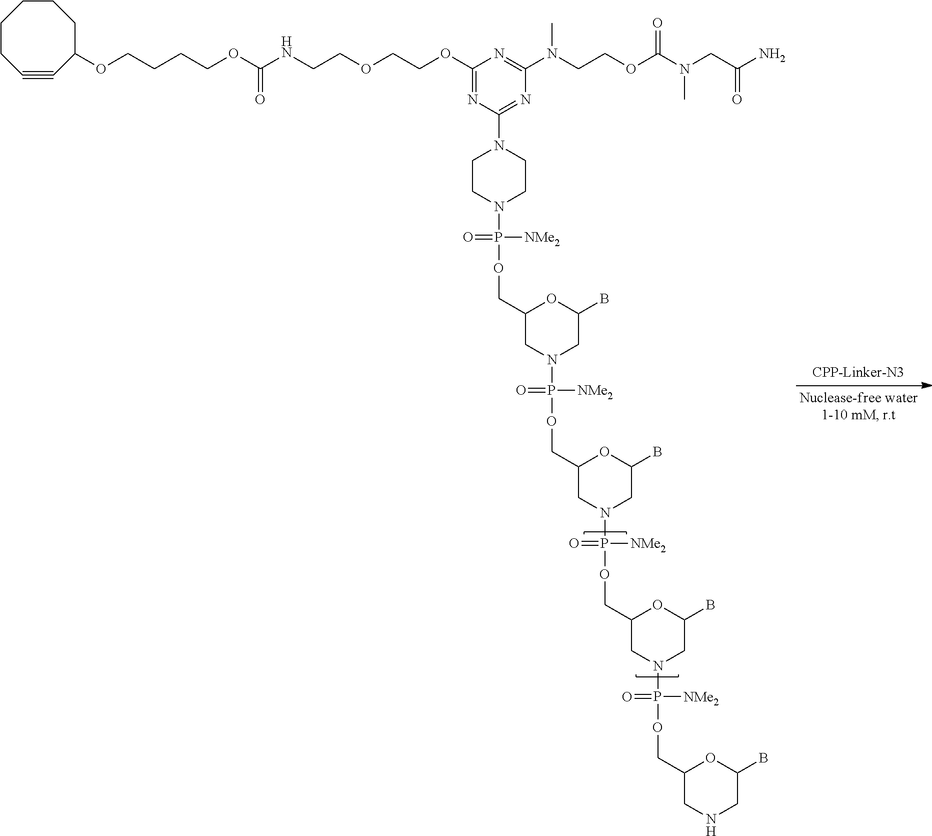

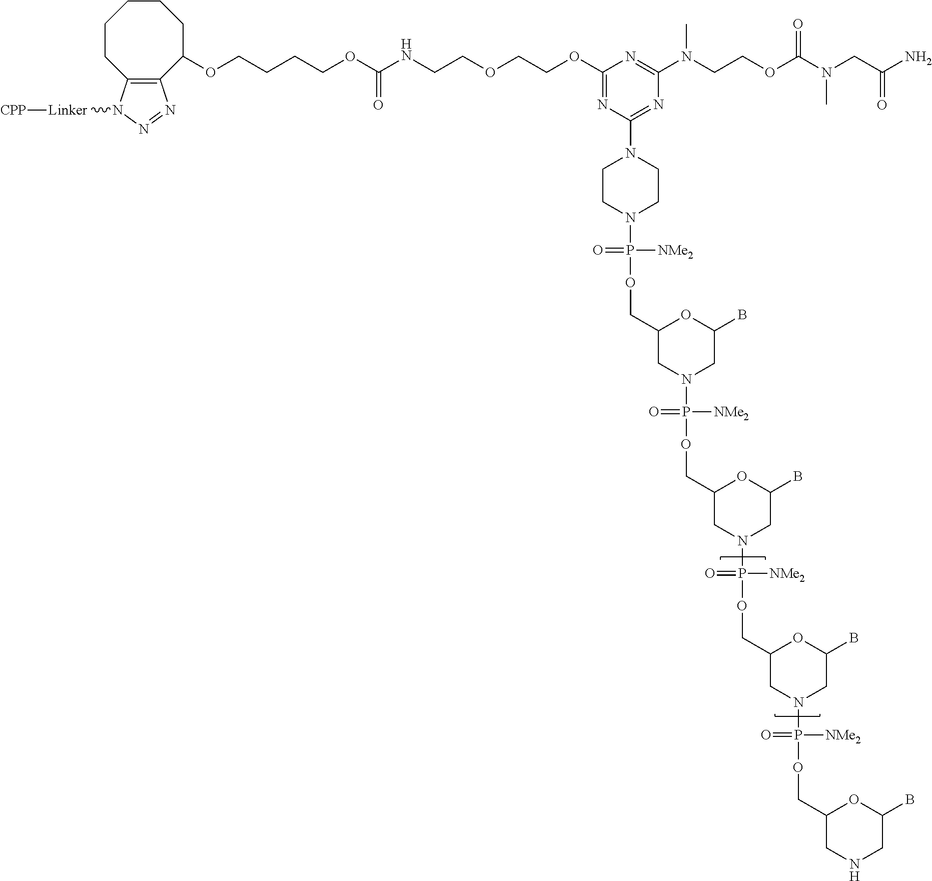

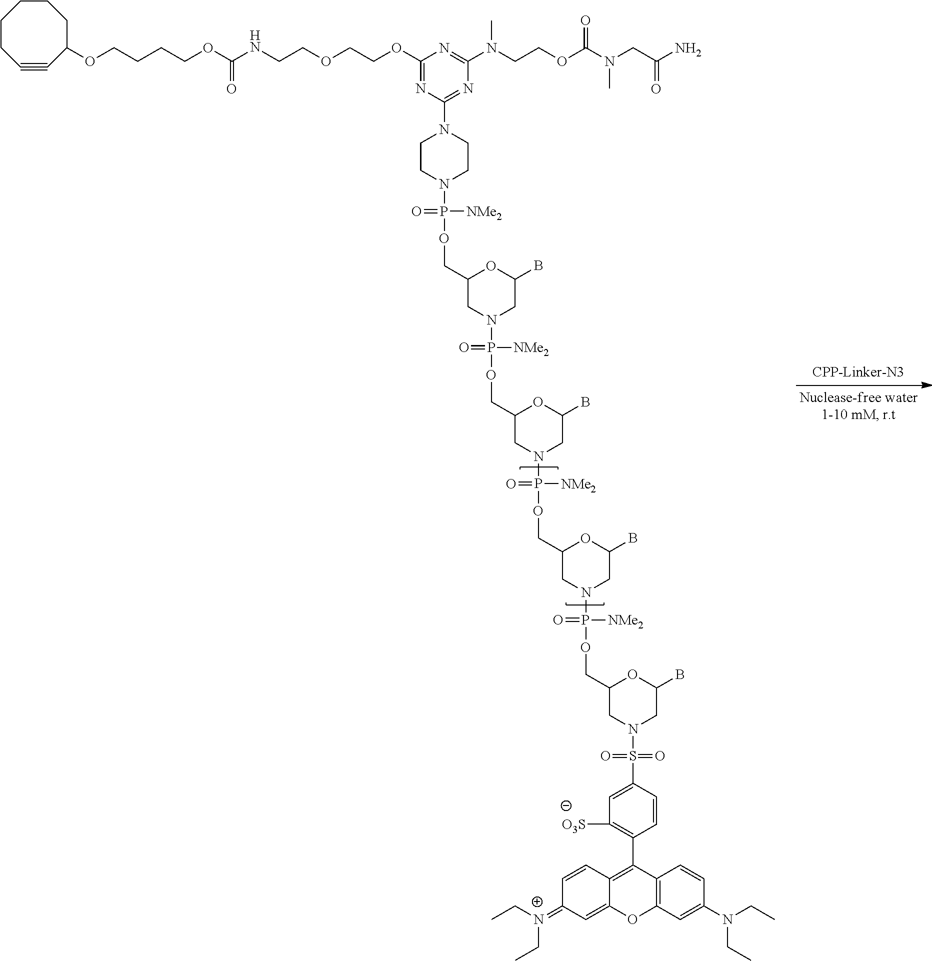

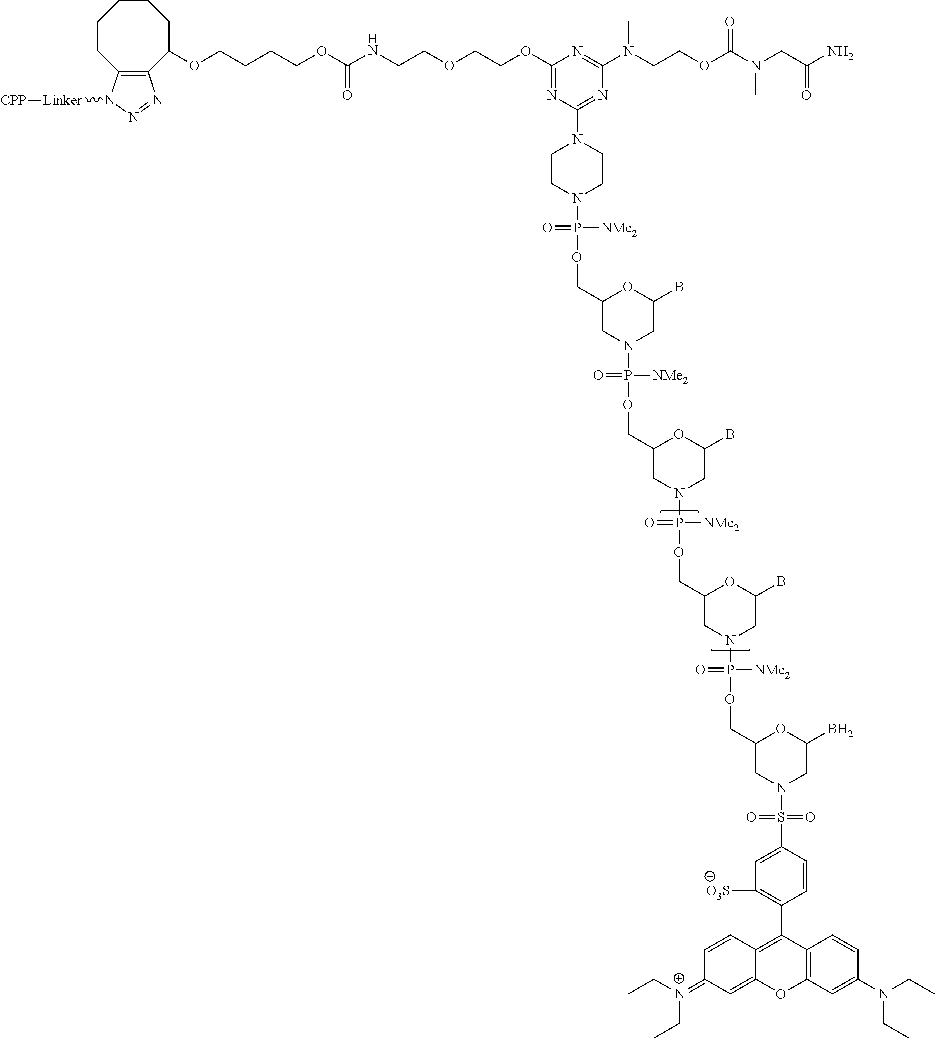

Methods of synthesizing oligomeric antisense compounds are known in the art. The present disclosure is not limited by the method of synthesizing the AC. In embodiments, provided herein are compounds having reactive phosphorus groups useful for forming internucleoside linkages including for example phosphodiester and phosphorothioate internucleoside linkages. Methods of preparation and/or purification of precursors or antisense compounds are not a limitation of the compositions or methods provided herein. Methods for synthesis and purification of DNA, RNA, and the antisense compounds are well known to those skilled in the art.

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Antisense compounds provided herein can be conveniently and routinely made through the well-known technique of solid phase synthesis. Equipment for such synthesis is sold by several vendors including, for example, Applied Biosystems (Foster City, CA). Any other means for such synthesis known in the art may additionally or alternatively be employed. It is well known to use similar techniques to prepare oligonucleotides such as the phosphorothioates and alkylated derivatives. The present disclosure is not limited by the method of antisense compound synthesis.

Methods of oligonucleotide purification and analysis are known to those skilled in the art. Analysis methods include capillary electrophoresis (CE) and electrospray-mass spectroscopy. Such synthesis and analysis methods can be performed in multi-well plates. The method of the invention is not limited by the method of oligomer purification.

### Diseases

In some embodiments, various diseases or conditions can be treated, prevented or ameliorated with administration of a composition that includes one or more of the compounds described herein. In embodiments, the disease to be treated, prevented, or ameliorated with a composition of the present disclosure is associated with dysregulation of splicing, protein expression, and/or protein activity.

In embodiments, the compounds disclosed herein are used for treating, preventing, or ameliorating a disease or condition. Illustrative diseases or conditions that can be treated, prevented or modulated using compounds of the present disclosure can include, but are not limited to cancers, including for example acute myeloid leukemia, B-cell leukemia/lymphoma, bladder cancer, breast cancer, chronic lymphocytic leukemia, colon cancer, colorectal cancer, Duchenne muscular dystrophy, esophageal squamous cell carcinoma, fanconi anemia, gastric cancer, glioblastoma, hepatocellular carcinoma, lung cancer, lynch syndrome, mantle cell lymphoma, melanoma, nasopharyngeal carcinoma, neuroblastoma, ovarian cancer, pancreatic ductal adenocarcinoma, proliferative conditions, prostate cancer, and small intestinal neuroendocrine cancer; cardiovascular conditions including for example atherosclerosis, cardiac hypertrophy, dilated cardiomyopathy, hypertension, ischemia/reperfusion injury, thrombosis (deep vein), and thrombosis (venous); congenital abnormalities including microphthalmia, mullerian aplasia, bone fragility (osteogenesis imperfecta), and rickets; endocrine disorders including neonatal diabetes and type 2 diabetes; hematological disorders including glanzmann thrombasthenia, α-thalassemia, and β-thalassemia; immunological disorders including IPEX syndrome, nasal polyps, severe combined immunodeficiency, systemic lupus erythematosus, and Wiskott-Aldrich syndrome; lung conditions including pulmonary fibrosis; musculoskeletal conditions including muscle fibrosis, facioscapulohumeral muscular dystrophy, oculopharyngeal muscular dystrophy, myotonic dystrophy, and oculopharyngeal muscular dystrophy; neurological conditions including Alzheimer disease, amyotrophic lateral sclerosis, anxiety disorders, fabry disease, fragile X syndrome, friedrich's ataxia, huntington's disease, metachromatic leukodystrophy, pseudodeficiency, neuropsychiatric disease, Parkinson disease, and suicidal behavior; stress; Zellweger syndrome; glycogen storage diseases such as Pompe disease; an combinations thereof.

In embodiments, the compounds disclosed herein are used for treating, preventing or ameliorating a disease associated with aberrant gene transcription, splicing and/or translation. In embodiments, the compounds disclosed herein are used for treating, preventing or ameliorating a disease associated with aberrant IRF-5, GYS1, and/or DUX4 transcription, splicing and/or translation. In embodiments, the compounds disclosed herein are used for treating, preventing or ameliorating a disease associated with IRF-5, GYS1, and/or DUX4 upregulation; IRF-5, GYS1, and/or DUX4 polymorphisms; accumulation of mutant IRF-5, GYS1, and/or DUX4; or combinations thereof.

### Glycogen Storage Diseases

Glycogen synthesis and degradation are multi-step processes involving many different enzymatic reactions. For example, alpha-glucosidase (GAA) catalyzes the hydrolysis of glycogen by cleaving a-1,4 and a-1,6 glycosidic linkages allowing glucose to be liberated into the cytoplasm. In the absence of GAA, glycogen accumulates within the lysosomes in various tissues, primarily cardiac and skeletal muscle. Conditions caused by a deficiency of this protein are referred to as Glycogen storage diseases (GSDs) (Douillard-Guilloux et al., Hum. Mol. Genet. (2010), 19(4):684-96).

GSDs are inherited metabolic disorders of glycogen metabolism. There are over 12 types of glycogen storage diseases, which are classified based on the enzyme deficiency and the affected tissue, primarily the liver or the muscle. Type 0 GSD is due to a deficiency in glycogen synthase. Type I is due to a deficiency in glucose-6-phosphatase a. Type II is due to a deficiency of alpha-glucosidase (GAA). Type III is due to a deficiency of the glycogen debranching enzyme (GDE). Type IV is due to a deficiency of glycogen branching activity. Type V is due to a deficiency in the muscle isoform of glycogen phosphorylase (encoded by PYGM). Type VI is due to a deficiency of the liver isoform of glycogen phosphorylase (encoded by PYGL). There is little information about the remaining GSD and several former GSDs have been classified into other disorders. A list of glycogen storage diseases is provided in Table 7 (Ellingwood S. et al., (2018), J. Endocrinol. 238(3): R131-R141. doi:10.1530/JOE-18-0120).

TABLE 7
Glycogen storage diseases

| | Alternate | | | OMIM* |
|---|---|---|---|---|
| | names or | Affected | | phenotype |
| Type | subtype | enzyme/pathway | Gene | no. |
| 0 | Oa | Liver glycogen | GYS2 | 240600 |
| | | synthase | | |
| | 0b | Muscle glycogen | GYS1 | 611556 |
| | | synthase | | |
| I | Ia; von Gierke | Glucose-6- | G6PC | 232200 |
| | | phosphatase α | | |
| | Ib; von Gierke | Glucose-6-phosphate | SLC37A4 | 232220 |
| | | transporter | | |
| II | Pompe | Acid α-glucosidase | GAA | 232300 |
| III | Cori/Forbes | Glycogen debranching | AGL | 232400 |
| | | enzyme | | |
| IV | Andersen | Glycogen branching | GBE1 | 232500 |
| | | enzyme | | |
| V | McArdle | Muscle glycogen | PYGM | 232600 |
| | | phosphorylase | | |
| VI | Hers | Liver glycogen | PYGL | 232700 |
| | | phosphorylase | | |
| VII | Tarui | Muscle | PFKM | 232800 |
| | | phosphofructose | | |
| | | kinase | | |
| IX | IXa | Phosphorylase kinase | PHKA2 | 306000 |
| | | (α2 subunit) | | |
| | IXb | Phosphorylase kinase | PHKB | 261750 |
| | | (β subunit) | | |
| | IXc | Phosphorylase kinase | PHKG2 | 613027 |
| | | (γ subunit) | | |
| | IXd | Phosphorylase kinase | PHKA1 | 300559 |
| | | (α1 subunit) | | |
| X | — | Muscle | PGAM2 | 261670 |
| | | phosphoglycerate | | |
| | | mutase | | |
| XI^{#} | Fanconi-Bickel | Glucose transporter 2 | SLC2A2 | 227810 |
| XII | — | Aldolase A | ALDOA | 611881 |
| XIII | — | β-Enolase | ENO3 | 612932 |
| XV | — | Glycogenin-1 | GYG1 | 603942 |
| Danon | | Lysosomal-associated | LAMP2 | 300257 |
| disease | | membrane protein 2 | | |
| Lafora | 2A | Laforin | EPM2A | 254780 |
| disease | | | | |
| | 2B | Malin | NHLRC1 | 254780 |

*OMIM (Online Mendelian Inheritance in Man);
^{#}in earlier sources, GSD type XI was associated with a deficiency in lactate dehydrogenase A (OMIM 612933).

Glycogen storage disease type II (GSDII) or Pompe disease is an autosomal recessive lysosomal storage disorder caused by a mutation in the gene that encodes for glucosidase alpha acid (GAA), which results in an absence or deficiency of GAA protein that is essential to the breakdown of complex sugar, glycogen. Normally, the body uses GAA to break down the complex carbohydrate glycogen and convert it into glucose. Failure to achieve proper breakdown and abnormalities in glycogen metabolism result in the excessive accumulation of glycogen in the body's cells, particularly in cardiac, smooth, and skeletal muscle cells, which can lead to impairment and degradation of normal tissue and organ function. Patients with Pompe disease experience serious muscle-related problems, including progressive muscle weakness throughout the body, especially in the legs, trunk, and diaphragm. As the disorder progresses, breathing problems can lead to respiratory failure. To date, more than 300 pathogenic mutations have been identified in GAA. Pompe disease is commonly estimated to affect between 5,000 and 10,000 patients in the aggregate in the United States and Europe; however, the advent of newborn screening suggests the disease is underdiagnosed.

Based on the age of onset and severity of symptoms, Pompe disease is typically classified as either infantile-onset Pompe disease (IOPD) or late-onset Pompe disease (LOPD). IOPD is characterized by severe muscle weakness and abnormally diminished muscle tone and usually manifests within the first few months of life. If left untreated, IOPD is often fatal due to progressive cardiac failure, respiratory distress or malnutrition resulting from feeding difficulties. LOPD presents in childhood, adolescence or adulthood. Patients with LOPD typically have milder symptoms, such as reduced mobility and respiratory problems. Patients with LOPD experience progressive difficulty walking and respiratory decline. Initial symptoms of LOPD may be subtle and go unrecognized for years.

At the time of filing, the only currently approved therapies for Pompe disease are alglucosidase alfa (Lumizyme in the United States, Myozyme in other geographies) and avalglucosidase alfa-ngpt (Nexviazyme in the United States), which are both forms of enzyme replacement therapy (ERT) delivered via IV infusions. Although infantile patients treated with ERT for Pompe disease have demonstrated improved survival, ERT is not curative, and many patients in long-term observational studies continue to have increased risk of both cardiomyopathy and heart failure. These patients also experience residual muscle weakness, including difficulties swallowing and the attendant increased risk of aspiration. ERT is particularly limited in its ability to improve skeletal muscle myopathy and respiratory dysfunction, primarily due to its inability to penetrate key tissues affected by the disease, a lack of activity in the cytosol and potential immunogenicity. Despite the availability of ERT, there remains significant unmet medical need in patients with either IOPD or LOPD.

GAA catalyzes the hydrolysis of glycogen by cleaving α-1,4 and α-1,6 glycosidic linkages allowing glucose to be liberated into the cytoplasm. In the absence of GAA, glycogen accumulates within the lysosomes in various tissues, primarily cardiac and skeletal muscle. Conditions caused by a deficiency of this protein are referred to as Glycogen storage diseases (GSDs) (Douillard-Guilloux (2010) Hum. Mol. Genet. 19(4):684-96).

One manner in which glycogen storage diseases can be treated is by downregulating glycogen synthesis, for example, by downregulating the expression and/or activity of glycogen synthase. There are two main isozymes of the glycogen synthase, GYS1 and GYS2. GYS1 is ubiquitously expressed in skeletal and cardia muscle (NCBI reference 2997). GYS2 is mainly expressed in the liver and fatty tissues (NCBI gene reference 2998). GYS1 functions to break down ingested glucose to provide a glycogen energy reserve for the muscles. In contrast, GYS2 functions to maintain blood glucose levels. Alignment of the mRNA of GYS1 and GYS2 shows that the 54% of the two isozymes share 71% homology.

It has been shown that downregulation glycogen synthase (GYS1) expression results in a reversal of glycogen accumulation (Douillard-Guilloux, et al., Hum. Mol. Genet. (2010), 19(4):684-96). The structure and mechanism of action of GYS1 has been reviewed (Palm, D. C., et al., FEBS (2013), 280(1), 2-27; and Baskaran S., et al., Proc. Natl. Acad. Sci. USA (2010) 107, 17563-17568. Due to the differences in functions of GYS1 and GYS1, it is important to selectively target GYS1 for downregulation.

In embodiments, a method is provided for treating a glycogen storage disease. In embodiments, the method includes administering a compound that downregulates glycogen synthesis. In embodiments, the method includes administering a compound that downregulates expression of glycogen synthase. In embodiments, the method includes administering a compound that downregulates expression of the muscle form of glycogen synthase (GYS1). In embodiments, the compound includes an AC. The AC may be any AC and have any AC characteristics as described elsewhere herein. In embodiments, the AC may bind to at least a portion of a SE or a SRE of a target transcript as described elsewhere herein. In embodiments, the AC may bind to in proximity to a SE or a SRE of a target transcript as described elsewhere herein. In embodiments, the AC is an ASO, In embodiments, the ASO is a PMO. The AC may bind to any splice element of an GYS1 target transcript as described elsewhere herein.

In embodiments, a method is provided for treating a glycogen storage disease. In embodiments, a method is provided for treating a glycogen storage disease associated with glycogen accumulation in muscle tissue. In embodiments, a method is provided for treating a glycogen storage disease associated with glycogen accumulation in cardiac muscle tissue. In embodiments, a method is provided for treating a glycogen storage disease associated with glycogen accumulation in skeletal muscle tissue. In embodiments, a method is provided for treating a type II glycogen storage disease. In embodiments, a method is provided for treating Pompe disease. In embodiments, a method is provided for treating Andersen disease. In embodiments, a method is provided for treating McArdle disease. In embodiments, a method is provided for treating Lafra disease. In embodiments, a method is provided for treating Tariu disease.

In embodiments, GYS1 is encoded by a nucleotide sequence encoding Isoform 1 or Isoform 2. The nucleotide sequences are publicly available, through the online NCBI database (Isoform 1=NM_002103.5; Isoform 2=NM_001161587.2) In embodiments, a nucleotide sequence encoding GYS1 differs by one or more nucleic acids from a nucleotide sequence encoding Isoform 1 or Isoform 2. In embodiments the nucleotide sequence encoding GYS1 differs by one or more polymorphisms (e.g., Single Nucleotide Polymorphisms or SNPs). In embodiments, the nucleotide sequence encoding GYS1 shares less than 100% sequence identity with a nucleotide sequence encoding Isoform 1 or Isoform 2, In embodiments, GYS1 is encoded by nucleotide sequence that is at least about 80%, about 85%, about 90%, about 91%, about 92%, about 93%, about 94%, about 95%, about 96%, about 97%, about 98%, or about 99% identical to a nucleic acid sequence encoding Isoform 1 or Isoform 2. In embodiments, GYS1 is encoded by nucleotide sequence that is 80% to 100%, 90% to 100%, 95% to 100%, or 99% to 100% identical to a nucleic acid sequence encoding isoform 1 or Isoform 2.

In embodiments, the method includes administering a compound that induces exon skipping of one or more exons in a GYS1 target transcript. In embodiments, the method includes administering a compound that includes an antisense compound (AC) that induces skipping of one or more exons in a GYS1 target transcript. In embodiments, hybridization of an AC to target nucleotide sequence of a GYS1 transcript results in inclusion or skipping of one or more exons in the target transcript. In embodiments, skipping or inclusion of one or more exons induces a frameshift in the GYS1 target transcript. In embodiments, the frameshift results in a GYS1 transcript that encodes glycogen synthase with decreased activity. In embodiments, the frameshift results in a truncated or non-functional glycogen synthase. In embodiments, the frameshift results in the introduction of a premature termination codon in the GYS1 transcript. In embodiments, the introduction of a premature termination codon results in degradation of the GYS1 mRNA transcript by nonsense-mediated decay.

In embodiments, a compound includes an antisense compound (AC) that induces skipping of one or more of exons 2, 3, 4, 5, 6, 7, 8, 9, 10, 11, 12, 13, 14, or 15 of human and/or mouse GYS1. In embodiments, a compound includes an AC that induces skipping of one or more exons to produce an out of frame frameshift leading to the GYS1 target transcript target transcript being degraded (e.g., nonsense mediate decay) or being translated into an GYS1 protein with reduce or no activity. In embodiments, a compound includes an AC that induces skipping of one or more of exons 2, 5, 6, 7, 8, 10, 12, and/or 14 to produce an out of frame frameshift. In embodiments, a compound includes an AC that induces skipping of one or more exons to produce an in-frame deletion in a GYS1 target transcript. In embodiments, a compound includes an AC that induces skipping of one or more of exons 3, 4, 9, 11, 13 and/or 15. In embodiments, a. compound includes an AC that induces skipping of one or more of exons 3, 4, 9, 11, 13 and/or 15 to produce an in-frame deletion in a GYS1 target transcript.

In embodiments, a compound includes an AC that binds to one or more exon/intron and/or intro n/exon junctions to induce exon skipping. In embodiments, the AC compound includes any of the following sequences in Table 8 where capital letters indicate exon nucleotides and lower-case letters indicate intron nucleotides. In Table 8, SEQ ID NOs:151-247 are designed to induce exon skipping to produce a frameshift alteration. In embodiments, the frameshift alteration results in a premature stop codon. In embodiments, the frameshift alteration results in nonsense mediated decay of the GYS1 target transcript. In Table 8, SEQ F-D NOs: 249-318 are designed to induce exon skipping to produce an in-frame deletion. In ACs listed in Table 8 are designed to bind to target nucleotide sequences that. include exons, exon/intron junctions, and/or intron/exon junctions.

TABLE 8
Various AC sequences for targeting GYS1

| | | | Exon |
|---|---|---|---|
| | | | targeted |
| SEQ ID | | | for |
| NO: | ID | Sequence (5′ to 3′) | skipping |
| 151 | GYS1-exon2-IEJ-1 | ctgtgggcccaagcgtgtgagggca | 2 |
| 152 | GYS1-exon2-IEJ-2 | ACCCActgtgggcccaagcgtgtga | 2 |
| 153 | GYS1-exon2-IEJ-3 | ATGCCACCCActgtgggcccaagcg | 2 |
| 154 | GYS1-exon2-IEJ-4 | TGTAGATGCCACCCActgtgggccc | 2 |
| 155 | GYS1-exon2-IEJ-5 | CACCGTGTAGATGCCACCCActgtg | 2 |
| 156 | GYS1-exon2-IEJ-6 | TGCAGCACCGTGTAGATGCCACCCA | 2 |
| 157 | GYS1-exon2-EIJ-1 | CTTGCAGCCCTTGCTGTTCATGGAA | 2 |
| 158 | GYS1-exon2-EIJ-2 | cccacCTTGCAGCCCTTGCTGTTCA | 2 |
| 159 | GYS1-exon2-EIJ-3 | cacgtcccacCTTGCAGCCCTTGCT | 2 |
| 160 | GYS1-exon2-EIJ-4 | tgggccacgtcccacCTTGCAGCCC | 2 |
| 161 | GYS1-exon2-EIJ-5 | tgggctgggccacgtcccacCTTGC | 2 |
| 162 | GYS1-exon2-EIJ-6 | tgccctgggctgggccacgtcccac | 2 |
| 163 | GYS1-exon5-IEJ-1 | ctgggtgggaggggacagcagtccg | 5 |
| 164 | GYS1-exon5-IEJ-2 | TTGAActgggtgggaggggacagca | 5 |
| 165 | GYS1-exon5-IEJ-3 | CCACGTTGAActgggtgggagggga | 5 |
| 166 | GYS1-exon5-IEJ-4 | CTTGTCCACGTTGAActgggtggga | 5 |
| 167 | GYS1-exon5-IEJ-5 | GCTTCCTTGTCCACGTTGAActggg | 5 |
| 168 | GYS1-exonS-IEJ-6 | CCCCTGCTTCCTTGTCCACGTTGAA | 5 |
| 169 | GYS1-exonS-EIJ-1 | CTGGTTTCCTCTTGAGCAAGTGCTG | 5 |
| 170 | GYS1-exon5-EIJ-2 | cctacCTGGTTTCCTCTTGAGCAAG | 5 |
| 171 | GYS1-exonS-EIJ-3 | cagcccctacCTGGTTTCCTCTTGA | 5 |
| 172 | GYS1-exon5-EIJ-4 | cagcccagcccctacCTGGTTTCCT | 5 |
| 173 | GYS1-exon5-EIJ-5 | gacttcagcccagcccctacCTGGT | 5 |
| 174 | GYS1-exon5-EIJ-6 | ctggggacttcagcccagcccctac | 5 |
| 175 | GYS1-exon6-IEJ-1 | ctgggattgggggtgagggtcccat | 6 |
| 176 | GYS1-exon6-IEJ-2 | AATATctgggattgggggtgagggt | 6 |
| 177 | GYS1-exon6-IEJ-3 | GTCACAATATctgggattgggggtg | 6 |
| 178 | GYS1-exon6-IEJ-4 | TGGGGGTCACAATATctgggattgg | 6 |
| 179 | GYS1-exon6-IEJ-5 | CCCATTGGGGGTCACAATATctggg | 6 |
| 180 | GYS1-exon6-IEJ-6 | TTCAGCCCATTGGGGGTCACAATAT | 6 |
| 181 | GYS1-exon6-EIJ-1 | CCATAAAAATGGCCCCGCACAAACT | 6 |
| 182 | GYS1-exon6-EIJ-2 | cgtacCCATAAAAATGGCCCCGCAC | 6 |
| 183 | GYS1-exon6-EIJ-3 | ccccacgtacCCATAAAAATGGCCC | 6 |
| 184 | GYS1-exon6-EIJ-4 | atatgccccacgtacCCATAAAAAT | 6 |
| 185 | GYS1-exon6-EIJ-5 | taggtatatgccccacgtacCCATA | 6 |
| 186 | GYS1-exon6-EIJ-6 | agacctaggtatatgccccacgtac | 6 |
| 187 | GYS1-exon7-IEJ-1 | ctaaagaacccacaaggcacggtaa | 7 |
| 188 | GYS1-exon7-IEJ-2 | GATGCctaaagaacccacaaggcac | 7 |
| 189 | GYS1-exon7-IEJ-3 | GTCCAGATGCctaaagaacccacaa | 7 |
| 190 | GYS1-exon7-IEJ-4 | TTGAAGTCCAGATGCctaaagaacc | 7 |
| 191 | GYS1-exon7-IEJ-5 | CCAAGTTGAAGTCCAGATGCctaaa | 7 |
| 192 | GYS1-exon7-IEJ-6 | CTTGTCCAAGTTGAAGTCCAGATGC | 7 |
| 193 | GYS1-exon7-EIJ-1 | TCTGAGCAGATAGTTGAGCCGAGCC | 7 |
| 194 | GYS1-exon7-EIJ-2 | ctcacTCTGAGCAGATAGTTGAGCC | 7 |
| 195 | GYS1-exon7-EIJ-3 | caggcctcacTCTGAGCAGATAGTT | 7 |
| 196 | GYS1-exon7-EIJ-4 | tagcccaggcctcacTCTGAGCAGA | 7 |
| 197 | GYS1-exon7-EIJ-5 | cctcatagcccaggcctcacTCTGA | 7 |
| 198 | GYS1-exon7-EIJ-6 | tgtcccctcatagcccaggcctcac | 7 |
| 199 | GYS1-exon8-IEJ-1 | ctgcgcagaaagaaaggagggggag | 8 |
| 200 | GYS1-exon8-IEJ-2 | TTCACctgcgcagaaagaaaggagg | 8 |
| 201 | GYS1-exon8-IEJ-3 | TGCCGTTCACctgcgcagaaagaaa | 8 |
| 202 | GYS1-exon8-IEJ-4 | CTCGCTGCCGTTCACctgcgcagaa | 8 |
| 203 | GYS1-exon8-IEJ-5 | GTCTGCTCGCTGCCGTTCACctgcg | 8 |
| 204 | GYS1-exon8-IEJ-6 | CCACTGTCTGCTCGCTGCCGTTCAC | 8 |
| 205 | GYS1-exon8-EIJ-1 | CAAAGCTGTTTGCGCACAGCTTGGC | 8 |
| 206 | GYS1-exon8-EIJ-2 | ctaacCAAAGCTGTTTGCGCACAGC | 8 |
| 207 | GYS1-exon8-EIJ-3 | ggctgctaacCAAAGCTGTTTGCGC | 8 |
| 208 | GYS1-exon8-EIJ-4 | gcgagggctgctaacCAAAGCTGTT | 8 |
| 209 | GYS1-exon8-EIJ-5 | ccggagcgagggctgctaacCAAAG | 8 |
| 210 | GYS1-exon8-EIJ-6 | aggggccggagcgagggctgctaac | 8 |
| 211 | GYS1-exon10-IEJ-1 | ctgcaaggcaagcaggggcatgcat | 10 |
| 212 | GYS1-exon10-IEJ-2 | TCCCActgcaaggcaagcaggggca | 10 |
| 213 | GYS1-exon10-IEJ-3 | AAGGCTCCCActgcaaggcaagcag | 10 |
| 214 | GYS1-exon10-IEJ-4 | TCGGGAAGGCTCCCActgcaaggca | 10 |
| 215 | GYS1-exon10-IEJ-5 | TCATGTCGGGAAGGCTCCCActgca | 10 |
| 216 | GYS1-exon10-IEJ-6 | CTTGTTCATGTCGGGAAGGCTCCCA | 10 |
| 217 | GYS1-exon10-EIJ-1 | CTGCGTTGCAAAGATGGCTCTCTTC | 10 |
| 218 | GYS1-exon10-EIJ-2 | catacCTGCGTTGCAAAGATGGCTC | 10 |
| 219 | GYS1-exon10-EIJ-3 | caatccatacCTGCGTTGCAAAGAT | 10 |
| 220 | GYS1-exon10-EIJ-4 | aggtccaatccatacCTGCGTTGCA | 10 |
| 221 | GYS1-exon10-EIJ-5 | cacagaggtccaatccatacCTGCG | 10 |
| 222 | GYS1-exon10-EIJ-6 | ctctgcacagaggtccaatccatac | 10 |
| 223 | GYS1-exon12-IEJ-1 | ctggtagtgaaaaagaaggactcag | 12 |
| 224 | GYS1-exon12-IEJ-2 | ATCACctggtagtgaaaaagaagga | 12 |
| 225 | GYS1-exon12-IEJ-3 | GGAAAATCACctggtagtgaaaaag | 12 |
| 226 | GYS1-exon12-IEJ-4 | CGGGTGGAAAATCACctggtagtga | 12 |
| 227 | GYS1-exon12-IEJ-5 | AACTCCGGGTGGAAAATCACctggt | 12 |
| 228 | GYS1-exon12-IEJ-6 | AGAGGAACTCCGGGTGGAAAATCAC | 12 |
| 229 | GYS1-exon12-EIJ-1 | CCGGTGTGTAGCCCCAAGGCTCATA | 12 |
| 230 | GYS1-exon12-EIJ-2 | ctcacCCGGTGTGTAGCCCCAAGGC | 12 |
| 231 | GYS1-exon12-EIJ-3 | ctacactcacCCGGTGTGTAGCCCC | 12 |
| 232 | GYS1-exon12-EIJ-4 | gcccactacactcacCCGGTGTGTA | 12 |
| 233 | GYS1-exon12-EIJ-5 | cccctgcccactacactcacCCGGT | 12 |
| 234 | GYS1-exon12-EIJ-6 | gctgtcccctgcccactacactcac | 12 |
| 235 | GYS1-exon14-IEJ-1 | ctgtggaggccaggacccaggttca | 14 |
| 236 | GYS1-exon14-IEJ-2 | GATACctgtggaggccaggacccag | 14 |
| 237 | GYS1-exon14-IEJ-3 | ATGTAGATACctgtggaggccagga | 14 |
| 238 | GYS1-exon14-IEJ-4 | CAAGAATGTAGATACctgtggaggc | 14 |
| 239 | GYS1-exon14-IEJ-5 | CCGGTCAAGAATGTAGATACctgtg | 14 |
| 240 | GYS1-exon14-IEJ-6 | AACCGCCGGTCAAGAATGTAGATAC | 14 |
| 241 | GYS1-exon14-EIJ-1 | CCGGCCTAGGTATTTCCAGTCCAGA | 14 |
| 242 | GYS1-exon14-EIJ-2 | cctacCCGGCCTAGGTATTTCCAGT | 14 |
| 243 | GYS1-exon14-EIJ-3 | ggggtcctacCCGGCCTAGGTATTT | 14 |
| 244 | GYS1-exon14-EIJ-4 | aagtgggggtcctacCCGGCCTAGG | 14 |
| 245 | GYS1-exon14-EIJ-5 | taggaaagtgggggtcctacCCGGC | 14 |
| 246 | GYS1-exon14-EIJ-6 | gaggataggaaagtgggggtcctac | 14 |
| 247 | GYS1-exon3-IEJ-1 | ctgggatggggttggggaggcacca | 3 |
| 248 | GYS1-exon3-IEJ-2 | TACACctgggatggggttggggagg | 3 |
| 249 | GYS1-exon3-IEJ-3 | CGAAATACACctgggatggggttgg | 3 |
| 250 | GYS1-exon3-IEJ-4 | GCGCCCGAAATACACctgggatggg | 3 |
| 251 | GYS1-exon3-IEJ-S | AGCCAGCGCCCGAAATACACctggg | 3 |
| 252 | GYS1-exon3-IEJ-6 | CGATCAGCCAGCGCCCGAAATACAC | 3 |
| 253 | GYS1-exon3-EIJ-1 | CTCACCCAGGAACCAGGTGGTCAGA | 3 |
| 254 | GYS1-exon3-EIJ-2 | cctacCTCACCCAGGAACCAGGTGG | 3 |
| 255 | GYS1-exon3-EIJ-3 | cggggcctacCTCACCCAGGAACCA | 3 |
| 256 | GYS1-exon3-EIJ-4 | tcagacggggcctacCTCACCCAGG | 3 |
| 257 | GYS1-exon3-EIJ-5 | atgtgtcagacggggcctacCTCAC | 3 |
| 258 | GYS1-exon3-EIJ-6 | gtggaatgtgtcagacggggcctac | 3 |
| 259 | GYS1-exon4-IEJ-1 | ctgtgggcaacagggacagggccac | 4 |
| 260 | GYS1-exon4-IEJ-2 | AGGAActgtgggcaacagggacagg | 4 |
| 261 | GYS1-exon4-IEJ-3 | GTGCCAGGAActgtgggcaacaggg | 4 |
| 262 | GYS1-exon4-IEJ-4 | ACTCTGTGCCAGGAActgtgggcaa | 4 |
| 263 | GYS1-exon4-IEJ-S | TCCTCACTCTGTGCCAGGAActgtg | 4 |
| 264 | GYS1-exon4-IEJ-6 | GCTTCTCCTCACTCTGTGCCAGGAA | 4 |
| 265 | GYS1-exon4-EIJ-1 | GTTCTCCAGGTTGTTGTAGAAGTCC | 4 |
| 266 | GYS1-exon4-EIJ-2 | ctcacGTTCTCCAGGTTGTTGTAGA | 4 |
| 267 | GYS1-exon4-EIJ-3 | cccagctcacGTTCTCCAGGTTGTT | 4 |
| 268 | GYS1-exon4-EIJ-4 | acggtcccagctcacGTTCTCCAGG | 4 |
| 269 | GYS1-exon4-EIJ-5 | ctgccacggtcccagctcacGTTCT | 4 |
| 270 | GYS1-exon4-EIJ-6 | cccatctgccacggtcccagctcac | 4 |
| 271 | GYS1-exon9-IEJ-1 | ctggaggaagcagagcaacagggtc | 9 |
| 272 | GYS1-exon9-IEJ-2 | TGTCCctggaggaagcagagcaaca | 9 |
| 273 | GYS1-exon9-IEJ-3 | GGCCGTGTCCctggaggaagcagag | 9 |
| 274 | GYS1-exon9-IEJ-4 | GTGTTGGCCGTGTCCctggaggaag | 9 |
| 275 | GYS1-exon9-IEJ-5 | TCACCGTGTTGGCCGTGTCCctgga | 9 |
| 276 | GYS1-exon9-IEJ-6 | TTCCTTCACCGTGTTGGCCGTGTCC | 9 |
| 277 | GYS1-exon9-EIJ-1 | ACCAGTAAGGATTCATAAAGCTTCC | 9 |
| 278 | GYS1-exon9-EIJ-2 | ctcacACCAGTAAGGATTCATAAAG | 9 |
| 279 | GYS1-exon9-EIJ-3 | gggcactcacACCAGTAAGGATTCA | 9 |
| 280 | GYS1-exon9-EIJ-4 | gggtggggcactcacACCAGTAAGG | 9 |
| 281 | GYS1-exon9-EIJ-5 | gctgagggtggggcactcacACCAG | 9 |
| 282 | GYS1-exon9-EIJ-6 | acagggctgagggtggggcactcac | 9 |
| 283 | GYS1-exon11-IEJ-1 | ctgcaggagccacaagaagggtaag | 11 |
| 284 | GYS1-exon11-IEJ-2 | TGCCGctgcaggagccacaagaagg | 11 |
| 285 | GYS1-exon11-IEJ-3 | AAGACTGCCGctgcaggagccacaa | 11 |
| 286 | GYS1-exon11-IEJ-4 | GGGGAAAGACTGCCGctgcaggagc | 11 |
| 287 | GYS1-exon11-IEJ-5 | ACAGGGGGGAAAGACTGCCGctgca | 11 |
| 288 | GYS1-exon11-IEJ-6 | TGCACACAGGGGGGAAAGACTGCCG | 11 |
| 289 | GYS1-exon11-EIJ-1 | CTTCACCCTGTCGGCACTGCTATTG | 11 |
| 290 | GYS1-exon11-EIJ-2 | ctcacCTTCACCCTGTCGGCACTGC | 11 |
| 291 | GYS1-exon11-EIJ-3 | atgccctcacCTTCACCCTGTCGGC | 11 |
| 292 | GYS1-exon11-EIJ-4 | accaaatgccctcacCTTCACCCTG | 11 |
| 293 | GYS1-exon11-EIJ-5 | ccctgaccaaatgccctcacCTTCA | 11 |
| 294 | GYS1-exon11-EIJ-6 | cccagccctgaccaaatgccctcac | 11 |
| 295 | GYS1-exon13-IEJ-1 | ctgcgggaaggcaggagagagaccc | 13 |
| 296 | GYS1-exon13-IEJ-2 | CTCAGctgcgggaaggcaggagaga | 13 |
| 297 | GYS1-exon13-IEJ-3 | GTGCACTCAGctgcgggaaggcagg | 13 |
| 298 | GYS1-exon13-IEJ-4 | TAACCGTGCACTCAGctgcgggaag | 13 |
| 299 | GYS1-exon13-IEJ-5 | TCCCATAACCGTGCACTCAGctgcg | 13 |
| 300 | GYS1-exon13-IEJ-6 | GGGATTCCCATAACCGTGCACTCAG | 13 |
| 301 | GYS1-exon13-EIJ-1 | CGTAAGCTGAGGGGTCTGCGATGTG | 13 |
| 302 | GYS1-exon13-EIJ-2 | ctgacCGTAAGCTGAGGGGTCTGCG | 13 |
| 303 | GYS1-exon13-EIJ-3 | gggcgctgacCGTAAGCTGAGGGGT | 13 |
| 304 | GYS1-exon13-ELJ-4 | ggcctgggcgctgacCGTAAGCTGA | 13 |
| 305 | GYS1-exon13-EIJ-5 | tcaggggcctgggcgctgacCGTAA | 13 |
| 306 | GYS1-exon13-EIJ-6 | aatcctcaggggcctgggcgctgac | 13 |
| 307 | GYS1-exon15-IEJ-1 | ctaggggaaaggaggagagggggca | 15 |
| 308 | GYS1-exon15-IEJ-2 | TAGTActaggggaaaggaggagagg | 15 |
| 309 | GYS1-exon15-IEJ-3 | ACATATAGTActaggggaaaggagg | 15 |
| 310 | GYS1-exon15-IEJ-4 | CGCAGACATATAGTActaggggaaa | 15 |
| 311 | GYS1-exon15-IEJ-5 | TGGCGCGCAGACATATAGTActagg | 15 |
| 312 | GYS1-exon15-IEJ-6 | CCATGTGGCGCGCAGACATATAGTA | 15 |
| 313 | GYS1-exon15-EIJ-1 | CGCATCCGCCTCGTTGGGCTCGTAG | 15 |
| 314 | GYS1-exon15-EIJ-2 | ctcacCGCATCCGCCTCGTTGGGCT | 15 |
| 315 | GYS1-exon15-EIJ-3 | gtccactcacCGCATCCGCCTCGTT | 15 |
| 316 | GYS1-exon15-EIJ-4 | ccagggtccactcacCGCATCCGCC | 15 |
| 317 | GYS1-exon15-EIJ-5 | gaaatccagggtccactcacCGCAT | 15 |
| 318 | GYS1-exon15-EIJ-6 | aagcagaaatccagggtccactcac | 15 |

In some embodiments, the AC includes a PMO sequence from U.S. application Ser. No. 16/867,261 and/or Clayton et. a., Molecular Therapy—Nucleic Acids (2014)3, e206, such as those listed in Table 9, or a portion thereof.

The PMO sequences are designed to induce exon skipping to result in a frameshift alteration. In embodiments, the frameshift alteration results in premature termination codon that leads to nonsense mediated decay of a GYS1 target transcript, SEQ ID NOs:321-327 are designed to bind to a target nucleotide sequence that includes an intron/exon and/or exon/intron junctions of a GYS1 target transcript. SEQ ID NOs:319 and 327 are designed to bind to target nucleotide sequences that include intronic sequences of the target GYS1 transcript.

TABLE 9
Various AC sequences for targeting GYS1

| SEQ | | Exon |
|---|---|---|
| ID | | to be |
| NO: | Sequence (5′ to 3′) | skipped |
| 319 | TCAGGGTTGTGGACTCAATCATGCC | 8 |
| 320 | AAGGACCAGGGTAAGACTAGGGACT | 5 |
| 321 | GTCCTGGACAAGGATTGCTGACCAT | 8 |
| 322 | CTGCTTCCTTGTCTACATTGAACTG | 5 |
| 323 | ATACCCGGCCCAGGTACTTCCAATC | 14 |
| 324 | CTGGACAAGGATTGCTGACCATAGT | 8 |
| 325 | TCCCACCGAGCAGGCCTTACTCTGA | 7 |
| 326 | GACCACAGCTCAGACCCTACCTGGT | 5 |
| 327 | TCACTGTCTGGCTCACATACCCATA | 6 |

In embodiments the AC includes 10 or more, 15 or more, or 20 or more consecutive bases of any sequence in Table 8 and/or Table 9. In embodiments the AC includes 25 or less, 20 or less, or 15 or less consecutive bases of any sequence in in Table 8 and/or Table 9. In embodiments, the AC includes 10 to 25, 10 to 20, or 10 to 15 consecutive bases of any sequence in in Table 8 and/or Table 9. In embodiments, the AC includes 15 to 25 or 15 to 20 consecutive bases of any sequence in Table 8 and/or Table 9. In embodiments, the AC includes 20-25 consecutive bases of any sequence in in Table 8 and/or Table 9.

In embodiments, a mouse model using mouse GYS1 is used to study the effects of compounds that induce exon skipping in a GYS1 target transcript. Mouse and human GYS1 two have 97% homology in chromosome 19. Additionally, mouse and human GYS1 both have 16 exons and the same splicing pattern that results in a full-length protein that is 737 amino acids long.

In embodiments, a compound includes an antisense compound (AC) that induces downregulation of human and/or mouse GYS1 by targeting a start codon thereof. Examples of such sequences include those in Table 10.

TABLE 10
Various AC sequences for targeting GYS1

| | ENTR | |
|---|---|---|
| No | Oligo # | Sequence (5′-3′) |
| 328 | GYS1-SC-1 | GCGGTTTAAAGGCATGGCTGGCGCA |
| 329 | GYS1-SC-2 | TGGACAAAGTGCGGTTTAAAGGCAT |
| 330 | GYS1-SC-3 | GTGAGGACATGGACAAAGTGCGGTT |
| 331 | GYS1-SC-4 | CAGTCCTGGCAGTGAGGACATGGAC |
| 332 | GYS1-SC-5 | CCAGTCCTCCAGTCCTGGCAGTGAG |
| 333 | GYS1-SC-6 | CACCTGGGATTCTTAAATATAGATG |

### Interferon Regulatory Factor-5 (IRF-5)

In embodiments, a compound is provided for modulating the activity of Interferon Regulatory Factor-5 (IRF-5). IRE-5 is a member of the IRF family of transcription factors that is highly expressed in monocytes, macrophages, B cells, and dendritic cells and its expression can be induced in other cell types by type I interferons (Almuttaqi and Udalova, FEBS J. (2018), 286:1624-1637). IRF-5 is involved in innate and adaptive immunity, antiviral defense, production of proinflammatory cytokines, macrophage polarization, cell growth regulation, and differentiation and apoptosis.

Aberrant IRF-5 expression is associated with a variety of diseases. In addition, increased IRF5 mRNA level is strongly correlated with disease pathology. For example, upregulation of IRF-5 can lead to increased production of IFNs, which is linked to the development of numerous inflammatory diseases, including autoimmune disease, infectious disease, cancer, obesity, neuropathic pain, cardiovascular disease (e.g., artherosclerosis), and metabolic dysfunction (Banga et al., Sci. Adv. (2020), 6:eaay1057). Additionally, IRF-5 gene polymorphisms related to higher IRF-5 expression are associated with susceptibility to inflammatory and autoimmune diseases including rheumatoid arthritis (RA), inflammatory bowel disease (IBD), multiple sclerosis (MS) inflammatory bowel disease (IBD), systemic lupus erythematosus (SLE) and Sjögrens syndrome (Almuttaqi and Udalova (2018) FEBS J. 286:1624-1637; Thompson et al., Front. Immunol., 2018, 9:2622; Ban et al., International Immunology (2018), 30, 11: 529-536; Chehimi et al., J. Clin. Med. (2017), 6, 712, doi.org/10.3390/jcm6070068). Furthermore, IRF-5 is involved in Type I interferon and Toll-like receptor signaling pathways and is a downstream mediator of cytokine expression (Krisjansdottir et al., J. Med. Genet. (2008), 45:362-369).

IRF-5 exists in multiple isoforms that are generated by three alternative non-coding 5′ exons and at least nine alternatively spliced mRNAs. The sequences for the IRF-5 isoforms are publicly available, for example, through the online NCBI database. The isoforms show cell-type specific expression, subcellular localization and function. Some isoforms are associated with risk of autoimmune disease. For example, Isoform 2 is linked to overexpression of IRF-5 and susceptibility to autoimmune disease such as systemic lupus erythematosus. Additionally, polymorphisms, including single nucleotide polymorphisms, in the gene encoding IRF-5 that led to higher mRNA expression are associated with many autoimmune diseases (Krausgruber et al., Nat. Immunol. (2010), 12(3):231-238); Kozyrev et al., Arthritis and Rheumatology (2007), 56(4):1234-1241).

IRF-5 activation, mechanisms of action, signaling pathway, and regulatory elements have been reviewed (Song et al., J. Clin. Invest. (2020), 130(12):6700-6717; Almutaqqi and Udalova FEBS J. (2018), 286:1624-1637; Banga et al., Sci. Adv. (2020), 6:eaay1057; Thompson et al., Front. Immunol. (2018), 9:2622).

The gene encoding IRF-5 includes 9 exons (exon 1, exon 2, exon 3, exon 4, exon 5, exon 6, exon 7, exon 8, and exon 9). Exon 1 is in the 5′-untranslated region (5′-UTR) and has three variants, exon 1A, exon 1B, exon 1C, and exon 1D. The predominant isoform includes Exon 1A. Exon 1B is associated with IRF-5 hyperactivation and disease progression. Single-nucleotide polymorphisms (SNP) (e.g., rs2004640) that introduce a donor splice site can lead to increased expression of exon 1B transcripts and reduced expression of exon 1C-derived transcripts. Other SNPs (e.g., rs2280714) are also associated with elevated IRF-5 expression (Kozyrev et al., Arthritis and Rheumatology (2007), 56(4):1234-1241).

Six isoforms of IRF-5 are provided below.

HUMAN Interferon regulatory factor-5 (IRF-5)(Isoform 1)
(SEQ ID NO: 334)
MNQSIPVAPTPPRRVRLKPWLVAQVNSCQYPGLQWVNGEKKLFCIPWRHATRHGPSQDGDNTIF
KAWAKETGKYTEGVDEADPAKWKANLRCALNKSRDFRLIYDGPRDMPPQPYKIYEVCSNGPAPT
DSQPPEDYSFGAGEEEEEEEELQRMLPSLSLTEDVKWPPTLQPPTLRPPTLQPPTLQPPVVLGP
PAPDPSPLAPPPGNPAGFRELLSEVLEPGPLPASLPPAGEQLLPDLLISPHMLPLTDLEIKFQY
RGRPPRALTISNPHGCRLFYSQLEATQEQVELFGPISLEQVRFPSPEDIPSDKQRFYTNQLLDV
LDRGLILQLQGQDLYAIRLCQCKVFWSGPCASAHDSCPNPIQREVKTKLFSLEHFLNELILFQK
GQTNTPPPFEIFFCFGEEWPDRKPREKKLITVQVVPVAARLLLEMESGELSWSADSIRLQISNP
DLKDRMVEQFKELHHIWQSQQRLQPVAQAPPGAGLGVGQGPWPMHPAGM
HUMAN Interferon regulatory factor-5 (IRF-5)(Isoform 2)
(SEQ ID NO: 335)
MNQSIPVAPTPPRRVRLKPWLVAQVNSCQYPGLQWVNGEKKLFCIPWRHATRHGPSQDGDNTIF
KAWAKETGKYTEGVDEADPAKWKANLRCALNKSRDFRLIYDGPRDMPPQPYKIYEVCSNGPAPT
DSQPPEDYSFGAGEEEEEEEELQRMLPSLSLTDAVQSGPHMTPYSLLKEDVKWPPTLQPPTLRP
PTLQPPTLQPPVVLGPPAPDPSPLAPPPGNPAGFRELLSEVLEPGPLPASLPPAGEQLLPDLLI
SPHMLPLTDLEIKFQYRGRPPRALTISNPHGCRLFYSQLEATQEQVELFGPISLEQVREPSPED
IPSDKQRFYTNQLLDVLDRGLILQLQGQDLYAIRLCQCKVFWSGPCASAHDSCPNPIQREVKTK
LESLEHFLNELILFQKGQTNTPPPFEIFFCFGEEWPDRKPREKKLITVQVVPVAARLLLEMESG
ELSWSADSIRLQISNPDLKDRMVEQFKELHHIWQSQQRLQPVAQAPPGAGLGVGQGPWPMHPAG
MQ
HUMAN Interferon regulatory factor-5 (IRF-5)(Isoform 3)
(SEQ ID NO: 336)
MNQSIPVAPTPPRRVRLKPWLVAQVNSCQYPGLQWVNGEKKLFCIPWRHATRHGPSQDGDNTIF
KAWAKETGKYTEGVDEADPAKWKANLRCALNKSRDFRLIYDGPRDMPPQPYKIYEVCSNGPAPT
DSQPPEDYSFGAGEEEEEEEELQRMLPSLSLTDAVQSGPHMTPYSLLKEDVKWPPTLQPPTLQP
PVVLGPPAPDPSPLAPPPGNPAGFRELLSEVLEPGPLPASLPPAGEQLLPDLLISPHMLPLTDL
EIKFQYRGRPPRALTISNPHGCRLFYSQLEATQEQVELFGPISLEQVRFPSPEDIPSDKQRFYT
NQLLDVLDRGLILQLQGQDLYAIRLCQCKVFWSGPCASAHDSCPNPIQREVKTKLFSLEHELNE
LILFQKGQTNTPPPFEIFFCFGEEWPDRKPREKKLITVQVVPVAARLLLEMFSGELSWSADSIR
LQISNPDLKDRMVEQFKELHHIWQSQQRLQPVAQAPPGAGLGVGQGPWPMHPAGMQ
HUMAN Interferon regulatory factor-5 (IRF-5)(Isoform 4)
(SEQ ID NO: 337)
MNQSIPVAPTPPRRVRLKPWLVAQVNSCQYPGLQWVNGEKKLFCIPWRHATRHGPSQDGDNTIF
KAWAKETGKYTEGVDEADPAKWKANLRCALNKSRDFRLIYDGPRDMPPQPYKIYEVCSNGPAPT
DSQPPEDYSFGAGEEEEEEEELQRMLPSLSLTEDVKWPPTLQPPTLQPPVVLGPPAPDPSPLAP
PPGNPAGFRELLSEVLEPGPLPASLPPAGEQLLPDLLISPHMLPLTDLEIKFQYRGRPPRALTI
SNPHGCRLFYSQLEATQEQVELEGPISLEQVRFPSPEDIPSDKQRFYTNQLLDVLDRGLILQLQ
GQDLYAIRLCQCKVFWSGPCASAHDSCPNPIQREVKTKLFSLEHFLNELILFQKGQTNTPPPFE
IFFCFGEEWPDRKPREKKLITVQVVPVAARLLLEMFSGELSWSADSIRLQISNPDLKDRMVEQF
KELHHIWQSQQRLQPVAQAPPGAGLGVGQGPWPMHPAGMQ
HUMAN Interferon regulatory factor-5 (IRF-5)(Isoform 5)
(SEQ ID NO: 338)
MNQSIPVAPTPPRRVRLKPWLVAQVNSCQYPGLQWVNGEKKLFCIPWRHATRHGPSQDGDNTIE
KAWAKETGKYTEGVDEADPAKWKANLRCALNKSRDFRLIYDGPRDMPPQPYKIYEVCSNGPAPT
DSQPPEDYSFGAGEEEEEEEELQRMLPSLSLTVTDLEIKFQYRGRPPRALTISNPHGCRLFYSQ
LEATQEQVELFGPISLEQVRFPSPEDIPSDKQRFYTNQLLDVLDRGLILQLQGQDLYAIRLCQC
KVFWSGPCASAHDSCPNPIQREVKIKLFSLEHFLNELILFQKGQTNTPPPFEIFFCFGEEWPDR
KPREKKLITVQVVPVAARLLLEMESGELSWSADSIRLQISNPDLKDRMVEQFKELHHIWQSQQR
LQPVAQAPPGAGLGVGQGPWPMHPAGMQ
HUMAN Interferon regulatory factor-5 (IRF-5)(Isoform 6)
(SEQ ID NO: 339)
MNQSIPVAPTPPRRVRLKPWLVAQVNSCQYPGLQWVNGEKKLFCIPWRHATRHGPSQDGDNTIF
KAWAKETGKYTEGVDEADPAKWKANLRCALNKSRDFRLIYDGPRDMPPQPYKIYETPSPIRITL
LVQERRRKKRKSCRGCCQA

In embodiments, IRE-5 is encoded by a nucleotide sequence encoding IRE-5 Isoform 1, IRF-5 Isoform 2, IRF-5 Isoform 3, IRF-5 Isoform 4, IRF-5 Isoform 5, or IRF-5 Isoform 6. In embodiments, a nucleotide sequence encoding IRF-5 differs by one or more nucleic acids from a nucleotide sequence encoding IRF-5 Isoform 1, IRF-5 Isoform 2, IRF-5 Isoform 3, IRF-5 Isoform 4, IRE-5 Isoform 5, or IRF-5 Isoform 6. In embodiments the nucleotide sequence encoding IRE-5 differs by one or more polymorphisms (e.g., Single Nucleotide Polymorphisms or SNPs). In embodiments, the nucleotide sequence encoding IRF-5 shares less than 100% sequence identity with a nucleotide sequence encoding IRF-5 Isoform 1, IRF-5 Isoform 2, IRF-5 Isoform 3, IRF-5 Isoform 4, IRF-5 Isoform 5, or IRF-5 Isoform 6. In embodiments, IRF-5 is encoded by nucleotide sequence that is at least about 80%, about 85%, about 90%, about 91%, about 92%, about 93%, about 94%, about 95%, about 96%, about 97%, about 98%, or about 99% identical to a nucleic acid sequence encoding IRF-5 Isoform 1, IRF-5 Isoform 2, IRF-5 Isoform 3, IRF-5 Isoform 4, IRE-5 Isoform 5, or IRE-5 Isoform 6. In embodiments, IRE-5 is encoded by nucleotide sequence that is 80% to 100%, 90% to 100%, 95% to 100%, or 99% to 100% identical to a nucleic acid sequence encoding IRF-5 Isoform 1, IRF-5 Isoform 2, IRE-5 Isoform 3, IRF-5 Isoform 4, IRE-S Isoform 5, or IRF-5 Isoform 6

IRF-5 has been shown to influence inflammatory macrophage phenotype (Almuttaqi and Udalova, FEBS J. (2018), 286:1624-1637). Macrophages can be classified as M1 (classically activated macrophages) or M2 (alternatively activated macrophages) and can be converted to each other depending on the tissue microenvironment. There are three classes of alternately activated macrophages (M2a, M2b and M2c). In normal tissue, the ratio of M1 to M2 macrophages is highly regulated. An imbalance between M1 and M2 macrophages can result in pathologies such as asthma, chronic pulmonary disease, artherosclerosis, or osteoclastogenesis in rheumatoid arthritis. IRF-5 is a major regulator of proinflammatory M1 macrophage polarization (Weiss et al Mediators of Inflammation (2013) Dx.doi.org/10.1155/2013/245804).

Exposure of naïve monocytes or recruited macrophages to the Th1 cytokine IFN-γ, TNF, or LPS, promotes M1 development, which secrete proinflammatory cytokines such as TNF, IL-1β, IL-6, IL-12, IL-23, and promote the development of Th1 lymphocytes. Exposure of monocytes to IL-4 and IL-13 promotes the M2a phenotype, which express chemokines that promote the accrual of Th2 cells, eosinophils, and basophils. M2b macrophages are induced by a combination of LPS, immune complexes, apoptotic cells, and IL-1Ra. M2b macrophages secrete high levels of IL-10, and proinflammatory cytokines TNF and IL-6 and express iNOS. M2c macrophages are induced by a combination of IL-10, TGF-β, and glucocorticoids and secrete IL-10 and TGF-β, which promote the development of Th2 lymphocytes (Duque and Descoteaux. (2014) Front. Immunol. 5:491. Doi:10.3389/fimmu.2014.00491).

IRF-5 expression in macrophages is reversibly induced by inflammatory stimulate and contributes to macrophage polarization. IRF-5 upregulates expression of M1 macrophages and downregulates expression of M2 macrophages (Krausgruber et al, Nat. Immunol. (2010), 12(3):231-238).

In embodiments, a method is provided for treating an inflammatory disease. In embodiments, the disease is associated with aberrant expression of IRF-5. In embodiments, the disease is associated with IRF-5 overexpression. In embodiments, the method includes administering a compound that downregulates IRE-5 expression. In embodiments, the compound includes an AC. The AC may be any AC and have any AC characteristics as described elsewhere herein. In embodiments, the AC may bind to at least a portion of a SE or a SRE of a target transcript as described elsewhere herein. In embodiments, the AC may bind to in proximity to a SE or a SRE of a target transcript as described elsewhere herein. In embodiments, the AC is an ASO. In embodiments, the ASO is a PMO. The AC may bind to any splicing element (SE) of an IRF-5 target transcript as described elsewhere herein.

In embodiments, the method includes administering a compound that induces exon skipping of one or more exons in an IRF-5 mRNA transcript. In embodiments, the method includes administering a compound that includes an antisense compound (AC) that induces skipping of one or more exons in an IRF-5 target transcript. In embodiments, hybridization of an AC to a target nucleotide sequence that includes at least a portion of an IRF-5 target transcript results in inclusion or skipping of one or more exons in the mRNA transcript. In embodiments, skipping or inclusion of one or more exons induces a frameshift in the IRF-5 target transcript. In embodiments, the frameshift results in an IRE-5 target transcript that encodes a protein with decreased activity. In embodiments, the frameshift results in a truncated or non-functional IRF-5. In embodiments, the frameshift results in the introduction of a premature termination codon in the IRF-5 mRNA transcript. In embodiments, the frameshift results in degradation of the IRF-5 mRNA transcript by nonsense-mediated decay. In embodiments, a compound includes an antisense compound (AC) that induces skipping of one or more of exons 2, 3, 4, 5, 6, 7, and/or 8 of human and/or mouse IRF-5. In embodiments, a compound includes an AC that induces skipping of one or more exons to produce an out of frame frameshift leading to the IRF-5 target transcript being degraded (e.g., nonsense mediate decay), or being translated into an IRF-5 protein with reduce or no activity. In embodiments, a compound includes an AC that induces skipping of one or more of exons 3, 4, 5, and/or 8 produce an out of frame frameshift.

In embodiments, the AC includes any one of SEQ ID NOs:157-161 in Table 11. In embodiments the AC includes 10 to 25, 10 to 20, or 10 to 15 consecutive bases of anyone of the sequences in Table 11. In embodiments, SEQ ID NOs:340, 365, 369 or a fragment thereof, induces skipping of exon 4 to produce a premature termination codon in exon 5. In embodiments, SEQ ID NOs:340, 365, 369, or a fragment thereof induce exon skipping of exon 4 leading to nonsense mediated decay of the IRF-5 target transcript. In embodiments, SEQ ID NOs:340 and 365, or a fragment thereof, induces skipping of exon 4 to produce a premature stop codon. In embodiments, SEQ ID NOs:366 to 368, or a fragment thereof induce exon skipping of exon 5 resulting in a premature termination codon in exon 6. In embodiments, SEQ ID NOs:366-368, or a fragment thereof induce exon skipping of exon 5 leading to nonsense mediated decay of the IRF-5 target transcript.

TABLE 11
AC sequences for inducing exon skipping

| SEQ | | Exon |
|---|---|---|
| ID | | to be |
| NO: | Sequence 5′-3′ | skipped |
| 340 | AGA ACG TAA TCA TCA GTG GGT TGG C | 4 |
| 365 | ACG TAA TCA TCA GTG GGT TGG CTC T | 4 |
| 366 | TTGGCAACATCCTCTGCAGCTGAAG | 5 |
| 367 | GCAACATCCTCTGCAGCTG | 5 |
| 368 | TCAGGCTTGGCAACATCCTCTGCAG | 5 |
| 369 | TAATCATCAGTGGGTTGGCTCTCTG | 4 |

In embodiments, a compound includes an AC that induces skipping of one or more exons to produce an in-frame deletion in an IRF-5 target transcript. In embodiments, a compound includes an AC that induces skipping of one or more of exons 6 and/or 7 to produce an in-frame deletion in an IRF-5 target transcript.

In embodiments, a method is provided for treating a disease or disorder associated with IRF-5. In embodiments, the disease or disorder is associated with IRF-5 genetic variation. In embodiments, the disease or disorder is associated with a genetic mutation in the IRF-5 gene. In embodiments, the genetic mutation in IRF-5 results IRF-5 overexpression. In embodiments, the genetic mutation results in alternate isoform expression. In embodiments, the disease or disorder is associated with IRF-5 overexpression. In embodiments, the disease or disorder is associated with IRF-5 isoform expression.

In embodiments, a method is provided for treating inflammation, autoantibody production, inflammatory cell infiltration, collagen deposits, or inflammatory cytokine production in a patient.

IRF-5 involvement in various diseases has been document (see for example, Graham et al., Nat Genet. (2006), 38(5):550-5; Rueda et al., Arthritis Rheum. (2006), 54(12):3815-9; Henrique da Mota, Clin Rheumatol. (2015), 34(9):1495-501; Sigurdsson et al., Hum Mol Genet. (2008), 17(6):872-81; Peng, et al., Nephrology (Carlton) (2010), 15(7):710-3; Ishimura et al., J Clin Immunol. (2011), 31(6): 946-51; Summers et al., J Rheumatol. (2008), 35(11):2106-18; Ni et al., Inflammation (2019), 2(5):1821-1829; Dideberg et al., Plum Mol Genet. (2007), 16(24):3008-16; Lim et al., J. Dig. Dis. (2015), 16(4):205-16; Nordal et al., Ann. Rheum. Dis. (2012), 71(7):1197-202; Rebora, Int. J. Dermatol. (2016), 55(4):408-16; Zhao et al., Rheumatol. Int (2017), 37(8):1303-131; Carmona et al., PLoS One (2013), 8(1):e54419; Flesch et al, Tissue Antigens (2011), 78(1):65-8; Heijde et al., Arthritis Rheum. (2007), 56(12):3989-94; Hailer et al., Genes Immun. (2009), 10(1):68-76; Balasa et al., Eur. Cytokine Netw. (2012), 23(4):166-72; Byre et al., Mucosal Immunol. (2017), 10(3):716-726; Wang et al., Gene (2012), 10, 504(2):220-5; Pimenta et al., Mol Cancer (2015), 14(1):32; Rambod et al., Clin Rheumatol. (2018), 37(10):2661-2665; Davi et al., J Rheumatol. (2011), 38(4):769-74; Zimmerman et al., Kidney 360 (2020), 1(3): 179-190; Pandey et al., Mucosal. Immunol. (2019), 12(4):874-887; Masuda et al., Nat Commun. (2014), 5: 3771; Alzaid et al., JCI Insight (2016), 1(20): e88689; Senevirante et al., Circulation (2017), 136(12): 1140-1154; Cevik et al., J. Biol. Chem. (2017), 292(52)21676-21689; Sharif et al., Ann. Rheum. Dis. (2012), 71(7):1197-1202; and Yang et al., J Pediatr. Surg. (2017), 52(12):1984-1988).

In embodiments, a method of downregulating IRF-5 expression in a patient is provided using one or more of the compounds disclosed herein. In embodiments, IRF-5 expression in a macrophage is reduced. In embodiments, IRE-5 expression in a Kupffer cell is reduced. In embodiments, IRF-5 expression in the gastrointestinal tract is reduced. In embodiments, expression of IRE-5 in the liver is reduced. In embodiments, expression of IRF-5 in the lungs is reduced. In embodiments, expression of IRF-5 in the kidneys is reduced. In embodiments, expression of IRF-5 in the joints is reduced. In embodiments, expression of IRF-5 in the central nervous system is reduced.

In embodiments, the compounds disclosed herein are used for treating a disease associated with IRF-5. Examples of diseases associated with IRE-5 include, but are not limited to, inflammatory bowel disease (IBD), ulcerative colitis, Crohn's disease, systemic lupus erythematosus (SLE), rheumatoid arthritis, primary biliary cirrhosis, systemic sclerosis, Sjogren's syndrome, multiple sclerosis, scleroderma, interstitial lung disease (SSc-ILD), polycystic kidney disease (PKD), chronic kidney disease (CKD), Nonalcoholic steatohepatitis (NASH), liver fibrosis, asthma, severe asthma, and combinations thereof. In embodiments, the compounds disclosed herein are used to reduce inflammation, cirrhosis, fibrosis, proteinuria, joint inflammation, autoantibody production, inflammatory cell infiltration, collagen deposits, inflammatory cytokine production in a patient, or combinations thereof. In embodiments, the compounds disclosed herein are used to reduce inflammation in the gastrointestinal tract, diarrhea, pain, fatigue, abdominal cramping, blood in the stool, intestinal inflammation, disruption of the epithelial barrier of the gastrointestinal tract, dysbiosis, increased bowel frequency, tenesmus or painful spasms of the anal sphincter, constipation, unintended weight loss, or combinations thereof.

In embodiments, the compounds disclosed herein are used for treating an inflammatory disease. “Inflammatory disease” refers to diseases in which activation of the innate or adaptive immune response is a prominent contributor to the clinical condition. Inflammatory diseases include, but are not limited to, acne vulgaris, asthma, COPD, autoimmune diseases, celiac disease, chronic (plaque) prostatitis, glomerulonephritis, hypersensitivities, inflammatory bowel diseases (IBD, Crohn's disease, ulcerative colitis), pelvic inflammatory disease, reperfusion injury, rheumatoid arthritis, sarcoidosis, transplant rejection, vasculitis, interstitial cystitis, atherosclerosis, allergies (type 1, 2, and 3 hypersensitivity, hay fever), inflammatory myopathies, as systemic sclerosis, and include dermatomyositis, polymyositis, inclusion body myositis, Chediak-Higashi syndrome, chronic granulomatous disease, Vitamin A deficiency, cancer (solid tumor, gallbladder carcinoma), periodontitis, granulomatous inflammation (tuberculosis, leprosy, sarcoidosis, and syphilis), fibrinous inflammation, purulent inflammation, serous inflammation, ulcerative inflammation, and ischemic heart disease, type I diabetes, diabetic nephropathy, and combinations thereof.

In embodiments, the compounds disclosed herein are used for treating an autoimmune disease. “Autoimmune disease” refers to a disease or disorder in which a patient's immune system attacks the patient's own tissues. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); systemic sclerosis (scleroderma); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, dermatomyositis; granulomatosis and vasculitis; primary biliary cirrhosis; pernicious anemia (Addison's disease); autoimmune gastritis; autoimmune hepatitis; diseases involving leukocyte diapedesis; central nervous system (CNS) inflammatory disorder; vitiligo; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia; autoimmune encephalomyelitis; nonalcoholic steatohepatitis (NASH); ankylosing spondylitis; pulmonary fibrosis; or combinations thereof.

In embodiments, the compounds disclosed herein are used for treating an autoimmune disease such as systemic lupus erythematosus (SLE), systemic sclerosis (scleroderma), polymyositis/dermatomyositis, Crohn's disease, ulcerative colitis, rheumatoid arthritis, Sjogren's syndrome, autoimmune encephalomyelitis, nonalcoholic steatohepatitis (NASH), sarcoidosis, Behcet's disease, myasthenia gravis, lupus nephritis, inflammatory bowel disease (IBD), ankylosing spondylitis, primary biliary cirrhosis, colitis, pulmonary fibrosis, antiphospholipid syndrome, or psoriasis

In embodiments, the compounds disclosed herein are used for treating cardiovascular disease. In embodiments, the cardiovascular disease is associated with inflammation. In embodiments, the cardiovascular disease includes systemic scleroderma. In embodiments, the cardiovascular disease includes aneurysm; angina; atherosclerosis; cerebrovascular accident (Stroke); cerebrovascular disease; congestive heart failure; coronary artery disease; myocardial infarction (heart attack); peripheral vascular disease; or combinations thereof. In embodiments, the cardiovascular disease includes atherosclerosis.

In embodiments, the compounds disclosed herein are used for treating a gastrointestinal disease. In embodiments, the gastrointestinal disease includes Crohn's disease, primary biliary cirrhosis, sclerosing cholangitis, ulcerative colitis, inflammatory bowel disease, \Sjögren's syndrome or combinations thereof.

In embodiments, the compounds disclosed herein are used for treating a urinary system disease. In embodiments, the urinary system disease includes systemic lupus erythematosus, systemic scleroderma, or combinations thereof.

In embodiments, the compounds disclosed herein are used for treating a genetic, familial, or congenital disease. In embodiments, the genetic, familial or congenital disease includes Crohn's disease, primary biliary cirrhosis, systemic scleroderma, systemic lupus erythematosus, ulcerative colitis, psoriasis, inflammatory bowel disease, or combinations thereof.

In embodiments, the compounds disclosed herein are used for treating an endocrine system disease. In embodiments, the endocrine system disease includes thyroid gland adenocarcinoma, primary biliary cirrhosis, sclerosing cholangitis, hypothyroidism, or combinations thereof.

In embodiments, the compounds disclosed herein are used for treating a cell proliferation disorder. In embodiments, the cell proliferation disorder includes primary biliary cirrhosis, thyroid gland adenocarcinoma, neoplasm, or combinations thereof.

In embodiments, the compounds disclosed herein are used for treating an immune system disease. In embodiments, the immune system disease includes Sjögren's syndrome, inflammatory bowel disease, psoriasis, myositis, systemic scleroderma, autoimmune disease, systemic lupus erythematosus, rheumatoid arthritis, Crohn's disease, ulcerative colitis, ankylosing spondylitis, or combinations thereof.

In embodiments, the compounds disclosed herein are used for treating a hematologic disease. In embodiments, the hematologic disease includes systemic lupus erythematosus.

In embodiments, the compounds disclosed herein are used for treating a musculoskeletal or connective tissue disease. In embodiments, the musculoskeletal or connective tissue disease includes myositis, systemic scleroderma, systemic lupus erythematosus, rheumatoid arthritis, ankylosing spondylitis, adolescent idiopathic scoliosis, or combinations thereof.

In embodiments, the compounds disclosed herein are used for treating neuroinflammatory disease. In embodiments, the neuroinflammatory disease or disorder includes inflammation due to traumatic brain injury, acute disseminated encephalomyelitis (ADEM), autoimmune encephalitis, acute optic neuritis (AON), chronic meningitis, anti-myelin oligodendrocyte glycoprotein (MOG) disease, transverse myelitis, neuromyelitis optica (NMO), Alzheimer's disease, Parkinson's disease, multiple sclerosis (MS), or combinations thereof.

In embodiments, the compounds disclosed herein are used for treating inflammation due to infection by microorganisms such as viruses, bacteria, fungi, parasites, or combinations thereof.

In embodiments, the compounds disclosed herein are used for treating a disease associated with fibrosis, which is referred to herein as a fibrotic disease. “Fibrosis” refers to a pathological formation of fibrous connective tissue, for example, due to injury, irritation, or chronic inflammation and includes fibroblast accumulation and collagen deposition in excess of normal amounts in a tissue. “Fibrotic disease” refers to a disease associated with pathological fibrosis. Examples of fibrotic disease include, but are not limited to, idiopathic pulmonary fibrosis; scleroderma; scleroderma of the skin; scleroderma of the lungs; a collagen vascular disease (e.g., lupus; rheumatoid arthritis; scleroderma); genetic pulmonary fibrosis (e.g., Hermansky-Pudlak Syndrome); radiation pneumonitis; asthma; asthma with airway remodeling; chemotherapy-induced pulmonary fibrosis (e.g., bleomycin, methotrexate, or cyclophosphamide-induced); radiation fibrosis: Gaucher's disease; interstitial lung disease; retroperitoneal fibrosis; myelofibrosis; interstitial or pulmonary vascular disease; fibrosis or interstitial lung disease associated with drug exposure; interstitial lung disease associated with exposures such as asbestosis, silicosis, and grain exposure; chronic hypersensitivity pneumonitis; an adhesion; an intestinal or abdominal adhesion; cardiac fibrosis; kidney fibrosis; cirrhosis; nonalcoholic steatohepatitis (NASH)-induced fibrosis; or combinations thereof. In embodiments, the fibrotic disease includes non-alcoholic steatohepatitis NASH.

In embodiments, the compounds disclosed herein are used for treating a respiratory or thoracic disease such as systemic scleroderma. In embodiments, the compounds disclosed herein are used for treating an integumentary system disease such as psoriasis or systemic scleroderma. In embodiments, the compounds disclosed herein are used for treating a disease of the visual system such as Sjögren's syndrome or systemic scleroderma. In embodiments, the compounds disclosed herein are used for treating a disease associated with eosinophil count, glomerular filtration rate, systolic blood pressure, eosinophil percentage of leukocytes, or combinations thereof. In embodiments, the compounds disclosed herein are used for treating an ulcer disease or an oral ulcer.

### Inflammatory Bowel Disease-(IBD)

Inflammatory bowel disease (IBD) refers to two conditions characterized by chronic inflammation of the gastrointestinal (GI) tract: Crohn's disease and ulcerative colitis. Common symptoms of IBD include persistent diarrhea, abdominal pain, rectal bleeding/bloody stool, weight loss and fatigue. In 2015, an estimated 1.3% of US adults (3 million) reported being diagnosed with IBD (either Crohn's disease or ulcerative colitis). IBD is associated with an inflammatory macrophage phenotype in intestinal macrophages that is promoted by IRF-5.

### Rheumatoid Arthritis (RA)

Rheumatoid arthritis (RA) is an autoimmune disease that affects 0.5% to 1% of the population worldwide. It causes joint pain and damage throughout a patient's body. Treatment for RA typically includes the use of medications that slow disease and prevent joint deformity, called disease-modifying antirheumatic drugs (DMARDs) and biologics (antibody) that target parts of the immune system that trigger inflammation that causes joint and tissue damage. IRE-5 polymorphisms have been identified as risk factors for RA. Reduced IRF-5 levels is associated with reduced disease phenotype, IRF-5 activation of TLR3 and TLR7 promotes inflammatory cytokine and chemokine production.

### Sjögren's Syndrome (SS)

Sjögren's syndrome (SS) is an immune disorder identified by dry eyes and a dry mouth. The condition often accompanies other immune system disorders, such as rheumatoid arthritis (RA) and systemic lupus erythematosus (SLE). The disease predominantly affects females between the age of 40-60. The prevalence of primary SS in the US was estimated to be between 2 and 10 per 10,000 inhabitants. Existing therapies for SS include treating symptoms of dry eyes and a dry mouth. There is no disease modifying therapy. IRE-5 rs2004640T allele, and CGGGG insertion/deletion have been associated with SS in multiple studies.

### Multiple Sclerosis (MS)

Multiple sclerosis (MS) is a debilitating disease of the central nervous system (the brain and spinal cord). In MS, the immune system attacks the protective sheath (myelin) that covers nerve fibers and causes communication problems between the brain and body of a patient. Multiple sclerosis causes a broad spectrum of neurological symptoms, including sensory or motor pareses, visual disturbances, ataxia, impaired coordination, pain, cognitive dysfunction and fatigue. Current estimates suggest that 300,000 to 400,000 individuals are affected in the United States and over 2 million individuals worldwide. Treatment for MS is generally limited to Corticosteroids and plasma replacement therapies.

Two single nucleotide polymorphisms (SNPs) (rs4728142, rs3807306), and a 5 bp insertion-deletion polymorphism located in the promoter and first intron of the IRF-5 gene are strongly associated with MS. Kristjansdottir et al. (2008) “Interferon regulatory factor 5 (RF-5) gene variants are associated with multiple sclerosis in three distinct populations,” J. Med. Genet. 45(6):362-369.

### Scleroderma or Systemic Sclerosis (SSc)

Scleroderma is a chronic connective tissue disease associated with wide-spread fibrosis of skin and internal organs, small-vessel vasculopathy and immune dysregulation with production of autoantibodies. Sharif et al. (2012) “IRF-5 polymorphism predicts prognosis in patients with systemic sclerosis,” Ann. Rheum. Dis. 71(7):1197-1202.

IRF-5 variant rs4728142 is associated with longer survival of SSc patients and lower IRF-5 transcript levels and was predictive of longer survival and milder interstitial lung disease (ILD) in SSc patients. Patients with no copies of IRE-5 rs4728142 had increased IRF-5 expression levels and experienced more severe ILD and shorter survival. Additional single nucleotide polymorphisms (rs10488631 and rs12537284) were identified in a genome-wide association study (GWAS) of systemic sclerosis (SS). Sharif et al. (2012) “IRF-5 polymorphism predicts prognosis in patients with systemic sclerosis,” Ann Rheum Dis. 71(7):1197-202.

### Double Homeobox 4 (DUX4) Gene

Facioscapulohumeral muscular dystrophy (FSHD) is the third most common form of inherited muscular dystrophy. It is caused by incomplete repression of the transcription factor double homeobox (DUX4) in skeletal muscle. DUX4 overexpression in myogenic cells induces different toxic cascades including an increase in oxidative stress, nonsense-mediated decay inhibition, and inhibition of myogenesis (Bouwnan et al., Curr. Opin. Neurol. (2020), 33(5):635-640).

The DUX4 gene is located near the end of chromosome 4 in a region known as D4Z4. The noted region contains from 11 to more than 100 repeated segments, each of which is about 3,300 DNA bases (3.3 kb) long. Each of the repeated segments in the D4Z4 region contains a copy of the DUX4 gene. The copy closest to the end of the chromosome is called DUX4, while the other copies are referred to as “DUX4-like” or DUX4L.

DUXc has also been identified to be upregulated in FSHD (Ansseau et al., PLoS One (2009), 4(10):e7482, doi:10.1371/journal.pone.0007182). DUXc has been mapped to a 42 kb centromeric of the D4Z4 region. DUX4c encodes a 47 kb protein that is identical to DUX4 except in the carboxy-terminal region.

FSHD is characterized by the contraction of the D4Z4 array located in the sub-telomeric region of chromosome 4, leading to aberrant expression of the DUX4 transcription factor and the mis-regulation of hundreds of genes (Marsollier et al., (Int. J. Mol. Sci. (2018), 19, 1347, doi:10.3390/ijms19051347).

There are four variants of the Human DUX4 gene the nucleotide sequence of which is publicly available through the NCBI data base: variant 1 (NM_001306068.3), variant 2 (NM_001293798.3), variant 3 (NR_137167.1), and variant 4 (NM_001363820.2). Both DUX4 variant 1 and variant 2 encode full length DUX4 (DUX4-fl). Over expression of full length DUX4 has been associated with FSHD. The difference between variant 1 and variant 2 is that variant 2 lacks an alternate segment in the 3′ UTR compared to variant 1. DUX4 variant 3 has multiple differences in the 3′ end compared to variant 1, including a distinct 3′ terminus. This variant is represented as non-coding because the use of the 5′-most expected translational start codon renders the transcript a candidate for nonsense-mediated mRNA decay (NMD). Variant 4 lacks a large portion of the coding region compared to variant 1. The resulting truncated DUX4 isoform (DUX4-s) has a shorter and distinct C-terminus compared to isoform DUX4-fl. The DUX4-s protein has been shown to be nontoxic to cells.

DUX4 includes three exons. Exon one is the coding exon for the DUX4 protein and exons 2 and 3 are untranslated. The full length DUX4 protein includes two DNA binding domains and a C-terminal transactivation domain. The truncated isoform of DUX4 includes the two protein binding domains but not the C-terminal transactivation domain. The first exon includes two 5′ splicing sites. Depending on which 5′ss is used, a transcript encoding for the full length or truncated DUX4 protein is produced. To produce the full-length isoform, the first 5′ss, located at the 3′ end of the first exon, is used. To produce the truncated isoform, a second 5′ss is used that is located within exon 1 and is closer to the 5′ end of the transcript than first 5′ss.

Variants 1, 2 and 4 share the last exon. The sequences for variants 1, 2 and 4 are shown below.

Variant 1 (DUX4-fl2):
(SEQ ID NO: 341)
cgcgcagtgcgcaccccggctgacgtgcaagggagctcgctggcctctct
gtgcccttgttcttccgtgaaattctggctgaatgtctccccccaccttc
cgacgctgtctaggcaaacctggattagagttacatctcctggatgatta
gttcagagatatattaaaatgccccctccctgtggatcctatag
Variant 2 (DUX4-fl1):
(SEQ ID NO: 342))
acctgcgcgcagtgcgcaccccggctgacgtgcaagggagctcgctggcc
tctctgtgcccttgttcttccgtgaaattctggctgaatgtctcccccca
ccttccgacgctgtctaggcaaacctggattagagttacatctcctggat
gattagttcagagatatattaaaatgccccctccctgtggatcctatag
Variant 4 (DUX4-s):
(SEQ ID NO: 343)
acctgcgcgcagtgcgcaccccggctgacgtgcaagggagctcgctggcc
tctctgtgcccttgttcttccgtgaaattctggctgaatgtctcccccca
ccttccgacgctgtctaggcaaacctggattagagttacatctcctggat
gattagttcagagatatattaaaatgccccctccctgtggatcctatag

Because FSHD is caused by a gain of function mutation, DUX4 and/or DUX4c suppression is a promising treatment strategy. Additionally, or alternatively, because DUX4-s has been shown to be nontoxic, downregulating the expression of DUX4-fl by upregulating the expression of DUX4-s is a possible treatment strategy. However, numerous highly homologous copies of DUX4 can be found in the human genome, and the D4Z4 repeat is extremely GC-rich, making DUX4 and DUX4c difficult targets. At this time, there is no therapy that prevents or delays disease progression in patients with FSHD (Bouwman et al., Curr. Opin. Neurol. (2020), 33(5):635-640).

U.S. Pat. No. 10,907,157 and Canadian Patent No. 2999192 describe the use of antisense agents and RNA interference agents to decrease expression of DUX4 or DUX4c. Published PCT US2017/019422 has used small nuclear RNAs to induce exon skipping of DUX4 resulting int the expression of DUX4-s. Phosphorodiamidate morpholino oligomers targeting various SE of DUX4 have demonstrated the ability to alter the expression of DUX4 downstream genes (Marsollier et al., Human molecular genetics (2016), 25(8), 1468-1478; and Lu-Nguyen et al., Hum Mol Genet. (2021), 30(15): 1398-1412).

Provided herein are compositions and methods for modulating DUX4 and/or DUX4c expression. In embodiments, compounds are provided for treating FSHD. In embodiments, compounds are provided that induce exon skipping in DUX4 transcripts resulting in the expression of DUX4-s and not DUX4-fl. In embodiments, the compound includes at least one AC and at least one CPP.

In embodiments, and AC hybridizes to a target nucleotide sequence that includes at least a portion of a splicing element of a DUX4 transcript. In embodiments, and AC hybridizes to a target nucleotide sequence that includes at least a portion of a DUX4 transcript and induces exon skipping to produce a transcript that encodes for DUX4-s. In embodiments, the exon skipping upregulates the expression of DUX4-s. in embodiments, the exon skipping downregulates the expression of DUX4-4.

In embodiments, compounds and methods are provided to induce alternative splicing of a DUX4 target transcript. In embodiments, compounds and methods are provided to shift the splicing of DUX4 to the second 5′ss to produce a transcript that encodes for the truncated DUX4 protein. In embodiments, compounds and methods are provided to downregulate the production of the full length DUX4 mRNA transcript and/or protein. In embodiments, compounds and methods are provided to upregulate the production of the truncated DUX4 mRNA transcript and/or protein. In embodiments, the compound includes and AC. The AC may be any AC and have any AC characteristics as described elsewhere herein. In embodiments, the AC is an ASO. In embodiments, the ASO is a PMO. The AC may bind to any splice element of an DUX4 target transcript as described elsewhere herein.

In embodiments, the AC includes any portion of the small nuclear RNAs in Published PCT US2017/019422 (U.S. Pat. No. 11,180,755). In embodiments, the AC includes any portion of the sequences in Table 12.

TABLE 12
Various AC sequences for targeting DUX4

| SEQ ID | | |
|---|---|---|
| NO: | ID | Sequence (5′ to 3′) |
| 344 | 779 | AGC TCC TCC AGC AGA GCC CGG TAT TCT |
| | | TCC TC |
| 345 | 778 | CGA ACC ACC CGA CCC CGT CCC AAC CCC |
| | | GCG TCC TA |
| 346 | 779-1 | AGC TCC TCC AGC AGA GCC CGG TAT T |
| 347 | 799-2 | GCT CCT CCA GCA GAG CCC GGT ATT C |
| 348 | 799-3 | CTC CTC CAG CAG AGC CCG GTA TTC T |
| 349 | 799-4 | TCC TCC AGC AGA GCC CGG TAT TCT T |
| 350 | 799-5 | CCT CCA GCA GAG CCC GGT ATT CTT C |
| 351 | 799-6 | CTC CAG CAG AGC CCG GTA TTC TTC C |
| 352 | 799-7 | TCC AGC AGA GCC CGG TAT TCT TCC T |
| 353 | 799-8 | CCA GCA GAG CCC GGT ATT CTT CCT C |
| 354 | 778-1 | CGA ACC ACC CGA CCC CGT CCC AAC C |
| 355 | 778-2 | GAA CCA CCC GAC CCC GTC CCA ACC C |
| 356 | 778-3 | AAC CAC CCG ACC CCG TCC CAA CCC C |
| 357 | 778-4 | ACC ACC CGA CCC CGT CCC AAC CCC G |
| 358 | 778-5 | CCA CCC GAC CCC GTC CCA ACC CCG C |
| 359 | 778-6 | CAC CCG ACC CCG TCC CAA CCC CGC G |
| 360 | 778-7 | ACC CGA CCC CGT CCC AAC CCC GCG T |
| 361 | 778-8 | CCC GAC CCC GTC CCA ACC CCG CGT C |
| 362 | 778-9 | CCG ACC CCG TCC CAA CCC CGC CTC C |
| 363 | 778-10 | CGA CCC CGT CCC AAC CCC GCG TCC T |
| 364 | 778-11 | GAC CCC GTC CCA ACC CCG CGT CCT A |

In embodiments the AC may include 10 or more, 15 or more, or 20 or more consecutive bases of any one of the sequences in Table 12. In embodiments, the AC may include 25 or less, 20 or less, or 15 or less consecutive bases of any one of the sequences in Table 12. In embodiments, the AC may include 10-25, 10-20, or 10-15 consecutive bases of any one of the sequences in Table 12. In embodiments, the AC may include 15-25 or 10-20 consecutive bases of any one of the sequences in Table 12. In embodiments, the AC may include 20-25 consecutive bases of any one of the sequences in Table 12.

### Methods of Treatment

The present disclosure provides a method of treating disease in a patient in need thereof, that includes administering a compound disclosed herein. In embodiments, the disease is any of the diseases provided in the present disclosure. In embodiments, a method of treating a disease includes administering to the patient a compound disclosed herein, thereby treating the disease. In embodiments, a method of treating a disease associated with IRF-5, GYS1, or DUX4 includes administering to the patient a compound disclosed herein, thereby treating the disease.

In embodiments, the patient is identified as having, or at risk of having, a disease associated with IRF-5, GYS1, or DUX4. In embodiments, the disease or disorder is associated with IRF-5, GYS1, or DUX4 genetic variation. In embodiments, the disease or disorder is associated with a genetic mutation in the IRF-5 gene, GYS1-gene, or DUX4 gene. In embodiments, the genetic mutation results in overexpression of IRF-5, GYS1, or DUX4 (e.g., DUX4-fl). In embodiments, the genetic mutation results in the expression of an alternate isoform of an IRF-5, CYS1, or DUX4. In embodiments, the disease or disorder is associated with over expression of IRF-5, GYS1, or DUX4 (e.g., DUX4-fl).

In various embodiments, treatment refers to partial or complete alleviation, amelioration, relief, inhibition, delaying onset, reducing severity and/or incidence of one or more symptoms in a patient.

In embodiments, a method is provided for treating a disease or disorder by downregulating expression of a target protein. In embodiments, expression of a target protein is downregulated by inducing exon skipping. In embodiments, exon skipping induces a frameshift that results in reduced expression or activity of a target protein. In embodiments, exon skipping results in a premature termination codon and the degradation of the target transcript. In embodiments, treatment results in reduced expression of a protein isoform.

In embodiments, treatment modulates activity of IRF-5 in a patient in need thereof. In embodiments, treatment modulates activity of IRF-5 in a cell of a patient. In embodiments, treatment modulates activity of IRF-5 in an immune cell of a patient. In embodiments, immune cell is a monocyte, a lymphocyte or a dendritic cell. In embodiments, the lymphocyte is a B-lymphocyte. In embodiments, the monocyte is a macrophage. In embodiments, the macrophage is a resident tissue macrophage. In embodiments, the macrophage is a monocyte-derived macrophage. In embodiments, the macrophage is a Kupffer cell, an intraglomerular mesangial cell, an alveolar macrophage, a sinus histiocyte, a hofbauer cell, microglia or langerhan cell. In embodiments, the immune cell is a Kupffer cell.

In embodiments, treatment modulates activity of DUX4 in a patient in need thereof. In embodiments, treatment modulates activity of DUX4 in a cell of a patient. In embodiments, treatment modulates activity of DUX4 in a muscle cell of a patient. In embodiments, muscle cell is a skeletal muscle cell.

In embodiments, treatment according to the present disclosure results in decreased RF-5, DUX4-fl, or GYS1 activity and/or expression in a patient by 5% to 10%, 5% to 20%, 5% to 30%, % to 40%, 5% to 50%, 5% to 60%, 5% to 70%, 5% to 80%, 5% to 90%, or 5% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4-fl, or GYS1 activity and/or expression in a patient by 10% to 20%, 10% to 30%, 10% to 40%, 10% to 50%, 10% to 60%, 10% to 70%, 10% to 80%, 10% to 90%, or 10% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4-fl, or GYS1 activity and/or expression in a patient by 20% to 30%, 20% to 40%, 20% to 50%, 20% to 60%, 20% to 70%, 20% to 80%, 20% to 90%, or 20% to 100% as compared to the average level and/or activity of the target protein in the patient before the treatment, of one or more control individuals with similar disease without treatment, or compared to treatment with a therapeutic moiety not conjugated to a CPP disclosed herein. In embodiments, treatment according to the present disclosure results in decreased IRF-5, DUX4-fl, or GYS1 activity and/or expression in a patient by 30% to 40%, 30% to 50%, 30% to 60%, 30% to 70%, 30% to 80%, 30% to 90%, or 30% to 100% as compared to the average level and/or activity of the target protein in the patient before the treatment, of one or more control individuals with similar disease without treatment, or compared to treatment with a therapeutic moiety not conjugated to a CPP disclosed herein. In embodiments, treatment according to the present disclosure results in decreased IRF-5, DUX4-fl, or GYS1 activity and/or expression in a patient by 40% to 50%, 40% to 60%, 40% to 70%, 40% to 80%, 40% to 90%, or 40% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4-fl, or GYS1 activity and/or expression in a patient 50% to 60%, 50% to 70%, 50% to 80%, 50% to 90%, or 50% to 100% as compared to the average level and/or activity of the target protein in the patient before the treatment, of one or m ore control individuals with similar disease without treatment, or compared to treatment with an therapeutic moiety not conjugated to a CPP disclosed herein. In embodiments, treatment according to the present disclosure results in decreased IRF-5, DUX4-fl, or GYS1 activity and/or expression in a patient 60% to 70%, 60% to 80%, 60% to 90%, or 60% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4-fl, or GYS1 activity and/or expression in a patient 70% to 80%, 70% to 90%, or 70% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4-fl, or GYS1 activity and/or expression in a patient 80% to 90% or 80% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4-fl, or GYS1 activity and/or expression in a patient 90% to 100% as compared to the average level and/or activity of the target protein in the patient before the treatment, of one or more control individuals with similar disease without treatment, or compared to treatment with a therapeutic moiety not conjugated to a CPP disclosed herein.

The terms, “improve,” “increase,” “reduce,” “decrease,” and the like, as used herein, indicate values that are relative to a control. In embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with the same disease, who is about the same age and/or gender as the individual being treated (to ensure that the stages of the disease in the treated individual and the control individual(s) are comparable).

The individual (also referred to as “patient” or “subject”) being treated is an individual (fetus, infant, child, adolescent, or adult human) having a disease or having the potential to develop a disease. The individual may have a disease mediated by aberrant gene expression or aberrant gene splicing. In various embodiments, the individual having the disease may have wild type target protein expression or activity levels that are less than about 1-99% of normal protein expression or activity levels in an individual not afflicted with the disease. In embodiments, the range includes, but is not limited to less than about 80-99%, less than about 65-80%, less than about 50-65%, less than about 30-50%, less than about 25-30%, less than about 20-25%, less than about 15-20%, less than about 10-15%, less than about 5-10%, less than about 1-5% of normal thymidine phosphorylase expression or activity levels. In embodiments, the individual may have target protein expression or activity levels that are 1-500% higher than normal wild type target protein expression or activity levels. In embodiments, the range includes, but is not limited to, greater than about 1-10%, about 10-50%, about 50-100%, about 100-200%, about 200-300%, about 300-400%, about 400-500%, or about 500-1000%.

In embodiments, the individual is a patient who has been recently diagnosed with the disease. Typically, early treatment (treatment commencing as soon as possible after diagnosis) reduces the effects of the disease and to increase the benefits of treatment.

### Compositions and Methods of Administration

In embodiments, compositions are provided that include one or more of the compounds described herein.

In embodiments, pharmaceutically acceptable salts and/or prodrugs of the disclosed compounds are provided. Pharmaceutically acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically acceptable base addition salts include sodium, potassium, calcium, ammonium, or magnesium salt. Examples of physiologically acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral and parenteral routes of administration. As used herein, the term parenteral includes subcutaneous, intradermal, intravenous, intramuscular, intraperitoneal, intrasternal, and intrathecal administration, such as by injection. Administration of the disclosed compounds or compositions can be a single administration, or at continuous or distinct intervals as can be readily determined by a person skilled in the art.

The compounds disclosed herein, and compositions that include them, can also be administered utilizing liposome technology, slow-release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The form depends on the intended mode of administration and therapeutic application. The compositions can also include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously include between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions that include them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell includes attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy)propane:sebacic acid] in a 20:80 molar ratio (as used in GLIADEL); chondroitin; chitin; and chitosan.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders that include the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium that includes, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it may be desirable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a patient's skin to reduce the size (and can include complete removal) of malignant or benign growths, or to treat an infection site. Compounds and agents disclosed herein can be applied directly to the growth or infection site. In embodiments, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to improve the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that include a compound disclosed herein in combination with a pharmaceutically acceptable carrier. In embodiments, the pharmaceutical composition is adapted for oral, topical or parenteral administration. The dose administered to a patient, particularly a human, should be sufficient to achieve a therapeutic response in the patient over a reasonable time frame, without lethal toxicity, and without causing more than an acceptable level of side effects or morbidity. One skilled in the art will recognize that dosage will depend upon a variety of factors including the condition (health) of the patient, the body weight of the patient, kind of concurrent treatment, if any, frequency of treatment, therapeutic ratio, as well as the severity and stage of the pathological condition.

Also disclosed are kits that include a compound disclosed herein in one or more containers. The disclosed kits can optionally include pharmaceutically acceptable carriers and/or diluents. In embodiments, a kit includes one or more other components, adjuncts, or adjuvants as described herein. in another embodiment, a kit includes one or more anti-cancer agents, such as those agents described herein. In embodiments, a kit includes instructions or packaging materials that describe how to administer a compound or composition of the kit. Containers of the kit can be of any suitable material, e.g., glass, plastic, metal, etc, and of any suitable size, shape, or configuration. In embodiments, a compound and/or agent disclosed herein is provided in the kit as a solid, such as a tablet, pill, or powder form. In another embodiment, a compound and/or agent disclosed herein is provided in the kit as a liquid or solution. In embodiments, the kit includes an ampoule or syringe containing a compound and/or agent disclosed herein in liquid or solution form.

### Certain Definitions

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

The term “about” when immediately preceding a numerical value means a range (e.g., plus or minus 10% of that value). For example, “about 50” can mean 45 to 55, “about 25,000” can mean 22,500 to 2⁷,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, “cell penetrating peptide” or “CPP” refers to a peptide that facilitates delivery of a cargo, e.g., a therapeutic moiety (TM) into a cell. In embodiments, the CPP is cyclic, and is represented as “cCPP”. In embodiments, the cCPP is capable of directing a therapeutic moiety to penetrate the membrane of a cell. In embodiments, the cCPP delivers the therapeutic moiety to the cytosol of the cell. In embodiments, the cCPP delivers an antisense compound (AC) to a cellular location where a pre-mRNA is located.

As used herein, the term “endosomal escape vehicle” (FEV) refers to a cCPP that is conjugated by a chemical linkage (i.e., a covalent bond or non-covalent interaction) to a linker and/or an exocyclic peptide (EP). The EEV can be an EEV of Formula (B).

As used herein, the term “EEV-conjugate” refers to an endosomal escape vehicle defined herein conjugated by a chemical linkage (i.e., a covalent bond or non-covalent interaction) to a cargo. The cargo can be a therapeutic moiety (e.g., an oligonucleotide, peptide, or small molecule) that can be delivered into a cell by the EEV. The EEV-conjugate can be an EEV-conjugate of Formula (C).

As used herein, the term “exocyclic peptide” (EP) and “modulatory peptide” (MP) may be used interchangeably to refer to two or more amino acid residues linked by a peptide bond that can be conjugated to a cyclic cell penetrating peptide (cCPP) disclosed herein. The EP, when conjugated to a cyclic peptide disclosed herein, may alter the tissue distribution and/or retention of the compound. Typically, the EP comprises at least one positively charged amino acid residue, e.g., at least one lysine residue and/or at least one arginine residue. Non-limiting examples of EP are described herein. The EP can be a peptide that has been identified in the art as a “nuclear localization sequence” (NLS). Non-limiting examples of nuclear localization sequences include the nuclear localization sequence of the SV40 virus large T-antigen, the minimal functional unit of which is the seven amino acid sequence PKKKRKV (SEQ ID NO:42), the nucleoplasmin bipartite NLS with the sequence NLSKRPAAIKKAGQAKKKK (SEQ ID NO:52), the c-myc nuclear localization sequence having the amino acid sequence PAAKRVKLD (SEQ ID NO:53) or RQRRNELKRSF (SEQ ID NO:54), the sequence RMRKFKNKKDTAELRRRRVEVSVELRKAKKDEQILKRRNV (SEQ ID NO:50) of the IBB domain from importin-alpha, the sequences VSRKRPRP (SEQ ID NO:57) and PPKKARED (SEQ ID NO:58) of the myoma T protein, the sequence PQPKKKPL (SEQ ID NO:59) of human p53, the sequence SALIKKKKKMAP (SEQ ID NO:60) of mouse c-abl IV, the sequences DRLRR (SEQ ID NO:61) and PKQKKRK (SEQ ID NO:62) of the influenza virus NS1 the sequence RKLKKKIKKL (SEQ ID NO:63) of the Hepatitis virus delta antigen and the sequence REKKKFLKRR (SEQ ID NO:64) of the mouse Mxl protein, the sequence KRKGDEVDGVDEVAKKKSKK (SEQ ID NO:65) of the human poly(ADP-ribose) polymerase and the sequence RKCLQAGMNLEARKTKK (SEQ ID NO:66) of the steroid hormone receptors (human) glucocorticoid. International Publication No. 2001/038547 describes additional examples of NLSs and is incorporated by reference herein in its entirety.

As used herein, “linker” or “L” refers to a moiety that covalently bonds one or more moieties (e.g., an exocyclic peptide (EP) and a cargo, e.g., an oligonucleotide, peptide or small molecule) to the cyclic cell penetrating peptide (cCPP). The linker can comprise a natural or non-natural amino acid or polypeptide. The linker can be a synthetic compound containing two or more appropriate functional groups suitable to bind the cCPP to a cargo moiety, to thereby form the compounds disclosed herein. The linker can comprise a polyethylene glycol (PEG) moiety. The linker can comprise one or more amino acids. The cCPP may be covalently bound to a cargo via a linker.

The terms “peptide,” “protein,” and “polypeptide” are used interchangeably to refer to a natural or synthetic molecule comprising two or more amino acids linked by the carboxyl group of one amino acid to the alpha amino group of another. Two or more amino acid residues can be linked by the carboxyl group of one amino acid to the alpha amino group. Two or more amino acids of the polypeptide can be joined by a peptide bond. The polypeptide can include a peptide backbone modification in which two or more amino acids are covalently attached by a bond other than a peptide bond. The polypeptide can include one or more non-natural amino acids, amino acid analogs, or other synthetic molecules that are capable of integrating into a polypeptide. The term polypeptide includes naturally occurring and artificially occurring amino acids. The term polypeptide includes peptides, for example, that include from about 2 to about 100 amino acid residues as well as proteins, that include more than about 100 amino acid residues, or more than about 1000 amino acid residues, including, but not limited to therapeutic proteins such as antibodies, enzymes, receptors, soluble proteins and the like.

As used herein, the term “contiguous” refers to two amino acids, which are connected by a covalent bond. For example, in the context of a representative cyclic cell penetrating peptide (cCPP) such as

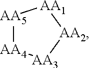

AA₁/AA₂, AA₂/AA₃, AA₃/AA₄ and AA₅/AA₁ exemplify pairs of contiguous amino acids.

A residue of a chemical species, as used herein, refers to a derivative of the chemical species that is present in a particular product. To form the product, at least one atom of the species is replaced by a bond to another moiety, such that the product contains a derivative, or residue, of the chemical species. For example, the cyclic cell penetrating peptides (cCPP) described herein have amino acids (e.g., arginine) incorporated therein through formation of one or more peptide bonds. The amino acids incorporated into the cCPP may be referred to residues, or simply as an amino acid. Thus, arginine or an arginine residue refers to

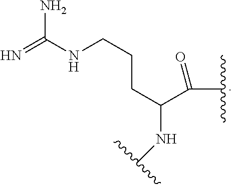 [SMILES: *NC(CCCNC(=N)N)C(*)=O]

The term “protonated form thereof” refers to a protonated form of an amino acid. For example, the guanidine group on the side chain of arginine may be protonated to form a guanidinium group. The structure of a protonated form of arginine is

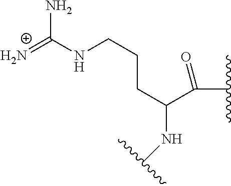 [SMILES: *NC(CCCNC(N)=[NH2+])C(*)=O]

As used herein, the term “chirality” refers to a molecule that has more than one stereoisomer that differs in the three-dimensional spatial arrangement of atoms, in which one stereoisomer is a non-superimposable mirror image of the other. Amino acids, except for glycine, have a chiral carbon atom adjacent to the carboxyl group. The term “enantiomer” refers to stereoisomers that are chiral. The chiral molecule can be an amino acid residue having a “D” and “L” enantiomer. Molecules without a chiral center, such as glycine, can be referred to as “achiral.”

As used herein, the term “hydrophobic” refers to a moiety that is not soluble in water or has minimal solubility in water. Generally, neutral moieties and/or non-polar moieties, or moieties that are predominately neutral and/or non-polar are hydrophobic. Hydrophobicity can be measured by one of the methods disclosed herein.

As used herein “aromatic” refers to an unsaturated cyclic molecule having 4n+2π electrons, wherein n is any integer. The term “non-aromatic” refers to any unsaturated cyclic molecule which does not fall within the definition of aromatic.

“Alkyl”, “alkyl chain” or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” “alkylene chain” or “alkylene group” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl”, “alkenyl chain” or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl, A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, I-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene”, “alkenylene chain” or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” or “alkoxy group” refers to the group —OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkylcarbamoyl” or “alkylcarbamoyl group” refers to the group —O—C(O)—NRₐR_{b}, where Rₐ and R_{b} are the same or different and are independently an alkyl, alkenyl, alkynyl, aryl, heteroaryl, as defined herein, or RₐR_{b} can be taken together to form a cycloalkyl group or heterocyclyl group, as defined herein. Unless stated otherwise specifically in the specification, an alkylcarbamoyl group can be optionally substituted.

“Alkylcarboxamidyl” or “alkylcarboxamidyl group” refers to the group —C(O)—NRₐR_{b}, where Rₐ and R_{b} are the same or different and are independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, an alkylcarboxamidyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, I-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diaryl amines, N-oxides, imides, and enamines, a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

As used herein, the symbol

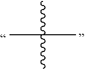

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

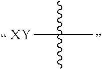

indicates that the chemical entity “XY” is bonded to another chemical entity via the point of attachment bond. Furthermore, the specific point of attachment to the non-depicted chemical entity can be specified by inference. For example, the compound CH₃—R³, wherein RU is H or

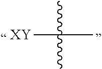

infers that when R³ is “XY”, the point of attachment bond is the same bond as the bond by which R³ is depicted as being bonded to CH₃.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “pharmaceutically acceptable carrier” refers to a carrier suitable for administration to a patient. A pharmaceutically acceptable carrier can be a sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol. polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chloro butanol, phenol, sorbic acid and the like. It can also be desirable to include isotonic agents such as sugars, sodium chloride and the like. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammoniumn, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “dose” refers to a specified quantity of a pharmaceutical agent provided in a single administration. In embodiments, a dose may be administered in two or more boluses, tablets, or injections. In embodiments, where subcutaneous administration is desired, the desired dose requires a volume not easily accommodated by a single injection. In such embodiments, two or more injections may be used to achieve the desired dose. In embodiments, a dose may be administered in two or more injections to reduce injection site reaction in a patient.

As used herein, the term “dosage unit” refers to a form in which a pharmaceutical agent is provided. In embodiments, a dosage unit is a vial that includes lyophilized antisense oligonucleotide. In embodiments, a dosage unit is a vial that includes reconstituted antisense oligonucleotide.

The term “therapeutic moiety” (TM) refers to a compound that can be used for treating at least one symptom of a disease or disorder and can include, but is not limited to, therapeutic polypeptides, oligonucleotides, small molecules and other agents that can be used to treat at least one symptom of a disease or disorder. In embodiments, the therapeutic moiety modulates expression or activity of a target protein. In embodiments, the therapeutic moiety modulates splicing. In embodiments, the therapeutic moiety induces exon skipping in a target mRNA transcript. In embodiments, the therapeutic moiety downregulates expression or activity of a target protein. In embodiments, the therapeutic moiety downregulates expression or activity of a target protein by inducing exon skipping in a target transcript.

The terms “modulate”, “modulating” and “modulation” refer to a perturbation of expression, function or activity when compared to the level of expression, function or activity prior to modulation. Modulation can include an increase (stimulation or induction) or a decrease (inhibition or reduction) in expression, function or activity. In embodiments, the compound disclosed herein includes a therapeutic moiety (TM) that downregulates expression, function and/or activity of a target protein. In embodiments, the compound disclosed herein includes a therapeutic moiety that upregulates expression, function and/or activity of a target protein.

“Amino acid” refers to an organic compound that includes an amino group and a carboxylic acid group and has the general formula

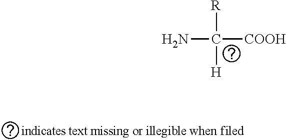

where R can be any organic group. An amino acid may be a naturally occurring amino acid or non-naturally occurring amino acid. An amino acid may be a proteogenic amino acid or a non-proteogenic amino acid. An amino acid can be an L-amino acid or a D-amino acid. The term “amino acid side chain” or “side chain” refers to the characterizing substituent (“R”) bound to the α-carbon of a natural or non-natural α-amino acid. An amino acid may be incorporated into a polypeptide via a peptide bond.

As used herein, the term “sequence identity” refers to the percentage of nucleic acids or amino acids between two oligonucleotide or polypeptide sequences, respectively, that are the same and in the same relative position. As such one sequence has a certain percentage of sequence identity compared to another sequence. For sequence comparison, typically one sequence acts as a reference sequence, to which test sequences are compared. Those of ordinary skill in the art will appreciate that two sequences are generally considered to be “substantially identical” if they contain identical residues in corresponding positions. In embodiments, the sequence identity between sequences may be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., Trends Genet. (2000), 16: 276-277), in the version that exists as of the date of filing. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows: (identical Residues×100)/(Length of Alignment-Total Number of Gaps in Alignment)

In other embodiments, sequence identity may be determined using the Smith-Waterman algorithm, in the version that exists as of the date of filing.

As used herein, “sequence homology” refers to the percentage of amino acids between two polypeptide sequences that are homologous and in the same relative position. As such one polypeptide sequence has a certain percentage of sequence homology compared to another polypeptide sequence. As will be appreciated by those of ordinary skill in the art, two sequences are generally considered to be “substantially homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains, and substitution of one amino acid for another of the same type may often be considered a “homologous” substitution.

As is well known in this art, amino acid sequences may be compared using any of a variety of algorithms, including those available in commercial computer programs such as BLASTP. gapped BLAST, and PSI-BLAST, in existence as of the date of filing. Such programs are described in Altschul, et al., J. Mol. Biol., (1990),215(3): 403-410; Altschul, et al., Nucleic Acids Res. (1997), 25:3389-3402; Baxevanis et al., Bioinformatics A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology.

As used herein, “cell targeting moiety” refers to a molecule or macromolecule that specifically binds to a molecule, such as a receptor, on the surface of a target cell. In embodiments, the cell surface molecule is expressed only on the surface of a target cell. In embodiments, the cell surface molecule is also present on the surface of one or more non-target cells, but the amount of cell surface molecule expression is higher on the surface of the target cells. Examples of a cell targeting moiety include, but are not limited to, an antibody, a peptide, a protein, an aptamer or a small molecule.

As used herein, the terms “antisense compound” and “AC” are used interchangeably to refer to a polymeric nucleic acid structure which is at least partially complementary to a target nucleic acid molecule to which it (the AC) hybridizes. The AC may be a short (in embodiments, less than 50 bases) polynucleotide or polynucleotide homologue that includes a sequence complimentary to a target sequence. In embodiments, the AC is a polynucleotide or polynucleotide homologue that includes a sequence complimentary to a target sequence in a target pre-mRNA strand. The AC may be formed of natural nucleic acids, synthetic nucleic acids, nucleic acid homologues, or any combination thereof. In embodiments, the AC includes oligonucleosides. In embodiments, AC includes antisense oligonucleotides. In embodiments, the AC includes conjugate groups. Nonlimiting examples of ACs include, but are not limited to, primers, probes, antisense oligonucleotides, external guide sequence (EGS) oligonucleotides, siRNAs, oligonucleotides, oligonucleosides, oligonucleotide analogs, oligonucleotide mimetics, and chimeric combinations of these. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound. In embodiments, an AC modulates (increases, decreases, or changes) expression of a target nucleic acid.

As used herein, the terms “targeting” or “targeted to” refer to the association of a therapeutic moiety, for example, an antisense compound, with a target nucleic acid molecule or a region of a target nucleic acid molecule. In embodiments, the therapeutic moiety includes an antisense compound that is capable of hybridizing to a target nucleic acid under physiological conditions. In embodiments, the antisense compound targets a specific portion or site within the target nucleic acid, for example, a portion of the target nucleic acid having at least one identifiable structure, function, or characteristic such as a particular exon or intron, or selected nucleobases or motifs within an exon or intron, such as a splice element or cis-acting splice regulatory element.

As used herein, the terms “target nucleic acid sequence” and “target nucleotide sequence” refer to the nucleic acid sequence or the nucleotide sequence to which a therapeutic moiety, such as an antisense compound, binds or hybridizes. Target nucleic acids include, but are not limited. to a portion of a target transcript, target RNA (including, but not limited to pre-mRNA and mRNA. or portions thereof), a portion of target cDNA derived from such RNA, as well as a portion of target non-translated RNA, such as miRNA. For example, in embodiments, a target nucleic acid can be a portion of a target cellular gene (or mRNA transcribed from such gene) whose expression is associated with a particular disorder or disease state. The term “portion” refers to a defined number of contiguous (i.e., linked) nucleotides of a nucleic acid.

As used herein, the term “transcript” or “gene transcript” refers an RNA molecule transcribed from DNA and includes, but is not limited to mRNA, pre-mRNA, and partially processed RNA.

The terms “target transcript” and “target RNA” refer to the pre-mRNA or mRNA transcript that is bound by the therapeutic moiety. The target transcript may include a target nucleotide sequence. In one embodiment, the target transcript includes a splice site.

The term “target gene” and “gene of interest” refer to the gene of which modulation of the expression and/or activity is desired or intended. The target gene may be transcribed into a target transcript that includes a target nucleotide sequence. The target transcript may be translated into a protein of interest.

The term “target protein” refers to the polypeptide or protein encoded by the target transcript (e.g., target mRNA).

As used herein, the term “mRNA” refers to an RNA molecule that encodes a protein and includes pre-mRNA and mature mRNA. “Pre-mRNA” refers to a newly synthesized eukaryotic mRNA molecule directly after DNA transcription. In embodiments, a pre-mRNA is capped with a 5′ cap, modified with a 3′ poly-A tail, and/or spliced to produce a mature mRNA sequence. In embodiments, pre-mRNA includes one or more introns. In one embodiment, the pre-mRNA undergoes a process known as splicing to remove introns and join exons. In embodiments, pre-mRNA includes one or more splicing elements or splice regulatory elements. In embodiments, pre-mRNA includes a polyadenylation site.

As used herein, the term “expression,” “gene expression,” “expression of a gene,” or the like refers to all the functions and steps by which information encoded in a gene is converted into a functional gene product, such as a polypeptide or a non-coding RNA, in a cell. Examples of non-coding RNA include transfer RNA (tRNA) and ribosomal RNA. Gene expression of a polypeptide includes transcription of the gene to form a pre-mRNA, processing of the pre-mRNA to form a mature mRNA, translocating the mature mRNA from the nucleus to the cytoplasm, translation of the mature mRNA into the polypeptide, and assembly of the encoded polypeptide. Expression includes partial expression. For example, expression of a gene may be referred to herein as generation of a gene transcript. Translation of a mature mRNA may be referred to herein as expression of the mature mRNA.

As used herein, “modulation of gene expression” or the like refers to modulation of one or more of the processes associated with gene expression. For example, modification of gene expression may include modification of one or more of gene transcription, RNA processing, RNA translocation from the nucleus to the cytoplasm, and translation of mRNA into a protein.

As used herein, the term “gene” refers to a nucleic acid sequence that encompasses a 5′ promoter region associated with the expression of the gene product, and any intron and exon regions and 3′ untranslated regions (“UTR”) associated with the expression of the gene product.

The term “immune cell” refers to a cell of hematopoietic origin and that plays a role in the immune response. Immune cells include, but are not limited to, lymphocytes (e.g., B cells and T cells), natural killer (NK) cells, and myeloid cells. The term “myeloid cells” includes monocytes, macrophages and granulocytes (e.g., basophils, neutrophils, eosinophils and mast cells). Monocytes are lymphocytes that circulate through the blood for 1-3 days, after which time, they either migrate into tissues and differentiate into macrophages or inflammatory dendritic cells or die. The term “macrophage” as used herein includes fetal-derived macrophages (which also can be referred to as resident tissue macrophages) and macrophages derived from monocytes that have migrated from the bloodstream into a tissue in the body (which can be referred to as monocyte-derived macrophages). Depending on which tissue the macrophage is located, it be referred to as a Kupffer cell (liver), an intraglomular mesangial cell (kidney), an alveolar macrophage (lungs), a sinus histiocyte (lymph nodes), a hofbauer cell (placenta), microglia (brain and spinal cord), or langerhans (skin), among others.

As used herein, “proximate” with respect to an AC and a splice regulatory element means that the AC binds to a nucleic acid sequence that is within about 25, about 20, about 15, about 10, about 5, about 4, about 3, about 2 or about 1 nucleotides of a splice regulatory element, including, for example, a 5′ splice site (5′ss), a branchpoint sequence (BPS), a polypyrimidine (Py) tract, or a 3′ splice site (3′ ss).

As used herein, “splice regulatory element (SRE),” “splicing element (SE),” and “splice element (SE)” are used interchangeably and refer to any nucleotide sequence within the transcript at which splicing occurs or that promotes, inhibits, or alters splicing. Examples of splice elements include terminal stem loop sequence (TLS), branchpoint sequence (BPS), polypyrimidine sequence (Py), 5′ splice site (5′ss), 3′ splice site (3′ss), and cis-regulatory elements such as intronic splicing silencer (ISS) sequences, intronic splicing enhancer (ISE) sequences, exon splicing enhancer (ESE) sequences, exonic splicing silencer (ESS) sequences, and sequences that include an exon/intron junction.

As used herein, the terms “splicing” refers to the modification of a pre-mRNA following transcription, in which introns are removed and exons are joined. Splicing occurs in a series of reactions that are catalyzed by a large RNA-protein complex that includes five small nuclear ribonucleoproteins (snRNPs), referred to as a spliceosome. Splice regulatory elements include a 3′ splice site, a 5′ splice site, and a branch site. The 5′ splice site is bound by the U1 snRNP and subsequently by the 16 snRNP. The RNA binding protein SF1 binds the branch point sequence but is later displaced by the U2 snRNP (See, for example, Ward and Cooper (2011) “The pathobiology of splicing,” J. Pathol. 220(2):152-163).

As used herein, “splice site” refers to the junction between an exon and an intron in a pre-mRNA molecule. A “cryptic splice site” is a splice site that is not typically used but may be used when the usual splice site is blocked or unavailable or when a mutation causes a normally dormant site to become an active splice site. An “aberrant splice site” is a splice site that results from a mutation in the native DNA and mRNA. An antisense compound that is “targeted to a splice site” refers to a compound that hybridizes with at least a portion of a target nucleotide sequence that includes a splice site or a compound that hybridizes with an intron or exon in proximity to a splice site, such that splicing of the mRNA is modulated. The targeted splice site may be a usual splice site, a cryptic splice site, or an aberrant splice site.

As used herein “splice donor site” can be used interchangeably with the term “5′ splice site” to refer to the nucleotide sequence immediately surrounding the exon-intron boundary at the 5′ end of the intron. The term “splice acceptor site” can be used interchangeably with the term “3′ splice site” to refer to the nucleic acid sequence immediately surrounding the intron-exon boundary at the 3′ end of the intron Many splice donor and acceptor sites have been characterized (See, for example, Ohshima et al. (1987) “Signals for the selection of a splice site in pre-mRNA: computer analysis of splice junction sequences and like sequences,” J. Mol. Biol., 195:247-259(1987)).

As used herein, the term “oligonucleotide” refers to an oligomeric compound comprising a plurality of linked nucleotides or nucleosides. One or more nucleotides of an oligonucleotide can be modified. An oligonucleotide can comprise ribonucleic acid (RNA) or deoxyribonucleic acid (DNA). Oligonucleotides can be composed of natural and/or modified nucleobases, sugars and covalent internucleoside linkages, and can further include non-nucleic acid conjugates.

As used herein, the term “nucleoside” refers to a glycosylamine that includes a nucleobase and a sugar. Nucleosides include, but are not limited to, natural nucleosides, abasic nucleosides, modified nucleosides, and nucleosides having mimetic bases and/or sugar groups. A “natural nucleoside” or “unmodified nucleoside” is a nucleoside that includes a natural nucleobase and a natural sugar. Natural nucleosides include RNA and DNA nucleosides.

As used herein, the term “natural sugar” refers to a sugar of a nucleoside that is unmodified from its naturally occurring form in RNA (2′-OH) or DNA (2′-H).

As used herein, the term “nucleotide” refers to a nucleoside having a phosphate group covalently linked to the sugar. Nucleotides may be modified with any of a variety of substituents.

As used herein, the term “nucleobase” refers to the base portion of a nucleoside or nucleotide. A nucleobase may include any atom or group of atoms capable of hydrogen bonding to a base of another nucleic acid. A natural nucleobase is a nucleobase that is unmodified from its naturally occurring form in RNA or DNA.

As used herein, the term “heterocyclic base moiety” refers to a nucleobase that includes a heterocycle.

As used herein “internucleoside linkage” refers to a covalent linkage between adjacent nucleosides.

As used herein “natural internucleoside linkage” refers to a 3′ to 5′ phosphodiester linkage.

As used herein, the term “modified internucleoside linkage” refers to any linkage between nucleosides or nucleotides other than a naturally occurring internucleoside linkage.

As used herein the term “chimeric antisense compound” refers to an antisense compound, having at least one sugar, nucleobase and/or internucleoside linkage that is differentially modified as compared to the other sugars, nucleobases and internucleoside linkages within the same oligomeric compound. The remainder of the sugars, nucleobases and internucleoside linkages can be independently modified or unmodified. In general, a chimeric oligomeric compound will have modified nucleosides that can be in isolated positions or grouped together in regions that will define a particular motif. Any combination of modifications and or mimetic groups can include a chimeric oligomeric compound as described herein.

As used herein, the term “mixed-backbone antisense oligonucleotide” refers to an antisense oligonucleotide wherein at least one internucleoside linkage of the antisense oligonucleotide is different from at least one other internucleoside linkage of the antisense oligonucleotide.

As used herein, the term “nucleobase complementarity” refers to a nucleobase that is capable of base pairing with another nucleobase. For example, in DNA, adenine (A) is complementary to thymine (T). For example, in RNA, adenine (A) is complementary to uracil (U). In embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, the term “non-complementary nucleobase” refers to a pair of nucleobases that do not form hydrogen bonds with one another or otherwise support hybridization.

As used herein, the term “complementary” refers to the capacity of an oligomeric compound to hybridize to another oligomeric compound or nucleic acid through nucleobase complementarity. In embodiments, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are antisense compounds that may include up to about 20% nucleotides that are mismatched (i.e., are not nucleobase complementary to the corresponding nucleotides of the target). In embodiments, the antisense compounds contain no more than about 15%, for example, not more than about 10%, for example, not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% nucleobase complementary to a target nucleic acid.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur tinder varying circumstances.

As used herein, the term “specifically hybridizes” refers to the ability of an oligomeric compound to hybridize to one nucleic acid site with greater affinity than it hybridizes to another nucleic acid site. In embodiments, an antisense oligonucleotide specifically hybridizes to more than one target site. In embodiments, an oligomeric compound specifically hybridizes with its target under stringent hybridization conditions.

“Stringent hybridization conditions” and “stringent hybridization wash conditions” in the context of nucleic acid hybridization are sequence dependent and are different under different environmental parameters. An extensive guide to the hybridization of nucleic acids is found in Tijssen, Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleotide sequences which have more than 100 complementary residues on a filter in a Southern or Northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook and Russel, Molecular Cloning: A laboratory Manual, 3^{rd} ed., Cold Spring Harbor Laboratory Press, 2001 for a description of SSC buffer), Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide.

As used herein, the term “2′-modified” or “2′-substituted” means a sugar that includes substituent at the 2′ position other than H or OH. 2′-modified monomers, include, but are not limited to, BNA's and monomers (e.g., nucleosides and nucleotides) with 2′-substituents, such as allyl, amino, azido, thio, O-allyl, O—C1-C10 alkyl, —OCF3, O—(CH2)2-O—CH3, 2′-O(CH2)2SCH3, O—(CH2)2-O—N(Rm)(Rn), or O-CH2-C(═O)—N(Rm)(Rn), where each Rm and Rn is, independently, H or substituted or unsubstituted C1-C10 alkyl.

As used herein, the term “MOE” refers to a 2′-O-methoxyethyl substituent.

As used herein, the term “high-affinity modified nucleotide” refers to a nucleotide having at least one modified nucleobase, internucleoside linkage or sugar moiety, such that the modification increases the affinity of an antisense compound that includes the modified nucleotide to a target nucleic acid. High-affinity modifications include, but are not limited to, BNAs, LNAs and 2′-MOE.

As used herein the term “mimetic” refers to groups that are substituted for a sugar, a nucleobase, and/or internucleoside linkage in an AC. Generally, a mimetic is used in place of the sugar or sugar-internucleoside linkage combination, and the nucleobase is maintained for hybridization to a selected target. Representative examples of a sugar mimetic include, but are not limited to, cyclohexenyl or morpholino. Representative examples of a mimetic for a sugar-internucleoside linkage combination include, but are not limited to, peptide nucleic acids (PNA.) and morpholino groups linked by uncharged achiral linkages. In some instances, a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

As used herein, the term “bicyclic nucleoside” or “BNA” refers to a nucleoside wherein the furanose portion of the nucleoside includes a bridge connecting two atoms on the furanose ring, thereby forming a bicyclic ring system. BNAs include, but are not limited to, α-L-LNA, β-D-LNA, ENA, Oxyamino BNA (2′-O—N(CH3)-CH2-4′) and Aminooxy BNA (2′-N(CH3)-O-CH2-4′).

As used herein, the term “4′ to 2′ bicyclic nucleoside” refers to a BNA wherein the bridge connecting two atoms of the furanose ring bridges the 4′ carbon atom and the 2′ carbon atom of the furanose ring, thereby forming a bicyclic ring system.

As used herein, a “locked nucleic acid” or “LNA” refers to a nucleotide modified such that the 2′-hydroxyl group of the ribosyl sugar ring is linked to the 4′ carbon atom of the sugar ring via a methylene groups, thereby forming a 2′-C,4′-C-oxymethylene linkage. LNAs include, but are not limited to, α-L-LNA, and β-D-LNA.

As used herein, the term “cap structure” or “terminal cap moiety” refers to chemical modifications, which have been incorporated at either end of an AC.

All publications, patents and patent applications mentioned in the specification are indicative of the level of skill of those skilled in the art to which this invention pertains. All publications, patents and patent applications are herein incorporated by reference to the same extent as if each individual publication or patent application was specifically and individually indicated to be incorporated by reference.

### EXAMPLES

### Example 1. Construction of a Cell-Penetrating Peptide—Antisense Compound Conjugate

An antisense compound (AC) of any one or SEQ ID NOs: 155 to 333 and/or 340 designed to bind to and block expression of IRF-5 and/or GYS1 is constructed as a phosphorodiamidate morpholino oligomer (PMO) with a C6-thiol 5′ modification. An antisense compound of any one of SEQ IS NOs: 344 to 364 designed to generate a non-toxic isoform of DUX4 is constructed as a phosphorodiamidate morpholino oligomer (PMO) with a C6-thiol 5′ modification

An EEV is formulated that includes a CCP. A cell-penetrating peptide is formulated using Fmoc chemistry and conjugated to the AC, for example, as described in International Application No. PCT/US20/66459, filed by Entrada Therapeutics, Inc., on Dec. 21, 2021, entitled “COMPOSITIONS FOR DELIVERY OF ANTISENSE COMPOUNDS,” the disclosure of which is hereby incorporated in its entirety herein. In embodiments, the cCPP includes the amino acid sequence FfΦRrRrQ (SEQ ID NO:78). In embodiments EEV includes an exocyclic peptide having the sequence KKKRKV (SEQ ID NO:33). In embodiments, the EEV includes KKKRKV-PEG₂-K-(cyclo(FfΦRrRrQ))-PEG-K(N3) (SEQ ID NO:33-PEG2-K-(cyclo(SEQ ID NO:78))-PEG₁₂-K(N₃)). In embodiments, the AC compound is conjugated to the EEV using click chemistry. In embodiments, the compound includes KKKRKV-PEG₂-K-(cyclo(FfΦRrRrQ))-PEG₁₂-K-linker-3′-AC-5′ (SEQ ID NO:33-PEG₂-K-(cyclo(SEQ ID NO:78))-PEG₁₂-K-linker-3′-AC-5′) where the linker includes the product of a strain promoted click reaction between an azide and a cyclooctyne. The linker may also include other groups such as a carbon chain, PEG chain, carbamate, urea, and the like.

### Example 2. Knockdown of GYS1 Expression Via Exon Skipping

An EEV-PMO is used to induce exon skipping of exon 6 leading to a premature termination codon and nonsense mediated decay of the GYS1 target transcript. The EEV used was (Ac-PKKKRKV-PEG₂-K(cyclo[Ff-Nal-GrGrQ])-PEG₂-K(N₃)—NH₂) (SEQ LD NO:42-PEG₂-K(cyclo[SEQ ID NO:135])-PEG₂-K(N₃)—NH₂)). The PMO sequence was TCACTGTCTGGC TCA CATACC CATA (SEQ ID NO:327). The PMO and EEV were conjugated using azide-alkyne click chemistry.

GYS1/GAA double knockout mice, when compared to the GAA single knockout mice, have exhibited a profound reduction in the amount of glycogen in the heart and skeletal muscles, a significant decrease in lysosomal swelling and autophagic build-up. These cellular-level changes lead to cardiomegaly correction, normalization of glucose metabolism and correction of muscle atrophy. Despite the absence of GAA, the elimination of GYS1 may play an important role iGAA knockout mice (GAA^{-/-}) were injected with a single IV dose of either 13.5 mg/kg of EEV-PMO, 27 mg/kg of EEV-PMO, 27 mg/kg of PMO, or a negative control (vehicle). GYS1 mRNA and protein levels were measured one-week post-injection. Levels of GYS1 were also assessed at one week, two weeks, four weeks, and eight weeks post IV dose of 13.5 mg/kg EEV-PMO.

FIG. 7A-7D show a significant knockdown GYS1 expression in the diaphragm and cardiac muscle in both the EEV-PMO arms, but not in the PMO only arm. This pharmacodynamic result is notable given that this is a single dose experiment administered at very low doses, and it suggests that GYS1 is an addressable target. Additionally, GYS1 protein levels and mRNA are sustained for up to eight weeks for injection in the heart, diaphragm, quadriceps, and triceps (FIGS. 8A-8D and FIGS. 9A-9D). The protein level is relative to total protein. The mRNA level is relative to mouse beta-actin and mouse GAPDH, two control housekeeping genes.

### Example 3. Knockdown of IRF5 Expression Via Exon Skipping

Four EEV-PMO conjugates were used to induce exon skipping of exon 4 to introduce a premature termination codon resulting in nonsense mediated decay of the IRF-5 target transcript. The PMO sequence for each of the four conjugates was 5′-AGA ACG TAA TCA TCA TG GGT TGG C-3′ (SEQ ID NO:340). The EEVs used were Ac-PKKKRKV-miniPEG₂-K(cyclo[FGFGRGRQ])-PEG12-OH (EEV #1, 1120) (Ac-SEQ ID NO:42-miniPEG₂-K(cyclo[SEQ ID NO:82])-PEG12-OH); Ac-PKKKRKV-miniPEG₂-K(cyclo[Ff-Nal-GrGrQ])-PEG₁₂-OH (EEV #2, 1113) (Ac-SEQ ID NO:42-miniPEG₂-K(cyclo[SEQ ID NO:135])-PEG₁₂-OH); Ac-PKKKRKV-miniPEG₂-K(cyclo[FGFGRRRQ])-PEG₁₂-OH (EEV #4; 1184) (Ac-SEQ ID NO:42-miniPEG₂-K(cyclo[SEQ ID NO:84])-PEG₁₂-OH); and 1185: Ac-PKKKRKV-miniPEG₂-K(cyclo[FGFRRRRQ])-PEG₁₂-OH (EEV #4, 1185) (Ac-SEQ ID NO:42-miniPEG₂-K(cyclo[SEQ ID NO:85])-PEG₁₂-OH). EEVs were conjugated to the PMOs using amide conjugation chemistry.

For the in vivo studies, wild type mice were treated with two doses of EEV #1-PMO on Days 0 and 3. Samples were collected on Day 7 for qPCR to measure mRNA levels. For the in vitro studies, mouse macrophage cells treated with the EEV #1-PMO or were pre-treated with 2 μM of EEV-PMOs #1-4 for 4 hours, followed by stimulation with R848, an imidazoquinoline compound that is a specific activator of toll-like receptor (TLR) 7/8, overnight. At 24 hours post treatment, cells were harvested and evaluated by Western Blot.

A significant knockdown of IRF5 levels was observed in the liver (FIG. 10A), small intestine (FIG. 10B), and tibias anterior (FIG. 10C) after treatment with EEV #1-PMO. In all tissues the knockdown was dose dependent. Additionally, mouse macrophage cells treated with the EEV #1-PMO had a statistically significant reduction of IRF5 protein levels at doses of 30 μM, 10 μM and 3 μM (FIG. 11A). However, mouse macrophage cells pretreated with EEV #2-PMO, EEV #3-PMO, and EEV #4-PMO followed by stimulation with R848, had significant improvement in relative potency when compared to EEV #1-PMO, as measured by IRF-5 protein expression (FIG. 11B). mRNA levels are relative to the vehicle control which was set to 100%.

### Example 4. Knockdown of GYS1 Via Exon Skipping In Vitro

The effectiveness of PMO 220 and PMO-EEV 220-814 at knocking down mGYS1 levels in mouse cell lines was evaluated. The PMO was designed to induce exon skipping of exon 6 of mouse GYS1 to introduce a premature termination codon resulting in nonsense mediated decay of the IRF-5 target transcript. Construct 220-814 is PMO 220 (5′-TCACTGTCTGGCTCACATACCCATA-3′; SEQ ID NO: 327) conjugated to an EEV with the sequence of Ac-PKKKR-KV-PEG₂-K(cyclo[FfΦCit-r-Cit-rQ])-PEG₁₂-K(N₃)—NH₂ (EEV 814) (Ac-SEQ ID NO:42-PEG₂-K(cyclo[SEQ ID NO:79])-PEG₁₂-K(N₃)—NH₂). The PMO was conjugated to the EEV using click chemistry.

Wild type mouse myoblast cell line C2C12 and wild type mouse fibroblast cell line 3T3 were treated with various concentrations of PMO 220 and PMO-EEV 220-814 via Endoporter transfection (6 μL/ml; 6 μM). Two days post treatment, the cell lines were evaluated for the level of GYS1 mRNA.

Both PMO 220 and PMO-EEV 220-814 showed a decrease in GYS1 mRNA levels in the C2C12 myoblast (FIG. 12 and FIG. 13A) cell line. PMO 220 also showed a decrease in GYS1 mRNA levels in the 3T3 fibroblast cell line (FIG. 13B).

### Example 5. In Vivo Evaluation of a PMO-EEV Construct Targeting GYS1

A Pompe disease mimicking mouse model was used to determine the in vivo effectiveness of an PMO-EEV construct (EEV-PMO 220-814) at modulating GYS1 levels and the Pompe phenotype. PMO-EEV 220 is PMO 220 (5′-TCACTGTCTGGCTCACATACCCATA-3′; SEQ ID NO: 327) conjugated to an EEV 814 (Ac-PKKKRKV-PEG₂-K(cyclo[FfΦCit-r-Cit-rQ])-PEG₁₂-K(N₃)—NH₂) (Ac-SEQ ID NO:42-PEG₂-K(cyclo[SEQ ID NO:79])-PEG₁₂-K(N₃)—NH₂).

Wild type 1B6-129 mice and acid α-glucosidase (GAA) knockout mice (GAA^{-/-}) were treated with a single dose of 27 mpk PMO 220, 20 mpk PMO-EEV 220-814, or 40 mpk PMO-EEV 220-814 via intravenous injection One week post treatment, mice were sacrificed, and tissue harvested for western blot and RT-qPCR analysis. The PMO was conjugated to the EEV using click chemistry.

Efficient knockdown of mouse GYS1 mRNA in cardiac and skeletal muscle was observed after treatment with PMO-EEV 220-814 but not after treatment with just PMO 220 (FIGS. 14A-14D).

GYS2 is the most prevalent GYS protein in the liver. PMO 220 and PMO-EEV 220-814 treatment did not affect the level of GYS2 mRNA at treatment levels below 30 mpk (FIG. 15) indicating selective GYS1 target engagement The 40 mpk 220-814 treatment group showed higher levels of GYS2 mRNA in the liver. This may be due to a feedback loop between GYS1 and GYS2 where downregulation of GYS leads to upregulation of GYS2.

A GYS antibody that is not specific to GYS1 was used to measure GYS1 levels in the quadriceps and triceps (FIGS. 25A-B). GYS1 protein reduction in the quadriceps was observed after treatment with the EEV-PMO construct (FIG. 25A). No conclusion can be made for the GYS1 protein levels in the triceps due to gel inconsistences in loading (FIG. 25B).

A GYS1 specific antibody was also used to measure GYS1 protein levels in the diaphragm, heart, and triceps (FIG. 26). A clear trend in GYS1 reduction in the diaphragm and heart is observed after treatment with the EEV-PMO construct. There is no clear reduction in GYS21 levels in the triceps. Of note, there was large variability in protein levels of untreated animals and no GYS1 signal was observed in the liver using the GYS1 specific antibody, likely due to low GYS1 expression in the liver. Example 6. In vivo evaluation of a second PMO-EEV construct targeting GYS1

A Pompe disease mimicking mouse model was used to determine the in vivo effectiveness of the PMO-EEV construct 220-1055. Construct 220-1055 is PMO 220 (5′-TCACTGTCTGGCTCACATACCCATA-3′; SEQ ID NO:327) conjugated to EEV 1055 (Ac-PKKKRKV-K(cyclo[FfΦGrGrQ])-PEG₂-K(N₃)—NH₂) (Ac-SEQ ID NO:42-K(SEQ ID NO:77)-PEG₂-K(N₃)—NH₂). The PMO was conjugated to the EEV using click chemistry.

The same knockout mice model as Example 5 was used. Mice were treated with a single dose of 15 mpk PMO 220, 10 mpk PMO-EEV 220-1055, or 20 mpk PMO-EEV 220-1055 via intravenous injection. Mice were sacrificed one week, 2 weeks, 4 weeks, or 8 weeks post treatment. Tissue was harvested for western blot, RT-qPCR, and glycogen storage analysis. Prior to sacrifice at 2 weeks and 8 weeks, the mice were fasted overnight.

A reduction in the Gys1 mRNA level was observed in the heart (FIG. 16A) at 1 week, 2 weeks, and 4 weeks post treatment PMO-EEV 220-1055. A slight reduction in the GYS1 mRNA level was observed 8-weeks after treatment with PMO-EEV 220-1055 in the heart (FIG. 16A). A reduction in GYS1 mRNA levels in the diaphragm (FIG. 16B), quadriceps (FIG. 16C), and triceps (FIG. 16D) was observed for 1 week, 2 weeks, 4 weeks, and 8 weeks, post treatment with PMO-EEV 220-1055.

Similar to the RNA levels, the heart (FIG. 17A), diaphragm (FIG. 17B), triceps (FIG. 17C), and the quadriceps (FIG. 17D) all showed a strong reduction in GYS1 protein levels at 2 weeks and 4 weeks post treatment of PMO-EEV 220-1055 The reduction in GYS1 seems to get stronger with time until 4 weeks post treatment and then get weaker.

The drug exposure in the heart (FIG. 18A), diaphragm (FIG. 18B), triceps (FIG. 18C), and quadriceps (FIG. 18D) decreased form 1 week to 8 weeks post treatment with PMO-EEV 220-1055. However, drug exposure can be detected 8 weeks post injection for all muscle tissues analyzed.

The AMPLEX red glucose/glucose oxidase assay kit (available from ThermoFischer in Waltham, MA) was used to determine glycogen storage levels in select tissues. Glycogen levels were determined by subtracting the glycose levels subtracted from the glucose levels from the same sample digested with α-amyloglucosidase.

A slight, but not a significant reduction of glycogen was observed in the heart, diaphragm, triceps, and quadriceps 1 week, 2 weeks, and 4 weeks post treatment with PMO-EEV 220-1055. A similar observation was made 8 weeks post treatment. The lack of reduction in glycogen levels may be due to the glycogen storage phenotype not being fully developed when the mice were treated. For example, a comparison of wild type mice and GAA knockout mice shows that glycogen storage levels in the heart, diaphragm, quadriceps, and triceps increase with age. This finding is consistent with literature (Raben, N., et. al., Journal of Biological Chemistry (1998), 273(30), pg. 19086). If the glycogen phenotype was still developing when the mice were treated, the mice may not have accurately reflected the Pompe disease model at the time of testing.

### Example 7. In Vivo Evaluation of a Third PMO-EEV Construct Targeting GYS1

A Pompe disease mimicking mouse model was used to determine the in vivo effectiveness of the PMO-EEV construct 220-1120. Construct 220-1120 is PMO 220 (5′-TCACTGTCTGGCTCACATACCCATA-3′; SEQ ID NO:327) conjugated to EEV 1120 Ac-PKKKRKV-AEEA-Lys(cyclo[FGFGRGRQ]-PEG₁₂-OH) (Ac-SEQ ID NO:42-AEEA-Lys(cyclo[SEQ ID NO:82]-PEG₁₂-OH)). The PMO was conjugated to the EEV using amide conjugation chemistry.

The same mice model as Example 5 was used. Wild type mice and (GA A knockout mice were treated with a single dose of 40 mpk PMO 220, 5 mpk PMO-EEV 220-1120, 10 mpk PMO-EEV 220-1120, 20 mpk PMO-EEV 220-1120, or 40 mpk PMO-EEV 220-1120 via intravenous injection. Mice were sacrificed 2 weeks post treatment. Tissue was harvested for western blot analysis, RT-qPCR, and glycogen storage. Mice were fasted overnight prior to sacrifice.

A dose dependent knockdown of GYS1 mRNA (FIG. 19) and protein levels (FIG. 20) was observed in the triceps (C) and quadriceps (D) of mice treated with PMO-EEV 220-1120. A modest knockdown of GYS1 mRNA and protein levels were observed in the heart (A) and diaphragm (B), perhaps because of the limited number of mice tested.

A similar study was done but with mice receiving multiple doses of PMO-EEV 220-1120. Briefly, GAA knockout mice were dosed with 7 mpk PMO 220 or 10 mpk PMO-EEV 220-1120 every two weeks for 8 weeks via intravenous injection (total dose for PMO=5 mpk; total dose for PMO-EEV=50 mpk). Ten weeks post the first treatment, mice were sacrificed. Tissue harvested for western blot., RT-qPCR, and glycogen storage analysis. Before the first treatment, after the third treatment, and after the last treatment, mice were probed for grip strength, wire hang time, and heart function (via echocardiography).

Robust GYS1 mRNA knockdown was observed in both cardiac and skeletal muscles (FIGS. 21A-C). The mRNA levels of GYS1 and GYS2 were unaffected in the liver (FIGS. 22A-B.

Robust glycogen synthase activity knockdown was observed in the heart and quadriceps. However, repeated doses of EEV-PMO 220-1120 did not reduce tissue glycogen storage in skeletal and muscle tissues. Additionally, no significant change in the grip strength or forelimb hang time was observed for treated mice versus untreated mice.

### Example 8: IFR-5 Ablation Using Two Doses of EEV-PMO Mouse Study

A two-dose mouse study was used to study the effectiveness of PM-EEV 278-1120. Mice were dosed with either 40 milligrams per kilogram (mpk) or 20 mpk of PMO 278 (AGA ACG TAA TCA TCA GTG GGT TGG C; SEQ ID NO:340) or EEV-PMO compound 278-1120 at day zero and again at day three. PMO-EEV 278-1120 includes PMO 278 conjugated to EEV 1120 (Ac-PKKKRKV-PEG₂-K(cyclo[FGFGRGRQ])-PEG₁₂-OH) (Ac-SEQ ID NO:42-PEG₂-K(cyclo[SEQ ID NO:82])-PEG₁₂-OH). Five days following the second dose, the mice were sacrificed, and the blood and tissue collected.

FIG. 23A-C shows the IRF-5 expression levels in various tissues after treatment. IRF-5 expression knockdown was observed in mouse TiA and liver tissues, but not in the small intestine tissue.

### Example 9: IFR-5 Ablation Using a Single Dose of EEV-PMO Mouse Study

A single dose mouse study was used to study the effectiveness of PM-EEV 278-1120. PMO-EEV 278-1120 is PMO 278 (AGA ACG TAA TCA TCA GTG GGT TGG C; SEQ ID NO:340) conjugated to EEV 1120 (Ac-PKKKRKV-PEG₂-K(cyclo[FGFGRGRQ])-PEG₁₂-OH) (Ac-SEQ ID NO:42-PEG₂-K(cyclo[SEQ ID NO.82])-PEG₁₂-OH). At day zero, mice were dosed with 80 milligrams per kilogram (mpk) PMO 278; 40 mpk or 80 mpk of PMO-EEV 278-1120 via IV; or 120 mpk PMO-EEV 278-1120 subcutaneously (SC). Seven days following the second dose, the mice were sacrificed, and the blood and tissue collected.

FIG. 24 shows IRF-5 expression levels in the liver (A), kidney (B), and tibialis anterior (C) tissues where 80 mpk PMO 278; A is 80 mpk PMO, B is 40 mpk PMO-EEV 278-1120 delivered via IV; C is 80 mpk PMO-EEV 278-1120 delivered via IV; and D is 120 mpk PMO-EEV 278-1120 delivered subcutaneously. There was a significant decrease in IRF-5 protein expression in the liver tissue of mice with a single dose administration of 278-1120 at both 40 and 80 mpk, corresponding to a 40% and 53% reduction, respectively (A). The IRF-5 levels in the kidney tissue were low compared to other tissues examined. The data shows variability, likely due to difficulty quantifying band intensity vs background. Additionally, variability in the tibialis anterior tissue data was observed due to samples that did not run well on the gel (data not shown). Overall, the data shows a similar trend in the kidney as in the liver; there is a significant reduction in IRF-5 protein levels with a single dose administration

### Example 10: Evaluation of In Vitro Exon Skipping of Various EEV-PMOs Targeting IRF-5

Unstimulated RAW 264.7 monocyte/macrophage cells were used to evaluate IRF-5 expression and exon skipping after treatment with two EEV-PMO compounds 277-1120 and 278-1120. PMO-EEV 277-1120 is PMO sequence ACG TAA TCA TCA GTG GGT TGG CTC T (SEQ ID NO:365) conjugated to EEV 1120 Ac-PKKKRKV-AEEA-Lys-(cyclo[FGFGRGRQ])-PEG12-OH (Ac-SEQ ID NO:42-AEEA-Lys-cyclo(SEQ ID NO:82)-PEG₁₂-OH) through amide conjugation chemistry. PMO-EEV 278-1120 is PMO sequence AGA ACG TAA TCA TCA GTC GGT TGG C (SEQ ID NO:340) conjugated to EEV 1120 Ac-PKKKRKV-AEEA-Lys-(cyclo[FGFGRGRQ])-PEG12-OH (Ac-SEQ ID NO:42-AEEA-Lys-cyclo(SEQ ID NO:82)-PEG₁₂-OH) through amide conjugation chemistry. Briefly, 150K cells/well were seeded in a 24 well plate in 0.5 ml DMEM. After 4 hours, the EEV-PMO compounds were added to the cells giving a total volume of 500 μL, The cells were then incubated for 24 hours. Following incubation, the cell culture media was collected for cytokines, IL6, and TNF-α detection. The RNA was extracted and used for IRF-5 transcript quantification. The protein lysates were used to measure IRF-5 protein level changes. IRF-5 expression levels were determined relative to β-tubulin.

For the exon skipping study, the cells were treated as described above. After incubation with the EEV-PMO compounds, the cells were washed with fresh media then incubated overnight. Following the second incubation, the RNA was harvested, and RT-PCR was done using primers that detect exon 5 skipping in the RF—S gene.

Both 277-1120 and 278-1120 showed target engagement in the RAW 264.7 mouse macrophages/monocytes and significantly reduced IRF-5 protein levels in a dose dependent fashion (FIG. 27A). Compound 277-1120 significantly depleted IRF-5 protein levels by 80% at 30 μM, ˜50% at 10 μM, and no substantial changes were observed with lower dosage of 3.3 μM. Compound 278-1120 had stronger effect on IRF-5 depletion than 277-1120. Compound 278-1120 reduced IRF-5 protein levels by ˜80% at 30 μM and ˜65% at 10 μM. Even at lower dosage of 3.3 μM, 278-1120, had an IRF-5 protein depletion of level of ˜40%.

The EEV-PMO compound 0278-1120 induced partial exon skipping as soon as 30 min after exposure with efficacy increasing as exposure time increases (FIG. 27B).

A similar experiment was conducted was additional EEV-PMO compounds where the PMO was PMO 278 (AGA ACG TAA TCA TCA GTG GGT TGG C; SEQ ID NO:340). The PMO 278 was conjugated to EEV various EEVs including Ac-PKKKRKV-PEG₂-K(cyclo[FGFGRGRQ])-PEG₁₂-OH (EEV #1, 1120, Ac-SEQ ID NO:42-PEG₂-K(cyclo[SEQ ID NO:82])-PEG₁₂-OH); Ac-PKKKRKV-PEG₂-K(cyclo[Ff-Nal-GrGrQ])-PEG₁₂-OH (EEV #2, 1113, Ac-SEQ ID NO:42-PEG₂-K(cyclo[SEQ ID NO:135])-PEG₁₂-OH); Ac-PKKKRKV-PEG₂-K(cyclo[FGFGRRRQ])-PEG₁₂-OH (EEV #3; 1184, Ac-SEQ ID NO:42-PEG₂-K(cyclo[SEQ ID NO:84])-PEG₁₂-OH); and Ac-PKKKRKV-PEG₂-K(cyclo[FGFRRRRQ])-PEG₁₂-OH (EEV #4, 1185, Ac-SEQ ID NO:42-PEG2-K(cyclo[SEQ ID NO:85])-PEG₁₂-OH) using amide conjugation chemistry.

Similar methods were used as described above except that the cells were pre-treated with the EEV-PMO compound followed by stimulation with R848 overnight. R484 is a Toll-like receptor agonist and leads to the induction of IRF-5 expression. The total treatment time was 24 hours.

R848 significantly increases IRE-5 Protein expression in RAW264.7 cells. All EEV-PMO treated samples at all the tested concentrations showed a significant reduction in IRF-5 protein expression when compared to cells stimulated with R848 (FIG. 28A). EEV-PMO compounds 278-1113, 278-1184, and 278-1185 were on average 5-fold more efficacious than 278-1120 with about 80% IRF-5 protein reduction at concentrations as low as 2 μM when compared to IRE-5 levels in cells stimulation with R848

EEV-PMO compounds 278-1113, 278-1184, and 278-1185 exhibited higher exon skipping at 5 μM than 278-1120 (FIG. 12B). No substantial difference in exon skipping was observed between 278-1113, 278-1184, and 278-1185.

### Example 11: Evaluation of Various EEV-PMO Compounds in Human THP1 Cells

Human THP1 cells were used to evaluate RF-5 expression and exon skipping after treatment with various PMO compounds and various EEV-PMO compounds. The PMO compounds tested include 344 (TTGGCAACATCCTCTGCAGCTGAAG; SEQ ID NO:366, Hs-IRF-5-E4N6); 345 (GCAACATCCTCTGCAGCTG SEQ ID NO:367, Hs-IRF-5-E4N3); 346 (TCAGGCTTGGCAACATCCTCTGCAG; SEQ ID NO:368, Hs-IRF-5-E5P0; IRF5-E4N3 (TAATCATCAGTGGGTTGGCTCTCTG, SEQ ID NO: 369): 278 (AGA ACG TAA TCA TCA GTG GGT TGG C; SEQ ID NO:340, Hs-IRF-5-E4P3), and 277 (ACG TAA TCA TCA GTG GGT TGG CTC T; SEQ ID NO:365, Hs-IRF-5-E4PO). The EEV-PMO compounds included PMOs 344, 345, and 346 were individually conjugated via amide conjugation chemistry to EEV 1120 (Ac-PKKKRKV-AEEA-Lys-(cyclo[FGFGRGRQ])-PEG12-OH) (Ac-SEQ ID NO:42-AEEA-Lys-cyclo(SEQ ID NO:82)-PEG₁₂-OH).

Briefly, for the PMO only study, the nucleofection method was used to transfect PMO compounds into the THP1 cells. Cells were plated after nucleofection in PMA containing media and incubated for 24 hours before harvest. The RNA was harvested, and RT-PCR was done using primers that detect both exon 4 and exon 5 skipping in the IRF-5 gene.

Briefly, for the EEV-PMO study, the THP1 cells were differentiated by PMA overnight. The cells were then treated with various EEV-PMO conjugates and incubated for 24 hours before harvest. The RNA was harvested, and RT-PCR was done using primers that detect exon 5 skipping in the IRE-5 gene.

FIG. 29A shows the exon 4 and exon 5 skipping levels after treatment with various PMO compounds. The PMO compounds that worked well in mouse cells do not necessarily translate to human cells. For example, low levels of exon skipping were observed for Hs-IRF-5-E4P3 (PMO 278) and Hs-IRF-5-E4PO (PMO 277). Exon skipping was observed for the Hs-IRF-5-E5N6 (PMO 344), Hs-IRF-5-E5N3 (PMO 345), and Hs-IRF-5-E5P0 (PMO 346).

FIG. 29B shows the exon 5 skipping levels after treatment with various PMO-EEV compounds. The results indicate that EEV-PMO conjugates can induce exon skipping and downregulation of target gene in THP1 cells.

A number of embodiments have been described herein. Nevertheless, it will be understood that various modifications may be made without departing from the spirit and scope of the invention. Accordingly, other embodiments are within the scope of the following claims.

1-134. (canceled)

135. A compound comprising:
(a) a cyclic cell penetrating peptide of the formula: 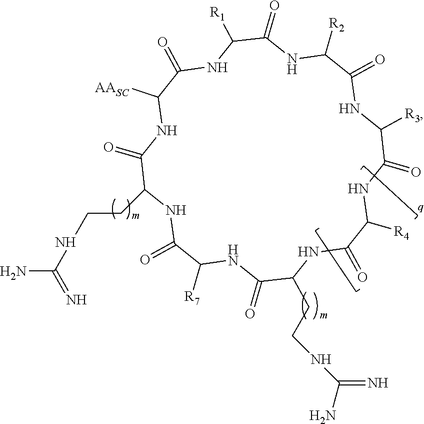
or a protonated form or salt thereof, wherein:
R₁, R₂, and R₃ are each independently H or an aromatic or heteroaromatic side chain of an amino acid;
at least two of R₁, R₂, and R₃ are the side chain of phenylalanine;
R₄ and R₇ are independently H or an amino acid side chain;
each m is independently an integer from 0 to 3;
AAsc is an amino acid side chain; and
q is 1, 2, 3, or 4;
(b) an exocyclic peptide comprising from 2 to 10 amino acid residues, wherein 2, 3, or 4 of the residues are lysine residues;
(c) a linker of formula: 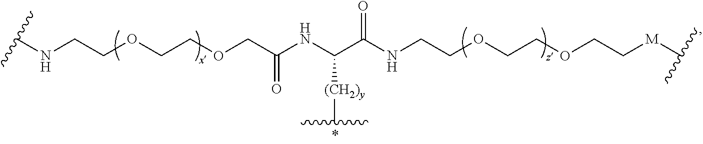
wherein:
x′ is an integer from 1-23;
y is an integer from 1-5;
z′ is an integer from 1-23;
* is the point of attachment to the AA_{SC} of the cyclic peptide;
and M is a bonding group; and
(d) an antisense compound (AC) comprising a nucleotide sequence that is complementary to a target nucleotide sequence of a target transcript of a target gene, wherein the AC specifically hybridizes to the target nucleotide sequence and modulates splicing of the target transcript to downregulate expression or activity of a protein expressed from the target transcript.

136. The compound of claim 135, wherein the exocyclic peptide comprises at least 2 amino acid residues with a hydrophobic side chain.

137. The compound of claim 135, wherein the exocyclic peptide comprises PKKKRKV.

138. The compound of claim 135, wherein z′ is 11.

139. The compound of claim 135, wherein x′ is 1.

140. The compound of claim 135, wherein M comprises

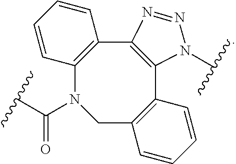 [SMILES: *C(=O)N1Cc2ccccc2-c2c(nnn2*)-c2ccccc21]

141. The compound of claim 135, wherein M comprises

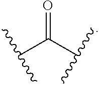 [SMILES: *C(*)=O]

142. The compound of claim 135, wherein the cyclic cell penetrating peptide is selected from: 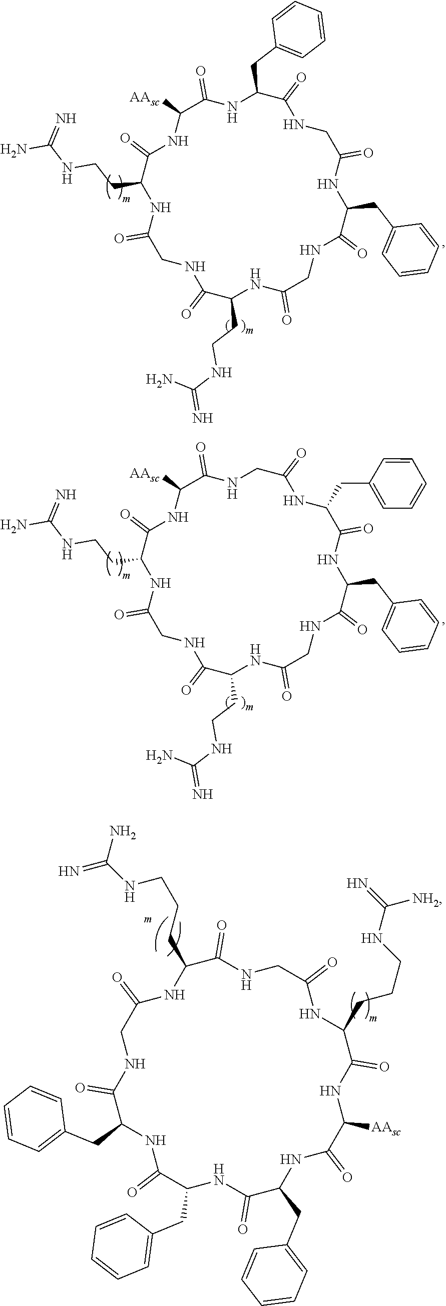 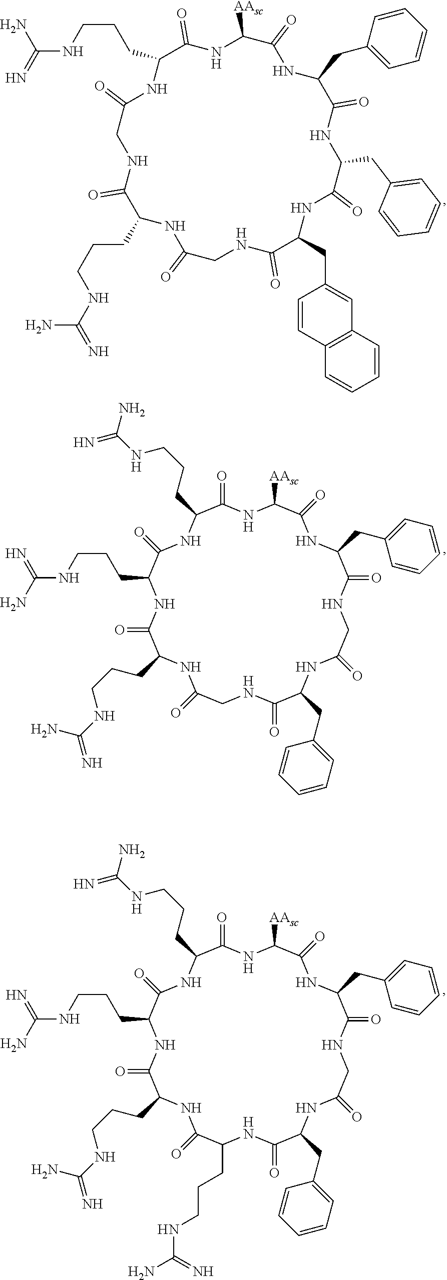 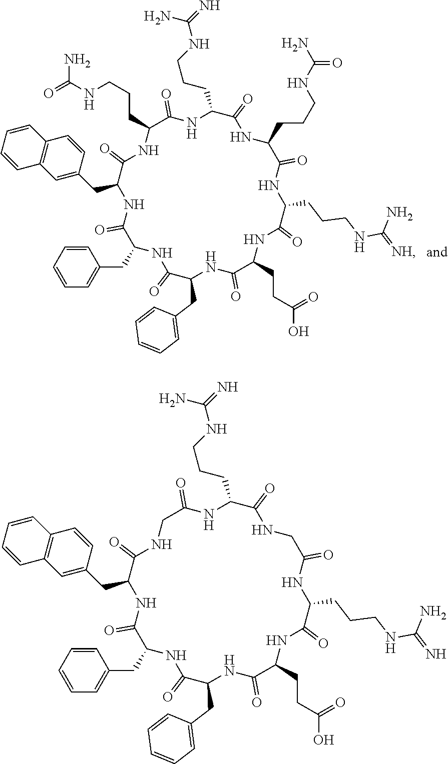 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O ; "and" ; N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]
or a protonated form or salt thereof, wherein if the structure does not include AA_{sc} then at least one atom of an amino acid side chain is replaced by the linker or at least one lone pair forms a bond to the linker.

143. The compound of claim 135, comprising an oligonucleotide conjugated to an endosomal escape vehicle with a sequence selected from:
Ac-PKKKRKV-PEG₂-K(cyclo[Ff-Nal-Cit-r-Cit-r-Q])-PEG₁₂-Lys(N₃)-NH₂;
Ac-PKKKRKV-K(cyclo[ Ff-Nal-GrGrQI)-PEG₁₂-Lys(N₃)-NH₂;
Ac-PKKKRKV-miniPEG₂-Lys(cyclo(FfFGRGRQ)-PEG₂-K(N₃)-NH₂;;
Ac-PKKKRKV-PEG₂-K(cyclo[FGFGRGRQ])-PEG₂-Lys(N₃)-NH₂;
Ac-PKKKRKV-PEG₂-K(cyclo[GfFGrGrQ])-PEG₂-Lys(N₃)-NH₂;
Ac-PKKKRKV-PEG₂-Lys(cyclo[FfFGRGRQ)-miniPEG₂-K(N₃)-NH₂;
Ac-PKKKRKV-PEG₂-K(cyclo(Ff-Nal-GrGrQ)-PEG₁₂-OH;
Ac-PKKKRKV-PEG₂-K(cyclo[FGFGRGRQ])-PEG₁₂-OH;
Ac-PKKKRKV-PEG₂-K(cyclo[GfFGrGrQ])-PEG₁₂-OH;
Ac-PKKKRKV-PEG₂-K(cyclo[FGFGRRRQ])-PEG₁₂-OH; and
Ac-PKKKRKV-PEG₂-K(cyclo[FGFRRRRQ])-PEG₁₂-OH.

144. The compound of claim 135, wherein the antisense compound comprises a phosphorodiamidate morpholino nucleotide.

145. The compound of claim 135, wherein the antisense compound comprises from 10 to 50 nucleotides in length.

146. The compound of claim 135, wherein the antisense compound induces exon skipping and exon skipping induces a frameshift in the target transcript.

147. The compound of claim 146, wherein the frameshift introduces a premature stop codon in the target transcript.

148. The compound of claim 135, wherein the target transcript is a glycogen synthase transcript and wherein the frameshift results in a GYS1 transcript that encodes a truncated GYS1 protein.

149. The compound of claim 135, wherein the target transcript is GYS1 and the antisense compound comprises any one of SEQ ID NO: 151 to SEQ ID NO: 318.

150. The compound of claim 135, wherein the target transcript is a double homeobox 4 (DUX4) transcript, and the antisense compound downregulates the expression of DUX4-fl.

151. The compound of claim 135, wherein the target transcript is a DUX4 transcript, and the antisense compound comprises any one of SEQ ID NO:344 to SEQ ID NO:364.

152. A pharmaceutical composition comprising the compound of claim 135 and a pharmaceutically acceptable carrier.

153. A method of treating a disease or disorder associated with a target gene in a patient, comprising administering to the patient a therapeutically effective amount of the compound of claim 135 to the patient.

154. The method of claim 153, wherein the target gene comprises glycogen synthase (GYS1).

155. The method of claim 154, where the disease comprises type II glycogen storage disease, Pompe disease, Anderson disease, McArdle disease, Lafra disease, or Tariu disease.
